SEQUENCE LISTING (746 sequences)

SEQ ID NO: 1 - DNA, 15 bp, Artificial Sequence
  Synthetic Oligonucleotide
tgacccaagggagac

SEQ ID NO: 2 - DNA, 15 bp, Artificial Sequence
  Synthetic Oligonucleotide
tggccactggccgca

SEQ ID NO: 3 - DNA, 15 bp, Artificial Sequence
  Synthetic Oligonucleotide
cggcgacccctggtg

SEQ ID NO: 4 - DNA, 15 bp, Artificial Sequence
  Synthetic Oligonucleotide
cgccctccagcgctg

SEQ ID NO: 5 - DNA, 15 bp, Artificial Sequence
  Synthetic Oligonucleotide
cgctccgccctccag

SEQ ID NO: 6 - DNA, 17 bp, Artificial Sequence
  Synthetic Oligonucleotide
tgacccaagggagaccc

SEQ ID NO: 7 - DNA, 17 bp, Artificial Sequence
  Synthetic Oligonucleotide
tggccactggccgcacc

SEQ ID NO: 8 - DNA, 17 bp, Artificial Sequence
  Synthetic Oligonucleotide
cggcgacccctggtgcc

SEQ ID NO: 9 - DNA, 17 bp, Artificial Sequence
  Synthetic Oligonucleotide
cgccctccagcgctgcc

SEQ ID NO: 10 - DNA, 17 bp, Artificial Sequence
  Synthetic Oligonucleotide
cgctccgccctccagcc

SEQ ID NO: 11 - DNA, 24 bp, Artificial Sequence
  Synthetic Oligonucleotide
tgacccaagggagacggaaaccac

SEQ ID NO: 12 - DNA, 24 bp, Artificial Sequence
  Synthetic Oligonucleotide
tggccactggccgcaggaaaccac

SEQ ID NO: 13 - DNA, 24 bp, Artificial Sequence
  Synthetic Oligonucleotide
cggcgacccctggtgggaaacctc

SEQ ID NO: 14 - DNA, 24 bp, Artificial Sequence
  Synthetic Oligonucleotide
cgccctccagcgctgggaaacctc

SEQ ID NO: 15 - DNA, 24 bp, Artificial Sequence
  Synthetic Oligonucleotide
cgctccgccctccagccaaaggtc

SEQ ID NO: 16 - DNA, 15 bp, Artificial Sequence
  Synthetic Oligonucleotide
ggtttttaaggcttt

SEQ ID NO: 17 - DNA, 15 bp, Artificial Sequence
  Synthetic Oligonucleotide
ggggtcttggcctga

SEQ ID NO: 18 - DNA, 15 bp, Artificial Sequence
  Synthetic Oligonucleotide
cataatgaagctggg

SEQ ID NO: 19 - DNA, 15 bp, Artificial Sequence
  Synthetic Oligonucleotide
aggaggcaacacatt

SEQ ID NO: 20 - DNA, 15 bp, Artificial Sequence
  Synthetic Oligonucleotide
attattttgcttttt

SEQ ID NO: 21 - DNA, 15 bp, Artificial Sequence
  Synthetic Oligonucleotide
cattttccctcctgg

SEQ ID NO: 22 - DNA, 15 bp, Artificial Sequence
  Synthetic Oligonucleotide
gtaggctacccttta

SEQ ID NO: 23 - DNA, 15 bp, Artificial Sequence
  Synthetic Oligonucleotide
gaggcttgttgcttt

SEQ ID NO: 24 - DNA, 15 bp, Artificial Sequence
  Synthetic Oligonucleotide
catgtatgatgttat

SEQ ID NO: 25 - DNA, 20 bp, Artificial Sequence
  Synthetic Oligonucleotide
tttttggtttttaaggcttt

SEQ ID NO: 26 - DNA, 20 bp, Artificial Sequence
  Synthetic Oligonucleotide
tttttggggtcttggcctga

SEQ ID NO: 27 - DNA, 20 bp, Artificial Sequence
  Synthetic Oligonucleotide
tttttcataatgaagctggg

SEQ ID NO: 28 - DNA, 20 bp, Artificial Sequence
  Synthetic Oligonucleotide
tttttaggaggcaacacatt

SEQ ID NO: 29 - DNA, 20 bp, Artificial Sequence
  Synthetic Oligonucleotide
tttttattattttgcttttt

SEQ ID NO: 30 - DNA, 20 bp, Artificial Sequence
  Synthetic Oligonucleotide
tttttcattttccctcctgg

SEQ ID NO: 31 - DNA, 20 bp, Artificial Sequence
  Synthetic Oligonucleotide
tttttgtaggctacccttta

SEQ ID NO: 32 - DNA, 20 bp, Artificial Sequence
  Synthetic Oligonucleotide
tttttgaggcttgttgcttt

SEQ ID NO: 33 - DNA, 20 bp, Artificial Sequence
  Synthetic Oligonucleotide
tttttcatgtatgatgttat

SEQ ID NO: 34 - DNA, 20 bp, Artificial Sequence
  Synthetic Oligonucleotide
cggcgcccgagagtccacat

SEQ ID NO: 35 - DNA, 20 bp, Artificial Sequence
  Synthetic Oligonucleotide
ccaggaggccggctactgcg

SEQ ID NO: 36 - DNA, 20 bp, Artificial Sequence
  Synthetic Oligonucleotide
ctgggctgggctgggtgacg

SEQ ID NO: 37 - DNA, 20 bp, Artificial Sequence
  Synthetic Oligonucleotide
acccgggtgagggtctgggc

SEQ ID NO: 38 - DNA, 20 bp, Artificial Sequence
  Synthetic Oligonucleotide
ccaactctgccggccgcggg

SEQ ID NO: 39 - DNA, 20 bp, Artificial Sequence
  Synthetic Oligonucleotide
acggcggccgcagagtgggg

SEQ ID NO: 40 - DNA, 20 bp, Artificial Sequence
  Synthetic Oligonucleotide
tcgatgtcggtgcgcaggcc

SEQ ID NO: 41 - DNA, 20 bp, Artificial Sequence
  Synthetic Oligonucleotide
ggcggggcgtgcaggtcgca

SEQ ID NO: 42 - DNA, 20 bp, Artificial Sequence
  Synthetic Oligonucleotide
acgttggttcgaacttgcgc

SEQ ID NO: 43 - DNA, 20 bp, Artificial Sequence
  Synthetic Oligonucleotide
ttccaaatctggttgaggcc

SEQ ID NO: 44 - DNA, 20 bp, Artificial Sequence
  Synthetic Oligonucleotide
agacactctgctttttgaca

SEQ ID NO: 45 - DNA, 20 bp, Artificial Sequence
  Synthetic Oligonucleotide
tttcctcaaattcatcaaat

SEQ ID NO: 46 - DNA, 20 bp, Artificial Sequence
  Synthetic Oligonucleotide
gggtggcccaaagttccaga

SEQ ID NO: 47 - DNA, 20 bp, Artificial Sequence
  Synthetic Oligonucleotide
tggtctcatctagagagcct

SEQ ID NO: 48 - DNA, 20 bp, Artificial Sequence
  Synthetic Oligonucleotide
ctctgctagtctttcatagg

SEQ ID NO: 49 - DNA, 20 bp, Artificial Sequence
  Synthetic Oligonucleotide
gctaaagagtccagcgtttc

SEQ ID NO: 50 - DNA, 21 bp, Artificial Sequence
  Synthetic Oligonucleotide
gcaaggtcttcaaaaaactct

SEQ ID NO: 51 - DNA, 22 bp, Artificial Sequence
  Synthetic Oligonucleotide
ctcaaacgtgtatggcttgtct

SEQ ID NO: 52 - DNA, 21 bp, Artificial Sequence
  Synthetic Oligonucleotide
cccaaaggagacatcatagtc

SEQ ID NO: 53 - DNA, 24 bp, Artificial Sequence
  Synthetic Oligonucleotide
cagtttgacagttaagacaccact

SEQ ID NO: 54 - DNA, 21 bp, Artificial Sequence
  Synthetic Oligonucleotide
ataggttcctagatctccacc

SEQ ID NO: 55 - DNA, 20 bp, Artificial Sequence
  Synthetic Oligonucleotide
ggcgtctgcttgttgatcac

SEQ ID NO: 56 - DNA, 23 bp, Artificial Sequence
  Synthetic Oligonucleotide
aagatagccagatttgcttgttt

SEQ ID NO: 57 - DNA, 24 bp, Artificial Sequence
  Synthetic Oligonucleotide
ggtccactacatacctggatggag

SEQ ID NO: 58 - DNA, 21 bp, Artificial Sequence
  Synthetic Oligonucleotide
cccagtccagtcataacgctt

SEQ ID NO: 59 - DNA, 22 bp, Artificial Sequence
  Synthetic Oligonucleotide
cgtgggagtacacccagttttt

SEQ ID NO: 60 - DNA, 18 bp, Artificial Sequence
  Synthetic Oligonucleotide
catggagggacacgccgt

SEQ ID NO: 61 - DNA, 22 bp, Artificial Sequence
  Synthetic Oligonucleotide
gtgagctctgcggccagcagct

SEQ ID NO: 62 - DNA, 21 bp, Artificial Sequence
  Synthetic Oligonucleotide
agtttggtttttaaggcttta

SEQ ID NO: 63 - DNA, 21 bp, Artificial Sequence
  Synthetic Oligonucleotide
taggccaaggaagacaagtcc

SEQ ID NO: 64 - DNA, 19 bp, Artificial Sequence
  Synthetic Oligonucleotide
tcaagcatcttttccggaa

SEQ ID NO: 65 - DNA, 21 bp, Artificial Sequence
  Synthetic Oligonucleotide
tccttaaaacggggctgggca

SEQ ID NO: 66 - DNA, 21 bp, Artificial Sequence
  Synthetic Oligonucleotide
ttggcctgatagcttttaatg

SEQ ID NO: 67 - DNA, 27 bp, Artificial Sequence
  Synthetic Oligonucleotide
cctcagctgcataatgaagctggggtc

SEQ ID NO: 68 - DNA, 25 bp, Artificial Sequence
  Synthetic Oligonucleotide
aacaacaacaacaacaaaaaacaga

SEQ ID NO: 69 - DNA, 26 bp, Artificial Sequence
  Synthetic Oligonucleotide
cctcaaaagcaggaataaaaaaaata

SEQ ID NO: 70 - DNA, 23 bp, Artificial Sequence
  Synthetic Oligonucleotide
gctgtgacacatagcccaactgt

SEQ ID NO: 71 - DNA, 25 bp, Artificial Sequence
  Synthetic Oligonucleotide
ggaggcaacacattctttctacaga

SEQ ID NO: 72 - DNA, 15 bp, Artificial Sequence
  Synthetic Oligonucleotide
ctattaatattactg

SEQ ID NO: 73 - DNA, 16 bp, Artificial Sequence
  Synthetic Oligonucleotide
cattatgtgtatgtat

SEQ ID NO: 74 - DNA, 15 bp, Artificial Sequence
  Synthetic Oligonucleotide
tttatctatgttatt

SEQ ID NO: 75 - DNA, 15 bp, Artificial Sequence
  Synthetic Oligonucleotide
ctaatttgaagttct

SEQ ID NO: 76 - DNA, 15 bp, Artificial Sequence
  Synthetic Oligonucleotide
ttcgaacttgcgcgg

SEQ ID NO: 77 - DNA, 14 bp, Artificial Sequence
  Synthetic Oligonucleotide
tagagagcctgggt

SEQ ID NO: 78 - DNA, 15 bp, Artificial Sequence
  Synthetic Oligonucleotide
acaccactcccaaag

SEQ ID NO: 79 - DNA, 15 bp, Artificial Sequence
  Synthetic Oligonucleotide
aggtccactacatac

SEQ ID NO: 80 - DNA, 15 bp, Artificial Sequence
  Synthetic Oligonucleotide
cgttaacctggatgg

SEQ ID NO: 81 - DNA, 15 bp, Artificial Sequence
  Synthetic Oligonucleotide
aaagccttaaaaacc

SEQ ID NO: 82 - DNA, 15 bp, Artificial Sequence
  Synthetic Oligonucleotide
tcaggccaagacccc

SEQ ID NO: 83 - DNA, 15 bp, Artificial Sequence
  Synthetic Oligonucleotide
cccagcttcattatg

SEQ ID NO: 84 - DNA, 15 bp, Artificial Sequence
  Synthetic Oligonucleotide
aatgtgttgcctcct

SEQ ID NO: 85 - DNA, 15 bp, Artificial Sequence
  Synthetic Oligonucleotide
aaaaagcaaaataat

SEQ ID NO: 86 - DNA, 15 bp, Artificial Sequence
  Synthetic Oligonucleotide
ccaggagggaaaatg

SEQ ID NO: 87 - DNA, 15 bp, Artificial Sequence
  Synthetic Oligonucleotide
taaagggtagcctac

SEQ ID NO: 88 - DNA, 15 bp, Artificial Sequence
  Synthetic Oligonucleotide
aaagcaacaagcctc

SEQ ID NO: 89 - DNA, 15 bp, Artificial Sequence
  Synthetic Oligonucleotide
ataacatcatacatg

SEQ ID NO: 90 - DNA, 15 bp, Artificial Sequence
  Synthetic Oligonucleotide
gatactatcttcctc

SEQ ID NO: 91 - DNA, 15 bp, Artificial Sequence
  Synthetic Oligonucleotide
atgggggacggggca

SEQ ID NO: 92 - DNA, 15 bp, Artificial Sequence
  Synthetic Oligonucleotide
ggttgagactgggtg

SEQ ID NO: 93 - DNA, 15 bp, Artificial Sequence
  Synthetic Oligonucleotide
agactgaagaggtgc

SEQ ID NO: 94 - DNA, 15 bp, Artificial Sequence
  Synthetic Oligonucleotide
cgggacggctgtgtt

SEQ ID NO: 95 - DNA, 15 bp, Artificial Sequence
  Synthetic Oligonucleotide
tctgtgtgggcagca

SEQ ID NO: 96 - DNA, 15 bp, Artificial Sequence
  Synthetic Oligonucleotide
aaagccttaaaaacc

SEQ ID NO: 97 - DNA, 15 bp, Artificial Sequence
  Synthetic Oligonucleotide
tcaggccaagacccc

SEQ ID NO: 98 - DNA, 15 bp, Artificial Sequence
  Synthetic Oligonucleotide
cccagcttcattatg

SEQ ID NO: 99 - DNA, 15 bp, Artificial Sequence
  Synthetic Oligonucleotide
aatgtgttgcctcct

SEQ ID NO: 100 - DNA, 15 bp, Artificial Sequence
  Synthetic Oligonucleotide
aaaaagcaaaataat

SEQ ID NO: 101 - DNA, 15 bp, Artificial Sequence
  Synthetic Oligonucleotide
ccaggagggaaaatg

SEQ ID NO: 102 - DNA, 15 bp, Artificial Sequence
  Synthetic Oligonucleotide
taaagggtagcctac

SEQ ID NO: 103 - DNA, 15 bp, Artificial Sequence
  Synthetic Oligonucleotide
aaagcaacaagcctc

SEQ ID NO: 104 - DNA, 15 bp, Artificial Sequence
  Synthetic Oligonucleotide
ataacatcatacatg

SEQ ID NO: 105 - DNA, 15 bp, Artificial Sequence
  Synthetic Oligonucleotide
gatactatcttcctc

SEQ ID NO: 106 - DNA, 15 bp, Artificial Sequence
  Synthetic Oligonucleotide
atgggggacggggca

SEQ ID NO: 107 - DNA, 15 bp, Artificial Sequence
  Synthetic Oligonucleotide
ggttgagactgggtg

SEQ ID NO: 108 - DNA, 15 bp, Artificial Sequence
  Synthetic Oligonucleotide
agactgaagaggtgc

SEQ ID NO: 109 - DNA, 15 bp, Artificial Sequence
  Synthetic Oligonucleotide
cgggacggctgtgtt

SEQ ID NO: 110 - DNA, 15 bp, Artificial Sequence
  Synthetic Oligonucleotide
tctgtgtgggcagca

SEQ ID NO: 111 - DNA, 15 bp, Artificial Sequence
  Synthetic Oligonucleotide
gaagaagaagaagaa

SEQ ID NO: 112 - DNA, 15 bp, Artificial Sequence
  Synthetic Oligonucleotide
ttcttcttcttcttc

SEQ ID NO: 113 - DNA, 20 bp, Artificial Sequence
  Synthetic Oligonucleotide
cggcgcccgagagtccacat

SEQ ID NO: 114 - DNA, 20 bp, Artificial Sequence
  Synthetic Oligonucleotide
acggcggccgcagagtgggg

SEQ ID NO: 115 - DNA, 26 bp, Artificial Sequence
  Synthetic Oligonucleotide
cctcaaaagcaggaataaaaaaaata

SEQ ID NO: 116 - DNA, 15 bp, Artificial Sequence
  Synthetic Oligonucleotide
atgggggacggggca

SEQ ID NO: 117 - DNA, 15 bp, Artificial Sequence
  Synthetic Oligonucleotide
ggttgagactgggtg

SEQ ID NO: 118 - DNA, 15 bp, Artificial Sequence
  Synthetic Oligonucleotide
atgggggacggggca

SEQ ID NO: 119 - DNA, 35 bp, Artificial Sequence
  Synthetic Oligonucleotide
tgacccaagggagactttttggtttttaaggcttt

SEQ ID NO: 120 - DNA, 35 bp, Artificial Sequence
  Synthetic Oligonucleotide
tggccactggccgcatttttggtttttaaggcttt

SEQ ID NO: 121 - DNA, 35 bp, Artificial Sequence
  Synthetic Oligonucleotide
cggcgacccctggtgtttttggtttttaaggcttt

SEQ ID NO: 122 - DNA, 35 bp, Artificial Sequence
  Synthetic Oligonucleotide
cgccctccagcgctgtttttggtttttaaggcttt

SEQ ID NO: 123 - DNA, 35 bp, Artificial Sequence
  Synthetic Oligonucleotide
cgctccgccctccagtttttggtttttaaggcttt

SEQ ID NO: 124 - DNA, 35 bp, Artificial Sequence
  Synthetic Oligonucleotide
tgacccaagggagactttttggggtcttggcctga

SEQ ID NO: 125 - DNA, 35 bp, Artificial Sequence
  Synthetic Oligonucleotide
tggccactggccgcatttttggggtcttggcctga

SEQ ID NO: 126 - DNA, 35 bp, Artificial Sequence
  Synthetic Oligonucleotide
cggcgacccctggtgtttttggggtcttggcctga

SEQ ID NO: 127 - DNA, 35 bp, Artificial Sequence
  Synthetic Oligonucleotide
cgccctccagcgctgtttttggggtcttggcctga

SEQ ID NO: 128 - DNA, 35 bp, Artificial Sequence
  Synthetic Oligonucleotide
cgctccgccctccagtttttggggtcttggcctga

SEQ ID NO: 129 - DNA, 35 bp, Artificial Sequence
  Synthetic Oligonucleotide
tgacccaagggagactttttcataatgaagctggg

SEQ ID NO: 130 - DNA, 35 bp, Artificial Sequence
  Synthetic Oligonucleotide
tggccactggccgcatttttcataatgaagctggg

SEQ ID NO: 131 - DNA, 35 bp, Artificial Sequence
  Synthetic Oligonucleotide
cggcgacccctggtgtttttcataatgaagctggg

SEQ ID NO: 132 - DNA, 35 bp, Artificial Sequence
  Synthetic Oligonucleotide
cgccctccagcgctgtttttcataatgaagctggg

SEQ ID NO: 133 - DNA, 35 bp, Artificial Sequence
  Synthetic Oligonucleotide
cgctccgccctccagtttttcataatgaagctggg

SEQ ID NO: 134 - DNA, 35 bp, Artificial Sequence
  Synthetic Oligonucleotide
tgacccaagggagactttttaggaggcaacacatt

SEQ ID NO: 135 - DNA, 35 bp, Artificial Sequence
  Synthetic Oligonucleotide
tggccactggccgcatttttaggaggcaacacatt

SEQ ID NO: 136 - DNA, 35 bp, Artificial Sequence
  Synthetic Oligonucleotide
cggcgacccctggtgtttttaggaggcaacacatt

SEQ ID NO: 137 - DNA, 35 bp, Artificial Sequence
  Synthetic Oligonucleotide
cgccctccagcgctgtttttaggaggcaacacatt

SEQ ID NO: 138 - DNA, 35 bp, Artificial Sequence
  Synthetic Oligonucleotide
cgctccgccctccagtttttaggaggcaacacatt

SEQ ID NO: 139 - DNA, 35 bp, Artificial Sequence
  Synthetic Oligonucleotide
tgacccaagggagactttttattattttgcttttt

SEQ ID NO: 140 - DNA, 35 bp, Artificial Sequence
  Synthetic Oligonucleotide
tggccactggccgcatttttattattttgcttttt

SEQ ID NO: 141 - DNA, 35 bp, Artificial Sequence
  Synthetic Oligonucleotide
cggcgacccctggtgtttttattattttgcttttt

SEQ ID NO: 142 - DNA, 35 bp, Artificial Sequence
  Synthetic Oligonucleotide
cgccctccagcgctgtttttattattttgcttttt

SEQ ID NO: 143 - DNA, 35 bp, Artificial Sequence
  Synthetic Oligonucleotide
cgctccgccctccagtttttattattttgcttttt

SEQ ID NO: 144 - DNA, 35 bp, Artificial Sequence
  Synthetic Oligonucleotide
tgacccaagggagactttttcattttccctcctgg

SEQ ID NO: 145 - DNA, 35 bp, Artificial Sequence
  Synthetic Oligonucleotide
tggccactggccgcatttttcattttccctcctgg

SEQ ID NO: 146 - DNA, 35 bp, Artificial Sequence
  Synthetic Oligonucleotide
cggcgacccctggtgtttttcattttccctcctgg

SEQ ID NO: 147 - DNA, 35 bp, Artificial Sequence
  Synthetic Oligonucleotide
cgccctccagcgctgtttttcattttccctcctgg

SEQ ID NO: 148 - DNA, 35 bp, Artificial Sequence
  Synthetic Oligonucleotide
cgctccgccctccagtttttcattttccctcctgg

SEQ ID NO: 149 - DNA, 35 bp, Artificial Sequence
  Synthetic Oligonucleotide
tgacccaagggagactttttgtaggctacccttta

SEQ ID NO: 150 - DNA, 35 bp, Artificial Sequence
  Synthetic Oligonucleotide
tggccactggccgcatttttgtaggctacccttta

SEQ ID NO: 151 - DNA, 35 bp, Artificial Sequence
  Synthetic Oligonucleotide
cggcgacccctggtgtttttgtaggctacccttta

SEQ ID NO: 152 - DNA, 35 bp, Artificial Sequence
  Synthetic Oligonucleotide
cgccctccagcgctgtttttgtaggctacccttta

SEQ ID NO: 153 - DNA, 35 bp, Artificial Sequence
  Synthetic Oligonucleotide
cgctccgccctccagtttttgtaggctacccttta

SEQ ID NO: 154 - DNA, 35 bp, Artificial Sequence
  Synthetic Oligonucleotide
tgacccaagggagactttttgaggcttgttgcttt

SEQ ID NO: 155 - DNA, 35 bp, Artificial Sequence
  Synthetic Oligonucleotide
tggccactggccgcatttttgaggcttgttgcttt

SEQ ID NO: 156 - DNA, 35 bp, Artificial Sequence
  Synthetic Oligonucleotide
cggcgacccctggtgtttttgaggcttgttgcttt

SEQ ID NO: 157 - DNA, 35 bp, Artificial Sequence
  Synthetic Oligonucleotide
cgccctccagcgctgtttttgaggcttgttgcttt

SEQ ID NO: 158 - DNA, 35 bp, Artificial Sequence
  Synthetic Oligonucleotide
cgctccgccctccagtttttgaggcttgttgcttt

SEQ ID NO: 159 - DNA, 35 bp, Artificial Sequence
  Synthetic Oligonucleotide
tgacccaagggagactttttcatgtatgatgttat

SEQ ID NO: 160 - DNA, 35 bp, Artificial Sequence
  Synthetic Oligonucleotide
tggccactggccgcatttttcatgtatgatgttat

SEQ ID NO: 161 - DNA, 35 bp, Artificial Sequence
  Synthetic Oligonucleotide
cggcgacccctggtgtttttcatgtatgatgttat

SEQ ID NO: 162 - DNA, 35 bp, Artificial Sequence
  Synthetic Oligonucleotide
cgccctccagcgctgtttttcatgtatgatgttat

SEQ ID NO: 163 - DNA, 35 bp, Artificial Sequence
  Synthetic Oligonucleotide
cgctccgccctccagtttttcatgtatgatgttat

SEQ ID NO: 164 - DNA, 25 bp, Artificial Sequence
  Synthetic Oligonucleotide
cgccctccagtttttggtttttaag

SEQ ID NO: 165 - DNA, 25 bp, Artificial Sequence
  Synthetic Oligonucleotide
cgccctccagtttttggggtcttgg

SEQ ID NO: 166 - DNA, 25 bp, Artificial Sequence
  Synthetic Oligonucleotide
cgccctccagtttttcataatgaag

SEQ ID NO: 167 - DNA, 25 bp, Artificial Sequence
  Synthetic Oligonucleotide
cgccctccagtttttaggaggcaac

SEQ ID NO: 168 - DNA, 25 bp, Artificial Sequence
  Synthetic Oligonucleotide
cgccctccagtttttattattttgc

SEQ ID NO: 169 - DNA, 25 bp, Artificial Sequence
  Synthetic Oligonucleotide
cgccctccagtttttcattttccct

SEQ ID NO: 170 - DNA, 25 bp, Artificial Sequence
  Synthetic Oligonucleotide
cgccctccagtttttgtaggctacc

SEQ ID NO: 171 - DNA, 25 bp, Artificial Sequence
  Synthetic Oligonucleotide
cgccctccagtttttgaggcttgtt

SEQ ID NO: 172 - DNA, 25 bp, Artificial Sequence
  Synthetic Oligonucleotide
cgccctccagtttttcatgtatgat

SEQ ID NO: 173 - DNA, 27 bp, Artificial Sequence
  Synthetic Oligonucleotide
tgacccaagggagactttttttttttt

SEQ ID NO: 174 - DNA, 27 bp, Artificial Sequence
  Synthetic Oligonucleotide
tggccactggccgcatttttttttttt

SEQ ID NO: 175 - DNA, 27 bp, Artificial Sequence
  Synthetic Oligonucleotide
cggcgacccctggtgtttttttttttt

SEQ ID NO: 176 - DNA, 27 bp, Artificial Sequence
  Synthetic Oligonucleotide
cgccctccagcgctgtttttttttttt

SEQ ID NO: 177 - DNA, 27 bp, Artificial Sequence
  Synthetic Oligonucleotide
cgctccgccctccagtttttttttttt

SEQ ID NO: 178 - DNA, 15 bp, Artificial Sequence
  Synthetic Oligonucleotide
aaaataaacaacaac

SEQ ID NO: 179 - DNA, 16 bp, Artificial Sequence
  Synthetic Oligonucleotide
aggaataaaaaaaata

SEQ ID NO: 180 - DNA, 15 bp, Artificial Sequence
  Synthetic Oligonucleotide
tcaaaagcaggaata

SEQ ID NO: 181 - DNA, 15 bp, Artificial Sequence
  Synthetic Oligonucleotide
actgtcctcaaaagc

SEQ ID NO: 182 - DNA, 15 bp, Artificial Sequence
  Synthetic Oligonucleotide
agcccaactgtcctc

SEQ ID NO: 183 - DNA, 15 bp, Artificial Sequence
  Synthetic Oligonucleotide
tgacacatagcccaa

SEQ ID NO: 184 - DNA, 15 bp, Artificial Sequence
  Synthetic Oligonucleotide
gagctgtgacacata

SEQ ID NO: 185 - DNA, 15 bp, Artificial Sequence
  Synthetic Oligonucleotide
tctgggcctgggctg

SEQ ID NO: 186 - DNA, 15 bp, Artificial Sequence
  Synthetic Oligonucleotide
ggtgagggtctgggc

SEQ ID NO: 187 - DNA, 15 bp, Artificial Sequence
  Synthetic Oligonucleotide
gggacccgggtgagg

SEQ ID NO: 188 - DNA, 15 bp, Artificial Sequence
  Synthetic Oligonucleotide
ccggccgcgggaccc

SEQ ID NO: 189 - DNA, 15 bp, Artificial Sequence
  Synthetic Oligonucleotide
caactctgccggccg

SEQ ID NO: 190 - DNA, 15 bp, Artificial Sequence
  Synthetic Oligonucleotide
agtggggccaactct

SEQ ID NO: 191 - DNA, 15 bp, Artificial Sequence
  Synthetic Oligonucleotide
ggccgcagagtgggg

SEQ ID NO: 192 - DNA, 15 bp, Artificial Sequence
  Synthetic Oligonucleotide
gccacggcggccgca

SEQ ID NO: 193 - DNA, 15 bp, Artificial Sequence
  Synthetic Oligonucleotide
gtgcgcaggccacgg

SEQ ID NO: 194 - DNA, 15 bp, Artificial Sequence
  Synthetic Oligonucleotide
gggggacggggcagg

SEQ ID NO: 195 - DNA, 15 bp, Artificial Sequence
  Synthetic Oligonucleotide
gggacggggcaggtt

SEQ ID NO: 196 - DNA, 15 bp, Artificial Sequence
  Synthetic Oligonucleotide
gacggggcaggttga

SEQ ID NO: 197 - DNA, 15 bp, Artificial Sequence
  Synthetic Oligonucleotide
cggggcaggttgaga

SEQ ID NO: 198 - DNA, 15 bp, Artificial Sequence
  Synthetic Oligonucleotide
gggcaggttgagact

SEQ ID NO: 199 - DNA, 15 bp, Artificial Sequence
  Synthetic Oligonucleotide
gcaggttgagactgg

SEQ ID NO: 200 - DNA, 15 bp, Artificial Sequence
  Synthetic Oligonucleotide
aggttgagactgggt

SEQ ID NO: 201 - DNA, 15 bp, Artificial Sequence
  Synthetic Oligonucleotide
ggaaaaattccagga

SEQ ID NO: 202 - DNA, 15 bp, Artificial Sequence
  Synthetic Oligonucleotide
aattccaggagggaa

SEQ ID NO: 203 - DNA, 15 bp, Artificial Sequence
  Synthetic Oligonucleotide
gagggaaaatgaatt

SEQ ID NO: 204 - DNA, 17 bp, Artificial Sequence
  Synthetic Oligonucleotide
gaaaatgaattgtcttc

SEQ ID NO: 205 - DNA, 15 bp, Artificial Sequence
  Synthetic Oligonucleotide
gggggacggggcagg

SEQ ID NO: 206 - DNA, 15 bp, Artificial Sequence
  Synthetic Oligonucleotide
gggacggggcaggtt

SEQ ID NO: 207 - DNA, 15 bp, Artificial Sequence
  Synthetic Oligonucleotide
gacggggcaggttga

SEQ ID NO: 208 - DNA, 15 bp, Artificial Sequence
  Synthetic Oligonucleotide
cggggcaggttgaga

SEQ ID NO: 209 - DNA, 15 bp, Artificial Sequence
  Synthetic Oligonucleotide
gggcaggttgagact

SEQ ID NO: 210 - DNA, 15 bp, Artificial Sequence
  Synthetic Oligonucleotide
gcaggttgagactgg

SEQ ID NO: 211 - DNA, 15 bp, Artificial Sequence
  Synthetic Oligonucleotide
aggttgagactgggt

SEQ ID NO: 212 - DNA, 15 bp, Artificial Sequence
  Synthetic Oligonucleotide
ggaaaaattccagga

SEQ ID NO: 213 - DNA, 15 bp, Artificial Sequence
  Synthetic Oligonucleotide
aattccaggagggaa

SEQ ID NO: 214 - DNA, 15 bp, Artificial Sequence
  Synthetic Oligonucleotide
gagggaaaatgaatt

SEQ ID NO: 215 - DNA, 17 bp, Artificial Sequence
  Synthetic Oligonucleotide
gaaaatgaattgtcttc

SEQ ID NO: 216 - DNA, 15 bp, Artificial Sequence
  Synthetic Oligonucleotide
ggtggtttcagttct

SEQ ID NO: 217 - DNA, 20 bp, Artificial Sequence
  Synthetic Oligonucleotide
tttttggtggtttcagttct

SEQ ID NO: 218 - DNA, 15 bp, Artificial Sequence
  Synthetic Oligonucleotide
agcgtgctatctggg

SEQ ID NO: 219 - DNA, 15 bp, Artificial Sequence
  Synthetic Oligonucleotide
tggcccagggactct

SEQ ID NO: 220 - DNA, 14 bp, Artificial Sequence
  Synthetic Oligonucleotide
tctgcggctctggc

SEQ ID NO: 221 - DNA, 15 bp, Artificial Sequence
  Synthetic Oligonucleotide
cggtccggctctggg

SEQ ID NO: 222 - DNA, 15 bp, Artificial Sequence
  Synthetic Oligonucleotide
tcatcccgggaagct

SEQ ID NO: 223 - DNA, 15 bp, Artificial Sequence
  Synthetic Oligonucleotide
ccccaagtccccgct

SEQ ID NO: 224 - DNA, 15 bp, Artificial Sequence
  Synthetic Oligonucleotide
ccaaccatgcaagca

SEQ ID NO: 225 - DNA, 17 bp, Artificial Sequence
  Synthetic Oligonucleotide
tggcccagggactcttc

SEQ ID NO: 226 - DNA, 18 bp, Artificial Sequence
  Synthetic Oligonucleotide
cggtccggctctgggttc

SEQ ID NO: 227 - DNA, 17 bp, Artificial Sequence
  Synthetic Oligonucleotide
ccaaccatgcaagcacc

SEQ ID NO: 228 - DNA, 26 bp, Artificial Sequence
  Synthetic Oligonucleotide
tggcccagggactctcacaaagtgac

SEQ ID NO: 229 - DNA, 26 bp, Artificial Sequence
  Synthetic Oligonucleotide
cggtccggctctgggaagaaactttc

SEQ ID NO: 230 - DNA, 26 bp, Artificial Sequence
  Synthetic Oligonucleotide
ccaaccatgcaagcactcaaagagtc

SEQ ID NO: 231 - DNA, 34 bp, Artificial Sequence
  Synthetic Oligonucleotide
tggcccagggactctttttggtggtttcagttct

SEQ ID NO: 232 - DNA, 35 bp, Artificial Sequence
  Synthetic Oligonucleotide
cggtccggctctgggtttttggtggtttcagttct

SEQ ID NO: 233 - DNA, 35 bp, Artificial Sequence
  Synthetic Oligonucleotide
ccaaccatgcaagcatttttggtggtttcagttct

SEQ ID NO: 234 - DNA, 24 bp, Artificial Sequence
  Synthetic Oligonucleotide
cagggactctttttggtggtttca

SEQ ID NO: 235 - DNA, 25 bp, Artificial Sequence
  Synthetic Oligonucleotide
cggctctgggtttttggtggtttca

SEQ ID NO: 236 - DNA, 25 bp, Artificial Sequence
  Synthetic Oligonucleotide
catgcaagcatttttggtggtttca

SEQ ID NO: 237 - DNA, 29 bp, Artificial Sequence
  Synthetic Oligonucleotide
tggcccagggactcggtggtttcagttct

SEQ ID NO: 238 - DNA, 30 bp, Artificial Sequence
  Synthetic Oligonucleotide
cggtccggctctggtggtggtttcagttct

SEQ ID NO: 239 - DNA, 30 bp, Artificial Sequence
  Synthetic Oligonucleotide
ccaaccatgcaagcaggtggtttcagttct

SEQ ID NO: 240 - DNA, 20 bp, Artificial Sequence
  Synthetic Oligonucleotide
tttttagataaaatattata

SEQ ID NO: 241 - DNA, 19 bp, Artificial Sequence
  Synthetic Oligonucleotide
tttttattcagataaaata

SEQ ID NO: 242 - DNA, 20 bp, Artificial Sequence
  Synthetic Oligonucleotide
tttttggtttatttaaaact

SEQ ID NO: 243 - DNA, 20 bp, Artificial Sequence
  Synthetic Oligonucleotide
tttttaaatttatattacat

SEQ ID NO: 244 - DNA, 20 bp, Artificial Sequence
  Synthetic Oligonucleotide
tttttcttaaatttatatta

SEQ ID NO: 245 - DNA, 20 bp, Artificial Sequence
  Synthetic Oligonucleotide
tttttcacaaaatgttcatt

SEQ ID NO: 246 - DNA, 15 bp, Artificial Sequence
  Synthetic Oligonucleotide
cctccgccttctccc

SEQ ID NO: 247 - DNA, 15 bp, Artificial Sequence
  Synthetic Oligonucleotide
tctggtcgggaaact

SEQ ID NO: 248 - DNA, 15 bp, Artificial Sequence
  Synthetic Oligonucleotide
gctacagccttttcc

SEQ ID NO: 249 - DNA, 16 bp, Artificial Sequence
  Synthetic Oligonucleotide
cctccgccttctcccc

SEQ ID NO: 250 - DNA, 17 bp, Artificial Sequence
  Synthetic Oligonucleotide
tctggtcgggaaactcc

SEQ ID NO: 251 - DNA, 16 bp, Artificial Sequence
  Synthetic Oligonucleotide
gctacagccttttccc

SEQ ID NO: 252 - DNA, 24 bp, Artificial Sequence
  Synthetic Oligonucleotide
cctccgccttctccctctttgatc

SEQ ID NO: 253 - DNA, 26 bp, Artificial Sequence
  Synthetic Oligonucleotide
tctggtcgggaaactcaattattgtc

SEQ ID NO: 254 - DNA, 24 bp, Artificial Sequence
  Synthetic Oligonucleotide
gctacagccttttccactttgttc

SEQ ID NO: 255 - DNA, 35 bp, Artificial Sequence
  Synthetic Oligonucleotide
cctccgccttctccctttttagataaaatattata

SEQ ID NO: 256 - DNA, 34 bp, Artificial Sequence
  Synthetic Oligonucleotide
tctggtcgggaaactttttagataaaatattata

SEQ ID NO: 257 - DNA, 35 bp, Artificial Sequence
  Synthetic Oligonucleotide
gctacagccttttcctttttagataaaatattata

SEQ ID NO: 258 - DNA, 35 bp, Artificial Sequence
  Synthetic Oligonucleotide
cctccgccttctccctttttggtttatttaaaact

SEQ ID NO: 259 - DNA, 34 bp, Artificial Sequence
  Synthetic Oligonucleotide
tctggtcgggaaactttttggtttatttaaaact

SEQ ID NO: 260 - DNA, 35 bp, Artificial Sequence
  Synthetic Oligonucleotide
gctacagccttttcctttttggtttatttaaaact

SEQ ID NO: 261 - DNA, 35 bp, Artificial Sequence
  Synthetic Oligonucleotide
cctccgccttctccctttttaaatttatattacat

SEQ ID NO: 262 - DNA, 34 bp, Artificial Sequence
  Synthetic Oligonucleotide
tctggtcgggaaactttttaaatttatattacat

SEQ ID NO: 263 - DNA, 35 bp, Artificial Sequence
  Synthetic Oligonucleotide
gctacagccttttcctttttaaatttatattacat

SEQ ID NO: 264 - DNA, 25 bp, Artificial Sequence
  Synthetic Oligonucleotide
gccttctccctttttagataaaata

SEQ ID NO: 265 - DNA, 24 bp, Artificial Sequence
  Synthetic Oligonucleotide
tcgggaaactttttagataaaata

SEQ ID NO: 266 - DNA, 25 bp, Artificial Sequence
  Synthetic Oligonucleotide
agccttttcctttttagataaaata

SEQ ID NO: 267 - DNA, 25 bp, Artificial Sequence
  Synthetic Oligonucleotide
gccttctccctttttggtttattta

SEQ ID NO: 268 - DNA, 24 bp, Artificial Sequence
  Synthetic Oligonucleotide
tcgggaaactttttggtttattta

SEQ ID NO: 269 - DNA, 25 bp, Artificial Sequence
  Synthetic Oligonucleotide
agccttttcctttttggtttattta

SEQ ID NO: 270 - DNA, 25 bp, Artificial Sequence
  Synthetic Oligonucleotide
gccttctccctttttaaatttatat

SEQ ID NO: 271 - DNA, 24 bp, Artificial Sequence
  Synthetic Oligonucleotide
tcgggaaactttttaaatttatat

SEQ ID NO: 272 - DNA, 25 bp, Artificial Sequence
  Synthetic Oligonucleotide
agccttttcctttttaaatttatat

SEQ ID NO: 273 - DNA, 15 bp, Artificial Sequence
  Synthetic Oligonucleotide
aggtgtgcactttta

SEQ ID NO: 274 - DNA, 15 bp, Artificial Sequence
  Synthetic Oligonucleotide
tcatttttaaggtgt

SEQ ID NO: 275 - DNA, 20 bp, Artificial Sequence
  Synthetic Oligonucleotide
tttttaggtgtgcactttta

SEQ ID NO: 276 - DNA, 19 bp, Artificial Sequence
  Synthetic Oligonucleotide
tttttcatttttaaggtgt

SEQ ID NO: 277 - DNA, 15 bp, Artificial Sequence
  Synthetic Oligonucleotide
cgcggtctcggcggt

SEQ ID NO: 278 - DNA, 15 bp, Artificial Sequence
  Synthetic Oligonucleotide
atcatccatggtgag

SEQ ID NO: 279 - DNA, 34 bp, Artificial Sequence
  Synthetic Oligonucleotide
cgcggtctcggcggtttttaggtgtgcactttta

SEQ ID NO: 280 - DNA, 35 bp, Artificial Sequence
  Synthetic Oligonucleotide
atcatccatggtgagtttttaggtgtgcactttta

SEQ ID NO: 281 - DNA, 33 bp, Artificial Sequence
  Synthetic Oligonucleotide
cgcggtctcggcggtttttcatttttaaggtgt

SEQ ID NO: 282 - DNA, 34 bp, Artificial Sequence
  Synthetic Oligonucleotide
atcatccatggtgagtttttcatttttaaggtgt

SEQ ID NO: 283 - DNA, 24 bp, Artificial Sequence
  Synthetic Oligonucleotide
tctcggcggtttttaggtgtgcac

SEQ ID NO: 284 - DNA, 25 bp, Artificial Sequence
  Synthetic Oligonucleotide
ccatggtgagtttttaggtgtgcac

SEQ ID NO: 285 - DNA, 23 bp, Artificial Sequence
  Synthetic Oligonucleotide
tctcggcggtttttcatttttaa

SEQ ID NO: 286 - DNA, 24 bp, Artificial Sequence
  Synthetic Oligonucleotide
ccatggtgagtttttcatttttaa

SEQ ID NO: 287 - DNA, 30 bp, Artificial Sequence
  Synthetic Oligonucleotide
cgcggtctcggcggtaggtgtgcactttta

SEQ ID NO: 288 - DNA, 30 bp, Artificial Sequence
  Synthetic Oligonucleotide
atcatccatggtgagaggtgtgcactttta

SEQ ID NO: 289 - DNA, 30 bp, Artificial Sequence
  Synthetic Oligonucleotide
cgcggtctcggcggttcatttttaaggtgt

SEQ ID NO: 290 - DNA, 30 bp, Artificial Sequence
  Synthetic Oligonucleotide
atcatccatggtgagtcatttttaaggtgt

SEQ ID NO: 291 - DNA, 15 bp, Artificial Sequence
  Synthetic Oligonucleotide
tggagccgagcgctg

SEQ ID NO: 292 - DNA, 15 bp, Artificial Sequence
  Synthetic Oligonucleotide
gggcctgcccctttg

SEQ ID NO: 293 - DNA, 15 bp, Artificial Sequence
  Synthetic Oligonucleotide
ccccaagtcacctga

SEQ ID NO: 294 - DNA, 15 bp, Artificial Sequence
  Synthetic Oligonucleotide
gacatcaatacctaa

SEQ ID NO: 295 - DNA, 15 bp, Artificial Sequence
  Synthetic Oligonucleotide
aaactttaccaagtc

SEQ ID NO: 296 - DNA, 17 bp, Artificial Sequence
  Synthetic Oligonucleotide
tggagccgagcgctgcc

SEQ ID NO: 297 - DNA, 17 bp, Artificial Sequence
  Synthetic Oligonucleotide
gggcctgcccctttgcc

SEQ ID NO: 298 - DNA, 17 bp, Artificial Sequence
  Synthetic Oligonucleotide
ccccaagtcacctgacc

SEQ ID NO: 299 - DNA, 17 bp, Artificial Sequence
  Synthetic Oligonucleotide
gacatcaatacctaacc

SEQ ID NO: 300 - DNA, 17 bp, Artificial Sequence
  Synthetic Oligonucleotide
aaactttaccaagtccc

SEQ ID NO: 301 - DNA, 24 bp, Artificial Sequence
  Synthetic Oligonucleotide
tggagccgagcgctgggaaaccac

SEQ ID NO: 302 - DNA, 24 bp, Artificial Sequence
  Synthetic Oligonucleotide
gggcctgcccctttgggaaaccac

SEQ ID NO: 303 - DNA, 24 bp, Artificial Sequence
  Synthetic Oligonucleotide
ccccaagtcacctgaggaaaccac

SEQ ID NO: 304 - DNA, 24 bp, Artificial Sequence
  Synthetic Oligonucleotide
gacatcaatacctaaggaaaccac

SEQ ID NO: 305 - DNA, 24 bp, Artificial Sequence
  Synthetic Oligonucleotide
aaactttaccaagtcggaaaccac

SEQ ID NO: 306 - DNA, 15 bp, Artificial Sequence
  Synthetic Oligonucleotide
actgcaatatatttc

SEQ ID NO: 307 - DNA, 15 bp, Artificial Sequence
  Synthetic Oligonucleotide
gtgttaaaattactt

SEQ ID NO: 308 - DNA, 20 bp, Artificial Sequence
  Synthetic Oligonucleotide
tttttactgcaatatatttc

SEQ ID NO: 309 - DNA, 20 bp, Artificial Sequence
  Synthetic Oligonucleotide
tttttgtgttaaaattactt

SEQ ID NO: 310 - DNA, 25 bp, Artificial Sequence
  Synthetic Oligonucleotide
ccgagcgctgtttttactgcaatat

SEQ ID NO: 311 - DNA, 25 bp, Artificial Sequence
  Synthetic Oligonucleotide
tgcccctttgtttttactgcaatat

SEQ ID NO: 312 - DNA, 25 bp, Artificial Sequence
  Synthetic Oligonucleotide
agtcacctgatttttactgcaatat

SEQ ID NO: 313 - DNA, 25 bp, Artificial Sequence
  Synthetic Oligonucleotide
caatacctaatttttactgcaatat

SEQ ID NO: 314 - DNA, 25 bp, Artificial Sequence
  Synthetic Oligonucleotide
ttaccaagtctttttactgcaatat

SEQ ID NO: 315 - DNA, 25 bp, Artificial Sequence
  Synthetic Oligonucleotide
ccgagcgctgtttttgtgttaaaat

SEQ ID NO: 316 - DNA, 25 bp, Artificial Sequence
  Synthetic Oligonucleotide
tgcccctttgtttttgtgttaaaat

SEQ ID NO: 317 - DNA, 25 bp, Artificial Sequence
  Synthetic Oligonucleotide
agtcacctgatttttgtgttaaaat

SEQ ID NO: 318 - DNA, 25 bp, Artificial Sequence
  Synthetic Oligonucleotide
caatacctaatttttgtgttaaaat

SEQ ID NO: 319 - DNA, 25 bp, Artificial Sequence
  Synthetic Oligonucleotide
ttaccaagtctttttgtgttaaaat

SEQ ID NO: 320 - DNA, 15 bp, Artificial Sequence
  Synthetic Oligonucleotide
tgtctgtagctccag

SEQ ID NO: 321 - DNA, 15 bp, Artificial Sequence
  Synthetic Oligonucleotide
tagctccagtgaggc

SEQ ID NO: 322 - DNA, 15 bp, Artificial Sequence
  Synthetic Oligonucleotide
tttcttctcccacca

SEQ ID NO: 323 - DNA, 17 bp, Artificial Sequence
  Synthetic Oligonucleotide
tgtctgtagctccagcc

SEQ ID NO: 324 - DNA, 17 bp, Artificial Sequence
  Synthetic Oligonucleotide
tagctccagtgaggccc

SEQ ID NO: 325 - DNA, 17 bp, Artificial Sequence
  Synthetic Oligonucleotide
tttcttctcccaccacc

SEQ ID NO: 326 - DNA, 24 bp, Artificial Sequence
  Synthetic Oligonucleotide
tgtctgtagctccagggaaaccac

SEQ ID NO: 327 - DNA, 24 bp, Artificial Sequence
  Synthetic Oligonucleotide
tagctccagtgaggcggaaaccac

SEQ ID NO: 328 - DNA, 24 bp, Artificial Sequence
  Synthetic Oligonucleotide
tttcttctcccaccaggaaaccac

SEQ ID NO: 329 - DNA, 20 bp, Artificial Sequence
  Synthetic Oligonucleotide
tttttgtgtgatctcttagc

SEQ ID NO: 330 - DNA, 20 bp, Artificial Sequence
  Synthetic Oligonucleotide
tttttgtgatctcttagcag

SEQ ID NO: 331 - DNA, 20 bp, Artificial Sequence
  Synthetic Oligonucleotide
ttttttgatctcttagcaga

SEQ ID NO: 332 - DNA, 15 bp, Artificial Sequence
  Synthetic Oligonucleotide
atttctctcaatcct

SEQ ID NO: 333 - DNA, 15 bp, Artificial Sequence
  Synthetic Oligonucleotide
ggcgtgtatattttt

SEQ ID NO: 334 - DNA, 15 bp, Artificial Sequence
  Synthetic Oligonucleotide
ggttatcgccctccc

SEQ ID NO: 335 - DNA, 15 bp, Artificial Sequence
  Synthetic Oligonucleotide
acgacttccgccgcc

SEQ ID NO: 336 - DNA, 17 bp, Artificial Sequence
  Synthetic Oligonucleotide
atttctctcaatcctcc

SEQ ID NO: 337 - DNA, 17 bp, Artificial Sequence
  Synthetic Oligonucleotide
ggcgtgtatatttttcc

SEQ ID NO: 338 - DNA, 17 bp, Artificial Sequence
  Synthetic Oligonucleotide
ggttatcgccctccccc

SEQ ID NO: 339 - DNA, 17 bp, Artificial Sequence
  Synthetic Oligonucleotide
acgacttccgccgcccc

SEQ ID NO: 340 - DNA, 24 bp, Artificial Sequence
  Synthetic Oligonucleotide
atttctctcaatcctggaaaccac

SEQ ID NO: 341 - DNA, 24 bp, Artificial Sequence
  Synthetic Oligonucleotide
ggcgtgtatatttttggaaaccac

SEQ ID NO: 342 - DNA, 24 bp, Artificial Sequence
  Synthetic Oligonucleotide
ggttatcgccctcccggaaaccac

SEQ ID NO: 343 - DNA, 24 bp, Artificial Sequence
  Synthetic Oligonucleotide
acgacttccgccgccggaaaccac

SEQ ID NO: 344 - DNA, 20 bp, Artificial Sequence
  Synthetic Oligonucleotide
ttttttaatttttttttaaa

SEQ ID NO: 345 - DNA, 20 bp, Artificial Sequence
  Synthetic Oligonucleotide
tttttatatgcaaaaaagaa

SEQ ID NO: 346 - DNA, 20 bp, Artificial Sequence
  Synthetic Oligonucleotide
tttttcaaaatatgggccaa

SEQ ID NO: 347 - DNA, 15 bp, Artificial Sequence
  Synthetic Oligonucleotide
ttcaccacatgtaaa

SEQ ID NO: 348 - DNA, 19 bp, Artificial Sequence
  Synthetic Oligonucleotide
ttttttcaccacatgtaaa

SEQ ID NO: 349 - DNA, 17 bp, Artificial Sequence
  Synthetic Oligonucleotide
aaatcagggcagaatgt

SEQ ID NO: 350 - DNA, 19 bp, Artificial Sequence
  Synthetic Oligonucleotide
aaatcagggcagaatgtcc

SEQ ID NO: 351 - DNA, 26 bp, Artificial Sequence
  Synthetic Oligonucleotide
aaatcagggcagaatgtccaaaggtc

SEQ ID NO: 352 - DNA, 36 bp, Artificial Sequence
  Synthetic Oligonucleotide
aaatcagggcagaatgtttttttcaccacatgtaaa

SEQ ID NO: 353 - DNA, 15 bp, Artificial Sequence
  Synthetic Oligonucleotide
ttattgtctgagccc

SEQ ID NO: 354 - DNA, 18 bp, Artificial Sequence
  Synthetic Oligonucleotide
tttttattgtctgagccc

SEQ ID NO: 355 - DNA, 15 bp, Artificial Sequence
  Synthetic Oligonucleotide
tcaggtgacggatgt

SEQ ID NO: 356 - DNA, 17 bp, Artificial Sequence
  Synthetic Oligonucleotide
tcaggtgacggatgtcc

SEQ ID NO: 357 - DNA, 24 bp, Artificial Sequence
  Synthetic Oligonucleotide
tcaggtgacggatgtccaaaggtc

SEQ ID NO: 358 - DNA, 33 bp, Artificial Sequence
  Synthetic Oligonucleotide
tcaggtgacggatgttttttattgtctgagccc

SEQ ID NO: 359 - DNA, 15 bp, Artificial Sequence
  Synthetic oligonucleotide
tgtggggagctcggc

SEQ ID NO: 360 - DNA, 15 bp, Artificial Sequence
  Synthetic oligonucleotide
ggggagctcggctgc

SEQ ID NO: 361 - DNA, 19 bp, Artificial Sequence
  Synthetic oligonucleotide
tttttgtggggagctcggc

SEQ ID NO: 362 - DNA, 19 bp, Artificial Sequence
  Synthetic oligonucleotide
ttttggggagctcggctgc

SEQ ID NO: 363 - DNA, 15 bp, Artificial Sequence
  Synthetic oligonucleotide
ttgtccaagggcagg

SEQ ID NO: 364 - DNA, 15 bp, Artificial Sequence
  Synthetic oligonucleotide
tcgatgagtgtgtgc

SEQ ID NO: 365 - DNA, 15 bp, Artificial Sequence
  Synthetic oligonucleotide
agaagaaaaaccacg

SEQ ID NO: 366 - DNA, 15 bp, Artificial Sequence
  Synthetic oligonucleotide
aatatgatttcttcc

SEQ ID NO: 367 - DNA, 15 bp, Artificial Sequence
  Synthetic oligonucleotide
gagatgggggacatg

SEQ ID NO: 368 - DNA, 15 bp, Artificial Sequence
  Synthetic oligonucleotide
ttcagtttattcaag

SEQ ID NO: 369 - DNA, 15 bp, Artificial Sequence
  Synthetic oligonucleotide
ctgtctccacttttt

SEQ ID NO: 370 - DNA, 14 bp, Artificial Sequence
  Synthetic oligonucleotide
tggaataaaacggg

SEQ ID NO: 371 - DNA, 15 bp, Artificial Sequence
  Synthetic oligonucleotide
acaattgagaaaaca

SEQ ID NO: 372 - DNA, 15 bp, Artificial Sequence
  Synthetic oligonucleotide
cagttttaagtggag

SEQ ID NO: 373 - DNA, 15 bp, Artificial Sequence
  Synthetic oligonucleotide
tgacaagaatgagac

SEQ ID NO: 374 - DNA, 15 bp, Artificial Sequence
  Synthetic oligonucleotide
ccgggcgaggggagg

SEQ ID NO: 375 - DNA, 15 bp, Artificial Sequence
  Synthetic oligonucleotide
ccgccggcctgcccg

SEQ ID NO: 376 - DNA, 15 bp, Artificial Sequence
  Synthetic oligonucleotide
cgagcgcgtatcctg

SEQ ID NO: 377 - DNA, 15 bp, Artificial Sequence
  Synthetic oligonucleotide
ctgcttctcctcagc

SEQ ID NO: 378 - DNA, 17 bp, Artificial Sequence
  Synthetic oligonucleotide
ttttcagtttattcaag

SEQ ID NO: 379 - DNA, 19 bp, Artificial Sequence
  Synthetic oligonucleotide
ttttctgtctccacttttt

SEQ ID NO: 380 - DNA, 18 bp, Artificial Sequence
  Synthetic oligonucleotide
tttttggaataaaacggg

SEQ ID NO: 381 - DNA, 19 bp, Artificial Sequence
  Synthetic oligonucleotide
ttttacaattgagaaaaca

SEQ ID NO: 382 - DNA, 19 bp, Artificial Sequence
  Synthetic oligonucleotide
ttttcagttttaagtggag

SEQ ID NO: 383 - DNA, 19 bp, Artificial Sequence
  Synthetic oligonucleotide
tttttgacaagaatgagac

SEQ ID NO: 384 - DNA, 15 bp, Artificial Sequence
  Synthetic oligonucleotide
aacagtcataataat

SEQ ID NO: 385 - DNA, 15 bp, Artificial Sequence
  Synthetic oligonucleotide
taatttaacagtcat

SEQ ID NO: 386 - DNA, 15 bp, Artificial Sequence
  Synthetic oligonucleotide
gcacgctataaagca

SEQ ID NO: 387 - DNA, 15 bp, Artificial Sequence
  Synthetic oligonucleotide
cccggggctgggctt

SEQ ID NO: 388 - DNA, 15 bp, Artificial Sequence
  Synthetic oligonucleotide
ccccgctccgcctcc

SEQ ID NO: 389 - DNA, 15 bp, Artificial Sequence
  Synthetic oligonucleotide
gcgcctccctgattt

SEQ ID NO: 390 - DNA, 15 bp, Artificial Sequence
  Synthetic oligonucleotide
tcgccgcggtggctg

SEQ ID NO: 391 - DNA, 15 bp, Artificial Sequence
  Synthetic oligonucleotide
cagcgaatggtcgcg

SEQ ID NO: 392 - DNA, 20 bp, Artificial Sequence
  Synthetic oligonucleotide
tttttaacagtcataataat

SEQ ID NO: 393 - DNA, 19 bp, Artificial Sequence
  Synthetic oligonucleotide
tttttaatttaacagtcat

SEQ ID NO: 394 - DNA, 15 bp, Artificial Sequence
  Synthetic oligonucleotide
gcggcggctgctcta

SEQ ID NO: 395 - DNA, 15 bp, Artificial Sequence
  Synthetic oligonucleotide
ttatcggccgctgcc

SEQ ID NO: 396 - DNA, 15 bp, Artificial Sequence
  Synthetic oligonucleotide
gcgtcggggacggct

SEQ ID NO: 397 - DNA, 15 bp, Artificial Sequence
  Synthetic oligonucleotide
gcggaggaaactgcg

SEQ ID NO: 398 - DNA, 15 bp, Artificial Sequence
  Synthetic oligonucleotide
gccgcacgcccgaca

SEQ ID NO: 399 - DNA, 15 bp, Artificial Sequence
  Synthetic oligonucleotide
cctgacccaccctcc

SEQ ID NO: 400 - DNA, 15 bp, Artificial Sequence
  Synthetic oligonucleotide
agggcaggccgcggc

SEQ ID NO: 401 - DNA, 15 bp, Artificial Sequence
  Synthetic oligonucleotide
ctgaatcaccccgcg

SEQ ID NO: 402 - DNA, 15 bp, Artificial Sequence
  Synthetic oligonucleotide
ggccccgagctccgc

SEQ ID NO: 403 - DNA, 15 bp, Artificial Sequence
  Synthetic oligonucleotide
gcggctgctctaata

SEQ ID NO: 404 - DNA, 15 bp, Artificial Sequence
  Synthetic oligonucleotide
cgccgcggcatgtgg

SEQ ID NO: 405 - DNA, 15 bp, Artificial Sequence
  Synthetic oligonucleotide
ccctcctcctcttgc

SEQ ID NO: 406 - DNA, 15 bp, Artificial Sequence
  Synthetic oligonucleotide
ggccgcgggctcgtg

SEQ ID NO: 407 - DNA, 15 bp, Artificial Sequence
  Synthetic oligonucleotide
gttatttttctctgt

SEQ ID NO: 408 - DNA, 15 bp, Artificial Sequence
  Synthetic oligonucleotide
atttaaaatgtttta

SEQ ID NO: 409 - DNA, 15 bp, Artificial Sequence
  Synthetic oligonucleotide
tctctgtccatttaa

SEQ ID NO: 410 - DNA, 15 bp, Artificial Sequence
  Synthetic oligonucleotide
tcatttggtcatgtg

SEQ ID NO: 411 - DNA, 15 bp, Artificial Sequence
  Synthetic oligonucleotide
tagttctctgtacat

SEQ ID NO: 412 - DNA, 15 bp, Artificial Sequence
  Synthetic oligonucleotide
tctgctggctcaact

SEQ ID NO: 413 - DNA, 15 bp, Artificial Sequence
  Synthetic oligonucleotide
atcatagaatagatt

SEQ ID NO: 414 - DNA, 15 bp, Artificial Sequence
  Synthetic oligonucleotide
ttatcatagaataga

SEQ ID NO: 415 - DNA, 15 bp, Artificial Sequence
  Synthetic oligonucleotide
aattgacatttagca

SEQ ID NO: 416 - DNA, 15 bp, Artificial Sequence
  Synthetic oligonucleotide
gacatttagcatttt

SEQ ID NO: 417 - DNA, 15 bp, Artificial Sequence
  Synthetic oligonucleotide
ttaaccattcaacac

SEQ ID NO: 418 - DNA, 15 bp, Artificial Sequence
  Synthetic oligonucleotide
cttggccggggaact

SEQ ID NO: 419 - DNA, 15 bp, Artificial Sequence
  Synthetic oligonucleotide
gccggggaactgccg

SEQ ID NO: 420 - DNA, 15 bp, Artificial Sequence
  Synthetic oligonucleotide
cgcccggagccgcgc

SEQ ID NO: 421 - DNA, 17 bp, Artificial Sequence
  Synthetic oligonucleotide
cttggccggggaactcc

SEQ ID NO: 422 - DNA, 16 bp, Artificial Sequence
  Synthetic oligonucleotide
gccggggaactgccgc

SEQ ID NO: 423 - DNA, 16 bp, Artificial Sequence
  Synthetic oligonucleotide
cgcccggagccgcgcc

SEQ ID NO: 424 - DNA, 24 bp, Artificial Sequence
  Synthetic oligonucleotide
cttggccggggaactataaaattc

SEQ ID NO: 425 - DNA, 34 bp, Artificial Sequence
  Synthetic oligonucleotide
cttggccggggaactttttgtcgttcagataaaa

SEQ ID NO: 426 - DNA, 32 bp, Artificial Sequence
  Synthetic oligonucleotide
cttggccggggaactttttcagataaaatatt

SEQ ID NO: 427 - DNA, 30 bp, Artificial Sequence
  Synthetic oligonucleotide
cttggccggggaactgtcgttcagataaaa

SEQ ID NO: 428 - DNA, 30 bp, Artificial Sequence
  Synthetic oligonucleotide
cttggccggggaactttcagataaaatatt

SEQ ID NO: 429 - DNA, 24 bp, Artificial Sequence
  Synthetic oligonucleotide
ccggggaactttttgtcgttcaga

SEQ ID NO: 430 - DNA, 21 bp, Artificial Sequence
  Synthetic oligonucleotide
cggggaactttttcagataaa

SEQ ID NO: 431 - DNA, 19 bp, Artificial Sequence
  Synthetic oligonucleotide
cggggaactgtcgttcaga

SEQ ID NO: 432 - DNA, 20 bp, Artificial Sequence
  Synthetic oligonucleotide
ccggggaactttcagataaa

SEQ ID NO: 433 - DNA, 15 bp, Artificial Sequence
  Synthetic oligonucleotide
gtcgttcagataaaa

SEQ ID NO: 434 - DNA, 15 bp, Artificial Sequence
  Synthetic oligonucleotide
ttcagataaaatatt

SEQ ID NO: 435 - DNA, 20 bp, Artificial Sequence
  Synthetic oligonucleotide
tttttgtcgttcagataaaa

SEQ ID NO: 436 - DNA, 18 bp, Artificial Sequence
  Synthetic oligonucleotide
tttttcagataaaatatt

SEQ ID NO: 437 - DNA, 15 bp, Artificial Sequence
  Synthetic oligonucleotide
ctccgcggccgctcc

SEQ ID NO: 438 - DNA, 15 bp, Artificial Sequence
  Synthetic oligonucleotide
gcccacatgctactc

SEQ ID NO: 439 - DNA, 15 bp, Artificial Sequence
  Synthetic oligonucleotide
tccgaacgcccacat

SEQ ID NO: 440 - DNA, 15 bp, Artificial Sequence
  Synthetic oligonucleotide
cgaggactcggtggt

SEQ ID NO: 441 - DNA, 15 bp, Artificial Sequence
  Synthetic oligonucleotide
ccagctccgcggccg

SEQ ID NO: 442 - DNA, 16 bp, Artificial Sequence
  Synthetic oligonucleotide
ctccgcggccgctccc

SEQ ID NO: 443 - DNA, 16 bp, Artificial Sequence
  Synthetic oligonucleotide
gcccacatgctactcc

SEQ ID NO: 444 - DNA, 24 bp, Artificial Sequence
  Synthetic oligonucleotide
ctccgcggccgctcctcaaagatc

SEQ ID NO: 445 - DNA, 24 bp, Artificial Sequence
  Synthetic oligonucleotide
gcccacatgctactcccaaaggtc

SEQ ID NO: 446 - DNA, 35 bp, Artificial Sequence
  Synthetic oligonucleotide
ctccgcggccgctcctttttgggagggaacacact

SEQ ID NO: 447 - DNA, 35 bp, Artificial Sequence
  Synthetic oligonucleotide
gcccacatgctactctttttgggagggaacacact

SEQ ID NO: 448 - DNA, 30 bp, Artificial Sequence
  Synthetic oligonucleotide
ctccgcggccgctccgggagggaacacact

SEQ ID NO: 449 - DNA, 30 bp, Artificial Sequence
  Synthetic oligonucleotide
gcccacatgctactcgggagggaacacact

SEQ ID NO: 450 - DNA, 20 bp, Artificial Sequence
  Synthetic oligonucleotide
cggccgctccgggagggaac

SEQ ID NO: 451 - DNA, 20 bp, Artificial Sequence
  Synthetic oligonucleotide
catgctactcgggagggaac

SEQ ID NO: 452 - DNA, 15 bp, Artificial Sequence
  Synthetic oligonucleotide
gggagggaacacact

SEQ ID NO: 453 - DNA, 15 bp, Artificial Sequence
  Synthetic oligonucleotide
ggggtcttcacctga

SEQ ID NO: 454 - DNA, 15 bp, Artificial Sequence
  Synthetic oligonucleotide
ggctgttatatcatg

SEQ ID NO: 455 - DNA, 15 bp, Artificial Sequence
  Synthetic oligonucleotide
ggcattttaagatgg

SEQ ID NO: 456 - DNA, 20 bp, Artificial Sequence
  Synthetic oligonucleotide
tttttgggagggaacacact

SEQ ID NO: 457 - DNA, 20 bp, Artificial Sequence
  Synthetic oligonucleotide
tttttggctgttatatcatg

SEQ ID NO: 458 - DNA, 18 bp, Artificial Sequence
  Synthetic oligonucleotide
ttttttttttggttttcc

SEQ ID NO: 459 - DNA, 15 bp, Artificial Sequence
  Synthetic oligonucleotide
tgtctcatttggaga

SEQ ID NO: 460 - DNA, 14 bp, Artificial Sequence
  Synthetic oligonucleotide
ataatgaagctggg

SEQ ID NO: 461 - DNA, 15 bp, Artificial Sequence
  Synthetic oligonucleotide
ttttccctcctggaa

SEQ ID NO: 462 - DNA, 15 bp, Artificial Sequence
  Synthetic oligonucleotide
tgcataatgaagctg

SEQ ID NO: 463 - DNA, 15 bp, Artificial Sequence
  Synthetic oligonucleotide
aaatccttcaaagaa

SEQ ID NO: 464 - DNA, 15 bp, Artificial Sequence
  Synthetic oligonucleotide
ttggaagattttttg

SEQ ID NO: 465 - DNA, 15 bp, Artificial Sequence
  Synthetic oligonucleotide
gcattcttgtagcag

SEQ ID NO: 466 - DNA, 15 bp, Artificial Sequence
  Synthetic oligonucleotide
acaacaaaaaacaga

SEQ ID NO: 467 - DNA, 15 bp, Artificial Sequence
  Synthetic oligonucleotide
tgaagctggggtctt

SEQ ID NO: 468 - DNA, 15 bp, Artificial Sequence
  Synthetic oligonucleotide
cctgaaaacatttgt

SEQ ID NO: 469 - DNA, 15 bp, Artificial Sequence
  Synthetic oligonucleotide
ttcattttccctcct

SEQ ID NO: 470 - DNA, 15 bp, Artificial Sequence
  Synthetic oligonucleotide
ttattattattatat

SEQ ID NO: 471 - DNA, 15 bp, Artificial Sequence
  Synthetic oligonucleotide
taactttgcatgaat

SEQ ID NO: 472 - DNA, 15 bp, Artificial Sequence
  Synthetic oligonucleotide
atacaaacatgtatg

SEQ ID NO: 473 - DNA, 15 bp, Artificial Sequence
  Synthetic oligonucleotide
attgtaaacctataa

SEQ ID NO: 474 - DNA, 15 bp, Artificial Sequence
  Synthetic oligonucleotide
tggagttggggttat

SEQ ID NO: 475 - DNA, 15 bp, Artificial Sequence
  Synthetic oligonucleotide
gttggggttatttag

SEQ ID NO: 476 - DNA, 13 bp, Artificial Sequence
  Synthetic oligonucleotide
ctccgccctccag

SEQ ID NO: 477 - DNA, 11 bp, Artificial Sequence
  Synthetic oligonucleotide
ccgccctccag

SEQ ID NO: 478 - DNA, 9 bp, Artificial Sequence
  Synthetic oligonucleotide
gccctccag

SEQ ID NO: 479 - DNA, 15 bp, Artificial Sequence
  Synthetic oligonucleotide
cccgctccgccctcc

SEQ ID NO: 480 - DNA, 13 bp, Artificial Sequence
  Synthetic oligonucleotide
cgctccgccctcc

SEQ ID NO: 481 - DNA, 11 bp, Artificial Sequence
  Synthetic oligonucleotide
ctccgccctcc

SEQ ID NO: 482 - DNA, 9 bp, Artificial Sequence
  Synthetic oligonucleotide
ccgccctcc

SEQ ID NO: 483 - DNA, 13 bp, Artificial Sequence
  Synthetic oligonucleotide
gccactggccgca

SEQ ID NO: 484 - DNA, 11 bp, Artificial Sequence
  Synthetic oligonucleotide
cactggccgca

SEQ ID NO: 485 - DNA, 13 bp, Artificial Sequence
  Synthetic oligonucleotide
gcgacccctggtg

SEQ ID NO: 486 - DNA, 11 bp, Artificial Sequence
  Synthetic oligonucleotide
gacccctggtg

SEQ ID NO: 487 - DNA, 13 bp, Artificial Sequence
  Synthetic oligonucleotide
ctggccgcaggca

SEQ ID NO: 488 - DNA, 13 bp, Artificial Sequence
  Synthetic oligonucleotide
ggccactggccgc

SEQ ID NO: 489 - DNA, 13 bp, Artificial Sequence
  Synthetic oligonucleotide
ctggtggccactg

SEQ ID NO: 490 - DNA, 13 bp, Artificial Sequence
  Synthetic oligonucleotide
gacccctggtggc

SEQ ID NO: 491 - DNA, 13 bp, Artificial Sequence
  Synthetic oligonucleotide
gcggcgacccctg

SEQ ID NO: 492 - DNA, 13 bp, Artificial Sequence
  Synthetic oligonucleotide
gtgctgcggcgac

SEQ ID NO: 493 - DNA, 13 bp, Artificial Sequence
  Synthetic oligonucleotide
gctgggtgctgcg

SEQ ID NO: 494 - DNA, 13 bp, Artificial Sequence
  Synthetic oligonucleotide
ccagcgctgggtg

SEQ ID NO: 495 - DNA, 13 bp, Artificial Sequence
  Synthetic oligonucleotide
gccctccagcgct

SEQ ID NO: 496 - DNA, 13 bp, Artificial Sequence
  Synthetic oligonucleotide
cgcccgctccgcc

SEQ ID NO: 497 - DNA, 35 bp, Artificial Sequence; identical to SEQ ID NO: 142
  Synthetic oligonucleotide

SEQ ID NO: 498 - DNA, 35 bp, Artificial Sequence; identical to SEQ ID NO: 143
  Synthetic oligonucleotide

SEQ ID NO: 499 - DNA, 17 bp, Artificial Sequence
  Synthetic oligonucleotide
caagtccagtttggttt

SEQ ID NO: 500 - DNA, 17 bp, Artificial Sequence
  Synthetic oligonucleotide
gaataggccaaggaaga

SEQ ID NO: 501 - DNA, 17 bp, Artificial Sequence
  Synthetic oligonucleotide
atcaagcatcttttccg

SEQ ID NO: 502 - DNA, 17 bp, Artificial Sequence
  Synthetic oligonucleotide
ttaaaacggggctgggc

SEQ ID NO: 503 - DNA, 17 bp, Artificial Sequence
  Synthetic oligonucleotide
gatagcttttaatgtcc

SEQ ID NO: 504 - DNA, 17 bp, Artificial Sequence
  Synthetic oligonucleotide
agctggggtcttggcct

SEQ ID NO: 505 - DNA, 17 bp, Artificial Sequence
  Synthetic oligonucleotide
cctcagctgcataatga

SEQ ID NO: 506 - DNA, 17 bp, Artificial Sequence
  Synthetic oligonucleotide
caacaacaaaaaacaga

SEQ ID NO: 507 - DNA, 17 bp, Artificial Sequence
  Synthetic oligonucleotide
aaaaaaataaacaacaa

SEQ ID NO: 508 - DNA, 17 bp, Artificial Sequence
  Synthetic oligonucleotide
cctcaaaagcaggaata

SEQ ID NO: 509 - DNA, 17 bp, Artificial Sequence
  Synthetic oligonucleotide
acacatagcccaactgt

SEQ ID NO: 510 - DNA, 17 bp, Artificial Sequence
  Synthetic oligonucleotide
ctttctacagagctgtg

SEQ ID NO: 511 - DNA, 17 bp, Artificial Sequence
  Synthetic oligonucleotide
gtaggaggcaacacatt

SEQ ID NO: 512 - DNA, 17 bp, Artificial Sequence
  Synthetic oligonucleotide
cagaacttgggggcaag

SEQ ID NO: 513 - DNA, 17 bp, Artificial Sequence
  Synthetic oligonucleotide
ccatagaaattaaaaat

SEQ ID NO: 514 - DNA, 17 bp, Artificial Sequence
  Synthetic oligonucleotide
acaatccaaaaaatctt

SEQ ID NO: 515 - DNA, 17 bp, Artificial Sequence
  Synthetic oligonucleotide
gtgagggaggaaatccg

SEQ ID NO: 516 - DNA, 17 bp, Artificial Sequence
  Synthetic oligonucleotide
aagataaggggtatcat

SEQ ID NO: 517 - DNA, 17 bp, Artificial Sequence
  Synthetic oligonucleotide
ggcataagacattataa

SEQ ID NO: 518 - DNA, 17 bp, Artificial Sequence
  Synthetic oligonucleotide
tgttatattcaggtata

SEQ ID NO: 519 - DNA, 17 bp, Artificial Sequence
  Synthetic oligonucleotide
tttgcttttttaaaggt

SEQ ID NO: 520 - DNA, 17 bp, Artificial Sequence
  Synthetic oligonucleotide
tttttccttcttattat

SEQ ID NO: 521 - DNA, 17 bp, Artificial Sequence
  Synthetic oligonucleotide
cattttccctcctggaa

SEQ ID NO: 522 - DNA, 17 bp, Artificial Sequence
  Synthetic oligonucleotide
gaagagtgaagacaatt

SEQ ID NO: 523 - DNA, 17 bp, Artificial Sequence
  Synthetic oligonucleotide
taaatccttcaaagaat

SEQ ID NO: 524 - DNA, 17 bp, Artificial Sequence
  Synthetic oligonucleotide
tcatgtacttcttgcag

SEQ ID NO: 525 - DNA, 17 bp, Artificial Sequence
  Synthetic oligonucleotide
ggttgaccagctgctct

SEQ ID NO: 526 - DNA, 17 bp, Artificial Sequence
  Synthetic oligonucleotide
agatagaacagtgagca

SEQ ID NO: 527 - DNA, 17 bp, Artificial Sequence
  Synthetic oligonucleotide
taatgtgtctcatttgg

SEQ ID NO: 528 - DNA, 17 bp, Artificial Sequence
  Synthetic oligonucleotide
atttgtaggctaccctt

SEQ ID NO: 529 - DNA, 17 bp, Artificial Sequence
  Synthetic oligonucleotide
gaaagaagcctgaaaac

SEQ ID NO: 530 - DNA, 17 bp, Artificial Sequence
  Synthetic oligonucleotide
agaagtgcttacacttt

SEQ ID NO: 531 - DNA, 17 bp, Artificial Sequence
  Synthetic oligonucleotide
tcaatgctaaagagctc

SEQ ID NO: 532 - DNA, 13 bp, Artificial Sequence
  Synthetic oligonucleotide
agtctgggtgtcc

SEQ ID NO: 533 - DNA, 13 bp, Artificial Sequence
  Synthetic oligonucleotide
ccgacagtctggg

SEQ ID NO: 534 - DNA, 13 bp, Artificial Sequence
  Synthetic oligonucleotide
ctccgacagtctg

SEQ ID NO: 535 - DNA, 13 bp, Artificial Sequence
  Synthetic oligonucleotide
gacagtctgggtg

SEQ ID NO: 536 - DNA, 11 bp, Artificial Sequence
  Synthetic oligonucleotide
cagtctgggtg

SEQ ID NO: 537 - DNA, 15 bp, Artificial Sequence
  Synthetic oligonucleotide
ctcagcctggccctg

SEQ ID NO: 538 - DNA, 15 bp, Artificial Sequence
  Synthetic oligonucleotide
agttcaaggatcagc

SEQ ID NO: 539 - DNA, 15 bp, Artificial Sequence
  Synthetic oligonucleotide
gctctccgacagtct

SEQ ID NO: 540 - DNA, 13 bp, Artificial Sequence
  Synthetic oligonucleotide
tctccgacagtct

SEQ ID NO: 541 - DNA, 11 bp, Artificial Sequence
  Synthetic oligonucleotide
tccgacagtct

SEQ ID NO: 542 - DNA, 15 bp, Artificial Sequence
  Synthetic oligonucleotide
cggagctctccgaca

SEQ ID NO: 543 - DNA, 13 bp, Artificial Sequence
  Synthetic oligonucleotide
gagctctccgaca

SEQ ID NO: 544 - DNA, 11 bp, Artificial Sequence
  Synthetic oligonucleotide
gctctccgaca

SEQ ID NO: 545 - DNA, 15 bp, Artificial Sequence
  Synthetic oligonucleotide
ctattccattttgga

SEQ ID NO: 546 - DNA, 13 bp, Artificial Sequence
  Synthetic oligonucleotide
ctattccattttg

SEQ ID NO: 547 - DNA, 15 bp, Artificial Sequence
  Synthetic oligonucleotide
attccattttggaaa

SEQ ID NO: 548 - DNA, 15 bp, Artificial Sequence
  Synthetic oligonucleotide
ccattttggaaaggt

SEQ ID NO: 549 - DNA, 13 bp, Artificial Sequence
  Synthetic oligonucleotide
ccattttggaaag

SEQ ID NO: 550 - DNA, 15 bp, Artificial Sequence
  Synthetic oligonucleotide
cattttggaaaggtt

SEQ ID NO: 551 - DNA, 13 bp, Artificial Sequence
  Synthetic oligonucleotide
cattttggaaagg

SEQ ID NO: 552 - DNA, 15 bp, Artificial Sequence
  Synthetic oligonucleotide
ggaaaggtttattgt

SEQ ID NO: 553 - DNA, 22 bp, Artificial Sequence
  Synthetic oligonucleotide
tccgacagtctccattttggaa

SEQ ID NO: 554 - DNA, 22 bp, Artificial Sequence
  Synthetic oligonucleotide
gctctccgacaccattttggaa

SEQ ID NO: 555 - DNA, 22 bp, Artificial Sequence
  Synthetic oligonucleotide
tccgacagtctcattttggaaa

SEQ ID NO: 556 - DNA, 22 bp, Artificial Sequence
  Synthetic oligonucleotide
gctctccgacacattttggaaa

SEQ ID NO: 557 - DNA, 15 bp, Artificial Sequence
  Synthetic oligonucleotide
cctcaaaagcaggaa

SEQ ID NO: 558 - DNA, 13 bp, Artificial Sequence
  Synthetic oligonucleotide
cctcaaaagcagg

SEQ ID NO: 559 - DNA, 11 bp, Artificial Sequence
  Synthetic oligonucleotide
cctcaaaagca

SEQ ID NO: 560 - DNA, 13 bp, Artificial Sequence
  Synthetic oligonucleotide
tcaaaagcaggaa

SEQ ID NO: 561 - DNA, 11 bp, Artificial Sequence
  Synthetic oligonucleotide
caaaagcagga

SEQ ID NO: 562 - DNA, 27 bp, Artificial Sequence
  Synthetic oligonucleotide
ccgccctccagcctcaaaagcaggaat

SEQ ID NO: 563 - DNA, 25 bp, Artificial Sequence
  Synthetic oligonucleotide
ccgccctccagcctcaaaagcagga

SEQ ID NO: 564 - DNA, 23 bp, Artificial Sequence
  Synthetic oligonucleotide
ccgccctccagcctcaaaagcag

SEQ ID NO: 565 - DNA, 21 bp, Artificial Sequence
  Synthetic oligonucleotide
ccgccctccagcctcaaaagc

SEQ ID NO: 566 - DNA, 25 bp, Artificial Sequence
  Synthetic oligonucleotide
gccctccagcctcaaaagcaggaat

SEQ ID NO: 567 - DNA, 23 bp, Artificial Sequence
  Synthetic oligonucleotide
gccctccagcctcaaaagcagga

SEQ ID NO: 568 - DNA, 21 bp, Artificial Sequence
  Synthetic oligonucleotide
gccctccagcctcaaaagcag

SEQ ID NO: 569 - DNA, 19 bp, Artificial Sequence
  Synthetic oligonucleotide
gccctccagcctcaaaagc

SEQ ID NO: 570 - DNA, 17 bp, Artificial Sequence
  Synthetic oligonucleotide
ccctccagcctcaaaag

SEQ ID NO: 571 - DNA, 15 bp, Artificial Sequence
  Synthetic oligonucleotide
cctccagcctcaaaa

SEQ ID NO: 572 - DNA, 21 bp, Artificial Sequence
  Synthetic oligonucleotide
gccctccagtcaaaagcagga

SEQ ID NO: 573 - DNA, 19 bp, Artificial Sequence
  Synthetic oligonucleotide
gccctccagcaaaagcagg

SEQ ID NO: 574 - DNA, 23 bp, Artificial Sequence
  Synthetic oligonucleotide
ccgccctccagtcaaaagcagga

SEQ ID NO: 575 - DNA, 21 bp, Artificial Sequence
  Synthetic oligonucleotide
ccgccctccagcaaaagcagg

SEQ ID NO: 576 - DNA, 13 bp, Artificial Sequence
  Synthetic oligonucleotide
ctccgccctccag

SEQ ID NO: 577 - DNA, 11 bp, Artificial Sequence
  Synthetic oligonucleotide
ccgccctccag

SEQ ID NO: 578 - DNA, 9 bp, Artificial Sequence
  Synthetic oligonucleotide
gccctccag

SEQ ID NO: 579 - DNA, 15 bp, Artificial Sequence
  Synthetic oligonucleotide
cccgctccgccctcc

SEQ ID NO: 580 - DNA, 13 bp, Artificial Sequence
  Synthetic oligonucleotide
cgctccgccctcc

SEQ ID NO: 581 - DNA, 11 bp, Artificial Sequence
  Synthetic oligonucleotide
ctccgccctcc

SEQ ID NO: 582 - DNA, 9 bp, Artificial Sequence
  Synthetic oligonucleotide
ccgccctcc

SEQ ID NO: 583 - DNA, 15 bp, Artificial Sequence
  Synthetic oligonucleotide
gcctttgagaaagca

SEQ ID NO: 584 - DNA, 15 bp, Artificial Sequence
  Synthetic oligonucleotide
gactgtggggccttt

SEQ ID NO: 585 - DNA, 15 bp, Artificial Sequence
  Synthetic oligonucleotide
aggaagtggaggact

SEQ ID NO: 586 - DNA, 15 bp, Artificial Sequence
  Synthetic oligonucleotide
tgcattttcactgaa

SEQ ID NO: 587 - DNA, 15 bp, Artificial Sequence
  Synthetic oligonucleotide
cattttcactgaagc

SEQ ID NO: 588 - DNA, 15 bp, Artificial Sequence
  Synthetic oligonucleotide
actgaagcatttatt

SEQ ID NO: 589 - DNA, 15 bp, Artificial Sequence
  Synthetic oligonucleotide
cacacaaatgtatgg

SEQ ID NO: 590 - DNA, 15 bp, Artificial Sequence
  Synthetic oligonucleotide
ggattttattgacaa

SEQ ID NO: 591 - DNA, 15 bp, Artificial Sequence
  Synthetic oligonucleotide
aaaacaacaaagttt

SEQ ID NO: 592 - DNA, 15 bp, Artificial Sequence
  Synthetic oligonucleotide
agtgccataaaaagt

SEQ ID NO: 593 - DNA, 15 bp, Artificial Sequence
  Synthetic oligonucleotide
tcaaatataaaaatt

SEQ ID NO: 594 - DNA, 15 bp, Artificial Sequence
  Synthetic oligonucleotide
ttccccccacccacc

SEQ ID NO: 595 - DNA, 15 bp, Artificial Sequence
  Synthetic oligonucleotide
catttgcttccaatt

SEQ ID NO: 596 - DNA, 15 bp, Artificial Sequence
  Synthetic oligonucleotide
gctcaaccctttttc

SEQ ID NO: 597 - DNA, 15 bp, Artificial Sequence
  Synthetic oligonucleotide
agacctactactctg

SEQ ID NO: 598 - DNA, 15 bp, Artificial Sequence
  Synthetic oligonucleotide
ccctccaccggaagt

SEQ ID NO: 599 - DNA, 15 bp, Artificial Sequence
  Synthetic oligonucleotide
gcccgcgctcgccgt

SEQ ID NO: 600 - DNA, 15 bp, Artificial Sequence
  Synthetic oligonucleotide
acgccccctggcagc

SEQ ID NO: 601 - DNA, 15 bp, Artificial Sequence
  Synthetic oligonucleotide
gctcagcccctcggc

SEQ ID NO: 602 - DNA, 15 bp, Artificial Sequence
  Synthetic oligonucleotide
agcagaggaagatca

SEQ ID NO: 603 - DNA, 15 bp, Artificial Sequence
  Synthetic oligonucleotide
cagaggaagatcaaa

SEQ ID NO: 604 - DNA, 15 bp, Artificial Sequence
  Synthetic oligonucleotide
cagatttttgaaact

SEQ ID NO: 605 - DNA, 15 bp, Artificial Sequence
  Synthetic oligonucleotide
cagactaattttttg

SEQ ID NO: 606 - DNA, 15 bp, Artificial Sequence
  Synthetic oligonucleotide
tttttgctttttcat

SEQ ID NO: 607 - DNA, 15 bp, Artificial Sequence
  Synthetic oligonucleotide
aattttttgcttttt

SEQ ID NO: 608 - DNA, 15 bp, Artificial Sequence
  Synthetic oligonucleotide
atgtttggcaatact

SEQ ID NO: 609 - DNA, 15 bp, Artificial Sequence
  Synthetic oligonucleotide
ttggcaatacttttt

SEQ ID NO: 610 - DNA, 15 bp, Artificial Sequence
  Synthetic oligonucleotide
gctgccctggccccg

SEQ ID NO: 611 - DNA, 15 bp, Artificial Sequence
  Synthetic oligonucleotide
cggacacacccctcg

SEQ ID NO: 612 - DNA, 15 bp, Artificial Sequence
  Synthetic oligonucleotide
acgggacgcgagtcc

SEQ ID NO: 613 - DNA, 15 bp, Artificial Sequence
  Synthetic oligonucleotide
gtctggggagaaagc

SEQ ID NO: 614 - DNA, 15 bp, Artificial Sequence
  Synthetic oligonucleotide
ccactcggtgggtct

SEQ ID NO: 615 - DNA, 15 bp, Artificial Sequence
  Synthetic oligonucleotide
tgatctgttatcatc

SEQ ID NO: 616 - DNA, 15 bp, Artificial Sequence
  Synthetic oligonucleotide
ctgttatcatctgta

SEQ ID NO: 617 - DNA, 15 bp, Artificial Sequence
  Synthetic oligonucleotide
gtgtataaagatttt

SEQ ID NO: 618 - DNA, 15 bp, Artificial Sequence
  Synthetic oligonucleotide
caatttacattttag

SEQ ID NO: 619 - DNA, 15 bp, Artificial Sequence
  Synthetic oligonucleotide
tacattttagaccat

SEQ ID NO: 620 - DNA, 15 bp, Artificial Sequence
  Synthetic oligonucleotide
tgctataagatgtaa

SEQ ID NO: 621 - DNA, 15 bp, Artificial Sequence
  Synthetic oligonucleotide
aaggaagccggcaag

SEQ ID NO: 622 - DNA, 15 bp, Artificial Sequence
  Synthetic oligonucleotide
cgccacaactcattc

SEQ ID NO: 623 - DNA, 15 bp, Artificial Sequence
  Synthetic oligonucleotide
atgggagcattgtgg

SEQ ID NO: 624 - DNA, 15 bp, Artificial Sequence
  Synthetic oligonucleotide
cgcccgcccagcccc

SEQ ID NO: 625 - DNA, 15 bp, Artificial Sequence
  Synthetic oligonucleotide
cccctcccccgcccg

SEQ ID NO: 626 - DNA, 15 bp, Artificial Sequence
  Synthetic oligonucleotide
cttccgctgctgctg

SEQ ID NO: 627 - DNA, 15 bp, Artificial Sequence
  Synthetic oligonucleotide
cttcttagtaccaac

SEQ ID NO: 628 - DNA, 15 bp, Artificial Sequence
  Synthetic oligonucleotide
tttagagcaaaatcg

SEQ ID NO: 629 - DNA, 15 bp, Artificial Sequence
  Synthetic oligonucleotide
ggtagttaaatgttt

SEQ ID NO: 630 - DNA, 15 bp, Artificial Sequence
  Synthetic oligonucleotide
tacttaagaaagaga

SEQ ID NO: 631 - DNA, 15 bp, Artificial Sequence
  Synthetic oligonucleotide
tatacttaagaaaga

SEQ ID NO: 632 - DNA, 15 bp, Artificial Sequence
  Synthetic oligonucleotide
cgccgccgacgccgg

SEQ ID NO: 633 - DNA, 15 bp, Artificial Sequence
  Synthetic oligonucleotide
ctctctccgagagga

SEQ ID NO: 634 - DNA, 15 bp, Artificial Sequence
  Synthetic oligonucleotide
cgccccgccctcttg

SEQ ID NO: 635 - DNA, 15 bp, Artificial Sequence
  Synthetic oligonucleotide
ccgcgcgctgctgca

SEQ ID NO: 636 - DNA, 15 bp, Artificial Sequence
  Synthetic oligonucleotide
cactttcacagagag

SEQ ID NO: 637 - DNA, 16 bp, Artificial Sequence
  Synthetic oligonucleotide
ctttcacatgtattaa

SEQ ID NO: 638 - DNA, 15 bp, Artificial Sequence
  Synthetic oligonucleotide
atgtattaaaaaact

SEQ ID NO: 639 - DNA, 15 bp, Artificial Sequence
  Synthetic oligonucleotide
gacatttttatgtaa

SEQ ID NO: 640 - DNA, 15 bp, Artificial Sequence
  Synthetic oligonucleotide
catttttatgtaaat

SEQ ID NO: 641 - DNA, 15 bp, Artificial Sequence
  Synthetic oligonucleotide
aaatttataaggcaa

SEQ ID NO: 642 - DNA, 15 bp, Artificial Sequence
  Synthetic oligonucleotide
aggcaaactctttat

SEQ ID NO: 643 - DNA, 15 bp, Artificial Sequence
  Synthetic oligonucleotide
gtctctggaacaatt

SEQ ID NO: 644 - DNA, 15 bp, Artificial Sequence
  Synthetic oligonucleotide
cagttcaaacacaga

SEQ ID NO: 645 - DNA, 15 bp, Artificial Sequence
  Synthetic oligonucleotide
caaacacagaagaga

SEQ ID NO: 646 - DNA, 15 bp, Artificial Sequence
  Synthetic oligonucleotide
aacacagaagagatt

SEQ ID NO: 647 - DNA, 15 bp, Artificial Sequence
  Synthetic oligonucleotide
gggggagaagaaagg

SEQ ID NO: 648 - DNA, 15 bp, Artificial Sequence
  Synthetic oligonucleotide
tcgtttttttttctt

SEQ ID NO: 649 - DNA, 15 bp, Artificial Sequence
  Synthetic oligonucleotide
cttttttttcttttt

SEQ ID NO: 650 - DNA, 15 bp, Artificial Sequence
  Synthetic oligonucleotide
cctatgctatggtta

SEQ ID NO: 651 - DNA, 15 bp, Artificial Sequence
  Synthetic oligonucleotide
agtttactgaaagaa

SEQ ID NO: 652 - DNA, 15 bp, Artificial Sequence
  Synthetic oligonucleotide
actgaaagaaaaaaa

SEQ ID NO: 653 - DNA, 15 bp, Artificial Sequence
  Synthetic oligonucleotide
ccttattcatatttt

SEQ ID NO: 654 - DNA, 15 bp, Artificial Sequence
  Synthetic oligonucleotide
cttccttattcatat

SEQ ID NO: 655 - DNA, 15 bp, Artificial Sequence
  Synthetic oligonucleotide
caatccttcaatatt

SEQ ID NO: 656 - DNA, 15 bp, Artificial Sequence
  Synthetic oligonucleotide
ggcatttcattttac

SEQ ID NO: 657 - DNA, 15 bp, Artificial Sequence
  Synthetic oligonucleotide
cattttacaaatatt

SEQ ID NO: 658 - DNA, 15 bp, Artificial Sequence
  Synthetic oligonucleotide
gaaatgaaataagta

SEQ ID NO: 659 - DNA, 15 bp, Artificial Sequence
  Synthetic oligonucleotide
agatatgcaagataa

SEQ ID NO: 660 - DNA, 15 bp, Artificial Sequence
  Synthetic oligonucleotide
gcgggcccagcaggt

SEQ ID NO: 661 - DNA, 15 bp, Artificial Sequence
  Synthetic oligonucleotide
cagtgagtgccgagt

SEQ ID NO: 662 - DNA, 15 bp, Artificial Sequence
  Synthetic oligonucleotide
gcccgggcagtgagt

SEQ ID NO: 663 - DNA, 15 bp, Artificial Sequence
  Synthetic oligonucleotide
tgtccgggcggcccg

SEQ ID NO: 664 - DNA, 15 bp, Artificial Sequence
  Synthetic oligonucleotide
cgcgcgtgtgcgagt

SEQ ID NO: 665 - DNA, 15 bp, Artificial Sequence
  Synthetic oligonucleotide
cttcagacaggctgc

SEQ ID NO: 666 - DNA, 15 bp, Artificial Sequence
  Synthetic oligonucleotide
acctctgcacttcag

SEQ ID NO: 667 - DNA, 15 bp, Artificial Sequence
  Synthetic oligonucleotide
cggcgcgggtccctt

SEQ ID NO: 668 - DNA, 15 bp, Artificial Sequence
  Synthetic oligonucleotide
tggtattcgaattat

SEQ ID NO: 669 - DNA, 15 bp, Artificial Sequence
  Synthetic oligonucleotide
cggcctgccctggta

SEQ ID NO: 670 - DNA, 15 bp, Artificial Sequence
  Synthetic oligonucleotide
tcagagattatgaaa

SEQ ID NO: 671 - DNA, 15 bp, Artificial Sequence
  Synthetic oligonucleotide
tgttttcagagatta

SEQ ID NO: 672 - DNA, 15 bp, Artificial Sequence
  Synthetic oligonucleotide
catgtagaaatgctt

SEQ ID NO: 673 - DNA, 15 bp, Artificial Sequence
  Synthetic oligonucleotide
aaacatgtagaaatg

SEQ ID NO: 674 - DNA, 15 bp, Artificial Sequence
  Synthetic oligonucleotide
ttgataccatttatg

SEQ ID NO: 675 - DNA, 15 bp, Artificial Sequence
  Synthetic oligonucleotide
gaactcaattattat

SEQ ID NO: 676 - DNA, 15 bp, Artificial Sequence
  Synthetic oligonucleotide
aaaacgactccacaa

SEQ ID NO: 677 - DNA, 15 bp, Artificial Sequence
  Synthetic oligonucleotide
ctccgaggaaaaacg

SEQ ID NO: 678 - DNA, 15 bp, Artificial Sequence
  Synthetic oligonucleotide
gctccgaggaaaaac

SEQ ID NO: 679 - DNA, 15 bp, Artificial Sequence
  Synthetic oligonucleotide
ctcggcgggagaaag

SEQ ID NO: 680 - DNA, 13 bp, Artificial Sequence
  Synthetic oligonucleotide
gaaccgaaatttt

SEQ ID NO: 681 - DNA, 15 bp, Artificial Sequence
  Synthetic oligonucleotide
gagaagggtgcagat

SEQ ID NO: 682 - DNA, 15 bp, Artificial Sequence
  Synthetic oligonucleotide
ctctccagatgagaa

SEQ ID NO: 683 - DNA, 15 bp, Artificial Sequence
  Synthetic oligonucleotide
caggggtccgctctc

SEQ ID NO: 684 - DNA, 15 bp, Artificial Sequence
  Synthetic oligonucleotide
tccgggcagccaggg

SEQ ID NO: 685 - DNA, 15 bp, Artificial Sequence
  Synthetic oligonucleotide
ggggctcgcctccgg

SEQ ID NO: 686 - DNA, 15 bp, Artificial Sequence
  Synthetic oligonucleotide
cccccgggaaggggc

SEQ ID NO: 687 - DNA, 15 bp, Artificial Sequence
  Synthetic oligonucleotide
cccaccccccgggaa

SEQ ID NO: 688 - DNA, 16 bp, Artificial Sequence
  Synthetic oligonucleotide
gcgttgccgcccccac

SEQ ID NO: 689 - DNA, 15 bp, Artificial Sequence
  Synthetic oligonucleotide
gctgggtcgcgcgtt

SEQ ID NO: 690 - DNA, 15 bp, Artificial Sequence
  Synthetic oligonucleotide
gcgcaggaccgctgg

SEQ ID NO: 691 - DNA, 15 bp, Artificial Sequence
  Synthetic oligonucleotide
aggagggagggtggg

SEQ ID NO: 692 - DNA, 15 bp, Artificial Sequence
  Synthetic oligonucleotide
cgctggaggcggagg

SEQ ID NO: 693 - DNA, 15 bp, Artificial Sequence
  Synthetic oligonucleotide
tggagccgagcgctg

SEQ ID NO: 694 - DNA, 15 bp, Artificial Sequence
  Synthetic oligonucleotide
ctgcccctttgttgg

SEQ ID NO: 695 - DNA, 15 bp, Artificial Sequence
  Synthetic oligonucleotide
ctccccgctgcgggc

SEQ ID NO: 696 - DNA, 15 bp, Artificial Sequence
  Synthetic oligonucleotide
cggctcctcctcctc

SEQ ID NO: 697 - DNA, 15 bp, Artificial Sequence
  Synthetic oligonucleotide
ggctcgctccttcgg

SEQ ID NO: 698 - DNA, 15 bp, Artificial Sequence
  Synthetic oligonucleotide
tttgtgcgcgagaga

SEQ ID NO: 699 - DNA, 15 bp, Artificial Sequence
  Synthetic oligonucleotide
acgactccacaactt

SEQ ID NO: 700 - DNA, 15 bp, Artificial Sequence
  Synthetic oligonucleotide
gcccgcttccctgct

SEQ ID NO: 701 - DNA, 15 bp, Artificial Sequence
  Synthetic oligonucleotide
cggccggctgctgct

SEQ ID NO: 702 - DNA, 15 bp, Artificial Sequence
  Synthetic oligonucleotide
gcgggagaaagcccg

SEQ ID NO: 703 - DNA, 15 bp, Artificial Sequence
  Synthetic oligonucleotide
cctcctcgcccctcg

SEQ ID NO: 704 - DNA, 15 bp, Artificial Sequence
  Synthetic oligonucleotide
agaggctcctcctcg

SEQ ID NO: 705 - DNA, 15 bp, Artificial Sequence
  Synthetic oligonucleotide
tcggcttctggagcc

SEQ ID NO: 706 - DNA, 15 bp, Artificial Sequence
  Synthetic oligonucleotide
ccgtgattccccaat

SEQ ID NO: 707 - DNA, 15 bp, Artificial Sequence
  Synthetic oligonucleotide
aggggggcgccgctc

SEQ ID NO: 708 - DNA, 15 bp, Artificial Sequence
  Synthetic oligonucleotide
aaatgacccaaaaga

SEQ ID NO: 709 - DNA, 15 bp, Artificial Sequence
  Synthetic oligonucleotide
gttttccgtttgcag

SEQ ID NO: 710 - DNA, 15 bp, Artificial Sequence
  Synthetic oligonucleotide
ccaaacgctacagag

SEQ ID NO: 711 - DNA, 15 bp, Artificial Sequence
  Synthetic oligonucleotide
caggcaccaactttg

SEQ ID NO: 712 - DNA, 15 bp, Artificial Sequence
  Synthetic oligonucleotide
cctggaaggggcgcg

SEQ ID NO: 713 - DNA, 15 bp, Artificial Sequence
  Synthetic oligonucleotide
cagtcaaagcgcaaa

SEQ ID NO: 714 - DNA, 15 bp, Artificial Sequence
  Synthetic oligonucleotide
ccaaaaacaaaacag

SEQ ID NO: 715 - DNA, 15 bp, Artificial Sequence
  Synthetic oligonucleotide
ttccgccaaaaacaa

SEQ ID NO: 716 - DNA, 15 bp, Artificial Sequence
  Synthetic oligonucleotide
ggaggagggagggtg

SEQ ID NO: 717 - DNA, 15 bp, Artificial Sequence
  Synthetic oligonucleotide
cgagcgctggaggcg

SEQ ID NO: 718 - DNA, 15 bp, Artificial Sequence
  Synthetic oligonucleotide
cctgcccctttgttg

SEQ ID NO: 719 - DNA, 15 bp, Artificial Sequence
  Synthetic oligonucleotide
ggcggctcctcctcc

SEQ ID NO: 720 - DNA, 15 bp, Artificial Sequence
  Synthetic oligonucleotide
tagacacttccagaa

SEQ ID NO: 721 - DNA, 15 bp, Artificial Sequence
  Synthetic oligonucleotide
ttccagaattgtcct

SEQ ID NO: 722 - DNA, 15 bp, Artificial Sequence
  Synthetic oligonucleotide
cagaattgtccttta

SEQ ID NO: 723 - DNA, 15 bp, Artificial Sequence
  Synthetic oligonucleotide
ctgctggaactcggc

SEQ ID NO: 724 - DNA, 15 bp, Artificial Sequence
  Synthetic oligonucleotide
ggccaggctcagctg

SEQ ID NO: 725 - DNA, 15 bp, Artificial Sequence
  Synthetic oligonucleotide
gcagccaggagcctg

SEQ ID NO: 726 - DNA, 15 bp, Artificial Sequence
  Synthetic oligonucleotide
actcggccaggctca

SEQ ID NO: 727 - DNA, 15 bp, Artificial Sequence
  Synthetic oligonucleotide
gctggcctgctggaa

SEQ ID NO: 728 - DNA, 15 bp, Artificial Sequence
  Synthetic oligonucleotide
tttaaattgtatcgg

SEQ ID NO: 729 - DNA, 15 bp, Artificial Sequence
  Synthetic oligonucleotide
attgtatcgggcaaa

SEQ ID NO: 730 - DNA, 15 bp, Artificial Sequence
  Synthetic oligonucleotide
gattaaaacaaaaga

SEQ ID NO: 731 - DNA, 15 bp, Artificial Sequence
  Synthetic oligonucleotide
aaaacaaaagaaacc

SEQ ID NO: 732 - DNA, 15 bp, Artificial Sequence
  Synthetic oligonucleotide
gggataaaggaaggg

SEQ ID NO: 733 - DNA, 15 bp, Artificial Sequence
  Synthetic oligonucleotide
cactgggataaagga

SEQ ID NO: 734 - DNA, 15 bp, Artificial Sequence
  Synthetic oligonucleotide
gagccgcccgctttg

SEQ ID NO: 735 - DNA, 14 bp, Artificial Sequence
  Synthetic oligonucleotide
tctgggccccactg

SEQ ID NO: 736 - DNA, 15 bp, Artificial Sequence
  Synthetic oligonucleotide
caaaaggtcttagct

SEQ ID NO: 737 - DNA, 15 bp, Artificial Sequence
  Synthetic oligonucleotide
tagctattattactg

SEQ ID NO: 738 - DNA, 15 bp, Artificial Sequence
  Synthetic oligonucleotide
actgttgttgtttta

SEQ ID NO: 739 - DNA, 15 bp, Artificial Sequence
  Synthetic oligonucleotide
accttagaggttgta

SEQ ID NO: 740 - DNA, 15 bp, Artificial Sequence
  Synthetic oligonucleotide
tacctgaaattgcag

SEQ ID NO: 741 - DNA, 15 bp, Artificial Sequence
  Synthetic oligonucleotide
gtcagaaaagctacc

SEQ ID NO: 742 - DNA, 15 bp, Artificial Sequence
  Synthetic oligonucleotide
cacgcttggtgtgca

SEQ ID NO: 743 - DNA, 15 bp, Artificial Sequence
  Synthetic oligonucleotide
ctgtgaatgtgtgaa

SEQ ID NO: 744 - DNA, 15 bp, Artificial Sequence
  Synthetic oligonucleotide
aacaggaagcacctg

SEQ ID NO: 745 - DNA, 39 bp, Artificial Sequence
  Synthetic oligonucleotide
cgctccgccctccagtttttttttaggaggcaacacatt

SEQ ID NO: 746 - DNA, 41 bp, Artificial Sequence
  Synthetic oligonucleotide
cgctccgccctccagcctttttttttaggaggcaacacatt

(19) United States
(12) Patent Application Publication
(10) Pub. No.: US 2015/0247145 A1
(43) Pub. Date: Sep. 3, 2015

(54) METHODS FOR MODULATING RNA USING 3' TARGETING OLIGONUCLEOTIDES

(71) Applicant: RaNA Therapeutics, Inc., Cambridge, MA (US)

(72) Inventor: FATIH OZSOLAK, Boston, MA (US)

(73) Assignee: RaNA Therapeutics, Inc., Cambridge, MA (US)

(21) Appl. No.: 14/700,395

(22) Filed: Apr. 30, 2015

Related U.S. Application Data
(63) Continuation of application No. 14/461,317, filed on Aug. 15, 2014.
(60) Provisional application No. 62/010,417, filed on Jun. 10, 2014.
(60) Provisional application No. 61/898,461, filed on Oct. 31, 2013.
(60) Provisional application No. 61/866,989, filed on Aug. 16, 2013.

(51) Int. Cl. C12N 15/113

(52) U.S. Cl. CPC C12N 15/113; C12N 2310/11

(57) ABSTRACT

Aspects of the invention relate to methods for increasing gene expression in a targeted manner. In some embodiments, methods and compositions are provided that are useful for posttranscriptionally altering protein and/or RNA levels in a targeted manner. Aspects of the invention disclosed herein provide methods and compositions that are useful for protecting RNAs from degradation (e.g., exonuclease mediated degradation).

### CROSS-REFERENCE TO RELATED APPLICATIONS

This application claims the benefit under 35 U.S.C. §119(e) of U.S. Provisional Application No. 62/010,417, entitled “COMPOSITIONS AND METHODS FOR MODULATING RNA STABILITY”, filed Jun. 10, 2014, of U.S. Provisional Application No. 61/898,461, entitled “COMPOSITIONS AND METHODS FOR MODULATING RNA STABILITY”, filed Oct. 31, 2013, and of U.S. Provisional Application No. 61/866,989, entitled “COMPOSITIONS AND METHODS FOR MODULATING RNA STABILITY”, filed Aug. 16, 2013, the contents of each of which are incorporated herein by reference in its entirety.

### FIELD OF THE INVENTION

The invention relates to oligonucleotide based compositions, as well as methods of using oligonucleotide based compositions for modulating nucleic acids.

### BACKGROUND OF THE INVENTION

A considerable portion of human diseases can be treated by selectively altering protein and/or RNA levels of disease-associated transcription units (noncoding RNAs, protein-coding RNAs or other regulatory coding or noncoding genomic regions). Methods for inhibiting the expression of genes are known in the art and include, for example, antisense, RNAi and miRNA mediated approaches. Such methods may involve blocking translation of mRNAs or causing degradation of target RNAs. However, limited approaches are available for increasing the expression of genes.

### SUMMARY OF THE INVENTION

Aspects of the invention disclosed herein relate to methods and compositions useful for modulating nucleic acids. In some embodiments, methods and compositions provided herein are useful for protecting RNAs (e.g., RNA transcripts) from degradation (e.g., exonuclease mediated degradation). In some embodiments, the protected RNAs are present outside of cells. In some embodiments, the protected RNAs are present in cells. In some embodiments, methods and compositions are provided that are useful for posttranscriptionally altering protein and/or RNA levels in a targeted manner. In some embodiments, methods disclosed herein involve reducing or preventing degradation or processing of targeted RNAs thereby elevating steady state levels of the targeted RNAs. In some embodiments, methods disclosed herein may also or alternatively involve increasing translation or increasing transcription of targeted RNAs, thereby elevating levels of RNA and/or protein levels in a targeted manner.

Aspects of the invention relate to a recognition that certain RNA degradation is mediated by exonucleases. In some embodiments, exonucleases may destroy RNA from its 3′ end and/or 5′ end. Without wishing to be bound by theory, in some embodiments, it is believed that one or both ends of RNA can be protected from exonuclease enzyme activity by contacting the RNA with oligonucleotides (oligos) that hybridize with the RNA at or near one or both ends, thereby increasing stability and/or levels of the RNA. The ability to increase stability and/or levels of a RNA by targeting the RNA at or near one or both ends, as disclosed herein, is surprising in part because of the presence of endonucleases (e.g., in cells) capable of destroying the RNA through internal cleavage. Moreover, in some embodiments, it is surprising that a 5′ targeting oligonucleotide is effective alone (e.g., not in combination with a 3′ targeting oligonucleotide or in the context of a pseudocircularization oligonucleotide) at stabilizing RNAs or increasing RNA levels because in cells, for example, 3′ end processing exonucleases may be dominant (e.g., compared with 5′ end processing exonucleases). However, in some embodiments, 3′ targeting oligonucleotides are used in combination with 5′ targeting oligonucleotides, or alone, to stabilize a target RNA.

In some embodiments, where a targeted RNA is protein-coding, increases in steady state levels of the RNA result in concomitant increases in levels of the encoded protein. Thus, in some embodiments, oligonucleotides (including 5′-targeting, 3′-targeting and pseudocircularization oligonucleotides) are provided herein that when delivered to cells increase protein levels of target RNAs. In some embodiments is notable that not only are target RNA levels increased but the resulting translation products are also increased. In some embodiments, this result is surprising in part because of an understanding that for translation to occur ribosomal machinery requires access to certain regions of the RNA (e.g., the 5′ cap region, start codon, etc.) to facilitate translation.

In some embodiments, where the targeted RNA is non-coding, increases in steady state levels of the non-coding RNA result in concomitant increases activity associated with the non-coding RNA. For example, in instances where the non-coding RNA is an miRNA, increases in steady state levels of the miRNA may result in increased degradation of mRNAs targeted by the miRNA.

In some embodiments, oligonucleotides are provided with chemistries suitable for delivery, hybridization and stability within cells to target and stabilize RNA transcripts. Furthermore, in some embodiments, oligonucleotide chemistries are provided that are useful for controlling the pharmacokinetics, biodistribution, bioavailability and/or efficacy of the oligonucleotides.

In some aspects of the invention, methods are provided for stabilizing a synthetic RNA (e.g., a synthetic RNA that is to be delivered to a cell). In some embodiments, the methods involve contacting a synthetic RNA with one or more oligonucleotides that bind to a 5′ region of the synthetic RNA and a 3′ region of the synthetic RNA and that when bound to the synthetic RNA form a circularized product with the synthetic RNA. In some embodiments, the synthetic RNA is contacted with the one or more oligonucleotides outside of a cell. In some embodiments, the methods further involve delivering the circularized product to a cell.

In some aspects of the invention, methods are provided for increasing expression of a protein in a cell that involve delivering to a cell a circularized synthetic RNA that encodes the protein, in which synthesis of the protein in the cell is increased following delivery of the circularized RNA to the cell. In some embodiments, the circularized synthetic RNA comprises one or more modified nucleotides. In some embodiments, methods are provided that involve delivering to a cell a circularized synthetic RNA that encodes a protein, in which synthesis of the protein in the cell is increased following delivery of the circularized synthetic RNA to the cell. In some embodiments, a circularized synthetic RNA is a single-stranded covalently closed circular RNA. In some embodiments, a single-stranded covalently closed circular RNA comprises one or more modified nucleotides. In some embodiments, the circularized synthetic RNA is formed by synthesizing an RNA that has a 5′ end and a 3′ and ligating together the 5′ and 3′ ends. In some embodiments, the circularized synthetic RNA is formed by producing a synthetic RNA (e.g., through in vitro transcription or artificial (non-natural) chemical synthesis) and contacting the synthetic RNA with one or more oligonucleotides that bind to a 5′ region of the synthetic RNA and a 3′ region of the synthetic RNA, and that when bound to the synthetic RNA form a circularized product with the synthetic RNA.

In some embodiments, methods for stabilizing a synthetic RNA are provided that involve contacting a synthetic RNA with a first stabilizing oligonucleotide that targets a 5′ region of the synthetic RNA and a second stabilizing oligonucleotide that targets the 3′ region of the synthetic RNA under conditions in which the first stabilizing oligonucleotide and second stabilizing oligonucleotide hybridize with target sequences on the synthetic RNA. In some embodiments, the first stabilizing oligonucleotide is covalently linked with the second stabilizing oligonucleotide such that the synthetic RNA when hybridized with the first and second stabilizing oligonucleotides forms a circularized product. In some embodiments, the synthetic RNA is contacted with the first and second stabilizing oligonucleotides outside of a cell.

In some embodiments, methods of delivering a synthetic RNA to a cell are provided that involve contacting a synthetic RNA with a first stabilizing oligonucleotide that targets a 5′ region of the synthetic RNA and a second stabilizing oligonucleotide that targets the 3′ region of the synthetic RNA under conditions in which the first stabilizing oligonucleotide and second stabilizing oligonucleotide hybridize with target sequences on the synthetic RNA; and delivering to the cell the circularized product. In some embodiments, the first stabilizing oligonucleotide is covalently linked with the second stabilizing oligonucleotide such that the synthetic RNA, when hybridized with the first and second stabilizing oligonucleotide, forms a circularized product. In some embodiments, the first stabilizing oligonucleotide and second stabilizing oligonucleotide are covalently linked through any appropriate linker disclosed herein (e.g., an oligonucleotide linker).

Aspects of the invention relate to methods of increasing stability of an RNA transcript in a cell. In some embodiments, methods provided herein involve delivering to a cell one or more oligonucleotides disclosed herein that stabilize an RNA transcript. In some embodiments, the methods involve delivering to a cell a first stabilizing oligonucleotide that targets a 5′ region of the RNA transcript and a second stabilizing oligonucleotide that targets the 3′ region of the RNA transcript. In some embodiments, the first stabilizing oligonucleotide is covalently linked with the second stabilizing oligonucleotide. In some embodiments, the first stabilizing oligonucleotide comprises a region of complementarity that is complementary with the RNA transcript at a position within 10 nucleotides of the first transcribed nucleotide at the 5′ end of the RNA transcript. In some embodiments, the RNA transcript comprises a 5′-methylguanosine cap, and the first stabilizing oligonucleotide comprises a region of complementarity that is complementary with the RNA transcript at a position within 10 nucleotides of the nucleotide immediately internal to the 5′-methylguanosine cap. In some embodiments, the second stabilizing oligonucleotide comprises a region of complementarity that is complementary with the RNA transcript at a position within 250 nucleotides of the 3′ end of the RNA transcript. In some embodiments, the RNA transcript comprises a 3′-poly(A) tail, and the second stabilizing oligonucleotide comprises a region of complementarity that is complementary with the RNA transcript at a position within 100 nucleotides of the polyadenylation junction of the RNA transcript. In some embodiments, the region of complementarity of the second stabilizing oligonucleotide is immediately adjacent to or overlapping the polyadenylation junction of the RNA transcript. In some embodiments, the cell is in vitro. In some embodiments, the cell is in vivo. In some embodiments, the second stabilizing oligonucleotide comprises a region of complementarity that is complementary with the RNA transcript at a position within the 3′-poly(a) tail. In some embodiments, the second stabilizing oligonucleotide comprises a region comprising 5 to 15 pyrimidine (e.g., thymine) nucleotides.

Further aspects of the invention relate to methods of treating a condition or disease associated with decreased levels of an RNA transcript in a subject. In some embodiments, the methods involve administering an oligonucleotide to the subject.

In some embodiments of the foregoing methods, the RNA transcript is an mRNA, non-coding RNA, long non-coding RNA, miRNA, snoRNA or any other suitable transcript.

In some embodiments, the RNA transcript is an mRNA expressed from a gene selected from the group consisting of: ABCA1, APOA1, ATP2A2, BDNF, FXN, HBA2, HBB, HBD, HBE1, HBG1, HBG2, SMN, UTRN, PTEN, MECP2, and FOXP3.

In some embodiments, the RNA transcript is an mRNA expressed from a gene selected from the group consisting of: ABCA4, ABCB11, ABCB4, ABCG5, ABCG8, ADIPOQ, ALB, APOE, BCL2L11, BRCA1, CD274, CEP290, CFTR, EPO, F7, F8, FLI1, FMR1, FNDC5, GCH1, GCK, GLP1R, GRN, HAMP, HPRT1, IDO1, IGF1, IL10, IL6, KCNMA1, KCNMB1, KCNMB2, KCNMB3, KCNMB4, KLF1, KLF4, LDLR, MSX2, MYBPC3, NANOG, NF1, NKX2-1, NKX2-1-AS1, PAH, PTGS2, RB1, RPS14, RPS19, SCARB1, SERPINF1, SIRT1, SIRT6, SMAD7, ST7, STAT3, TSIX, and XIST.

In some embodiments, the RNA transcript is a non-coding RNA selected from the group consisting of HOTAIR AND ANRIL.

In some embodiments, the RNA transcript is an mRNA expressed from a gene selected from the group consisting of: FXN, EPO, KLF4, ACTB, UTRN, HBF, SMN, FOXP3, PTEN, NFE2L2, and ATP2A2.

In some aspects of the invention, an oligonucleotide is provided that comprises a region of complementarity that is complementary with at least 5 contiguous nucleotides of an RNA transcript, in which the nucleotide at the 3′-end of the region of complementary is complementary with a nucleotide within 10 nucleotides of the transcription start site of the RNA transcript. In some embodiments, the oligonucleotide comprises nucleotides linked by at least one modified internucleoside linkage or at least one bridged nucleotide. In some embodiments, the oligonucleotide is 8 to 50 or 9 to 20 nucleotides in length.

In some aspects of the invention, an oligonucleotide is provided that comprises two regions of complementarity each of which is complementary with at least 5 contiguous nucleotides of an RNA transcript, in which the nucleotide at the 3′-end of the first region of complementary is complementary with a nucleotide within 100 nucleotides of the transcription start site of the RNA transcript and in which the second region of complementarity is complementary with a region of the RNA transcript that ends within 300 nucleotides of the 3′-end of the RNA transcript.

In some aspects of the invention, an oligonucleotide is provided that comprises the general formula 5′-X₁-X₂-3′, in which X₁ comprises 5 to 20 nucleotides that have a region of complementarity that is complementary with at least 5 contiguous nucleotides of an RNA transcript, in which the nucleotide at the 3′-end of the region of complementary of X₁ is complementary with the nucleotide at the transcription start site of the RNA transcript; and X₂ comprises 1 to 20 nucleotides. In some embodiments, the RNA transcript has a 7-methylguanosine cap at its 5′-end. In some embodiments, the RNA transcript has a 7-methylguanosine cap, and wherein the nucleotide at the 3′-end of the region of complementary of X₁ is complementary with the nucleotide of the RNA transcript that is immediately internal to the 7-methylguanosine cap. In some embodiments, at least the first nucleotide at the 5′-end of X₂ is a pyrimidine complementary with guanine. In some embodiments, the second nucleotide at the 5′-end of X₂ is a pyrimidine complementary with guanine. In some embodiments, X₂ comprises the formula 5′-Y₁-Y₂-Y₃-3′, in which X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, Y₁, Y₂ and Y₃ independently comprise 1 to 10 nucleotides. In some embodiments, Y₃ comprises, at a position immediately following the 3′-end of the stem region, a pyrimidine complementary with guanine. In some embodiments, Y₃ comprises 1-2 nucleotides following the 3′ end of the stem region. In some embodiments, the nucleotides of Y₃ following the 3′ end of the stem region are DNA nucleotides. In some embodiments, the stem region comprises 2-3 LNAs. In some embodiments, the pyrimidine complementary with guanine is cytosine. In some embodiments, the nucleotides of Y₂ comprise at least one adenine. In some embodiments, Y₂ comprises 3-4 nucleotides. In some embodiments, the nucleotides of Y₂ are DNA nucleotides. In some embodiments, Y₂ comprises 3-4 DNA nucleotides comprising at least one adenine nucleotide. It should be appreciated that one or more modified nucleotides (e.g., 2′-O-methyl, LNA nucleotides) may be present in Y₂. In some embodiments, X₂ comprises a region of complementarity that is complementary with at least 5 contiguous nucleotides of the RNA transcript that do not overlap the region of the RNA transcript that is complementary with the region of complementarity of X₁. In some embodiments, the region of complementarity of X₂ is within 100 nucleotides of a polyadenylation junction of the RNA transcript. In some embodiments, the region of complementarity of X₂ is complementary with the RNA transcript immediately adjacent to or overlapping the polyadenylation junction of the RNA transcript. In some embodiments, X₂ further comprises at least 2 consecutive pyrimidine nucleotides complementary with adenine nucleotides of the poly(A) tail of the RNA transcript. In some embodiments, the region of complementarity of X₂ is within the poly(a) tail. In some embodiments, the region of complementarity of X₂ comprises 5 to 15 pyrimidine (e.g., thymine) nucleotides. In some embodiments, the RNA transcript is an mRNA, non-coding RNA, long non-coding RNA, miRNA, snoRNA or any other suitable RNA transcript. In some embodiments, the RNA transcript is an mRNA transcript, and X₂ comprises a region of complementarity that is complementary with at least 5 contiguous nucleotides in the 3′-UTR of the transcript. In some embodiments, the RNA transcript is an mRNA expressed from a gene selected from the group consisting of: ABCA1, APOA1, ATP2A2, BDNF, FXN, HBA2, HBB, HBD, HBE1, HBG1, HBG2, SMN, UTRN, PTEN, MECP2, and FOXP3. In some embodiments, X₁ comprises the sequence 5′-CGCCCTCCAG-3′. In some embodiments, X₂ comprises the sequence CC. In some embodiments, X₂ comprises the sequence 5′-CCAAAGGTC-3′. In some embodiments, the oligonucleotide comprises the sequence 5′-CGCCCTCCAGCCAAAGGTC-3′. In some embodiments, the RNA transcript is an mRNA expressed from a gene selected from the group consisting of: ABCA4, ABCB11, ABCB4, ABCG5, ABCG8, ADIPOQ, ALB, APOE, BCL2L11, BRCA1, CD274, CEP290, CFTR, EPO, F7, F8, FLI1, FMR1, FNDC5, GCH1, GCK, GLP1R, GRN, HAMP, HPRT1, IDO1, IGF1, IL10, IL6, KCNMA1, KCNMB1, KCNMB2, KCNMB3, KCNMB4, KLF1, KLF4, LDLR, MSX2, MYBPC3, NANOG, NF1, NKX2-1, NKX2-1-AS1, PAH, PTGS2, RB1, RPS14, RPS19, SCARB1, SERPINF1, SIRT1, SIRT6, SMAD7, ST7, STAT3, TSIX, and XIST.

In some aspects of the invention, an oligonucleotide is provided that is 10 to 50 or 9 to 50 or 9 to 20 nucleotides in length and that has a first region complementary with at least 5 consecutive nucleotides of the 5′-UTR of an mRNA transcript, and a second region complementary with at least 5 consecutive nucleotides of the 3′-UTR, poly(A) tail, or overlapping the polyadenylation junction of the mRNA transcript. In some embodiments, the first of the at least 5 consecutive nucleotides of the 5′-UTR is within 10 nucleotides of the 5′-methylguanosine cap of the mRNA transcript. In some embodiments, the second region is complementary with at least 5 consecutive nucleotides overlapping the polyadenylation junction. In some embodiments, the second region is complementary with at least 5 consecutive nucleotides of the poly(a) tail. In some embodiments, the second region comprises 5 to 15 pyrimidine (e.g., thymine) nucleotides. In some embodiments, the oligonucleotide further comprises 2-20 nucleotides that link the 5′ end of the first region with the 3′ end of the second region. In some embodiments, the oligonucleotide further comprises 2-20 nucleotides that link the 3′ end of the first region with the 5′ end of the second region. In some embodiments, the oligonucleotide is 10 to 50 or 9 to 50 or 9 to 20 nucleotides in length.

In some aspects of the invention, an oligonucleotide is provided that comprises the general formula 5′-X₁-X₂-3′, in which X₁ comprises 2 to 20 pyrimidine nucleotides that form base pairs with adenine; and X₂ comprises a region of complementarity that is complementary with at least 3 contiguous nucleotides of a poly-adenylated RNA transcript, wherein the nucleotide at the 5′-end of the region of complementary of X₂ is complementary with the nucleotide of the RNA transcript that is immediately internal to the poly-adenylation junction of the RNA transcript. In some embodiments, X₁ comprises 2 to 20 thymidines or uridines.

In some embodiments, an oligonucleotide provided herein comprises at least one modified internucleoside linkage. In some embodiments, an oligonucleotide provided herein comprises at least one modified nucleotide. In some embodiments, at least one nucleotide comprises a 2′ O-methyl. In some embodiments, an oligonucleotide comprises at least one ribonucleotide, at least one deoxyribonucleotide, at least one 2′-fluoro-deoxyribonucleotides or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide. In some embodiments, the nucleotides of the oligonucleotide comprise alternating deoxyribonucleotides and 2′-fluoro-deoxyribonucleotides, 2′-O-methyl nucleotides, or bridged nucleotides. In some embodiments, an oligonucleotide provided herein is mixmer. In some embodiments, an oligonucleotide provided herein is morpholino.

In some aspects of the invention, an oligonucleotide is provided that comprises a nucleotide sequence as set forth in Table 3, 7, 8, or 9. In some aspects of the invention, an oligonucleotide is provided that comprises a fragment of at least 8 nucleotides of a nucleotide sequence as set forth in Table 3, 7, 8, or 9.

In some aspects of the invention, a composition is provided that comprises a first oligonucleotide having 5 to 25 nucleotides linked through internucleoside linkages, and a second oligonucleotide having 5 to 25 nucleotides linked through internucleoside linkages, in which the first oligonucleotide is complementary with at least 5 consecutive nucleotides within 100 nucleotides of the 5′-end of an RNA transcript and in which the second oligonucleotide is complementary with at least 5 consecutive nucleotides within 100 nucleotides of the 3′-end of an RNA transcript. In some embodiments, the first oligonucleotide and second oligonucleotide are joined by a linker that is not an oligonucleotide having a sequence complementary with the RNA transcript. In some embodiments, the linker is an oligonucleotide. In some embodiments, the linker is a polypeptide.

In some aspects of the invention, compositions are provided that comprise one or more oligonucleotides disclosed herein. In some embodiments, compositions are provided that comprise a plurality of oligonucleotides, in which each of at least 75% of the oligonucleotides comprise or consist of a nucleotide sequence as set forth in Table 3, 7, 8, or 9. In some embodiments, the oligonucleotide is complexed with a monovalent cation (e.g., Li+, Na+, K+, Cs+). In some embodiments, the oligonucleotide is in a lyophilized form. In some embodiments, the oligonucleotide is in an aqueous solution. In some embodiments, the oligonucleotide is provided, combined or mixed with a carrier (e.g., a pharmaceutically acceptable carrier). In some embodiments, the oligonucleotide is provided in a buffered solution. In some embodiments, the oligonucleotide is conjugated to a carrier (e.g., a peptide, steroid or other molecule). In some aspects of the invention, kits are provided that comprise a container housing the composition.

### BRIEF DESCRIPTION OF THE FIGURES

FIG. 1 is an illustration depicting exemplary oligo designs for targeting 3′ RNA ends. The first example shows oligos complementary to the 3′ end of RNA, before the polyA-tail. The second example shows oligos complementary to the 3′ end of RNA with a 5′ T-stretch to hybridize to a polyA tail.

FIG. 2 is an illustration depicting exemplary oligos for targeting 5′ RNA ends. The first example shows oligos complementary to the 5′ end of RNA. The second example shows oligos complementary to the 5′ end of RNA, the oligo having 3′ overhang residues to create a RNA-oligo duplex with a recessed end. Overhang can include a combination of nucleotides including, but not limited to, C to potentially interact with a 5′ methylguanosine cap and stabilize the cap further.

FIG. 3A is an illustration depicting exemplary oligos for targeting 5′ RNA ends and exemplary oligos for targeting 5′ and 3′ RNA ends. The example shows oligos with loops to stabilize a 5′ RNA cap or oligos that bind to a 5′ and 3′ RNA end to create a pseudo-circularized RNA.

FIG. 3B is an illustration depicting exemplary oligo-mediated RNA pseudo-circularization. The illustration shows an LNA mixmer oligo binding to the 5′ and 3′ regions of an exemplary RNA.

FIG. 4 is a diagram depicting Frataxin (FXN) 3′ polyA sites.

FIG. 5 is a diagram depicting FXN 5′ start sites.

FIG. 6 is a diagram depicting the location of the 5′ and 3′ oligonucleotides tested in the Examples.

FIG. 7 is a graph depicting the results of testing 3′ end oligos. The screen was performed in a GM03816 FRDA patient cell line and the level of FXN mRNA was measured at 1-3 days post-transfection. Oligo concentration used for transfection was 100 nM.

FIG. 8 is a graph depicting the results of testing 3′ end oligos. The screen was performed in a GM03816 FRDA patient cell line and the level of FXN mRNA was measured at 1-3 days post-transfection. Oligo concentration used for transfection was 400 nM.

FIG. 9 is a diagram depicting the location and sequences of FXN 3′ oligos 73, 75, 76, and 77, which were shown to upregulate FXN mRNA. The oligos all contained poly-T sequences. A schematic of the binding of each oligo to the mRNA is shown.

FIG. 10 is a graph depicting the results of testing 5′ end oligos. The screen was performed in a GM03816 FRDA patient cells and the level of FXN mRNA was measured at 2 days post-transfection. Oligo concentrations used for transfection were 100 nM (red bars, left bar in each pair) and 400 nM (blue bars, right bar in each pair). The lower response levels obtained with 400 nM level may be due to the oligo concentration being too high and reducing the transfection agent availability to properly coat each oligo for delivery.

FIG. 11 is a graph depicting the results of testing 5′ end oligos in combination with FXN 3′ oligo 75 in GM03816 FRDA patient cells. The level of FXN mRNA was measured at 2 and 3 days post-transfection. For Oligo A/B, Oligo A targets the 5′ end and OligoB targets the 3′ end. Oligo concentration used for transfection was 200 nM final=100 nM oligo A+100 nM oligo B).

FIG. 12 shows the same graph presented in FIG. 8. The boxes around bars indicate the 5′ and 3′ oligo pairs that were particularly effective in upregulating FXN in in GM03816 FRDA patient cells.

FIG. 13 is a diagram depicting the location and sequences of FXN 5′ oligos 51, 52, 57, and 62, which were shown to upregulate FXN mRNA. The oligos all contained the motif CGCCCTCCAG. A schematic of a stem-loop structure formed by oligo 62 is shown.

FIG. 14 is an illustration depicting the predicted structure of FXN oligo 62. Nucleotides 1-15 are complementary to the 5′ end of one of the FXN isoforms. The predicted loop shown in nucleotides 2-8 may not exist in the cells because this portion will hybridize to the RNA and thus the loop will open up and hybridize to RNA. Nucleotides 16-24 are the artificially added loop to place the 3′ most C residue in close proximity to the 5′ methylguanosine cap of FXN mRNA.

FIGS. 15A and 15B are graphs depicting cytoxicity (CTG) at two days of treatment. Treatment of the FRDA patient cell line GM03816 with oligos did not result in cytotoxicity during day 2 (FIG. 15A) and 3 (FIG. 15B) of oligo treatment at 100 and 400 nM.

FIG. 16 is a set of graphs showing testing of combinations of oligos from previous experiments in the GM03816 FRDA patient cell line. The FXN mRNA levels for several of the oligos approached the levels of FXN mRNA in the GM0321B normal fibroblast cells. For Oligo A/B, Oligo A targets the 5′ end and OligoB targets the 3′ end. Oligo concentration used for transfection was 200 nM final=100 nM oligo A+100 nM oligo B).

FIG. 17 is a graph depicting the levels of FXN mRNA at two and three days of treatment with oligos. Biological replicates of positive hits in previous experiments in GM03816 FRDA patient cells confirmed increased steady state FXN mRNA levels at 2-3 days. For Oligo A/B, Oligo A targets the 5′ end and OligoB targets the 3′ end. Oligo concentration used for transfection was 200 nM final=100 nM oligo A+100 nM oligo B).

FIG. 18 is a graph depicting testing of oligos in GM04078 FRDA patient fibroblasts.

FIG. 19 is a graph depicting testing of oligos in a ‘normal’ cell line, GM0321B fibroblasts. GM0321B cells express approximately 4-fold more FXN mRNA than FRDA patient cells

FIG. 20 is a graph depicting transfection dose-response testing for 5′ and 3′ FXN oligo combination 62/77. Biological replicates and doses response of FXN Oligo 62/77 combination in GM03816 FRDA patient cell line showed increased steady-state FXN mRNA levels in 2-3 days. For Oligo A/B, Oligo A targets the 5′ end and OligoB targets the 3′ end. The transfection reagent amount was kept constant across the different concentration of oligos, which may be the cause of relatively flat response to oligo treatment. Concentrations are in nM final (i.e. 10 nM final=5 nM oligo 62+5 nM oligo77).

FIG. 21 is a graph depicting FXN protein levels in GM03816 FRDA patient fibroblasts treated with oligos (single oligos at 100 nM) or in combination (two oligos at 200 nM final) and FXN protein levels in GM0321B normal fibroblasts.

FIG. 22 is a graph depicting levels of FXN protein with oligo treatment. FXN protein (100 nM, d3) n=2.

FIGS. 23A and 23B are graphs depicting the relative levels of mRNA with and without treatment with a combination of oligos 62 and 75 (also referred to, respectively, as oligos 385 and 398) in the presence of the de novo transcription inhibitor Actinomycin D (ActD). FIG. 23A depicts relative levels of MYC mRNA. FIG. 22B depicts relative levels of FXN mRNA. cMyc has a relatively short half-life (˜100 minutes) and was used as a positive control for ActD treatment.

FIG. 24 is a graph depicting oligos in GM03816 cells treated with Actinomycin D (ActD). FXN expression is depicted at 0, 2, 4 and 8 hours.

FIGS. 25A and 25B are graphs depicting FXN mRNA levels in GM15850 & GM15851 cells (FIG. 25A) or GM16209 & GM16222 (FIG. 25B) treated with combinations of 5′ and 3′ FXN oligos. This was a gymnotic experiment, with 10 micromolar of oligonucleotide.

FIG. 26 is a graph showing that treating cells with a combination of 5′ end targeting oligos, and 3′ end targeting oligos, and other FXN targeting oligos increases FXN mRNA levels.

FIG. 27 is a series of graphs showing the screening of 3′ end oligos. Cells were transfected with 10 or 40 nM of an oligo and FXN mRNA was measured at 2 days post-transfection.

FIG. 28 is a series of graphs showing the screening of 3′ end oligos. Cells were transfected with 10 or 40 nM of an oligo and FXN mRNA was measured at 3 days post-transfection.

FIG. 29 is a graph and a table showing the screening of 5′ end oligos. Cells were transfected with 10 or 40 nM of an oligo and FXN mRNA was measured at 2 days post-transfection.

FIG. 30 is a series of graphs showing the testing of combinations of 5′ and 3′ end oligos. Cells were transfected with 10 or 40 nM of an oligo combination and FXN mRNA was measured at 2 days post-transfection.

FIG. 31 is a series of graphs showing the testing of combinations of 5′ and 3′ end oligos. Cells were transfected with 10 or 40 nM of an oligo combination and FXN mRNA was measured at 3 days post-transfection.

FIG. 32 is a graph showing that steady state levels of FXN mRNA increase over time in cells treated with combinations of 5′ and 3′ end oligos. Cells were transfected with 10 nM of an oligo combination and FXN mRNA was measured at 2 and 3 days post-transfection.

FIG. 33 is a graph showing that steady state levels of FXN mRNA increase over time in cells treated with combinations of 5′ and 3′ end oligos. Cells were transfected with 40 nM of an oligo combination and FXN mRNA was measured at 2 and 3 days post-transfection.

FIG. 34 is a graph showing the results from a testing of other oligos that target FXN, e.g., internally, close to a poly-A tail, or spanning an exon.

FIG. 35 is a graph showing that FXN mRNA levels are increased using a single oligonucleotide. Cells were transfected with 10 nM of an oligo and FXN mRNA was measured at 2 and 3 days post-transfection.

FIG. 36 is a graph showing that FXN mRNA levels are increased using a single oligonucleotide. Cells were transfected with 40 nM of an oligo and FXN mRNA was measured at 2 and 3 days post-transfection.

FIG. 37 is a graph showing that FXN mRNA levels are increased using combinations of 5′ and 3′ oligonucleotides. Cells were transfected with 10 or 40 nM of an oligo combination and FXN mRNA was measured at 2 and 3 days post-transfection.

FIGS. 38A and 38B are graphs showing that transfection with 10 or 40 nM of an oligo is not cytoxic to the cells at day 2 (FIG. 38A) or day 3 (FIG. 38B) post-transfection.

FIGS. 39A and 39B are graphs showing that FXN protein levels (FIG. 39A) and mRNA levels (FIG. 39B) are increased in cells transfected with 10 nM of an oligo. Protein and mRNA levels were measured 2 or 3 days post-transfection.

FIGS. 40A and 40B are graphs showing that FXN protein levels (FIG. 40A) and mRNA levels (FIG. 40B) can be increased in cells transfected with 40 nM of an oligo. Protein and mRNA levels were measured 2 or 3 days post-transfection.

FIG. 41 is a graph depicting the expression level of KLF4 mRNA in cells treated with KLF4 5′ and 3′ end targeting oligos.

FIG. 42 is an image of a Western blot depicting the expression level of KLF4 protein in cells treated with KLF4 5′ and 3′ end targeting oligos.

FIG. 43 is a graph depicting the expression level of KLF4 mRNA in cells treated with KLF4 5′ and 3′ end targeting oligos, including circularized oligonucleotides targeting both 5′ and 3′ ends of KLF4, and individual oligonucleotides targeting 5′ and 3′ ends of KLF4.

FIGS. 44A and 44B are graphs depicting the expression level of PTEN mRNA at day3 in cells treated with PTEN oligos. GM04078 fibroblast cells were transfected with the oligos and lysates were collected at day3. Oligo sequences are provided in Table 9.

FIG. 45 is an image of a Western blot depicting the expression level of PTEN protein at day1 and day2 from GM04078 fibroblast cells treated with PTEN oligos PTEN-108 and PTEN-113, either alone or in combination. GM04078 fibroblast cells were transfected and lysates were collected at day1 & day2. Oligo sequences are provided in Table 9.

FIG. 46 is a graph depicting the expression level of mouse KLF4 mRNA at day3 in cells treated with KLF4 oligos. Hepal-6 cells were transfected with the oligos and lysate was collected at day3. Oligo sequences are provided in Table 9.

FIG. 47 is an image of a Western blot depicting the expression level of mouse KLF4 protein at day3 in cells treated with pseudo-circularization oligos. Hepal-6 cells were transfected with the oligos and lysate was collected at day3. The oligos tested were mouse KLF4-8, KLF4-9, KLF4-11, KLF4-12, KLF4-13, KLF4-14, and KLF4-15. Oligo sequences are provided in Table 9.

FIG. 48 is an image of a Western blot depicting the expression level of mouse KLF4 protein at day3 in cells treated with stability combination oligos. Hepal-6 cells were transfected with the oligos and lysate was collected at day3. The oligos tested were mouse KLF4-1, KLF4-2, KLF4-3, KLF4-16, KLF4-17, KLF4-18, and KLF4-19, in various combinations. Oligo sequences are provided in Table 9.

FIG. 49 is a graph showing human KLF4 stability measurements in the presence of absence of circularization and individual stability oligos used alone or in combination (indicated by “/”). Oligo sequences are provided in Table 7. 47=KLF4-47 m02, 48=KLF4-48 m02, 50=KLF4-50 m02, 51=KLF4-51 m02, 53=KLF4-53 m02.

FIG. 50 is a graph showing that 5′/3′ end oligo combinations and circularization oligos can be used to increase beta actin mRNA, which is known to have a long mRNA half-life.

FIG. 51 is a graph showing human FXN mRNA upregulation in GM03816 cells treated with FXN oligos either alone or in various combinations. Concentrations are indicated as total oligo concentration (e.g. 20 nM means 10 nM for each oligo).

FIGS. 52 and 53 are each a photograph of a Western blot showing protein levels of premature and mature FXN induced by various FXN oligos.

FIG. 54 is a series of graphs showing FXN mRNA upregulation in GM03816 cells treated with FXN oligos either alone or in various combinations. GAPDH gapmer values show GAPDH mRNA levels relative to FXN mRNA level. The rest of the values show FXN mRNA levels relative to GAPDH mRNA levels.

FIG. 55 a graph showing FXN mRNA upregulation in GM03816 cells treated with FXN oligos either alone or in various combinations. GAPDH gapmer values show GAPDH mRNA levels relative to FXN mRNA level. The rest of the values show FXN mRNA levels relative to GAPDH mRNA levels.

FIG. 56 provides a series of graphs showing mRNA levels of PPARGC1 and NFE2L2, candidate FXN downstream genes, in cells treated with various FXN oligos alone or in combination.

FIG. 57 is a graph showing FXN mRNA upregulation in GM03816 cells treated with FXN oligos either alone or in various combinations.

FIGS. 58A-58C are a series of graphs showing levels of FXN mRNA at day 4, day 7, and day 10, respectively, in FRDA mouse model fibroblasts treated with various FXN oligos alone or in combination.

FIGS. 59A and 59B are a series of graphs showing FXN mRNA levels in GM03816 cells treated with various FXN oligos in a dose-response study. For FIG. 59A, measurement was done at day3 and day5. For FIG. 59B, measurement was done at day5.

FIGS. 60A and 60B are a series of graphs showing levels of FXN mRNA in GM03816 cells treated with various 5′ FXN oligos combined with the FXN-532 oligo.

FIG. 61 is a photograph of a Western blot showing the levels of FXN protein in GM03816 cells treated with various FXN oligos.

FIG. 62 is a graph showing levels of UTRN protein quantified from the Western blot in FIG. 64.

FIG. 63 is a photograph of a Western blot showing the levels of UTRN protein in the supernatant from cells treated with various UTRN oligos.

FIG. 64A is a graph showing levels of UTRN protein quantified from the Western blot in FIGS. 64B and 64C. FIGS. 64B and 64C are each photographs of Western blots showing the levels of UTRN protein in the supernatant or pellet from cells treated with various UTRN oligos.

FIGS. 65A-65C are a series of graphs showing the level of mouse APOA1 mNRA levels in primary mouse hepatocytes treated with various APOA1 oligos.

FIG. 66 is a photograph of two Western blots showing the levels of APOA1 protein in primary mouse hepatocytes treated with various APOA1 oligos. Tubulin was used as loading control for the bottom photograph.

FIGS. 67A-67G are a series of graphs showing the level of Human Frataxin (A, B, E) or mouse Frataxin in a short arm (SA) or long arm (LA) study of oligo treatment in a mouse model of Friedreich's ataxia. FIGS. 67A-67E show heart data. FIGS. 67F & 67G show liver data. FIGS. 67C and 67E show the same long-arm heart human F×N values by averaging across the 5 mice in each group (FIG. 67C) and showing values in each individual mouse in the groups (FIG. 67E). The human FXN and mouse FXN in the hearts and livers of this model were measured with QPCR and normalized to the PBS group. Each treatment group had 5 mice (n=5).

FIG. 68 shows a series of diagrams that demonstrate the potential targeting of human FXN oligos to mouse FXN. The diagrams on the left show USCS genome views of mouse FXN genomic regions corresponding to human FXN-375 (top panels) and FXN-389 (bottom panels) potential interaction locations. The boxes show the oligos' mapping position relative to the mouse genome. The panels on the right show ClustalW alignment of human oligo sequences to the mouse genome.

FIG. 69 is a series of diagrams showing oligo positions relative to mRNA-Seq signal and ribosome positioning. The signal in the top panel of each diagram shows all ribosome positioning data (including initiating and elongating ribosomes). The signal in the bottom panel of each diagram shows mRNA-Seq data. The black bars in boxes show indicated oligo localization.

FIGS. 70A and 70B are a series of graphs showing APOA1 mRNA levels in the livers of mice treated with various 5′ and 3′ end APOA1 oligos. For FIG. 70A, collection of livers was done at day5, 2 days after the last dose of oligos or control (PBS). For FIG. 70B, collection of livers was done at day7, 4 days after the last dose of oligos or control (PBS).

FIGS. 70C and 70D are photographs of Western blots showing APOA1 protein levels in mice treated with various 5′ and 3′ end APOA1 oligos. For FIG. 70C, samples 1-5 are PBS-treated animals and samples 6-10 are from APOA1_mus-3+APOA1_mus-17 oligo-treated animals. Lane 10 blood sample, indicated by a star, contained hemolysis and therefore was omitted from analysis. For FIG. 70D, samples 1-5 are PBS-treated animals and samples 6-10 are from APOA1_mus-7+APOA1_mus-20 oligo-treated animals. The top blot in FIG. 70D shows pre-bleeding data from all 10 animals. The bottom plot shows plasma APOA1 levels after oligo treatment. Control treated sample 4 died during the study and therefore was omitted from the blot.

### DETAILED DESCRIPTION OF CERTAIN EMBODIMENTS OF THE INVENTION

Methods and compositions disclosed herein are useful in a variety of different contexts in which is it desirable to protect RNAs from degradation, including protecting RNAs inside or outside of cells. In some embodiments, methods and compositions are provided that are useful for posttranscriptionally altering protein and/or RNA levels in cells in a targeted manner. For example, methods are provided that involve reducing or preventing degradation or processing of targeted RNAs thereby elevating steady state levels of the targeted RNAs. In some embodiments, the stability of an RNA is increased by protecting one or both ends (5′ or 3′ ends) of the RNA from exonuclease activity, thereby increasing stability of the RNA.

In some embodiments, methods of increasing gene expression are provided. As used herein the term, “gene expression” refers generally to the level or representation of a product of a gene in a cell, tissue or subject. It should be appreciated that a gene product may be an RNA transcript or a protein, for example. An RNA transcript may be protein coding. An RNA transcript may be non-protein coding, such as, for example, a long non-coding RNA, a long intergenic non-coding RNA, a non-coding RNA, an miRNA, a small nuclear RNA (snRNA), or other functional RNA. In some embodiments, methods of increasing gene expression may involve increasing stability of a RNA transcript, and thereby increasing levels of the RNA transcript in the cell. Methods of increasing gene expression may alternatively or in addition involve increasing transcription or translation of RNAs. In some embodiments, other mechanisms of manipulating gene expression may be involved in methods disclosed herein.

In some embodiments, methods provided herein involve delivering to a cell one or more sequence specific oligonucleotides that hybridize with an RNA transcript at or near one or both ends, thereby protecting the RNA transcript from exonuclease mediated degradation. In embodiments where the targeted RNA transcript is protein-coding, increases in steady state levels of the RNA typically result in concomitant increases in levels of the encoded protein. In embodiments where the targeted RNA is non-coding, increases in steady state levels of the non-coding RNA typically result in concomitant increases activity associated with the non-coding RNA.

In some embodiments, approaches disclosed herein based on regulating RNA levels and/or protein levels using oligonucleotides targeting RNA transcripts by mechanisms that increase RNA stability and/or translation efficiency may have several advantages over other types of oligos or compounds, such as oligonucleotides that alter transcription levels of target RNAs using cis or noncoding based mechanisms. For example, in some embodiments, lower concentrations of oligos may be used when targeting RNA transcripts in the cytoplasm as multiple copies of the target molecules exist. In contrast, in some embodiments, oligos that target transcriptional processes may need to saturate the cytoplasm and before entering nuclei and interacting with corresponding genomic regions, of which there are only one/two copies per cell, in many cases. In some embodiments, response times may be shorter for RNA transcript targeting because RNA copies need not to be synthesized transcriptionally. In some embodiments, a continuous dose response may be easier to achieve. In some embodiments, well defined RNA transcript sequences facilitate design of oligonucleotides that target such transcripts. In some embodiments, oligonucleotide design approaches provided herein, e.g., designs having sequence overhangs, loops, and other features facilitate high oligo specificity and sensitivity compared with other types of oligonucleotides, e.g., certain oligonucleotides that target transcriptional processes.

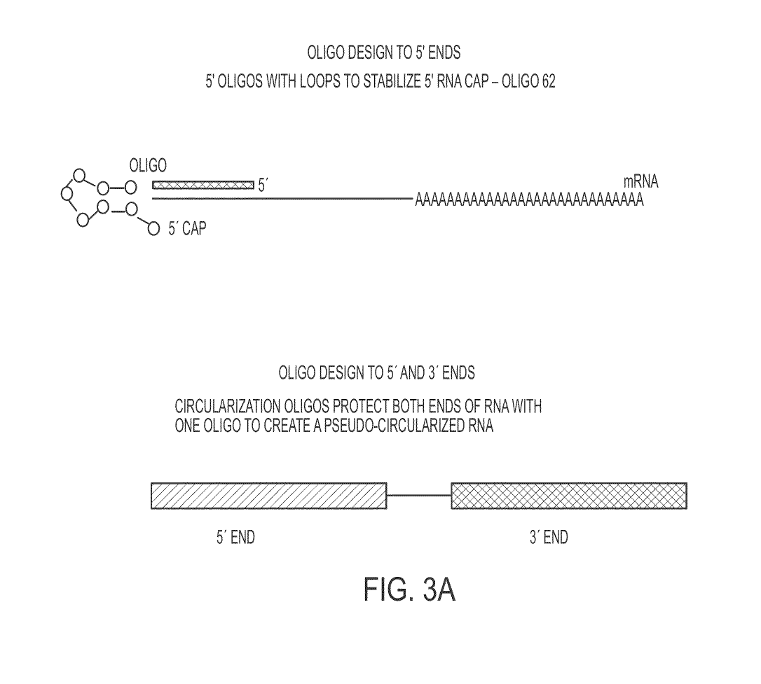

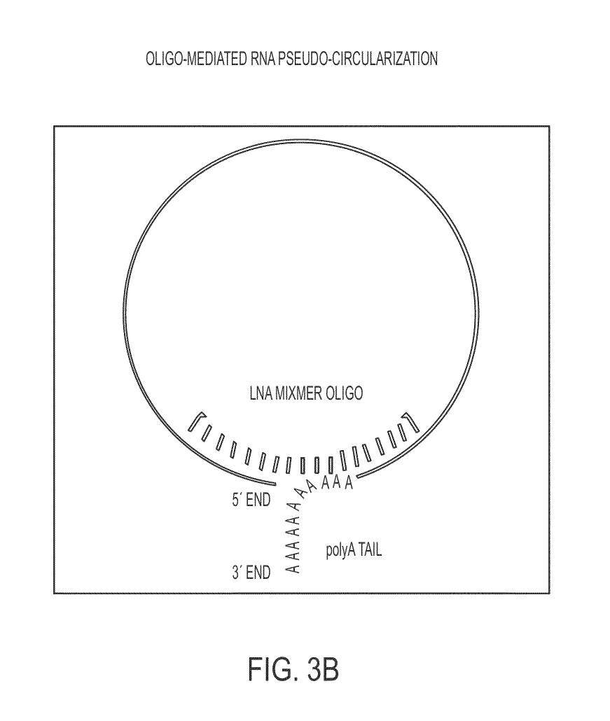

In some embodiments, methods provided herein involve use of oligonucleotides that stabilize an RNA by hybridizing at a 5′ and/or 3′ region of the RNA. In some embodiments, oligonucleotides that prevent or inhibit degradation of an RNA by hybridizing with the RNA may be referred to herein as “stabilizing oligonucleotides.” In some examples, such oligonucleotides hybridize with an RNA and prevent or inhibit exonuclease mediated degradation Inhibition of exonuclease mediated degradation includes, but is not limited to, reducing the extent of degradation of a particular RNA by exonucleases. For example, an exonuclease that processes only single stranded RNA may cleave a portion of the RNA up to a region where an oligonucleotide is hybridized with the RNA because the exonuclease cannot effectively process (e.g., pass through) the duplex region. Thus, in some embodiments, using an oligonucleotide that targets a particular region of an RNA makes it possible to control the extent of degradation of the RNA by exonucleases up to that region. For example, use of an oligonucleotide that hybridizes at an end of an RNA may reduce or eliminate degradation by an exonuclease that processes only single stranded RNAs from that end. For example, use of an oligonucleotide that hybridizes at the 5′ end of an RNA may reduce or eliminate degradation by an exonuclease that processes single stranded RNAs in a 5′ to 3′ direction. Similarly, use of an oligonucleotide that hybridizes at the 3′ end of an RNA may reduce or eliminate degradation by an exonuclease that processes single stranded RNAs in a 3′ to 5′ direction. In some embodiments, lower concentrations of an oligo may be used when the oligo hybridizes at both the 5′ and 3′ regions of the RNA. In some embodiments, an oligo that hybridizes at both the 5′ and 3′ regions of the RNA protects the 5′ and 3′ regions of the RNA from degradation (e.g., by an exonuclease). In some embodiments, an oligo that hybridizes at both the 5′ and 3′ regions of the RNA creates a pseudo-circular RNA (e.g., a circularized RNA with a region of the poly A tail that protrudes from the circle, see FIG. 3B). In some embodiments, a pseudo-circular RNA is translated at a higher efficiency than a non-pseudo-circular RNA.

In some embodiments, an oligonucleotide may be used that comprises multiple regions of complementarity with an RNA, such that at one region the oligonucleotide hybridizes at or near the 5′ end of the RNA and at another region it hybridizes at or near the 3′ end of the RNA, thereby preventing or inhibiting degradation of the RNA by exonucleases at both ends. In some embodiments, when an oligonucleotide hybridizes both at or near the 5′ end of an RNA and at or near the 3′ end of the RNA a circularized complex results that is protected from exonuclease mediated degradation. In some embodiments, when an oligonucleotide hybridizes both at or near the 5′ end of an mRNA and at or near the 3′ end of the mRNA, the circularized complex that results is protected from exonuclease mediated degradation and the mRNA in the complex retains its ability to be translated into a protein. As used herein the term, “synthetic RNA” refers to a RNA produced through an in vitro transcription reaction or through artificial (non-natural) chemical synthesis. In some embodiments, a synthetic RNA is an RNA transcript. In some embodiments, a synthetic RNA encodes a protein. In some embodiments, the synthetic RNA is a functional RNA (e.g., a 1 ncRNA, miRNA, etc.). In some embodimentst, a synthetic RNA comprises one or more modified nucleotides. In some embodiments, a synthetic RNA is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a synthetic RNA is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

As used herein, the term “RNA transcript” refers to an RNA that has been transcribed from a nucleic acid by a polymerase enzyme. An RNA transcript may be produced inside or outside of cells. For example, an RNA transcript may be produced from a DNA template encoding the RNA transcript using an in vitro transcription reaction that utilizes recombination or purified polymerase enzymes. An RNA transcript may also be produced from a DNA template (e.g., chromosomal gene, an expression vector) in a cell by an RNA polymerase (e.g., RNA polymerase I, II, or III). In some embodiments, the RNA transcript is a protein coding mRNA. In some embodiments, the RNA transcript is a non-coding RNA (e.g., a tRNA, rRNA, snoRNA, miRNA, ncRNA, long-noncoding RNA, shRNA). In some embodiments, RNA transcript is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a RNA transcript is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

In some embodiments, the RNA transcript is capped post-transcriptionally, e.g., with a 7′-methylguanosine cap. In some embodiments, the 7′-methylguanosine is added to the RNA transcript by a guanylyltransferase during transcription (e.g., before the RNA transcript is 20-50 nucleotides long.) In some embodiments, the 7 ‘-methylguanosine is linked to the first transcribed nucleotide through a 5’-5′ triphosphate bridge. In some embodiments, the nucleotide immediately internal to the cap is an adenosine that is N6 methylated. In some embodiments, the first and second nucleotides immediately internal to the cap of the RNA transcript are not 2′-O-methylated. In some embodiments, the first nucleotide immediately internal to the cap of the RNA transcript is 2′-O-methylated. In some embodiments, the second nucleotide immediately internal to the cap of the RNA transcript is 2′-O-methylated. In some embodiments, the first and second nucleotides immediately internal to the cap of the RNA transcript are 2′-O-methylated.

In some embodiments, the RNA transcript is a non-capped transcript (e.g., a transcript produced from a mitochondrial gene). In some embodiments, the RNA transcript is a nuclear RNA that was capped but that has been decapped. In some embodiments, decapping of an RNA is catalyzed by the decapping complex, which may be composes of Dcp1 and Dcp2, e.g., that may compete with eIF-4E to bind the cap. In some embodiments, the process of RNA decapping involves hydrolysis of the 5′ cap structure on the RNA exposing a 5′ monophosphate. In some embodiments, this 5′ monophosphate is a substrate for the exonuclease XRN1. Accordingly, in some embodiments, an oligonucleotide that targets the 5′ region of an RNA may be used to stabilize (or restore stability) to a decapped RNA, e.g., protecting it from degradation by an exonuclease such as XRN1.

In some embodiments, in vitro transcription (e.g., performed via a T7 RNA polymerase or other suitable polymerase) may be used to produce an RNA transcript. In some embodiments transcription may be carried out in the presence of anti-reverse cap analog (ARCA) (TriLink Cat. # N-7003). In some embodiments, transcription with ARCA results in insertion of a cap (e.g., a cap analog (mCAP)) on the RNA in a desirable orientation.

In some embodiments, transcription is performed in the presence of one or more modified nucleotides (e.g., pseudouridine, 5-methylcytosine, etc.), such that the modified nucleotides are incorporated into the RNA transcript. It should be appreciated that any suitable modified nucleotide may be used, including, but not limited to, modified nucleotides that reduced immune stimulation, enhance translation and increase nuclease stability. Non-limiting examples of modified nucleotides that may be used include: 2′-amino-2′-deoxynucleotide, 2′-azido-2′-deoxynucleotide, 2′-fluoro-2′-deoxynucleotide, 2′-O-methyl-nucleotide, 2′ sugar super modifier, 2′-modified thermostability enhancer, 2′-fluoro-2′-deoxyadenosine-5′-triphosphate, 2′-fluoro-2′-deoxycytidine-5′-triphosphate, 2′-fluoro-2′-deoxyguanosine-5′-triphosphate, 2′-fluoro-2′-deoxyuridine-5′-triphosphate, 2′-O-methyladenosine-5′-triphosphate, 2′-O-methylcytidine-5′-triphosphate, 2′-O-methylguanosine-5′-triphosphate, 2′-O-methyluridine-5′-triphosphate, pseudouridine-5′-triphosphate, 2′-O-methylinosine-5′-triphosphate, 2′-amino-2′-deoxycytidine-5′-triphosphate, 2′-amino-2′-deoxyuridine-5′-triphosphate, 2′-azido-2′-deoxycytidine-5′-triphosphate, 2′-azido-2′-deoxyuridine-5′-triphosphate, 2′-O-methylpseudouridine-5′-triphosphate, 2′-O-methyl-5-methyluridine-5′-triphosphate, 2′-azido-2′-deoxyadenosine-5′-triphosphate, 2′-amino-2′-deoxyadenosine-5′-triphosphate, 2′-fluoro-thymidine-5′-triphosphate, 2′-azido-2′-deoxyguanosine-5′-triphosphate, 2′-amino-2′-deoxyguanosine-5′-triphosphate, and N4-methylcytidine-5′-triphosphate. In one embodiment, RNA degradation or processing can be reduced/prevented to elevate steady state RNA and, at least for protein-coding transcripts, protein levels. In some embodiments, a majority of degradation of RNA transcripts is done by exonucleases. In such embodiments, these enzymes start destroying RNA from either their 3′ or 5′ ends. By protecting the ends of the RNA transcripts from exonuclease enzyme activity, for instance, by hybridization of sequence-specific blocking oligonucleotides with proper chemistries for proper delivery, hybridization and stability within cells, RNA stability may be increase, along with protein levels for protein-coding transcripts.

In some embodiments, for the 5′ end, oligonucleotides may be used that are fully/partly complementary to 10-20 nts of the RNA 5′ end. In some embodiments, such oligonucleotides may have overhangs to form a hairpin (e.g., the 3′ nucleotide of the oligonucleotide can be, but not limited to, a C to interact with the mRNA 5′ cap's G nucleoside) to protect the RNA 5′ cap. In some embodiments, all nucleotides of an oligonucleotide may be complementary to the 5′ end of an RNA transcript, with or without few nucleotide overhangs to create a blunt or recessed 5′RNA-oligo duplex. In some embodiments, for the 3′ end, oligonucleotides may be partly complementary to the last several nucleotides of the RNA 3′ end, and optionally may have a poly(T)-stretch to protect the poly(A) tail from complete degradation (for transcripts with a poly(A)-tail). In some embodiments, similar strategies can be employed for other RNA species with different 5′ and 3′ sequence composition and structure (such as transcripts containing 3′ poly(U) stretches or transcripts with alternate 5′ structures). In some embodiments, oligonucleotides as described herein, including, for example, oligonucleotides with overhangs, may have higher specificity and sensitivity to their target RNA end regions compared to oligonucleotides designed to be perfectly complementary to RNA sequences, because the overhangs provide a destabilizing effect on mismatch regions and prefer binding in regions that are at the 5′ or 3′ ends of the RNAs. In some embodiments, oligonucleotides that protect the very 3′ end of the poly(A) tail with a looping mechanism (e.g., TTTTTTTTTTGGTTTTCC, SEQ ID NO: 458). In some embodiments, this latter approach may nonspecifically target all protein-coding transcripts. However, in some embodiments, such oligonucleotides, may be useful in combination with other target-specific oligos.

In some embodiments, methods provided herein involve the use of an oligonucleotide that comprises a region of complementarity that is complementary with the RNA transcript at a position at or near the first transcribed nucleotide of the RNA transcript. In some embodiments, an oligonucleotide (e.g., an oligonucleotide that stabilizes an RNA transcript) comprises a region of complementarity that is complementary with the RNA transcript (e.g., with at least 5 contiguous nucleotides) at a position that begins within 100 nucleotides, within 50 nucleotides, within 30 nucleotides, within 20 nucleotides, within 10 nucleotides or within 5 nucleotides of the 5′-end of the transcript. In some embodiments, an oligonucleotide (e.g., an oligonucleotide that stabilizes an RNA transcript) comprises a region of complementarity that is complementary with the RNA transcript (e.g., with at least 5 contiguous nucleotides of the RNA transcript) at a position that begins at the 5′-end of the transcript. In some embodiments, an oligonucleotide (e.g., an oligonucleotide that stabilizes an RNA transcript) comprises a region of complementarity that is complementary with an RNA transcript at a position within a region of the 5′ untranslated region (5′ UTR) of the RNA transcript spanning from the transcript start site to 50, 100, 150, 200, 250, 500 or more nucleotides upstream from a translation start site (e.g., a start codon, AUG, arising in a Kozak sequence of the transcript).

In some embodiments, an RNA transcript is poly-adenylated. Polyadenylation refers to the post-transcriptional addition of a polyadenosine (poly(A)) tail to an RNA transcript. Both protein-coding and non-coding RNA transcripts may be polyadenylated. Poly(A) tails contain multiple adenosines linked together through internucleoside linkages. In some embodiments, a poly(A) tail may contain 10 to 50, 25 to 100, 50 to 200, 150 to 250 or more adenosines. In some embodiments, the process of polyadenlyation involves endonucleolytic cleavage of an RNA transcript at or near its 3′-end followed by one by one addition of multiple adenosines to the transcript by a polyadenylate polymerase, the first of which adenonsines is added to the transcript at the 3′ cleavage site. Thus, often a polyadenylated RNA transcript comprises transcribed nucleotides (and possibly edited nucleotides) linked together through internucleoside linkages that are linked at the 3′ end to a poly(A) tail. The location of the linkage between the transcribed nucleotides and poly(A) tail may be referred to herein as, a “polyadenylation junction.” In some embodiments, endonucleolytic cleavage may occur at any one of several possible sites in an RNA transcript. In such embodiments, the sites may be determined by sequence motifs in the RNA transcript that are recognized by endonuclease machinery, thereby guiding the position of cleavage by the machinery. Thus, in some embodiments, polyadenylation can produce different RNA transcripts from a single gene, e.g., RNA transcripts have different polyadenylation junctions. In some embodiments, length of a poly(A) tail may determine susceptibility of the RNA transcript to enzymatic degradation by exonucleases with 3′-5′ processing activity. In some embodiments, oligonucleotides that target an RNA transcript at or near its 3′ end target a region overlapping a polyadenylation junction. In some embodiments, such oligonucleotides may have at least 3, 4, 5, 6, 7, 8, 9, 10, 11, 12 or more nucleotides that are complementary with the transcribed portion of the transcript (5′ to the junction). In some embodiments, it is advantageous to have a limited number of nucleotides (e.g., T, U) complementary to the polyA side of the junction. In some embodiments, having a limited number of nucleotides complementary to the polyA side of the junction it is advantageous because it reduces toxicity associated with cross hybridization of the oligonucleotide to the polyadenylation region of non-target RNAs in cells. In some embodiments, the oligonucleotide has only 1, 2, 3, 4, 5, or 6 nucleotides complementary to the poly A region.

In some embodiments, methods provided herein involve the use of an oligonucleotide that hybridizes with a target RNA transcript at or near its 3′ end and prevents or inhibits degradation of the RNA transcript by 3′-5′ exonucleases. For example, in some embodiments, RNA stabilization methods provided herein involve the use of an oligonucleotide that comprises a region of complementarity that is complementary with the RNA transcript at a position within 100 nucleotides, within 50 nucleotides, within 30 nucleotides, within 20 nucleotides, within 10 nucleotides, within 5 nucleotides of the last transcribed nucleotide of the RNA transcript. In a case where the RNA transcript is a polyadenylated transcript, the last transcribed nucleotide of the RNA transcript is the first nucleotide upstream of the polyadenylation junction. In some embodiments, RNA stabilization methods provided herein involve the use of an oligonucleotide that comprises a region of complementarity that is complementary with the RNA transcript at a position immediately adjacent to or overlapping the polyadenylation junction of the RNA transcript. In some embodiments, RNA stabilization methods provided herein involve the use of an oligonucleotide that comprises a region of complementarity that is complementary with the RNA transcript within the poly(A) tail.

Methods for identifying transcript start sites and polyadenylation junctions are known in the art and may be used in selecting oligonucleotides that specifically bind to these regions for stabilizing RNA transcripts. In some embodiments, 3′ end oligonucleotides may be designed by identifying RNA 3′ ends using quantitative end analysis of poly-A tails. In some embodiments, 5′ end oligonucleotides may be designed by identifying 5′ start sites using Cap analysis gene expression (CAGE). Appropriate methods are disclosed, for example, in Ozsolak et al. Comprehensive Polyadenylation Site Maps in Yeast and Human Reveal Pervasive Alternative Polyadenylation. Cell. Volume 143, Issue 6, 2010, Pages 1018-1029; Shiraki, T, et al., Cap analysis gene expression for high-throughput analysis of transcriptional starting point and identification of promoter usage. Proc Natl Acad Sci USA. 100 (26): 15776-81. 2003-12-23; and Zhao, X, et al., (2011). Systematic Clustering of Transcription Start Site Landscapes. PLoS ONE (Public Library of Science) 6 (8): e23409, the contents of each of which are incorporated herein by reference. Other appropriate methods for identifying transcript start sites and polyadenylation junctions may also be used, including, for example, RNA-Paired-end tags (PET) (See, e.g., Ruan X, Ruan Y. Methods Mol Biol. 2012; 809:535-62); use of standard EST databases; RACE combined with microarray or sequencing, PAS-Seq (See, e.g., Peter J. Shepard, et al., RNA. 2011 April; 17(4): 761-772); and 3P-Seq (See, e.g., Calvin H. January, Nature. 2011 January 6; 469(7328): 97-101; and others.

In some embodiments, an RNA transcript targeted by an oligonucleotide disclosed herein is an RNA transcript of a eukaryotic cell. In some embodiments, an RNA transcript targeted by an oligonucleotide disclosed herein is an RNA transcript of a cell of a vertebrate. In some embodiments, an RNA transcript targeted by an oligonucleotide disclosed herein is an RNA transcript of a cell of a mammal, e.g., a primate cell, mouse cell, rat cell, or human cell. In some embodiments, an RNA transcript targeted by an oligonucleotide disclosed herein is an RNA transcript of a cardiomyocyte. In some embodiments, an RNA transcript targeted by an oligonucleotide disclosed herein is an RNA transcribed in the nucleus of a cell. In some embodiments, an RNA transcript targeted by an oligonucleotide disclosed herein is an RNA transcribed in a mitochondrion of a cell. In some embodiments, an RNA transcript targeted by an oligonucleotide disclosed herein is an RNA transcript transcribed by a RNA polymerase II enzyme.

In some embodiments, an RNA transcript targeted by an oligonucleotide disclosed herein is an mRNA expressed from a gene selected from the group consisting of: ABCA1, APOA1, ATP2A2, BDNF, FXN, HBA2, HBB, HBD, HBE1, HBG1, HBG2, SMN, UTRN, PTEN, MECP2, and FOXP3. In some embodiments, the RNA transcript targeted by an oligonucleotide disclosed herein is an mRNA expressed from a gene selected from the group consisting of: ABCA4, ABCB11, ABCB4, ABCG5, ABCG8, ADIPOQ, ALB, APOE, BCL2L11, BRCA1, CD274, CEP290, CFTR, EPO, F7, F8, FLI1, FMR1, FNDC5, GCH1, GCK, GLP1R, GRN, HAMP, HPRT1, IDO1, IGF1, IL10, IL6, KCNMA1, KCNMB1, KCNMB2, KCNMB3, KCNMB4, KLF1, KLF4, LDLR, MSX2, MYBPC3, NANOG, NF1, NKX2-1, NKX2-1-AS1, PAH, PTGS2, RB1, RPS14, RPS19, SCARB1, SERPINF1, SIRT1, SIRT6, SMAD7, ST7, STAT3, TSIX, and XIST. RNA transcripts for these and other genes may be selected or identified experimentally, for example, using RNA sequencing (RNA-Seq) or other appropriate methods. RNA transcripts may also be selected based on information in public databases such as in UCSC, Ensemble and NCBI genome browsers and others. Non-limiting examples of RNA transcripts for certain genes are listed in Table 1.

TABLE 1
Non-limiting examples of RNA transcripts for certain genes

| GENE | | | |
|---|---|---|---|
| SYMBOL | MRNA | SPECIES | GENE NAME |
| ABCA1 | NM_013454 | Mus | ATP-binding cassette, sub-family A (ABC1), |
| | | musculus | member 1 |
| ABCA1 | NM_005502 | Homo | ATP-binding cassette, sub-family A (ABC1), |
| | | sapiens | member 1 |
| ABCA4 | NM_007378 | Mus | ATP-binding cassette, sub-family A (ABC1), |
| | | musculus | member 4 |
| ABCA4 | NM_000350 | Homo | ATP-binding cassette, sub-family A (ABC1), |
| | | sapiens | member 4 |
| ABCB11 | NM_003742 | Homo | ATP-binding cassette, sub-family B |
| | | sapiens | (MDR/TAP), member 11 |
| ABCB11 | NM_021022 | Mus | ATP-binding cassette, sub-family B |
| | | musculus | (MDR/TAP), member 11 |
| ABCB4 | NM_018850 | Homo | ATP-binding cassette, sub-family B |
| | | sapiens | (MDR/TAP), member 4 |
| ABCB4 | NM_000443 | Homo | ATP-binding cassette, sub-family B |
| | | sapiens | (MDR/TAP), member 4 |
| ABCB4 | NM_018849 | Homo | ATP-binding cassette, sub-family B |
| | | sapiens | (MDR/TAP), member 4 |
| ABCB4 | NM_008830 | Mus | ATP-binding cassette, sub-family B |
| | | musculus | (MDR/TAP), member 4 |
| ABCG5 | NM_022436 | Homo | ATP-binding cassette, sub-family G (WHITE), |
| | | sapiens | member 5 |
| ABCG5 | NM_031884 | Mus | ATP-binding cassette, sub-family G (WHITE), |
| | | musculus | member 5 |
| ABCG8 | NM_026180 | Mus | ATP-binding cassette, sub-family G (WHITE), |
| | | musculus | member 8 |
| ABCG8 | NM_022437 | Homo | ATP-binding cassette, sub-family G (WHITE), |
| | | sapiens | member 8 |
| ADIPOQ | NM_009605 | Mus | adiponectin, C1Q and collagen domain |
| | | musculus | containing |
| ADIPOQ | NM_004797 | Homo | adiponectin, C1Q and collagen domain |
| | | sapiens | containing |
| ALB | NM_000477 | Homo | albumin |
| | | sapiens | |
| ALB | NM_009654 | Mus | albumin |
| | | musculus | |
| APOA1 | NM_000039 | Homo | apolipoprotein A-I |
| | | sapiens | |
| APOA1 | NM_009692 | Mus | apolipoprotein A-I |
| | | musculus | |
| APOE | NM_009696 | Mus | apolipoprotein E |
| | | musculus | |
| APOE | XM_001724655 | Homo | hypothetical LOC100129500; apolipoprotein |
| | | sapiens | E |
| APOE | XM_001722911 | Homo | hypothetical LOC100129500; apolipoprotein |
| | | sapiens | E |
| APOE | XM_001724653 | Homo | hypothetical LOC100129500; apolipoprotein |
| | | sapiens | E |
| APOE | NM_000041 | Homo | hypothetical LOC100129500; apolipoprotein |
| | | sapiens | E |
| APOE | XM_001722946 | Homo | hypothetical LOC100129500; apolipoprotein |
| | | sapiens | E |
| ATP2A2 | NM_009722 | Mus | ATPase, Ca++ transporting, cardiac muscle, |
| | | musculus | slow twitch 2 |
| ATP2A2 | NM_001110140 | Mus | ATPase, Ca++ transporting, cardiac muscle, |
| | | musculus | slow twitch 2 |
| ATP2A2 | NM_001135765 | Homo | ATPase, Ca++ transporting, cardiac muscle, |
| | | sapiens | slow twitch 2 |
| ATP2A2 | NM_170665 | Homo | ATPase, Ca++ transporting, cardiac muscle, |
| | | sapiens | slow twitch 2 |
| ATP2A2 | NM_001681 | Homo | ATPase, Ca++ transporting, cardiac muscle, |
| | | sapiens | slow twitch 2 |
| BCL2L11 | NM_006538 | Homo | BCL2-like 11 (apoptosis facilitator) |
| | | sapiens | |
| BCL2L11 | NM_207002 | Homo | BCL2-like 11 (apoptosis facilitator) |
| | | sapiens | |
| BCL2L11 | NM_138621 | Homo | BCL2-like 11 (apoptosis facilitator) |
| | | sapiens | |
| BCL2L11 | NM_207680 | Mus | BCL2-like 11 (apoptosis facilitator) |
| | | musculus | |
| BCL2L11 | NM_207681 | Mus | BCL2-like 11 (apoptosis facilitator) |
| | | musculus | |
| BCL2L11 | NM_009754 | Mus | BCL2-like 11 (apoptosis facilitator) |
| | | musculus | |
| BDNF | NM_001143816 | Homo | brain-derived neurotrophic factor |
| | | sapiens | |
| BDNF | NM_001143815 | Homo | brain-derived neurotrophic factor |
| | | sapiens | |
| BDNF | NM_001143814 | Homo | brain-derived neurotrophic factor |
| | | sapiens | |
| BDNF | NM_001143813 | Homo | brain-derived neurotrophic factor |
| | | sapiens | |
| BDNF | NM_001143812 | Homo | brain-derived neurotrophic factor |
| | | sapiens | |
| BDNF | NM_001143806 | Homo | brain-derived neurotrophic factor |
| | | sapiens | |
| BDNF | NM_001143811 | Homo | brain-derived neurotrophic factor |
| | | sapiens | |
| BDNF | NM_001143805 | Homo | brain-derived neurotrophic factor |
| | | sapiens | |
| BDNF | NM_001143810 | Homo | brain-derived neurotrophic factor |
| | | sapiens | |
| BDNF | NM_001709 | Homo | brain-derived neurotrophic factor |
| | | sapiens | |
| BDNF | NM_170735 | Homo | brain-derived neurotrophic factor |
| | | sapiens | |
| BDNF | NM_170734 | Homo | brain-derived neurotrophic factor |
| | | sapiens | |
| BDNF | NM_170733 | Homo | brain-derived neurotrophic factor |
| | | sapiens | |
| BDNF | NM_170732 | Homo | brain-derived neurotrophic factor |
| | | sapiens | |
| BDNF | NM_170731 | Homo | brain-derived neurotrophic factor |
| | | sapiens | |
| BDNF | NM_001143809 | Homo | brain-derived neurotrophic factor |
| | | sapiens | |
| BDNF | NM_001143807 | Homo | brain-derived neurotrophic factor |
| | | sapiens | |
| BDNF | NM_001143808 | Homo | brain-derived neurotrophic factor |
| | | sapiens | |
| BDNF | NM_007540 | Mus | brain derived neurotrophic factor |
| | | musculus | |
| BDNF | NM_001048141 | Mus | brain derived neurotrophic factor |
| | | musculus | |
| BDNF | NM_001048142 | Mus | brain derived neurotrophic factor |
| | | musculus | |
| BDNF | NM_001048139 | Mus | brain derived neurotrophic factor |
| | | musculus | |
| BRCA1 | NM_009764 | Mus | breast cancer 1 |
| | | musculus | |
| BRCA1 | NM_007296 | Homo | breast cancer 1, early onset |
| | | sapiens | |
| BRCA1 | NM_007300 | Homo | breast cancer 1, early onset |
| | | sapiens | |
| BRCA1 | NM_007297 | Homo | breast cancer 1, early onset |
| | | sapiens | |
| BRCA1 | NM_007303 | Homo | breast cancer 1, early onset |
| | | sapiens | |
| BRCA1 | NM_007298 | Homo | breast cancer 1, early onset |
| | | sapiens | |
| BRCA1 | NM_007302 | Homo | breast cancer 1, early onset |
| | | sapiens | |
| BRCA1 | NM_007299 | Homo | breast cancer 1, early onset |
| | | sapiens | |
| BRCA1 | NM_007304 | Homo | breast cancer 1, early onset |
| | | sapiens | |
| BRCA1 | NM_007294 | Homo | breast cancer 1, early onset |
| | | sapiens | |
| BRCA1 | NM_007305 | Homo | breast cancer 1, early onset |
| | | sapiens | |
| BRCA1 | NM_007295 | Homo | breast cancer 1, early onset |
| | | sapiens | |
| CD274 | NM_014143 | Homo | CD274 molecule |
| | | sapiens | |
| CD274 | NM_021893 | Mus | CD274 antigen |
| | | musculus | |
| CEP290 | NM_025114 | Homo | centrosomal protein 290 kDa |
| | | sapiens | |
| CEP290 | NM_146009 | Mus | centrosomal protein 290 |
| | | musculus | |
| CFTR | NM_000492 | Homo | cystic fibrosis transmembrane conductance |
| | | sapiens | regulator (ATP-binding cassette sub-family C, |
| | | | member 7) |
| CFTR | NM_021050 | Mus | cystic fibrosis transmembrane conductance |
| | | musculus | regulator homolog |
| EPO | NM_000799 | Homo | erythropoietin |
| | | sapiens | |
| EPO | NM_007942 | Mus | erythropoietin |
| | | musculus | |
| F7 | NM_000131 | Homo | coagulation factor VII (serum prothrombin |
| | | sapiens | conversion accelerator) |
| F7 | NM_019616 | Homo | coagulation factor VII (serum prothrombin |
| | | sapiens | conversion accelerator) |
| F7 | NM_010172 | Mus | coagulation factor VII |
| | | musculus | |
| F8 | NM_019863 | Homo | coagulation factor VIII, procoagulant |
| | | sapiens | component |
| F8 | NM_000132 | Homo | coagulation factor VIII, procoagulant |
| | | sapiens | component |
| F8 | NM_001161373 | Mus | coagulation factor VIII |
| | | musculus | |
| F8 | NM_001161374 | Mus | coagulation factor VIII |
| | | musculus | |
| F8 | NM_007977 | Mus | coagulation factor VIII |
| | | musculus | |
| FLI1 | NM_002017 | Homo | Friend leukemia virus integration 1 |
| | | sapiens | |
| FLI1 | NM_001167681 | Homo | Friend leukemia virus integration 1 |
| | | sapiens | |
| FLI1 | NM_008026 | Mus | |
| | | musculus | Friend leukemia integration 1 |
| FMR1 | NM_008031 | Mus | fragile X mental retardation syndrome 1 |
| | | musculus | homolog |
| FMR1 | NM_002024 | Homo | |
| | | sapiens | fragile X mental retardation 1 |
| FNDC5 | NM_001171941 | Homo | fibronectin type III domain containing 5 |
| | | sapiens | |
| FNDC5 | NM_153756 | Homo | fibronectin type III domain containing 5 |
| | | sapiens | |
| FNDC5 | NM_001171940 | Homo | fibronectin type III domain containing 5 |
| | | sapiens | |
| FNDC5 | NM_027402 | Mus | fibronectin type III domain containing 5 |
| | | musculus | |
| FOXP3 | NM_054039 | Mus | forkhead box P3 |
| | | musculus | |
| FOXP3 | NM_001114377 | Homo | forkhead box P3 |
| | | sapiens | |
| FOXP3 | NM_014009 | Homo | forkhead box P3 |
| | | sapiens | |
| FXN | NM_001161706 | Homo | frataxin |
| | | sapiens | |
| FXN | NM_181425 | Homo | frataxin |
| | | sapiens | |
| FXN | NM_000144 | Homo | frataxin |
| | | sapiens | |
| FXN | NM_008044 | Mus | frataxin |
| | | musculus | |
| GCH1 | NM_008102 | Mus | GTP cyclohydrolase 1 |
| | | musculus | |
| GCH1 | NM_000161 | Homo | GTP cyclohydrolase 1 |
| | | sapiens | |
| GCH1 | NM_001024070 | Homo | GTP cyclohydrolase 1 |
| | | sapiens | |
| GCH1 | NM_001024071 | Homo | GTP cyclohydrolase 1 |
| | | sapiens | |
| GCH1 | NM_001024024 | Homo | GTP cyclohydrolase 1 |
| | | sapiens | |
| GCK | NM_010292 | Mus | glucokinase |
| | | musculus | |
| GCK | NM_000162 | Homo | glucokinase (hexokinase 4) |
| | | sapiens | |
| GCK | NM_033508 | Homo | glucokinase (hexokinase 4) |
| | | sapiens | |
| GCK | NM_033507 | Homo | glucokinase (hexokinase 4) |
| | | sapiens | |
| GLP1R | NM_021332 | Mus | glucagon-like peptide 1 receptor; similar to |
| | | musculus | glucagon-like peptide-1 receptor |
| GLP1R | XM_001471951 | Mus | glucagon-like peptide 1 receptor; similar to |
| | | musculus | glucagon-like peptide-1 receptor |
| GLP1R | NM_002062 | Homo | glucagon-like peptide 1 receptor |
| | | sapiens | |
| GRN | NM_002087 | Homo | granulin |
| | | sapiens | |
| GRN | NM_008175 | Mus | granulin |
| | | musculus | |
| HAMP | NM_021175 | Homo | hepcidin antimicrobial peptide |
| | | sapiens | |
| HAMP | NM_032541 | Mus | hepcidin antimicrobial peptide |
| | | musculus | |
| HBA2 | NM_000517 | Homo | hemoglobin, alpha 2; hemoglobin, alpha 1 |
| | | sapiens | |
| HBA2 | NM_000558 | Homo | hemoglobin, alpha 2; hemoglobin, alpha 1 |
| | | sapiens | |
| HBB | NM_000518 | Homo | hemoglobin, beta |
| | | sapiens | |
| HBB | XM_921413 | Mus | hemoglobin beta chain complex |
| | | musculus | |
| HBB | XM_903245 | Mus | hemoglobin beta chain complex |
| | | musculus | |
| HBB | XM_921395 | Mus | hemoglobin beta chain complex |
| | | musculus | |
| HBB | XM_903244 | Mus | hemoglobin beta chain complex |
| | | musculus | |
| HBB | XM_903246 | Mus | hemoglobin beta chain complex |
| | | musculus | |
| HBB | XM_909723 | Mus | hemoglobin beta chain complex |
| | | musculus | |
| HBB | XM_921422 | Mus | hemoglobin beta chain complex |
| | | musculus | |
| HBB | XM_489729 | Mus | hemoglobin beta chain complex |
| | | musculus | |
| HBB | XM_903242 | Mus | hemoglobin beta chain complex |
| | | musculus | |
| HBB | XM_903243 | Mus | hemoglobin beta chain complex |
| | | musculus | |
| HBB | XM_921400 | Mus | hemoglobin beta chain complex |
| | | musculus | |
| HBD | NM_000519 | Homo | hemoglobin, delta |
| | | sapiens | |
| HBE1 | NM_005330 | Homo | hemoglobin, epsilon 1 |
| | | sapiens | |
| HBG1 | NM_000559 | Homo | hemoglobin, gamma A |
| | | sapiens | |
| HBG2 | NM_000184 | Homo | hemoglobin, gamma G |
| | | sapiens | |
| HPRT1 | NM_000194 | Homo | hypoxanthine phosphoribosyltransferase 1 |
| | | sapiens | |
| IDO1 | NM_008324 | Mus | indoleamine 2,3-dioxygenase 1 |
| | | musculus | |
| IDO1 | NM_002164 | Homo | indoleamine 2,3-dioxygenase 1 |
| | | sapiens | |
| IGF1 | NM_001111284 | Homo | insulin-like growth factor 1 (somatomedin C) |
| | | sapiens | |
| IGF1 | NM_001111285 | Homo | insulin-like growth factor 1 (somatomedin C) |
| | | sapiens | |
| IGF1 | NM_001111283 | Homo | insulin-like growth factor 1 (somatomedin C) |
| | | sapiens | |
| IGF1 | NM_000618 | Homo | insulin-like growth factor 1 (somatomedin C) |
| | | sapiens | |
| IGF1 | NM_001111274 | Mus | insulin-like growth factor 1 |
| | | musculus | |
| IGF1 | NM_010512 | Mus | insulin-like growth factor 1 |
| | | musculus | |
| IGF1 | NM_184052 | Mus | insulin-like growth factor 1 |
| | | musculus | |
| IGF1 | NM_001111276 | Mus | insulin-like growth factor 1 |
| | | musculus | |
| IGF1 | NM_001111275 | Mus | insulin-like growth factor 1 |
| | | musculus | |
| IL10 | NM_000572 | Homo | interleukin 10 |
| | | sapiens | |
| IL10 | NM_010548 | Mus | interleukin 10 |
| | | musculus | |
| IL6 | NM_031168 | Mus | interleukin 6 |
| | | musculus | |
| IL6 | NM_000600 | Homo | interleukin 6 (interferon, beta 2) |
| | | sapiens | |
| KCNMA1 | NM_002247 | Homo | potassium large conductance calcium- |
| | | sapiens | activated channel, subfamily M, alpha |
| | | | member 1 |
| KCNMA1 | NM_001161352 | Homo | potassium large conductance calcium- |
| | | sapiens | activated channel, subfamily M, alpha |
| | | | member 1 |
| KCNMA1 | NM_001014797 | Homo | potassium large conductance calcium- |
| | | sapiens | activated channel, subfamily M, alpha |
| | | | member 1 |
| KCNMA1 | NM_001161353 | Homo | potassium large conductance calcium- |
| | | sapiens | activated channel, subfamily M, alpha |
| | | | member 1 |
| KCNMA1 | NM_010610 | Mus | potassium large conductance calcium- |
| | | musculus | activated channel, subfamily M, alpha |
| | | | member 1 |
| KCNMB1 | NM_031169 | Mus | potassium large conductance calcium- |
| | | musculus | activated channel, subfamily M, beta |
| | | | member 1 |
| KCNMB1 | NM_004137 | Homo | potassium large conductance calcium- |
| | | sapiens | activated channel, subfamily M, beta |
| | | | member 1 |
| KCNMB2 | NM_028231 | Mus | potassium large conductance calcium- |
| | | musculus | activated channel, subfamily M, beta |
| | | | member 2 |
| KCNMB2 | NM_005832 | Homo | potassium large conductance calcium- |
| | | sapiens | activated channel, subfamily M, beta |
| | | | member 2 |
| KCNMB2 | NM_181361 | Homo | potassium large conductance calcium- |
| | | sapiens | activated channel, subfamily M, beta |
| | | | member 2 |
| KCNMB3 | NM_171829 | Homo | potassium large conductance calcium- |
| | | sapiens | activated channel, subfamily M beta member |
| | | | 3 |
| KCNMB3 | NM_171828 | Homo | potassium large conductance calcium- |
| | | sapiens | activated channel, subfamily M beta member |
| | | | 3 |
| KCNMB3 | NM_001163677 | Homo | potassium large conductance calcium- |
| | | sapiens | activated channel, subfamily M beta member |
| | | | 3 |
| KCNMB3 | NM_014407 | Homo | potassium large conductance calcium- |
| | | sapiens | activated channel, subfamily M beta member |
| | | | 3 |
| KCNMB3 | NM_171830 | Homo | potassium large conductance calcium- |
| | | sapiens | activated channel, subfamily M beta member |
| | | | 3 |
| KCNMB3 | XM_001475546 | Mus | potassium large conductance calcium- |
| | | musculus | activated channel, subfamily M, beta |
| | | | member 3 |
| KCNMB3 | XM_912348 | Mus | potassium large conductance calcium- |
| | | musculus | activated channel, subfamily M, beta |
| | | | member 3 |
| KCNMB4 | NM_021452 | Mus | potassium large conductance calcium- |
| | | musculus | activated channel, subfamily M, beta |
| | | | member 4 |
| KCNMB4 | NM_014505 | Homo | potassium large conductance calcium- |
| | | sapiens | activated channel, subfamily M, beta |
| | | | member 4 |
| KLF1 | NM_010635 | Mus | Kruppel-like factor 1 (erythroid) |
| | | musculus | |
| KLF1 | NM_006563 | Homo | Kruppel-like factor 1 (erythroid) |
| | | sapiens | |
| KLF4 | NM_010637 | Mus | Kruppel-like factor 4 (gut) |
| | | musculus | |
| KLF4 | NM_004235 | Homo | Kruppel-like factor 4 (gut) |
| | | sapiens | |
| LAMA1 | NM_005559.3 | Homo | laminin, alpha 1 |
| | | sapiens | |
| LAMA1 | NM_008480.2 | Mus | laminin, alpha 1 |
| | | musculus | |
| LDLR | NM_000527 | Homo | low density lipoprotein receptor |
| | | sapiens | |
| LDLR | NM_010700 | Mus | low density lipoprotein receptor |
| | | musculus | |
| MBNL1 | NM_021038.3, | Homo | muscleblind-like splicing regulator 1 |
| | NM_020007.3, | sapiens | |
| | NM_207293.1, | | |
| | NM_207294.1, | | |
| | NM_207295.1, | | |
| | NM_207296.1, | | |
| | NM_207297.1 | | |
| MBNL1 | NM_001253708.1, | Mus | muscleblind-like 1 (Drosophila) |
| | NM_001253709.1, | musculus | |
| | NM_001253710.1, | | |
| | NM_001253711.1, | | |
| | NM_001253713.1, | | |
| | NM_020007.3 | | |
| MECP2 | NM_010788 | Mus | methyl CpG binding protein 2 |
| | | musculus | |
| MECP2 | NM_001081979 | Mus | methyl CpG binding protein 2 |
| | | musculus | |
| MECP2 | NM_001110792 | Homo | methyl CpG binding protein 2 (Rett |
| | | sapiens | syndrome) |
| MECP2 | NM_004992 | Homo | methyl CpG binding protein 2 (Rett |
| | | sapiens | syndrome) |
| MERTK | NM_006343.2 | Homo | MER proto-oncogene, tyrosine kinase |
| | | sapiens | |
| MERTK | NM_008587.1 | Mus | c-mer proto-oncogene tyrosine kinase |
| | | musculus | |
| MSX2 | NM_013601 | Mus | similar to homeobox protein; homeobox, |
| | | musculus | msh-like 2 |
| MSX2 | XM_001475886 | Mus | similar to homeobox protein; homeobox, |
| | | musculus | msh-like 2 |
| MSX2 | NM_002449 | Homo | msh homeobox 2 |
| | | sapiens | |
| MYBPC3 | NM_008653 | Mus | myosin binding protein C, cardiac |
| | | musculus | |
| MYBPC3 | NM_000256 | Homo | myosin binding protein C, cardiac |
| | | sapiens | |
| NANOG | NM_024865 | Homo | Nanog homeobox pseudogene 8; Nanog |
| | | sapiens | homeobox |
| NANOG | XM_001471588 | Mus | similar to Nanog homeobox; Nanog |
| | | musculus | homeobox |
| NANOG | NM_028016 | Mus | similar to Nanog homeobox; Nanog |
| | | musculus | homeobox |
| NANOG | NM_001080945 | Mus | similar to Nanog homeobox; Nanog |
| | | musculus | homeobox |
| NF1 | NM_000267 | Homo | neurofibromin 1 |
| | | sapiens | |
| NF1 | NM_001042492 | Homo | neurofibromin 1 |
| | | sapiens | |
| NF1 | NM_001128147 | Homo | neurofibromin 1 |
| | | sapiens | |
| NF1 | NM_010897 | Mus | neurofibromatosis 1 |
| | | musculus | |
| NKX2-1 | NM_001079668 | Homo | NK2 homeobox 1 |
| | | sapiens | |
| NKX2-1 | NM_003317 | Homo | NK2 homeobox 1 |
| | | sapiens | |
| NKX2-1 | XM_002344771 | Homo | NK2 homeobox 1 |
| | | sapiens | |
| NKX2-1 | NM_009385 | Mus | NK2 homeobox 1 |
| | | musculus | |
| NKX2-1 | NM_001146198 | Mus | NK2 homeobox 1 |
| | | musculus | |
| PAH | NM_008777 | Mus | phenylalanine hydroxylase |
| | | musculus | |
| PAH | NM_000277 | Homo | phenylalanine hydroxylase |
| | | sapiens | |
| PTEN | NM_000314 | Homo | phosphatase and tensin homolog; |
| | | sapiens | phosphatase and tensin homolog |
| | | | pseudogene 1 |
| PTEN | NM_177096 | Mus | phosphatase and tensin homolog |
| | | musculus | |
| PTEN | NM_008960 | Mus | phosphatase and tensin homolog |
| | | musculus | |
| PTGS2 | NM_011198 | Mus | prostaglandin-endoperoxide synthase 2 |
| | | musculus | |
| PTGS2 | NM_000963 | Homo | prostaglandin-endoperoxide synthase 2 |
| | | sapiens | (prostaglandin G/H synthase and |
| | | | cyclooxygenase) |
| RB1 | NM_009029 | Mus | retinoblastoma 1 |
| | | musculus | |
| RB1 | NM_000321 | Homo | retinoblastoma 1 |
| | | sapiens | |
| RPS14 | NM_020600 | Mus | predicted gene 6204; ribosomal protein S14 |
| | | musculus | |
| RPS14 | NM_001025071 | Homo | ribosomal protein S14 |
| | | sapiens | |
| RPS14 | NM_005617 | Homo | ribosomal protein S14 |
| | | sapiens | |
| RPS14 | NM_001025070 | Homo | ribosomal protein S14 |
| | | sapiens | |
| RPS19 | XM_204069 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_991053 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_905004 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001005575 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | NM_023133 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_994263 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001481027 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_913504 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001479631 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_902221 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_893968 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | NM_001022 | Homo | ribosomal protein S19 pseudogene 3; |
| | | sapiens | ribosomal protein S19 |
| SCARB1 | NM_016741 | Mus | scavenger receptor class B, member 1 |
| | | musculus | |
| SCARB1 | NM_001082959 | Homo | scavenger receptor class B, member 1 |
| | | sapiens | |
| SCARB1 | NM_005505 | Homo | scavenger receptor class B, member 1 |
| | | sapiens | |
| SERPINF1 | NM_011340 | Mus | serine (or cysteine) peptidase inhibitor, clade |
| | | musculus | F, member 1 |
| SERPINF1 | NM_002615 | Homo | serpin peptidase inhibitor, Glade F (alpha-2 |
| | | sapiens | antiplasmin, pigment epithelium derived |
| | | | factor), member 1 |
| SIRT1 | NM_001159590 | Mus | sirtuin 1 (silent mating type information |
| | | musculus | regulation 2, homolog) 1 (S. cerevisiae) |
| SIRT1 | NM_019812 | Mus | sirtuin 1 (silent mating type information |
| | | musculus | regulation 2, homolog) 1 (S. cerevisiae) |
| SIRT1 | NM_001159589 | Mus | sirtuin 1 (silent mating type information |
| | | musculus | regulation 2, homolog) 1 (S. cerevisiae) |
| SIRT1 | NM_012238 | Homo | sirtuin (silent mating type information |
| | | sapiens | regulation 2 homolog) 1 (S. cerevisiae) |
| SIRT1 | NM_001142498 | Homo | sirtuin (silent mating type information |
| | | sapiens | regulation 2 homolog) 1 (S. cerevisiae) |
| SIRT6 | NM_016539 | Homo | sirtuin (silent mating type information |
| | | sapiens | regulation 2 homolog) 6 (S. cerevisiae) |
| SIRT6 | NM_001163430 | Mus | sirtuin 6 (silent mating type information |
| | | musculus | regulation 2, homolog) 6 (S. cerevisiae) |
| SIRT6 | NM_181586 | Mus | sirtuin 6 (silent mating type information |
| | | musculus | regulation 2, homolog) 6 (S. cerevisiae) |
| SMAD7 | NM_005904 | Homo | SMAD family member 7 |
| | | sapiens | |
| SMAD7 | NM_001042660 | Mus | MAD homolog 7 (Drosophila) |
| | | musculus | |
| SMN1 | NM_000344.3 | Homo | Survival Motor Neuron 1 |
| | | sapiens | |
| SMN1 | NM_022874.2 | Homo | Survival Motor Neuron 1 |
| | | sapiens | |
| SMN2 | NM_017411.3 | Homo | Survival Motor Neuron 2 |
| | NM_022875.2 | sapiens | |
| | NM_022876.2 | | |
| | NM_022877.2 | | |
| SSPN | NM_001135823.1, | Homo | sarcospan |
| | NM_005086.4 | sapiens | |
| SSPN | NM_010656.2 | Homo | sarcospan |
| | | sapiens | |
| ST7 | NM_021908 | Homo | suppression of tumorigenicity 7 |
| | | sapiens | |
| ST7 | NM_018412 | Homo | suppression of tumorigenicity 7 |
| | | sapiens | |
| STAT3 | NM_213660 | Mus | similar to Stat3B; signal transducer and |
| | | musculus | activator of transcription 3 |
| STAT3 | XM_001474017 | Mus | similar to Stat3B; signal transducer and |
| | | musculus | activator of transcription 3 |
| STAT3 | NM_213659 | Mus | similar to Stat3B; signal transducer and |
| | | musculus | activator of transcription 3 |
| STAT3 | NM_011486 | Mus | similar to Stat3B; signal transducer and |
| | | musculus | activator of transcription 3 |
| STAT3 | NM_213662 | Homo | signal transducer and activator of |
| | | sapiens | transcription 3 (acute-phase response factor) |
| STAT3 | NM_003150 | Homo | signal transducer and activator of |
| | | sapiens | transcription 3 (acute-phase response factor) |
| STAT3 | NM_139276 | Homo | signal transducer and activator of |
| | | sapiens | transcription 3 (acute-phase response factor) |
| UTRN | NM_007124 | Homo | utrophin |
| | | sapiens | |
| UTRN | NM_011682 | Mus | utrophin |
| | | musculus | |
| NFE2L2 | NM_001145412.2, | Homo | nuclear factor, erythroid 2-like 2 |
| | NM_001145413.2, | sapiens | |
| | NM_006164.4 | | |
| NFE2L2 | NM_010902.3 | Mus | nuclear factor, erythroid 2-like 2 |
| | | musculus | |
| ACTB | NM_001101.3 | Homo | actin, beta |
| | | sapiens | |
| ACTB | NM_007393.3 | Mus | actin, beta |
| | | musculus | |
| ANRIL | NR_003529.3, | Homo | CDKN2B antisense RNA 1 (also called |
| | NR_047532.1, | sapiens | CDKN2B) |
| | NR_047533.1, | | |
| | NR_047534.1, | | |
| | NR_047535.1, | | |
| | NR_047536.1, | | |
| | NR_047538.1, | | |
| | NR_047539.1, | | |
| | NR_047540.1, | | |
| | NR_047541.1, | | |
| | NR_047542.1, | | |
| | NR_047543.1 | | |
| HOTAIR | NR_003716.3, | Homo | HOX transcript antisense RNA |
| | NR_047517.1, | sapiens | |
| | NR_047518.1 | | |
| HOTAIR | NR_047528.1 | Mus | HOX transcript antisense RNA |
| | | musculus | |
| DINO | JX993265 | Homo | Damage Induced NOncoding |
| | | sapiens | |
| DINO | JX993266 | Mus | Damage Induced NOncoding |
| | | musculus | |
| HOTTIP | NR_037843.3 | Homo | HOXA distal transcript antisense RNA |
| | | sapiens | |
| HOTTIP | NR_110441.1, | Mus | Hoxa distal transcript antisense RNA |
| | NR_110442.1 | musculus | |
| NEST | NR_104124.1 | Homo | Homo sapiens IFNG antisense RNA 1 (IFNG- |
| | | sapiens | AS1), transcript variant 1, long non-coding |
| | | | RNA. |
| NEST | NR_104123.1 | Mus | Theiler's murine encephalomyelitis virus |
| | | musculus | persistence candidate gene 1 |

### Oligonucleotides

Oligonucleotides provided herein are useful for stabilizing RNAs by inhibiting or preventing degradation of the RNAs (e.g., degradation mediated by exonucleases). Such oligonucleotides may be referred to as “stabilizing oligonucleotides”. In some embodiments, oligonucleotides hybridize at a 5′ and/or 3′ region of the RNA resulting in duplex regions that stabilize the RNA by preventing degradation by exonucleotides having single strand processing activity.

In some embodiments, oligonucleotides are provided having a region complementary with at least 5 consecutive nucleotides of a 5′ region of an RNA transcript. In some embodiments, oligonucleotides are provided having a region complementary with at least 5 consecutive nucleotides of a 3′-region of an RNA transcript. In some embodiments, oligonucleotides are provided having a first region complementary with at least 5 consecutive nucleotides of a 5′ region of an RNA transcript, and a second region complementary with at least 5 consecutive nucleotides of a 3′-region of an RNA transcript.

In some embodiments, oligonucleotides are provided having a region complementary with at least 5 consecutive nucleotides of the 5′-UTR of an mRNA transcript. In some embodiments, oligonucleotides are provided having a region complementary with at least 5 consecutive nucleotides of the 3′-UTR, poly(A) tail, or overlapping the polyadenylation junction of the mRNA transcript. In some embodiments, oligonucleotides are provided having a first region complementary with at least 5 consecutive nucleotides of the 5′-UTR of an mRNA transcript, and a second region complementary with at least 5 consecutive nucleotides of the 3′-UTR, poly(A) tail, or overlapping the polyadenylation junction of the mRNA transcript.

In some embodiments, oligonucleotides are provided that have a region of complementarity that is complementary to an RNA transcript in proximity to the 5′-end of the RNA transcript. In such embodiments, the nucleotide at the 3′-end of the region of complementarity of the oligonucleotides may be complementary with the RNA transcript at a position that is within 10 nucleotides, within 20 nucleotides, within 30 nucleotides, within 40 nucleotides, within 50 nucleotides, or within 100 nucleotides, within 200 nucleotides, within 300 nucleotides, within 400 nucleotides or more of the transcription start site of the RNA transcript.

In some embodiments, oligonucleotides are provided that have a region of complementarity that is complementary to an RNA transcript in proximity to the 3′-end of the RNA transcript. In such embodiments, the nucleotide at the 3′-end and/or 5′ end of the region of complementarity may be complementary with the RNA transcript at a position that is within 10 nucleotides, within 20 nucleotides, within 30 nucleotides, within 40 nucleotides, within 50 nucleotides, within 100 nucleotides, within 200 nucleotides, within 300 nucleotides, within 400 nucleotides or more of the 3′-end of the RNA transcript. In some embodiments, if the target RNA transcript is polyadenylated, the nucleotide at the 3′-end of the region of complementarity of the oligonucleotide may be complementary with the RNA transcript at a position that is within 10 nucleotides, within 20 nucleotides, within 30 nucleotides, within 40 nucleotides, within 50 nucleotides, within 100 nucleotides, within 200 nucleotides, within 300 nucleotides, within 400 nucleotides or more of polyadenylation junction. In some embodiments, an oligonucleotide that targets a 3′ region of an RNA comprises a region of complementarity that is a stretch of pyrimidines (e.g., 4 to 10 or 5 to 15 thymine nucleotides) complementary with adenines.

In some embodiments, combinations of 5′ targeting and 3′ targeting oligonucleotides are contacted with a target RNA. In some embodiments, the 5′ targeting and 3′ targeting oligonucleotides a linked together via a linker (e.g., a stretch of nucleotides non-complementary with the target RNA). In some embodiments, the region of complementarity of the 5′ targeting oligonucleotide is complementary to a region in the target RNA that is at least 2, 5, 10, 20, 50, 100, 500, 1000, 5000, 10000 nucleotides upstream from the region of the target RNA that is complementary to the region of complementarity of the 3′ end targeting oligonucleotide.

In some embodiments, oligonucleotides are provided that have the general formula 5′-X₁-X₂-3′, in which X₁ has a region of complementarity that is complementary with an RNA transcript (e.g., with at least 5 contiguous nucleotides of the RNA transcript). In some embodiments, the nucleotide at the 3′-end of the region of complementary of X₁ may be complementary with a nucleotide in proximity to the transcription start site of the RNA transcript. In some embodiments, the nucleotide at the 3′-end of the region of complementary of X₁ may be complementary with a nucleotide that is present within 1, 2, 3, 4, 5, 6, 7, 8, 9, or 10 nucleotides of the transcription start site of the RNA transcript. In some embodiments, the nucleotide at the 3′-end of the region of complementary of X₁ may be complementary with the nucleotide at the transcription start site of the RNA transcript.

In some embodiments, X₁ comprises 5 to 10 nucleotides, 5 to 15 nucleotides, 5 to 25 nucleotides, 10 to 25 nucleotides, 5 to 20 nucleotides, or 15 to 30 nucleotides. In some embodiments, X₁ comprises 5, 6, 7, 8, 9, 10, 11, 12, 13, 14, 15, 16, 17, 18, 19, 20, 25, 30 or more nucleotides. In some embodiments, the region of complementarity of X₁ may be complementary with at least 4, at least 5, at least 6, at least 7, at least 8, at least 9, or at least 10 contiguous nucleotides of the RNA transcript. In some embodiments, the region of complementarity of X₁ may be complementary with 4, 5, 6, 7, 8, 9, 10, 11, 12, 13, 14, 15, 16, 17, 18, 19, 20 or more contiguous nucleotides of the RNA transcript.

In some embodiments, X₂ is absent. In some embodiments, X₂ comprises 1 to 10, 1 to 20 nucleotides, 1 to 25 nucleotides, 5 to 20 nucleotides, 5 to 30 nucleotides, 5 to 40 nucleotides, or 5 to 50 nucleotides. In some embodiments, X₂ comprises 5, 6, 7, 8, 9, 10, 11, 12, 13, 14, 15, 16, 17, 18, 19, 20, 25, 30, 35, 40, 45, 50 or more nucleotides. In some embodiments, X₂ comprises a region of complementarity complementary with at least 4, at least 5, at least 6, at least 7, at least 8, at least 9, or at least 10 contiguous nucleotides of the RNA transcript. In some embodiments, X₂ comprises a region of complementarity complementary with 4, 5, 6, 7, 8, 9, 10, 11, 12, 13, 14, 15, 16, 17, 18, 19, 20 or more contiguous nucleotides of the RNA transcript.

In some embodiments, the RNA transcript has a 7-methylguanosine cap at its 5′-end. In some embodiments, the nucleotide at the 3′-end of the region of complementary of X₁ is complementary with the nucleotide of the RNA transcript that is immediately internal to the 7-methylguanosine cap or in proximity to the cap (e.g., with 10 nucleotides of the cap). In some embodiments, at least the first nucleotide at the 5′-end of X₂ is a pyrimidine complementary with guanine (e.g., a cytosine or analogue thereof). In some embodiments, the first and second nucleotides at the 5′-end of X₂ are pyrimidines complementary with guanine. Thus, in some embodiments, at least one nucleotide at the 5′-end of X₂ is a pyrimidine that may form stabilizing hydrogen bonds with the 7-methylguanosine of the cap.

In some embodiments, X₂ forms a stem-loop structure. In some embodiments, X₂ comprises the formula 5′-Y₁-Y₂-Y₃-3′, in which X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, the stem region comprises 1-6, 1-5, 2-5, 1-4, 2-4 or 2-3 nucleotides. In some embodiments, the stem region comprises LNA nucleotides. In some embodiments, the stem region comprises 1-6, 1-5, 2-5, 1-4, 2-4 or 2-3 LNA nucleotides. In some embodiments, Y₁ and Y₃ independently comprise 2 to 10 nucleotides, 2 to 20 nucleotides, 2 to 25 nucleotides, or 5 to 20 nucleotides. In some embodiments, Y₁ and Y₃ independently comprise 2, 3, 4, 5, 6, 7, 8, 9, 10, 11, 12, 13, 14, 15, 16, 17, 18, 19, 20, 25 or more nucleotides. In some embodiments, Y₂ comprises 3 to 10 nucleotides, 3 to 15 nucleotides, 3 to 25 nucleotides, or 5 to 20 nucleotides. In some embodiments, Y₂ comprises 3, 4, 5, 6, 7, 8, 9, 10, 11, 12, 13, 14, 15, 16, 17, 18, 19, 20, 25 or more nucleotides. In some embodiments, Y₂ comprises 2-8, 2-7, 2-6, 2-5, 3-8, 3-7, 3-6, 3-5 or 3-4 nucleotides. In some embodiments, Y₂ comprises at least one DNA nucleotide. In some embodiments, the nucleotides of Y₂ comprise at least one (e.g., 1, 2, 3, 4, 5, 6, 7, 8, 9, 10, 11, 12, 13, 14, 15, 16, 17, 18, 19, 20, 25 or more adenines). In some embodiments, Y₃ comprises 1-5, 1-4, 1-3 or 1-2 nucleotides following the 3′ end of the stem region. In some embodiments, the nucleotides of Y₃ following the 3′ end of the stem region are DNA nucleotides. In some embodiments, Y₃ comprises a pyrimidine complementary with guanine (e.g., cytosine or an analogue thereof). In some embodiments, Y₃ comprises one or more (e.g., two) pyrimidines complementary with guanine at a position following the 3′-end of the stem region (e.g., 1, 2, 3 or more nucleotide after the 3′-end of the stem region). Thus, in embodiments where the RNA transcript is capped, Y₃ may have a pyrimidine that forms stabilizing hydrogen bonds with the 7-methylguanosine of the cap.

In some embodiments, X₁ and X₂ are complementary with non-overlapping regions of the RNA transcript. In some embodiments, X₁ comprises a region complementary with a 5′ region of the RNA transcript and X₂ comprises a region complementary with a 3′ region of the RNA transcript. For example, if the RNA transcript is polyadenylated, X₂ may comprise a region of complementarity that is complementary with the RNA transcript at a region within 100 nucleotides, within 50 nucleotides, within 25 nucleotides or within 10 nucleotides of the polyadenylation junction of the RNA transcript. In some embodiments, X₂ comprises a region of complementarity that is complementary with the RNA transcript immediately adjacent to or overlapping the polyadenylation junction of the RNA transcript. In some embodiments, X₂ comprises at least 2 consecutive pyrimidine nucleotides (e.g., 5 to 15 pyrimidine nucleotides) complementary with adenine nucleotides of the poly(A) tail of the RNA transcript.

In some embodiments, oligonucleotides are provided that comprise the general formula 5′-X₁-X₂-3′, in which X₁ comprises at least 2 nucleotides that form base pairs with adenine (e.g., thymidines or uridines or analogues thereof); and X₂ comprises a region of complementarity that is complementary with at least 3 contiguous nucleotides of a poly-adenylated RNA transcript, wherein the nucleotide at the 5′-end of the region of complementary of X₂ is complementary with the nucleotide of the RNA transcript that is immediately internal to the poly-adenylation junction of the RNA transcript. In such embodiments, X₁ may comprises 2 to 10, 2 to 20, 5 to 15 or 5 to 25 nucleotides and X₂ may independently comprises 2 to 10, 2 to 20, 5 to 15 or 5 to 25 nucleotides.

In some embodiments, compositions are provided that comprise a first oligonucleotide comprising at least 5 nucleotides (e.g., of 5 to 25 nucleotides) linked through internucleoside linkages, and a second oligonucleotide comprising at least 5 nucleotides (e.g., of 5 to 25 nucleotides) linked through internucleoside linkages, in which the the first oligonucleotide is complementary with at least 5 consecutive nucleotides in proximity to the 5′-end of an RNA transcript and the second oligonucleotide is complementary with at least 5 consecutive nucleotides in proximity to the 3′-end of an RNA transcript. In some embodiments, the 5′ end of the first oligonucleotide is linked with the 3′ end of the second oligonucleotide. In some embodiments, the 3′ end of the first oligonucleotide is linked with the 5′ end of the second oligonucleotide. In some embodiments, the 5′ end of the first oligonucleotide is linked with the 5′ end of the second oligonucleotide. In some embodiments, the 3′ end of the first oligonucleotide is linked with the 3′ end of the second oligonucleotide.

In some embodiments, the first oligonucleotide and second oligonucleotide are joined by a linker. The term “linker” generally refers to a chemical moiety that is capable of covalently linking two or more oligonucleotides. In some embodiments, a linker is resistant cleavage in certain biological contexts, such as in a mammalian cell extract, such as an endosomal extract. However, in some embodiments, at least one bond comprised or contained within the linker is capable of being cleaved (e.g., in a biological context, such as in a mammalian extract, such as an endosomal extract), such that at least two oligonucleotides are no longer covalently linked to one another after bond cleavage. In some embodiments, the linker is not an oligonucleotide having a sequence complementary with the RNA transcript. In some embodiments, the linker is an oligonucleotide (e.g., 2-8 thymines). In some embodiments, the linker is a polypeptide. Other appropriate linkers may also be used, including, for example, linkers disclosed in International Patent Application Publication WO 2013/040429 A1, published on Mar. 21, 2013, and entitled MULTIMERIC ANTISENSE OLIGONUCLEOTIDES. The contents of this publication relating to linkers are incorporated herein by reference in their entirety.

An oligonucleotide may have a region of complementarity with a target RNA transcript (e.g., a mammalin mRNA transcript) that has less than a threshold level of complementarity with every sequence of nucleotides, of equivalent length, of an off-target RNA transcript. For example, an oligonucleotide may be designed to ensure that it does not have a sequence that targets RNA transcripts in a cell other than the target RNA transcript. The threshold level of sequence identity may be 50%, 60%, 70%, 80%, 85%, 90%, 95%, 99% or 100% sequence identity.

An oligonucleotide may be complementary to RNA transcripts encoded by homologues of a gene across different species (e.g., a mouse, rat, rabbit, goat, monkey, etc.) In some embodiments, oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of an oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a target RNA. In some embodiments, the region of complementarity is complementary with at least 8 consecutive nucleotides of a target RNA.

Complementary, as the term is used in the art, refers to the capacity for precise pairing between two nucleotides. For example, if a nucleotide at a certain position of an oligonucleotide is capable of hydrogen bonding with a nucleotide at a corresponding position of a target RNA, then the nucleotide of the oligonucleotide and the nucleotide of the target RNA are complementary to each other at that position. The oligonucleotide and target RNA are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and target RNA. For example, if a base at one position of an oligonucleotide is capable of hydrogen bonding with a base at the corresponding position of a target RNA, then the bases are considered to be complementary to each other at that position. 100% complementarity is not required.

An oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a target RNA. In some embodiments an oligonucleotide may contain 1, 2 or 3 base mismatches compared to the portion of the consecutive nucleotides of the target RNA. In some embodiments an oligonucleotide may have up to 3 mismatches over 15 bases, or up to 2 mismatches over 10 bases.

In some embodiments, a complementary nucleic acid sequence need not be 100% complementary to that of its target to be specifically hybridizable. In some embodiments, an oligonucleotide for purposes of the present disclosure is specifically hybridizable with a target RNA when hybridization of the oligonucleotide to the target RNA prevents or inhibits degradation of the target RNA, and when there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80 or more nucleotides in length. In some embodiments, the oligonucleotide is 8 to 50, 10 to 30, 9 to 20, 15 to 30 or 8 to 80 nucleotides in length.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, an oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches. Contiguous runs of three or more Gs or Cs may not be preferable in some embodiments. Accordingly, in some embodiments, the oligonucleotide does not comprise a stretch of three or more guanosine nucleotides.

An oligonucleotide may have a sequence that is has greater than 30% G-C content, greater than 40% G-C content, greater than 50% G-C content, greater than 60% G-C content, greater than 70% G-C content, or greater than 80% G-C content. An oligonucleotide may have a sequence that has up to 100% G-C content, up to 95% G-C content, up to 90% G-C content, or up to 80% G-C content. In some embodiments, GC content of an oligonucleotide is preferably between about 30-60%.

It is to be understood that any oligonucleotide provided herein can be excluded.

In some embodiments, it has been found that oligonucleotides disclosed herein may increase stability of a target RNA by at least about 50% (i.e. 150% of normal or 1.5 fold), or by about 2 fold to about 5 fold. In some embodiments, stability (e.g., stability in a cell) may be increased by at least about 15 fold, 20 fold, 30 fold, 40 fold, 50 fold or 100 fold, or any range between any of the foregoing numbers. In some embodiments, increased mRNA stability has been shown to correlate to increased protein expression. Similarly, in some embodiments, increased stability of non-coding positively correlates with increased activity of the RNA.

It is understood that any reference to uses of oligonucleotides or other molecules throughout the description contemplates use of the oligonucleotides or other molecules in preparation of a pharmaceutical composition or medicament for use in the treatment of condition or a disease associated with decreased levels or activity of a RNA transcript. Thus, as one nonlimiting example, this aspect of the invention includes use of oligonucleotides or other molecules in the preparation of a medicament for use in the treatment of disease, wherein the treatment involves posttranscriptionally altering protein and/or RNA levels in a targeted manner.

### Oligonucleotide Modifications

In some embodiments, oligonucleotides are provided with chemistries suitable for delivery, hybridization and stability within cells to target and stabilize RNA transcripts. Furthermore, in some embodiments, oligonucleotide chemistries are provided that are useful for controlling the pharmacokinetics, biodistribution, bioavailability and/or efficacy of the oligonucleotides. Accordingly, oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not induce substantial cleavage or degradation of the target RNA; do not cause substantially complete cleavage or degradation of the target RNA; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; and may have improved endosomal exit.

Oligonucleotides that are designed to interact with RNA to modulate gene expression are a distinct subset of base sequences from those that are designed to bind a DNA target (e.g., are complementary to the underlying genomic DNA sequence from which the RNA is transcribed).

Any of the oligonucleotides disclosed herein may be linked to one or more other oligonucleotides disclosed herein by a linker, e.g., a cleavable linker.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-M0E), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

Any of the modified chemistries or formats of oligonucleotides described herein can be combined with each other, and that one, two, three, four, five, or more different types of modifications can be included within the same molecule.

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often an oligonucleotide has one or more nucleotide analogues. For example, an oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. An oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ligands of the asialoglycoprotein receptor (ASGPR), such as GalNac, or dynamic polyconjugates and variants thereof at its 5′ or 3′ end.

Preferably an oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, an oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′-alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and MM et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Hone et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

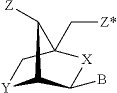

where X and Y are independently selected among the groups —O—,

—S—, —N(H)—, N(R)-, —CH₂- or —CH— (if part of a double bond),

—CH₂—O—, —CH₂—S—, —CH₂—N(H)-, —CH₂—N(R)-, —CH₂—CH₂- or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligonucleotides described herein comprises at least one LNA unit according any of the formulas

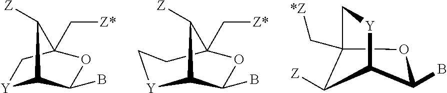

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from -0-P(O)₂—O—, —O—P(O,S)—O—, -0-P(S)₂—O—, —S—P(O)₂—O—, —S—P(O,S)—O—, —S—P(S)₂—O—, -0-P(O)₂—S—, —O—P(O,S)—S—, —S—P(O)₂—S—, —O—PO(R^{H})—O—, 0-PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}-, —NR^{H}—P(O)₂—O—, —NR^{H}—CO—O—, where R^{H} is selected from hydrogen and C₁₋₄-alkyl.

Other examples of LNA units are shown below:

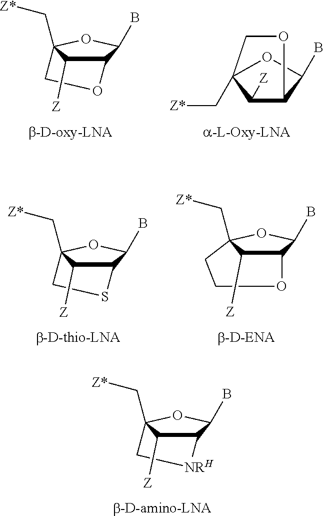

The term “thio-LNA” comprises a locked nucleotide in which at least one of X or Y in the general formula above is selected from S or —CH₂—S—. Thio-LNA can be in both beta-D to and alpha-L-configuration.

The term “amino-LNA” comprises a locked nucleotide in which at least one of X or Y in the general formula above is selected from —N(H)—, N(R)-, CH₂—N(H)-, and —CH₂—N(R)- where R is selected from hydrogen and C₁₋₄-alkyl Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Romberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an intemucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., EBBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, oligonucleotide modification include modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of an oligonucleotide. In some embodiments, an oligonucleotide comprises a biotin moiety conjugated to the 5′ nucleotide.

In some embodiments, an oligonucleotide comprises locked nucleic acids (LNA), ENA modified nucleotides, 2′-O-methyl nucleotides, or 2′-fluoro-deoxyribonucleotides. In some embodiments, an oligonucleotide comprises alternating deoxyribonucleotides and 2′-fluoro-deoxyribonucleotides. In some embodiments, an oligonucleotide comprises alternating deoxyribonucleotides and 2′-O-methyl nucleotides. In some embodiments, an oligonucleotide comprises alternating deoxyribonucleotides and ENA modified nucleotides.

In some embodiments, an oligonucleotide comprises alternating deoxyribonucleotides and locked nucleic acid nucleotides. In some embodiments, an oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, an oligonucleotide comprises phosphorothioate internucleotide linkages. In some embodiments, an oligonucleotide comprises phosphorothioate internucleotide linkages between at least two nucleotides. In some embodiments, an oligonucleotide comprises phosphorothioate internucleotide linkages between all nucleotides.

It should be appreciated that an oligonucleotide can have any combination of modifications as described herein.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

### Methods for Modulating Gene Expression

In one aspect, the invention relates to methods for modulating (e.g., increasing) stability of RNA transcripts in cells. The cells can be in vitro, ex vivo, or in vivo. The cells can be in a subject who has a disease resulting from reduced expression or activity of the RNA transcript or its corresponding protein product in the case of mRNAs. In some embodiments, methods for modulating stability of RNA transcripts in cells comprise delivering to the cell an oligonucleotide that targets the RNA and prevents or inhibits its degradation by exonucleases. In some embodiments, delivery of an oligonucleotide to the cell results in an increase in stability of a target RNA that is at least 5%, 10%, 20%, 30%, 40%, 50%, 60%, 70%, 80%, 90%, 100%, 200% or more greater than a level of stability of the target RNA in a control cell. An appropriate control cell may be a cell to which an oligonucleotide has not been delivered or to which a negative control has been delivered (e.g., a scrambled oligo, a carrier, etc.).

Another aspect of the invention provides methods of treating a disease or condition associated with low levels of a particular RNA in a subject. Accordingly, in some embodiments, methods are provided that comprise administering to a subject (e.g. a human) a composition comprising an oligonucleotide as described herein to increase mRNA stability in cells of the subject for purposes of increasing protein levels. In some embodiments, the increase in protein levels is at least 5%, 10%, 20%, 30%, 40%, 50%, 60%, 70%, 80%, 90%, 100%, 200%, or more, higher than the amount of a protein in the subject (e.g., in a cell or tissue of the subject) before administering or in a control subject which has not been administered the oligonucleotide or that has been administered a negative control (e.g., a scrambled oligo, a carrier, etc.). In some embodiments, methods are provided that comprise administering to a subject (e.g. a human) a composition comprising an oligonucleotide as described herein to increase stability of non-coding RNAs in cells of the subject for purposes of increasing activity of those non-coding RNAs.

A subject can include a non-human mammal, e.g. mouse, rat, guinea pig, rabbit, cat, dog, goat, cow, or horse. In preferred embodiments, a subject is a human. Oligonucleotides may be employed as therapeutic moieties in the treatment of disease states in animals, including humans. Oligonucleotides can be useful therapeutic modalities that can be configured to be useful in treatment regimes for the treatment of cells, tissues and animals, especially humans.

For therapeutics, an animal, preferably a human, suspected of having a disease associated with low levels of an RNA or protein is treated by administering oligonucleotide in accordance with this invention. For example, in one non-limiting embodiment, the methods comprise the step of administering to the animal in need of treatment, a therapeutically effective amount of an oligonucleotide as described herein. Table 1 listed examples of diseases or conditions that may be treated by targeting mRNA transcripts with stabilizing oligonucleotides. In some embodiments, cells used in the methods disclosed herein may, for example, be cells obtained from a subject having one or more of the conditions listed in Table 1, or from a subject that is a disease model of one or more of the conditions listed in Table 1.

TABLE 1
Examples of diseases or conditions treatable with oligonucleotides targeting
associated mRNA.

| Gene | Disease or conditions |
|---|---|
| FXN | Friedreich's Ataxia |
| SMN | Spinal muscular atrophy (SMA) types I-IV |
| UTRN | Muscular dystrophy (MD) (e.g., Duchenne's muscular dystrophy, |
| | Becker's muscular dystrophy, myotonic dystrophy) |
| HEMOGLOBIN | Anemia, microcytic anemia, sickle cell anemia and/or thalassemia (e.g., |
| | alpha-thalassemia, beta-thalaseemia, delta-thalessemia), beta-thalaseemia |
| | (e.g., thalassemia minor/intermedia/major) |
| ATP2A2 | Cardiac conditions (e.g., congenital heart disease, aortic aneurysms, |
| | aortic dissections, arrhythmia, cardiomyopathy, and congestive heart |
| | failure), Darier-White disease and Acrokeratosis verruciformi |
| APOA1/ | Dyslipidemia (e.g. Hyperlipidemia) and atherosclerosis (e.g. coronary |
| ABCA1 | artery disease (CAD) and myocardial infarction (MI)) |
| PTEN | Cancer, such as, leukemias, lymphomas, myelomas, carcinomas, |
| | metastatic carcinomas, sarcomas, adenomas, nervous system cancers and |
| | genito-urinary cancers. In some embodiments, the cancer is adult and |
| | pediatric acute lymphoblastic leukemia, acute myeloid leukemia, |
| | adrenocortical carcinoma, AIDS-related cancers, anal cancer, cancer of |
| | the appendix, astrocytoma, basal cell carcinoma, bile duct cancer, |
| | bladder cancer, bone cancer, osteosarcoma, fibrous histiocytoma, brain |
| | cancer, brain stem glioma, cerebellar astrocytoma, malignant glioma, |
| | ependymoma, medulloblastoma, supratentorial primitive |
| | neuroectodermal tumors, hypothalamic glioma, breast cancer, male |
| | breast cancer, bronchial adenomas, Burkitt lymphoma, carcinoid tumor, |
| | carcinoma of unknown origin, central nervous system lymphoma, |
| | cerebellar astrocytoma, malignant glioma, cervical cancer, childhood |
| | cancers, chronic lymphocytic leukemia, chronic myelogenous leukemia, |
| | chronic myeloproliferative disorders, colorectal cancer, cutaneous T-cell |
| | lymphoma, endometrial cancer, ependymoma, esophageal cancer, Ewing |
| | family tumors, extracranial germ cell tumor, extragonadal germ cell |
| | tumor, extrahepatic bile duct cancer, intraocular melanoma, |
| | retinoblastoma, gallbladder cancer, gastric cancer, gastrointestinal |
| | stromal tumor, extracranial germ cell tumor, extragonadal germ cell |
| | tumor, ovarian germ cell tumor, gestational trophoblastic tumor, glioma, |
| | hairy cell leukemia, head and neck cancer, hepatocellular cancer, |
| | Hodgkin lymphoma, non-Hodgkin lymphoma, hypopharyngeal cancer, |
| | hypothalamic and visual pathway glioma, intraocular melanoma, islet |
| | cell tumors, Kaposi sarcoma, kidney cancer, renal cell cancer, laryngeal |
| | cancer, lip and oral cavity cancer, small cell lung cancer, non-small cell |
| | lung cancer, primary central nervous system lymphoma, Waldenstrom |
| | macroglobulinema, malignant fibrous histiocytoma, medulloblastoma, |
| | melanoma, Merkel cell carcinoma, malignant mesothelioma, squamous |
| | neck cancer, multiple endocrine neoplasia syndrome, multiple myeloma, |
| | mycosis fungoides, myelodysplastic syndromes, myeloproliferative |
| | disorders, chronic myeloproliferative disorders, nasal cavity and |
| | paranasal sinus cancer, nasopharyngeal cancer, neuroblastoma, |
| | oropharyngeal cancer, ovarian cancer, pancreatic cancer, parathyroid |
| | cancer, penile cancer, pharyngeal cancer, pheochromocytoma, |
| | pineoblastoma and supratentorial primitive neuroectodermal tumors, |
| | pituitary cancer, plasma cell neoplasms, pleuropulmonary blastoma, |
| | prostate cancer, rectal cancer, rhabdomyosarcoma, salivary gland cancer, |
| | soft tissue sarcoma, uterine sarcoma, Sezary syndrome, non-melanoma |
| | skin cancer, small intestine cancer, squamous cell carcinoma, squamous |
| | neck cancer, supratentorial primitive neuroectodermal tumors, testicular |
| | cancer, throat cancer, thymoma and thymic carcinoma, thyroid cancer, |
| | transitional cell cancer, trophoblastic tumors, urethral cancer, uterine |
| | cancer, uterine sarcoma, vaginal cancer, vulvar cancer, or Wilms tumor |
| BDNF | Amyotrophic lateral sclerosis (ALS, also known as Lou Gehrig's |
| | disease), Alzheimer's Disease (AD), and Parkinson's Disease (PD), |
| | Neurodegeneration |
| MECP2 | Rett Syndrome, MECP2-related severe neonatal encephalopathy, |
| | Angelman syndrome, or PPM-X syndrome |
| FOXP3 | Diseases or disorders associated with aberrant immune cell (e.g., T cell) |
| | activation, e.g., autoimmune or inflammatory diseases or disorders. |
| | Examples of autoimmune diseases and disorders that may be treated |
| | according to the methods disclosed herein include, but are not limited to, |
| | Acute Disseminated Encephalomyelitis (ADEM), Acute necrotizing |
| | hemorrhagic leukoencephalitis, Addison's disease, |
| | Agammaglobulinemia, Alopecia areata, Amyloidosis, Ankylosing |
| | spondylitis, Anti-GBM/Anti-TBM nephritis, Antiphospholipid syndrome |
| | (APS), Autoimmune angioedema, Autoimmune aplastic anemia, |
| | Autoimmune dysautonomia, Autoimmune hepatitis, Autoimmune |
| | hyperlipidemia, Autoimmune immunodeficiency, Autoimmune inner ear |
| | disease (AIED), Autoimmune myocarditis, Autoimmune oophoritis, |
| | Autoimmune pancreatitis, Autoimmune retinopathy, Autoimmune |
| | thrombocytopenic purpura (ATP), Autoimmune thyroid disease, |
| | Autoimmune urticaria, Axonal & neuronal neuropathies, Balo disease, |
| | Behcet's disease, Bullous pemphigoid, Cardiomyopathy, Castleman |
| | disease, Celiac disease, Chagas disease, Chronic inflammatory |
| | demyelinating polyneuropathy (CIDP), Chronic recurrent multifocal |
| | ostomyelitis (CRMO), Churg-Strauss syndrome, Cicatricial |
| | pemphigoid/benign mucosal pemphigoid, inflammatory bowel disease |
| | (e.g., Crohn's disease or Ulcerative colitis), Cogans syndrome, Cold |
| | agglutinin disease, Congenital heart block, Coxsackie myocarditis, |
| | CREST disease, Essential mixed cryoglobulinemia, Demyelinating |
| | neuropathies, Dermatitis herpetiformis, Dermatomyositis, Devic's |
| | disease (neuromyelitis optica), Discoid lupus, Dressler's syndrome, |
| | Endometriosis, Eosinophilic esophagitis, Eosinophilic fasciitis, Erythema |
| | nodosum, Experimental allergic encephalomyelitis, Evans syndrome, |
| | Fibrosing alveolitis, Giant cell arteritis (temporal arteritis), Giant cell |
| | myocarditis, Glomerulonephritis, Goodpasture's syndrome, |
| | Granulomatosis with Polyangiitis (GPA) (formerly called Wegener's |
| | Granulomatosis), Graves' disease, Guillain-Barre syndrome, Hashimoto's |
| | encephalitis, Hashimoto's thyroiditis, Hemolytic anemia, Henoch- |
| | Schonlein purpura, Herpes gestationis, Hypogammaglobulinemia, |
| | Idiopathic thrombocytopenic purpura (ITP), IgA nephropathy, IgG4- |
| | related sclerosing disease, Immunoregulatory lipoproteins, Inclusion |
| | body myositis, Interstitial cystitis, IPEX (Immunodysregulation, |
| | Polyendocrinopathy, and Enteropathy, X-linked) syndrome, Juvenile |
| | arthritis, Juvenile diabetes (Type 1 diabetes), Juvenile myositis, |
| | Kawasaki syndrome, Lambert-Eaton syndrome, Leukocytoclastic |
| | vasculitis, Lichen planus, Lichen sclerosus, Ligneous conjunctivitis, |
| | Linear IgA disease (LAD), systemic lupus erythematosus (SLE), chronic |
| | Lyme disease, Meniere's disease, Microscopic polyangiitis, Mixed |
| | connective tissue disease (MCTD), Mooren's ulcer, Mucha-Habermann |
| | disease, Multiple sclerosis, Myasthenia gravis, Myositis, Narcolepsy, |
| | Neuromyelitis optica (Devic's), Neutropenia ,Ocular cicatricial |
| | pemphigoid, Optic neuritis, Palindromic rheumatism, PANDAS |
| | (Pediatric Autoimmune Neuropsychiatric Disorders Associated with |
| | Streptococcus), Paraneoplastic cerebellar degeneration, Paroxysmal |
| | nocturnal hemoglobinuria (PNH), Parry Romberg syndrome, |
| | Parsonnage-Turner syndrome, Pars planitis (peripheral uveitis), |
| | Pemphigus, Peripheral neuropathy, Perivenous encephalomyelitis, |
| | Pernicious anemia, POEMS syndrome, Polyarteritis nodosa, Type I, II, |
| | & III autoimmune polyglandular syndromes, Polymyalgia rheumatica, |
| | Polymyositis, Postmyocardial infarction syndrome, Postpericardiotomy |
| | syndrome, Progesterone dermatitis, Primary biliary cirrhosis, Primary |
| | sclerosing cholangitis, Psoriasis, Psoriatic arthritis, Idiopathic pulmonary |
| | fibrosis, Pyoderma gangrenosum, Pure red cell aplasia, Raynauds |
| | phenomenon, Reactive Arthritis, Reflex sympathetic dystrophy, Reiter's |
| | syndrome, Relapsing polychondritis, Restless legs syndrome, |
| | Retroperitoneal fibrosis, Rheumatic fever, Rheumatoid arthritis, |
| | Sarcoidosis, Schmidt syndrome, Scleritis, Scleroderma, Sjogren's |
| | syndrome, Sperm & testicular autoimmunity, Stiff person syndrome, |
| | Subacute bacterial endocarditis (SBE), Susac's syndrome, Sympathetic |
| | ophthalmia, Takayasu's arteritis, Temporal arteritis/Giant cell arteritis, |
| | Thrombocytopenic purpura (TTP), Tolosa-Hunt syndrome, Transverse |
| | myelitis, Type 1 diabetes, Undifferentiated connective tissue disease |
| | (UCTD), Uveitis, Vasculitis, Vesiculobullous dermatosis, Vitiligo, and |
| | Wegener's granulomatosis (also called Granulomatosis with Polyangiitis |
| | (GPA)). Further examples of autoimmune disease or disorder include |
| | inflammatory bowel disease (e.g., Crohn's disease or Ulcerative colitis), |
| | IPEX syndrome, Multiple sclerosis, Psoriasis, Rheumatoid arthritis, SLE |
| | or Type 1 diabetes. Examples of inflammatory diseases or disorders that |
| | may be treated according to the methods disclosed herein include, but are |
| | not limited to, Acne Vulgaris, Appendicitis, Arthritis, Asthma, |
| | Atherosclerosis, Allergies (Type 1 Hypersensitivity), Bursitis, Colitis, |
| | Chronic Prostatitis, Cystitis, Dermatitis, Glomerulonephritis, |
| | Inflammatory Bowel Disease, Inflammatory Myopathy (e.g., |
| | Polymyositis, Dermatomyositis, or Inclusion-body Myositis), |
| | Inflammatory Lung Disease, Interstitial Cystitis, Meningitis, Pelvic |
| | Inflammatory Disease, Phlebitis, Psoriasis, Reperfusion Injury, |
| | Rheumatoid Arthritis, Sarcoidosis, Tendonitis, Tonsilitis, Transplant |
| | Rejection, and Vasculitis. In some embodiments, the inflammatory |
| | disease or disorder is asthma. |

### Formulation, Delivery, And Dosing

The oligonucleotides described herein can be formulated for administration to a subject for treating a condition associated with decreased levels of expression of gene or instability or low stability of an RNA transcript that results in decreased levels of expression of a gene (e.g., decreased protein levels or decreased levels of functional RNAs, such as miRNAs, snoRNAs, 1 ncRNAs, etc.). It should be understood that the formulations, compositions and methods can be practiced with any of the oligonucleotides disclosed herein.

The formulations may conveniently be presented in unit dosage form and may be prepared by any methods well known in the art of pharmacy. The amount of active ingredient (e.g., an oligonucleotide or compound of the invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor regression.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated oligonucleotide composition can assume a variety of states. In some examples, the composition is at least partially crystalline, uniformly crystalline, and/or anhydrous (e.g., less than 80, 50, 30, 20, or 10% water). In another example, an oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, an oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated or administered (together or separately) in combination with another agent, e.g., another therapeutic agent or an agent that stabilizes an oligonucleotide, e.g., a protein that complexes with oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, an oligonucleotide preparation includes another oligonucleotide, e.g., a second oligonucleotide that modulates expression of a second gene or a second oligonucleotide that modulates expression of the first gene. Still other preparation can include at least 3, 5, ten, twenty, fifty, or a hundred or more different oligonucleotide species. Such oligonucleotides can mediated gene expression with respect to a similar number of different genes. In one embodiment, an oligonucleotide preparation includes at least a second therapeutic agent (e.g., an agent other than an oligonucleotide).

Any of the formulations, excipients, vehicles, etc. disclosed herein may be adapted or used to facilitate delivery of synthetic RNAs (e.g., circularized synthetic RNAs) to a cell. Formulations, excipients, vehicles, etc. disclosed herein may be adapted or used to facilitate delivery of a synthetic RNA to a cell in vitro or in vivo. For example, a synthetic RNA (e.g., a circularized synthetic RNA) may be formulated with a nanoparticle, poly(lactic-co-glycolic acid) (PLGA) microsphere, lipidoid, lipoplex, liposome, polymer, carbohydrate (including simple sugars), cationic lipid, a fibrin gel, a fibrin hydrogel, a fibrin glue, a fibrin sealant, fibrinogen, thrombin, rapidly eliminated lipid nanoparticles (reLNPs) and combinations thereof. In some embodiments, a synthetic RNA may be delivered to a cell gymnotically. In some embodiments, oligonucleotides or synthetic RNAs may be conjugated with factors that facilitate delivery to cells. In some embodiments, a synthetic RNA or oligonucleotide used to circularize a synthetic RNA is conjugated with a carbohydrate, such as GalNac, or other targeting moiety.

### Route of Delivery

A composition that includes an oligonucleotide can be delivered to a subject by a variety of routes. Exemplary routes include: intravenous, intradermal, topical, rectal, parenteral, anal, intravaginal, intranasal, pulmonary, ocular. The term “therapeutically effective amount” is the amount of oligonucleotide present in the composition that is needed to provide the desired level of gene expression (e.g., by stabilizing RNA transcripts) in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

An oligonucleotide molecules of the invention can be incorporated into pharmaceutical compositions suitable for administration. Such compositions typically include one or more species of oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering an oligonucleotide in aerosol form. The vascular endothelial cells could be targeted by coating a balloon catheter with an oligonucleotide and mechanically introducing the oligonucleotide.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. An oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. An oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm. The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide, e.g., a device can release insulin. In one embodiment, unit doses or measured doses of a composition that includes oligonucleotide are dispensed by an implanted device. The device can include a sensor that monitors a parameter within a subject. For example, the device can include pump, e.g., and, optionally, associated electronics.

Tissue, e.g., cells or organs can be treated with an oligonucleotide, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, an oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

In one embodiment, a contraceptive device is coated with or contains an oligonucleotide. Exemplary devices include condoms, diaphragms, IUD (implantable uterine devices, sponges, vaginal sheaths, and birth control devices.

Dosage

In one aspect, the invention features a method of administering an oligonucleotide (e.g., as a compound or as a component of a composition) to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with low levels of an RNA or protein. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of an oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some cases, a patient is treated with an oligonucleotide in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of an oligonucleotide composition is an amount sufficient to be effective in treating or preventing a disorder or to regulate a physiological condition in humans. The concentration or amount of oligonucleotide administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide can include a single treatment or, preferably, can include a series of treatments. It will also be appreciated that the effective dosage of an oligonucleotide used for treatment may increase or decrease over the course of a particular treatment. For example, the subject can be monitored after administering an oligonucleotide composition. Based on information from the monitoring, an additional amount of an oligonucleotide composition can be administered.

Dosing is dependent on severity and responsiveness of the disease condition to be treated, with the course of treatment lasting from several days to several months, or until a cure is effected or a diminution of disease state is achieved. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models.

In one embodiment, the administration of an oligonucleotide composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising an oligonucleotide. In some embodiments, the composition is a pharmaceutical composition comprising an oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Oligonucleotide for Targeting 5′ and 3′ Ends of RNAs

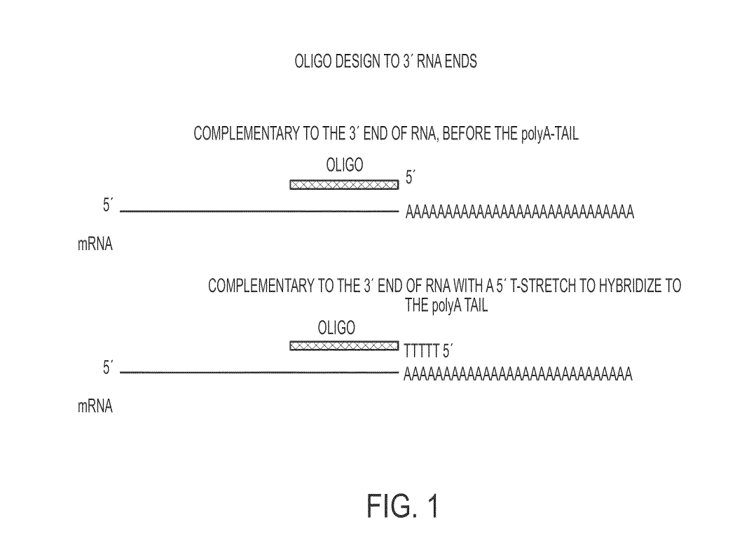

Several exemplary oligonucleotide design schemes are contemplated herein for increasing mRNA stability. With regard to oligonucleotides targeting the 3′ end of an RNA, at least two exemplary design schemes are contemplated. As a first scheme, an oligo nucleotide is designed to be complementary to the 3′ end of an RNA, before the poly-A tail (FIG. 1). As a second scheme, an oligonucleotide is designed to be complementary to the 3′ end of RNA with a 5′ poly-T region that hybridizes to a poly-A tail (FIG. 1).

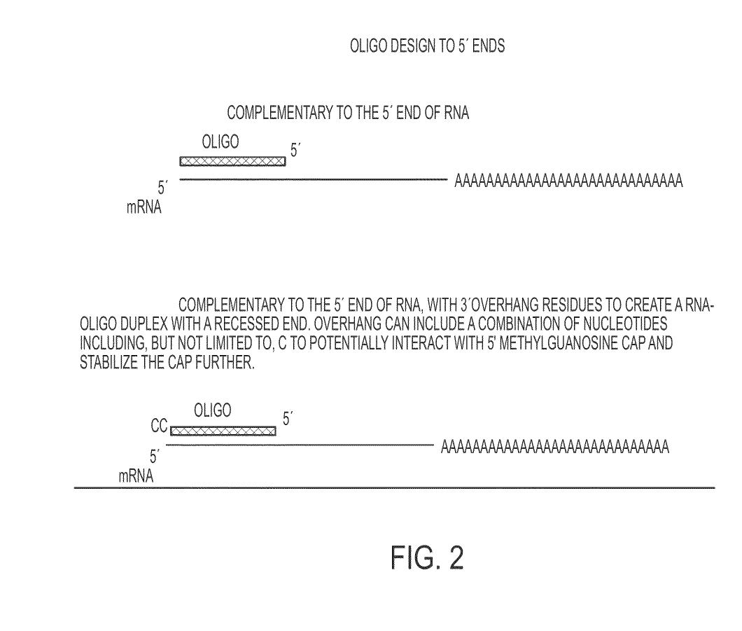

With regard to oligonucleotides targeting the 5′ end of an RNA, at least three exemplary design schemes are contemplated. For scheme one, an oligonucleotide is designed to be complementary to the 5′ end of RNA (FIG. 2). For scheme two, an oligonucleotide is designed to be complementary to the 5′ end of RNA and has a 3′ overhang to create a RNA-oligo duplex with a recessed end. In this example, the overhang is one or more C nucleotides, e.g., two Cs, which can potentially interact with a 5′ methylguanosine cap and stabilize the cap further (FIG. 2). The overhang could also potentially be another type of nucleotide, and is not limited to C. For scheme 3, an oligonucleotide is designed to include a loop region to stabilize 5′ RNA cap.

An oligonucleotide designed as described in Example 1 may be tested for its ability to upregulate RNA by increasing mRNA stability using the methods outlined in Example 2.

### Example 2

### Oligos for Targeting the 5′ and 3′ End of Frataxin

### Materials and Methods:

### Real Time PCR

RNA analysis, cDNA synthesis and QRT-PCR was done with Life Technologies Cells-to-Ct kit and StepOne Plus instrument. Baseline levels were also determined for mRNA of various housekeeping genes which are constitutively expressed. A “control” housekeeping gene with approximately the same level of baseline expression as the target gene was chosen for comparison purposes

### Western Blot

Western blots were performed as previously described. KLF4 antibody (Cell Signaling 4038S) was used at 1:1000 dilution. The images were taken on a UVP ChemicDoc-It instrument using fluorescently-labeled anti-rabbit antibodies.

### ELISA

ELISA assays were performed using the Abcam Frataxin ELISA kit (ab115346) following manufacturer's instructions.

### Cell Lines

Cells were cultured using conditions known in the art. Details of the cell lines used in the experiments described herein are provided in Table 2.

TABLE 2
Cells

| | Clinically | | # of GAA | |
|---|---|---|---|---|
| Cell lines | affected | Cell type | repeats | Notes |
| GM15850 | Y | B- | 650 & 1030 | 13 yr old white male, brother to |
| | | lymphoblast | | GM15851 |
| GM15851 | N | B- | <20 for both | 14 yr old white male, brother to |
| | | lymphoblast | | GM15850 |
| GM16209 | Y | B- | 800 for both | 41 yr old white female, half-sister |
| | | lymphoblast | | to GM16222 |
| GM16222 | N | B- | 830 & <20 | 59 yr old white female, half-sister |
| | | lymphoblast | | to GM16209 |
| GM03816 | Y | Fibroblast | 330/380 | 36 yr old white female, sister to |
| | | | | GM04078 |
| GM03816 | Y | Fibroblast | 541-420 | 30 yr old white male, brother to |
| | | | | GM03816 |
| GM0321B | N | Fibroblast | Not applicable | Healthy 40 yr old female |

### Actinomycin D Treatment

Actinomycin D (Life Technologies) was added to cell culture media at 10 microgram/ml concentration and incubated. RNA isolation was done using Trizol (Sigma) following manufacturer's instructions. FXN and c-Myc probes were purchased from Life Technologies.

### Oligonucleotide Design

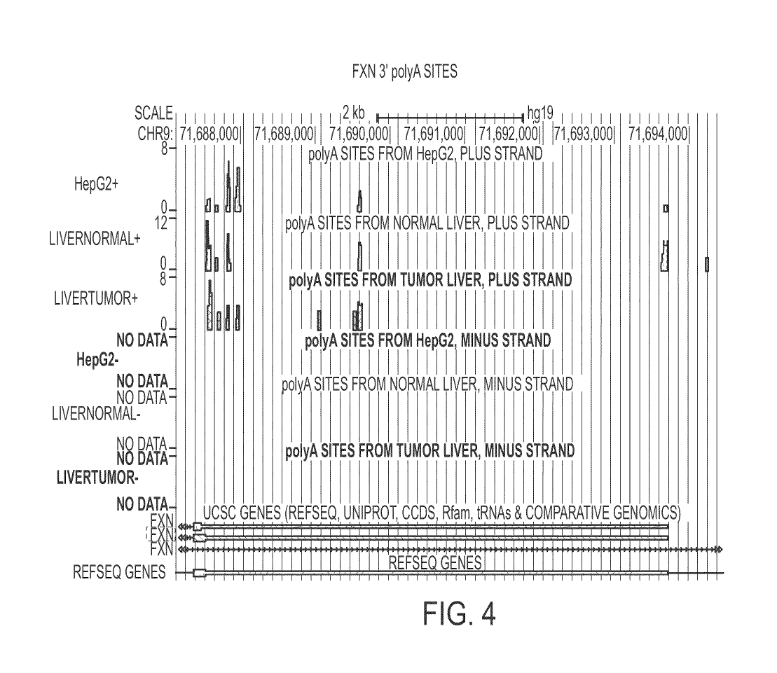

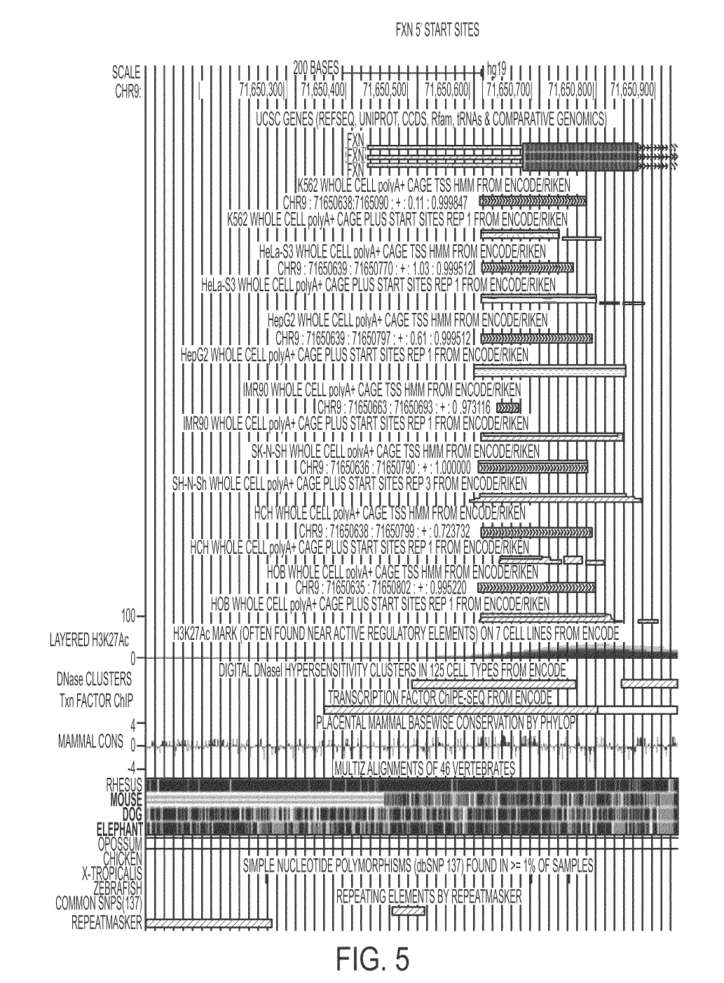

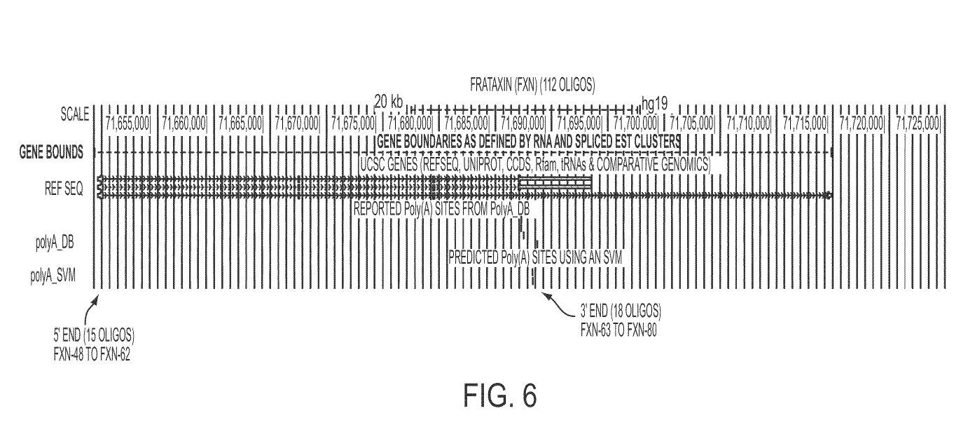

Oligonucleotides were designed to target the 5′ and 3′ ends of FXN mRNA. The 3′ end oligonucleotides were designed by identifying putative mRNA 3′ ends using quantitative end analysis of poly-A tails as described previously (see, e.g., Ozsolak et al. Comprehensive Polyadenylation Site Maps in Yeast and Human Reveal Pervasive Alternative Polyadenylation. Cell. Volume 143, Issue 6, 2010, Pages 1018-1029). FIG. 4 shows the identified poly-A sites. The 5′ end oligonucleotides were designed by identifying potential 5′ start sites using Cap analysis gene expression (CAGE) as previously described (see, e.g., Cap analysis gene expression for high-throughput analysis of transcriptional starting point and identification of promoter usage. Proc Natl Acad Sci USA. 100 (26): 15776-81. 2003-12-23 and Zhao, Xiaobei (2011). “Systematic Clustering of Transcription Start Site Landscapes”. PLoS ONE (Public Library of Science) 6 (8): e23409). FIG. 5 shows the identified 5′ start sites. FIG. 6 provides the location of the designed 5′ and 3′ end oligonucleotides.

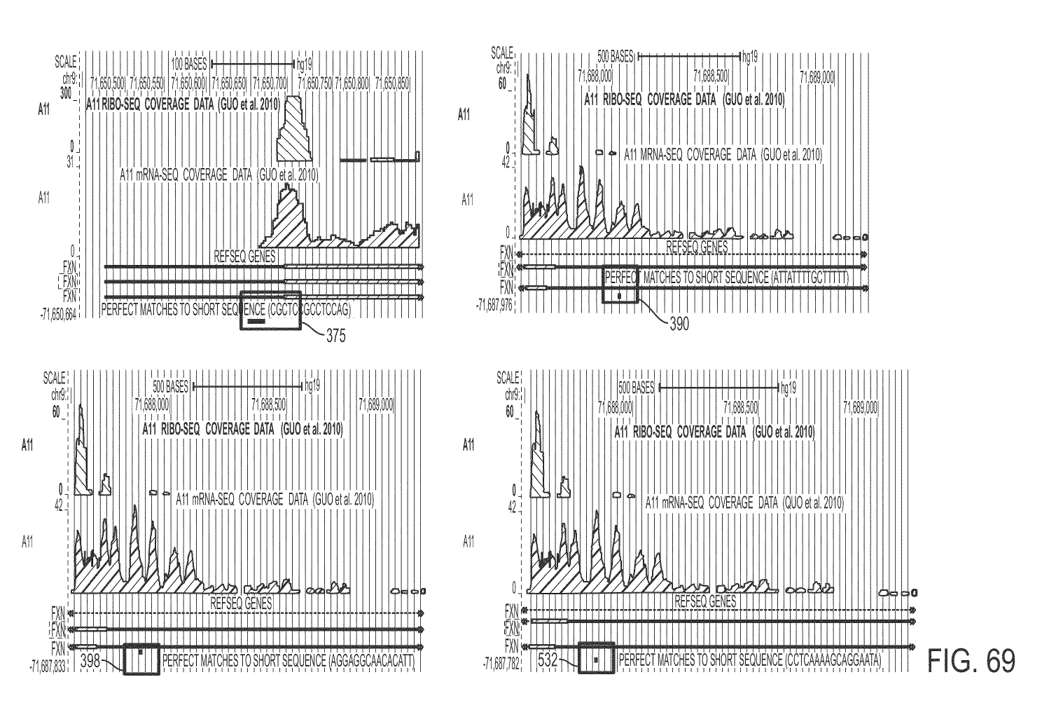

The oligonucleotide positions of certain designed oligonucleotides relative to mRNA-Seq signals and ribosome positioning was also calculated using public data sets (Guo, H., Ingolia, N. T., Weissman, J. S., & Bartel, D. P. (2010). Mammalian microRNAs predominantly act to decrease target mRNA levels. Nature, 466(7308), 835-40. doi:10.1038/nature09267). The oligonucleotide positions relative to these data sets are shown in FIG. 69.

The sequence and structure of each oligonucleotide is shown in Table 3. Table 5 provides a description of the nucleotide analogs, modifications and intranucleotide linkages used for certain oligonucleotides tested and described in Tables 3, 7, 8 9, 10, 11, and 12. Certain oligos in Table 3 and Table 4 have two oligo names the “Oligo Name” and the “Alternative Oligo Name”, which are used interchangeably herein and are to be understood to refer to the same oligo.

TABLE 3
Oligonucleotides targeting 5′ and 3′ ends of FXN

| SEQ | | Alternative | | | | | |
|---|---|---|---|---|---|---|---|
| ID | Oligo | Oligo | Base | Targeting | Gene | | |
| NO | Name | Name | Sequence | Region | Name | Organism | Formatted Sequence |
| 1 | Oligo48 | FXN-371 | TGACCCA | 5′-End | FXN | human | dTs; lnaGs; |
| | | | AGGGAGAC | | | | dAs; lnaCs; |
| | | | | | | | dCs; lnaCs; |
| | | | | | | | dAs; lnaAs; |
| | | | | | | | dGs; lnaGs; |
| | | | | | | | dGs; lnaAs; |
| | | | | | | | dGs; lnaAs; |
| | | | | | | | dC-Sup |
| 2 | Oligo49 | FXN-372 | TGGCCAC | 5′-End | FXN | human | dTs; lnaGs; |
| | | | TGGCCGCA | | | | dGs; lnaCs; |
| | | | | | | | dCs; lnaAs; |
| | | | | | | | dCs; lnaTs; |
| | | | | | | | dGs; lnaGs; |
| | | | | | | | dCs; lnaCs; |
| | | | | | | | dGs; lnaCs; |
| | | | | | | | dA-Sup |
| 3 | Oligo50 | FXN-373 | CGGCGAC | 5′-End | FXN | human | dCs; lnaGs; |
| | | | CCCTGGTG | | | | dGs; lnaCs; |
| | | | | | | | dGs; lnaAs; |
| | | | | | | | dCs; lnaCs; |
| | | | | | | | dCs; lnaCs; |
| | | | | | | | dTs; lnaGs; |
| | | | | | | | dGs; lnaTs; |
| | | | | | | | dG-Sup |
| 4 | Oligo51 | FXN-374 | CGCCCTCC | 5′-End | FXN | human | dCs; lnaGs; |
| | | | AGCGCTG | | | | dCs; lnaCs; |
| | | | | | | | dCs; lnaTs; |
| | | | | | | | dCs; lnaCs; |
| | | | | | | | dAs; lnaGs; |
| | | | | | | | dCs; lnaGs; |
| | | | | | | | dCs; lnaTs; |
| | | | | | | | dG-Sup |
| 5 | Oligo52 | FXN-375 | CGCTCCG | 5′-End | FXN | human | dCs; lnaGs; |
| | | | CCCTCCAG | | | | dCs; lnaTs; |
| | | | | | | | dCs; lnaCs; |
| | | | | | | | dGs; lnaCs; |
| | | | | | | | dCs; lnaCs; |
| | | | | | | | dTs; lnaCs; |
| | | | | | | | dCs; lnaAs; |
| | | | | | | | dG-Sup |
| 6 | Oligo53 | FXN-376 | TGACCCA | 5′-End | FXN | human | dTs; lnaGs; |
| | | | AGGGAGA | | | | dAs; lnaCs; |
| | | | CCC | | | | dCs; lnaCs; |
| | | | | | | | dAs; lnaAs; |
| | | | | | | | dGs; lnaGs; |
| | | | | | | | dGs; lnaAs; |
| | | | | | | | dGs; lnaAs; |
| | | | | | | | dCs; lnaCs; |
| | | | | | | | dC-Sup |
| 7 | Oligo54 | FXN-377 | TGGCCAC | 5′-End | FXN | human | dTs; lnaGs; |
| | | | TGGCCGC | | | | dGs; lnaCs; |
| | | | ACC | | | | dCs; lnaAs; |
| | | | | | | | dCs; lnaTs; |
| | | | | | | | dGs; lnaGs; |
| | | | | | | | dCs; lnaCs; |
| | | | | | | | dGs; lnaCs; |
| | | | | | | | dAs; lnaCs; |
| | | | | | | | dC-Sup |
| 8 | Oligo55 | FXN-378 | CGGCGAC | 5′-End | FXN | human | dCs; lnaGs; |
| | | | CCCTGGT | | | | dGs; lnaCs; |
| | | | GCC | | | | dGs; lnaAs; |
| | | | | | | | dCs; lnaCs; |
| | | | | | | | dCs; lnaCs; |
| | | | | | | | dTs; lnaGs; |
| | | | | | | | dGs; lnaTs; |
| | | | | | | | dGs; lnaCs; |
| | | | | | | | dC-Sup |
| 9 | Oligo56 | FXN-379 | CGCCCTCC | 5′-End | FXN | human | dCs; lnaGs; |
| | | | AGCGCTG | | | | dCs; lnaCs; |
| | | | CC | | | | dCs; lnaTs; |
| | | | | | | | dCs; lnaCs; |
| | | | | | | | dAs; lnaGs; |
| | | | | | | | dCs; lnaGs; |
| | | | | | | | dCs; lnaTs; |
| | | | | | | | dGs; lnaCs; |
| | | | | | | | dC-Sup |
| 10 | Oligo57 | FXN-380 | CGCTCCG | 5′-End | FXN | human | dCs; lnaGs; |
| | | | CCCTCCA | | | | dCs; lnaTs; |
| | | | GCC | | | | dCs; lnaCs; |
| | | | | | | | dGs; lnaCs; |
| | | | | | | | dCs; lnaCs; |
| | | | | | | | dTs; lnaCs; |
| | | | | | | | dCs; lnaAs; |
| | | | | | | | dGs; lnaCs; |
| | | | | | | | dC-Sup |
| 11 | Oligo58 | FXN-381 | TGACCCA | 5′-End | FXN | human | dTs; lnaGs; |
| | | | AGGGAGA | | | | dAs; lnaCs; |
| | | | CGGAAAC | | | | dCs; lnaCs; |
| | | | CAC | | | | dAs; lnaAs; |
| | | | | | | | dGs; lnaGs; |
| | | | | | | | dGs; lnaAs; |
| | | | | | | | dGs; lnaAs; |
| | | | | | | | dCs; lnaGs; |
| | | | | | | | dGs; dAs; |
| | | | | | | | dAs; dAs; |
| | | | | | | | dCs; lnaCs; |
| | | | | | | | dAs; lnaC-Sup |
| 12 | Oligo59 | FXN-382 | TGGCCAC | 5′-End | FXN | human | dTs; lnaGs; |
| | | | TGGCCGC | | | | dGs; lnaCs; |
| | | | AGGAAAC | | | | dCs; lnaAs; |
| | | | CAC | | | | dCs; lnaTs; |
| | | | | | | | dGs; lnaGs; |
| | | | | | | | dCs; lnaCs; |
| | | | | | | | dGs; lnaCs; |
| | | | | | | | dAs; lnaGs; |
| | | | | | | | dGs; dAs; |
| | | | | | | | dAs; dAs; |
| | | | | | | | dCs; lnaCs; |
| | | | | | | | dAs; lnaC-Sup |
| 13 | Oligo60 | FXN-383 | CGGCGAC | 5′-End | FXN | human | dCs; lnaGs; |
| | | | CCCTGGT | | | | dGs; lnaCs; |
| | | | GGGAAAC | | | | dGs; lnaAs; |
| | | | CTC | | | | dCs; lnaCs; |
| | | | | | | | dCs; lnaCs; |
| | | | | | | | dTs; lnaGs; |
| | | | | | | | dGs; lnaTs; |
| | | | | | | | dGs; lnaGs; |
| | | | | | | | dGs; dAs; |
| | | | | | | | dAs; dAs; |
| | | | | | | | dCs; lnaCs; |
| | | | | | | | dTs; lnaC-Sup |
| 14 | Oligo61 | FXN-384 | CGCCCTCC | 5′-End | FXN | human | dCs; lnaGs; |
| | | | AGCGCTG | | | | dCs; lnaCs; |
| | | | GGAAACC | | | | dCs; lnaTs; |
| | | | TC | | | | dCs; lnaCs; |
| | | | | | | | dAs; lnaGs; |
| | | | | | | | dCs; lnaGs; |
| | | | | | | | dCs; lnaTs; |
| | | | | | | | dGs; lnaGs; |
| | | | | | | | dGs; dAs; |
| | | | | | | | dAs; dAs; |
| | | | | | | | dCs; lnaCs; |
| | | | | | | | dTs; lnaC-Sup |
| 15 | Oligo62 | FXN-385 | CGCTCCG | 5′-End | FXN | human | dCs; lnaGs; |
| | | | CCCTCCA | | | | dCs; lnaTs; |
| | | | GCCAAAG | | | | dCs; lnaCs; |
| | | | GTC | | | | dGs; lnaCs; |
| | | | | | | | dCs; lnaCs; |
| | | | | | | | dTs; lnaCs; |
| | | | | | | | dCs; lnaAs; |
| | | | | | | | dGs; lnaCs; |
| | | | | | | | dCs; dAs; |
| | | | | | | | dAs; dAs; |
| | | | | | | | dGs; lnaGs; |
| | | | | | | | dTs; lnaC-Sup |
| 16 | Oligo63 | FXN-386 | GGTTTTTA | 3′-End | FXN | human | dGs; lnaGs; |
| | | | AGGCTTT | | | | dTs; lnaTs; |
| | | | | | | | dTs; lnaTs; |
| | | | | | | | dTs; lnaAs; |
| | | | | | | | dAs; lnaGs; |
| | | | | | | | dGs; lnaCs; |
| | | | | | | | dTs; lnaTs; |
| | | | | | | | dT-Sup |
| 17 | Oligo64 | FXN-387 | GGGGTCT | 3′-End | FXN | human | dGs; lnaGs; |
| | | | TGGCCTGA | | | | dGs; lnaGs; |
| | | | | | | | dTs; lnaCs; |
| | | | | | | | dTs; lnaTs; |
| | | | | | | | dGs; lnaGs; |
| | | | | | | | dCs; lnaCs; |
| | | | | | | | dTs; lnaGs; |
| | | | | | | | dA- |
| | | | | | | | Sup |
| 18 | Oligo65 | FXN-388 | CATAATG | 3′-End | FXN | human | dCs; lnaAs; |
| | | | AAGCTGGG | | | | dTs; lnaAs; |
| | | | | | | | dAs; lnaTs; |
| | | | | | | | dGs; lnaAs; |
| | | | | | | | dAs; lnaGs; |
| | | | | | | | dCs; lnaTs; |
| | | | | | | | dGs; lnaGs; |
| | | | | | | | dG-Sup |
| 19 | Oligo66 | FXN-389 | AGGAGGC | 3′-End | FXN | human | dAs; lnaGs; |
| | | | AACACATT | | | | dGs; lnaAs; |
| | | | | | | | dGs; lnaGs; |
| | | | | | | | dCs; lnaAs; |
| | | | | | | | dAs; lnaCs; |
| | | | | | | | dAs; lnaCs; |
| | | | | | | | dAs; lnaTs; |
| | | | | | | | dT- |
| | | | | | | | Sup |
| 20 | Oligo67 | FXN-390 | ATTATTTT | 3′-End | FXN | human | dAs; lnaTs; |
| | | | GCTTTTT | | | | dTs; lnaAs; |
| | | | | | | | dTs; lnaTs; |
| | | | | | | | dTs; lnaTs; |
| | | | | | | | dGs; lnaCs; |
| | | | | | | | dTs; lnaTs; |
| | | | | | | | dTs; lnaTs; |
| | | | | | | | dT-Sup |
| 21 | Oligo68 | FXN-391 | CATTTTCC | 3′-End | FXN | human | dCs; lnaAs; |
| | | | CTCCTGG | | | | dTs; lnaTs; |
| | | | | | | | dTs; lnaTs; |
| | | | | | | | dCs; lnaCs; |
| | | | | | | | dCs; lnaTs; |
| | | | | | | | dCs; lnaCs; |
| | | | | | | | dTs; lnaGs; |
| | | | | | | | dG-Sup |
| 22 | Oligo69 | FXN-392 | GTAGGCT | 3′-End | FXN | human | dGs; lnaTs; |
| | | | ACCCTTTA | | | | dAs; lnaGs; |
| | | | | | | | dGs; lnaCs; |
| | | | | | | | dTs; lnaAs; |
| | | | | | | | dCs; lnaCs; |
| | | | | | | | dCs; lnaTs; |
| | | | | | | | dTs; lnaTs; |
| | | | | | | | dA-Sup |
| 23 | Oligo70 | FXN-393 | GAGGCTT | 3′-End | FXN | human | dGs; lnaAs; |
| | | | GTTGCTTT | | | | dGs; lnaGs; |
| | | | | | | | dCs; lnaTs; |
| | | | | | | | dTs; lnaGs; |
| | | | | | | | dTs; lnaTs; |
| | | | | | | | dGs; lnaCs; |
| | | | | | | | dTs; lnaTs; |
| | | | | | | | dT-Sup |
| 24 | Oligo71 | FXN-394 | CATGTAT | 3′-End | FXN | human | dCs; lnaAs; |
| | | | GATGTTAT | | | | dTs; lnaGs; |
| | | | | | | | dTs; lnaAs; |
| | | | | | | | dTs; lnaGs; |
| | | | | | | | dAs; lnaTs; |
| | | | | | | | dGs; lnaTs; |
| | | | | | | | dTs; lnaAs; |
| | | | | | | | dT-Sup |
| 25 | Oligo72 | FXN-395 | TTTTTGGT | 3′-End | FXN | human | dTs; lnaTs; |
| | | | TTTTAAG | | | | dTs; lnaTs; |
| | | | GCTTT | | | | dTs; lnaGs; |
| | | | | | | | dGs; lnaTs; |
| | | | | | | | dTs; lnaTs; |
| | | | | | | | dTs; lnaTs; |
| | | | | | | | dAs; lnaAs; |
| | | | | | | | dGs; lnaGs; |
| | | | | | | | dCs; lnaTs; |
| | | | | | | | dTs; lnaT-Sup |
| 26 | Oligo73 | FXN-396 | TTTTTGG | 3′-End | FXN | human | dTs; lnaTs; |
| | | | GGTCTTG | | | | dTs; lnaTs; |
| | | | GCCTGA | | | | dTs; lnaGs; |
| | | | | | | | dGs; lnaGs; |
| | | | | | | | dGs; lnaTs; |
| | | | | | | | dCs; lnaTs; |
| | | | | | | | dTs; lnaGs; |
| | | | | | | | dGs; lnaCs; |
| | | | | | | | dCs; lnaTs; |
| | | | | | | | dGs; lnaA-Sup |
| 27 | Oligo74 | FXN-397 | TTTTTCAT | 3′-End | FXN | human | dTs; lnaTs; |
| | | | AATGAAG | | | | dTs; lnaTs; |
| | | | CTGGG | | | | dTs; lnaCs; |
| | | | | | | | dAs; lnaTs; |
| | | | | | | | dAs; lnaAs; |
| | | | | | | | dTs; lnaGs; |
| | | | | | | | dAs; lnaAs; |
| | | | | | | | dGs; lnaCs; |
| | | | | | | | dTs; lnaGs; |
| | | | | | | | dGs; lnaG-Sup |
| 28 | Oligo75 | FXN-398 | TTTTTAGG | 3′-End | FXN | human | dTs; lnaTs; |
| | | | AGGCAAC | | | | dTs; lnaTs; |
| | | | ACATT | | | | dTs; lnaAs; |
| | | | | | | | dGs; lnaGs; |
| | | | | | | | dAs; lnaGs; |
| | | | | | | | dGs; lnaCs; |
| | | | | | | | dAs; lnaAs; |
| | | | | | | | dCs; lnaAs; |
| | | | | | | | dCs; lnaAs; |
| | | | | | | | dTs; lnaT-Sup |
| 29 | Oligo76 | FXN-399 | TTTTTATT | 3′-End | FXN | human | dTs; lnaTs; |
| | | | ATTTTGCT | | | | dTs; lnaTs; |
| | | | TTTT | | | | dTs; lnaAs; |
| | | | | | | | dTs; lnaTs; |
| | | | | | | | dAs; lnaTs; |
| | | | | | | | dTs; lnaTs; |
| | | | | | | | dTs; lnaGs; |
| | | | | | | | dCs; lnaTs; |
| | | | | | | | dTs; lnaTs; |
| | | | | | | | dTs; lnaT-Sup |
| 30 | Oligo77 | FXN-400 | TTTTTCAT | 3′-End | FXN | human | dTs; lnaTs; |
| | | | TTTCCCTC | | | | dTs; lnaTs; |
| | | | CTGG | | | | dTs; lnaCs; |
| | | | | | | | dAs; lnaTs; |
| | | | | | | | dTs; lnaTs; |
| | | | | | | | dTs; lnaCs; |
| | | | | | | | dCs; lnaCs; |
| | | | | | | | dTs; lnaCs; |
| | | | | | | | dCs; lnaTs; |
| | | | | | | | dGs; lnaG-Sup |
| 31 | Oligo78 | FXN-401 | TTTTTGTA | 3′-End | FXN | human | dTs; lnaTs; |
| | | | GGCTACC | | | | dTs; lnaTs; |
| | | | CTTTA | | | | dTs; lnaGs; |
| | | | | | | | dTs; lnaAs; |
| | | | | | | | dGs; lnaGs; |
| | | | | | | | dCs; lnaTs; |
| | | | | | | | dAs; lnaCs; |
| | | | | | | | dCs; lnaCs; |
| | | | | | | | dTs; lnaTs; |
| | | | | | | | dTs; lnaA-Sup |
| 32 | Oligo79 | FXN-402 | TTTTTGAG | 3′-End | FXN | human | dTs; lnaTs; |
| | | | GCTTGTT | | | | dTs; lnaTs; |
| | | | GCTTT | | | | dTs; lnaGs; |
| | | | | | | | dAs; lnaGs; |
| | | | | | | | dGs; lnaCs; |
| | | | | | | | dTs; lnaTs; |
| | | | | | | | dGs; lnaTs; |
| | | | | | | | dTs; lnaGs; |
| | | | | | | | dCs; lnaTs; |
| | | | | | | | dTs; lnaT-Sup |
| 33 | Oligo80 | FXN-403 | TTTTTCAT | 3′-End | FXN | human | dTs; lnaTs; |
| | | | GTATGAT | | | | dTs; lnaTs; |
| | | | GTTAT | | | | dTs; lnaCs; |
| | | | | | | | dAs; lnaTs; |
| | | | | | | | dGs; lnaTs; |
| | | | | | | | dAs; lnaTs; |
| | | | | | | | dGs; lnaAs; |
| | | | | | | | dTs; lnaGs; |
| | | | | | | | dTs; lnaTs; |
| | | | | | | | dAs; lnaT-Sup |

TABLE 4
Other oligonucleotides targeting FXN

| SEQ | | Alternative | | | | | |
|---|---|---|---|---|---|---|---|
| ID | Oligo | Oligo | Base | Targeting | Gene | | Formatted |
| NO | Name | Name | Sequence | Region | Name | Organism | Sequence |
| 34 | Oligo1 | FXN-324 | CGGCGCC | Internal | FXN | human | dCs; lnaGs; |
| | | | CGAGAGT | | | | dGs; lnaCs; |
| | | | CCACAT | | | | dGs; lnaCs; |
| | | | | | | | dCs; lnaCs; |
| | | | | | | | dGs; lnaAs; |
| | | | | | | | dGs; lnaAs; |
| | | | | | | | dGs; lnaTs; |
| | | | | | | | dCs; lnaCs; |
| | | | | | | | dAs; lnaCs; |
| | | | | | | | dAs; lnaT- |
| | | | | | | | Sup |
| 35 | Oligo2 | FXN-325 | CCAGGAG | Internal | FXN | human | dCs; lnaCs; |
| | | | GCCGGCT | | | | dAs; lnaGs; |
| | | | ACTGCG | | | | dGs; lnaAs; |
| | | | | | | | dGs; lnaGs; |
| | | | | | | | dCs; lnaCs; |
| | | | | | | | dGs; lnaGs; |
| | | | | | | | dCs; lnaTs; |
| | | | | | | | dAs; lnaCs; |
| | | | | | | | dTs; lnaGs; |
| | | | | | | | dCs; lnaG- |
| | | | | | | | Sup |
| 36 | Oligo3 | FXN-326 | CTGGGCT | Internal | FXN | human | dCs; lnaTs; |
| | | | GGGCTGG | | | | dGs; lnaGs; |
| | | | GTGACG | | | | dGs; lnaCs; |
| | | | | | | | dTs; lnaGs; |
| | | | | | | | dGs; lnaGs; |
| | | | | | | | dCs; lnaTs; |
| | | | | | | | dGs; lnaGs; |
| | | | | | | | dGs; lnaTs; |
| | | | | | | | dGs; lnaAs; |
| | | | | | | | dCs; lnaG- |
| | | | | | | | Sup |
| 37 | Oligo4 | FXN-327 | ACCCGGG | Internal | FXN | human | dAs; lnaCs; |
| | | | TGAGGGT | | | | dCs; lnaCs; |
| | | | CTGGGC | | | | dGs; lnaGs; |
| | | | | | | | dGs; lnaTs; |
| | | | | | | | dGs; lnaAs; |
| | | | | | | | dGs; lnaGs; |
| | | | | | | | dGs; lnaTs; |
| | | | | | | | dCs; lnaTs; |
| | | | | | | | dGs; lnaGs; |
| | | | | | | | dGs; lnaC- |
| | | | | | | | Sup |
| 38 | Oligo5 | FXN-328 | CCAACTCT | Internal | FXN | human | dCs; lnaCs; |
| | | | GCCGGCC | | | | dAs; lnaAs; |
| | | | GCGGG | | | | dCs; lnaTs; |
| | | | | | | | dCs; lnaTs; |
| | | | | | | | dGs; lnaCs; |
| | | | | | | | dCs; lnaGs; |
| | | | | | | | dGs; lnaCs; |
| | | | | | | | dCs; lnaGs; |
| | | | | | | | dCs; lnaGs; |
| | | | | | | | dGs; lnaG- |
| | | | | | | | Sup |
| 39 | Oligo6 | FXN-329 | ACGGCGG | Internal | FXN | human | dAs; lnaCs; |
| | | | CCGCAGA | | | | dGs; lnaGs; |
| | | | GTGGGG | | | | dCs; lnaGs; |
| | | | | | | | dGs; lnaCs; |
| | | | | | | | dCs; lnaGs; |
| | | | | | | | dCs; lnaAs; |
| | | | | | | | dGs; lnaAs; |
| | | | | | | | dGs; lnaTs; |
| | | | | | | | dGs; lnaGs; |
| | | | | | | | dGs; lnaG- |
| | | | | | | | Sup |
| 40 | Oligo7 | FXN-330 | TCGATGT | Internal | FXN | human | dTs; lnaCs; |
| | | | CGGTGCG | | | | dGs; lnaAs; |
| | | | CAGGCC | | | | dTs; lnaGs; |
| | | | | | | | dTs; lnaCs; |
| | | | | | | | dGs; lnaGs; |
| | | | | | | | dTs; lnaGs; |
| | | | | | | | dCs; lnaGs; |
| | | | | | | | dCs; lnaAs; |
| | | | | | | | dGs; lnaGs; |
| | | | | | | | dCs; lnaC- |
| | | | | | | | Sup |
| 41 | Oligo8 | FXN-331 | GGCGGGG | Internal | FXN | human | dGs; lnaGs; |
| | | | CGTGCAG | | | | dCs; lnaGs; |
| | | | GTCGCA | | | | dGs; lnaGs; |
| | | | | | | | dGs; lnaCs; |
| | | | | | | | dGs; lnaTs; |
| | | | | | | | dGs; lnaCs; |
| | | | | | | | dAs; lnaGs; |
| | | | | | | | dGs; lnaTs; |
| | | | | | | | dCs; lnaGs; |
| | | | | | | | dCs; lnaA- |
| | | | | | | | Sup |
| 42 | Oligo9 | FXN-332 | ACGTTGG | Internal | FXN | human | dAs; lnaCs; |
| | | | TTCGAACT | | | | dGs; lnaTs; |
| | | | TGCGC | | | | dTs; lnaGs; |
| | | | | | | | dGs; lnaTs; |
| | | | | | | | dTs; lnaCs; |
| | | | | | | | dGs; lnaAs; |
| | | | | | | | dAs; lnaCs; |
| | | | | | | | dTs; lnaTs; |
| | | | | | | | dGs; lnaCs; |
| | | | | | | | dGs; lnaC- |
| | | | | | | | Sup |
| 43 | Oligo10 | FXN-333 | TTCCAAAT | Internal | FXN | human | dTs; lnaTs; |
| | | | CTGGTTG | | | | dCs; lnaCs; |
| | | | AGGCC | | | | dAs; lnaAs; |
| | | | | | | | dAs; lnaTs; |
| | | | | | | | dCs; lnaTs; |
| | | | | | | | dGs; lnaGs; |
| | | | | | | | dTs; lnaTs; |
| | | | | | | | dGs; lnaAs; |
| | | | | | | | dGs; lnaGs; |
| | | | | | | | dCs; lnaC- |
| | | | | | | | Sup |
| 44 | Oligo11 | FXN-334 | AGACACT | Internal | FXN | human | dAs; lnaGs; |
| | | | CTGCTTTT | | | | dAs; lnaCs; |
| | | | TGACA | | | | dAs; lnaCs; |
| | | | | | | | dTs; lnaCs; |
| | | | | | | | dTs; lnaGs; |
| | | | | | | | dCs; lnaTs; |
| | | | | | | | dTs; lnaTs; |
| | | | | | | | dTs; lnaTs; |
| | | | | | | | dGs; lnaAs; |
| | | | | | | | dCs; lnaA- |
| | | | | | | | Sup |
| 45 | Oligo12 | FXN-335 | TTTCCTCA | Internal | FXN | human | dTs; lnaTs; |
| | | | AATTCATC | | | | dTs; lnaCs; |
| | | | AAAT | | | | dCs; lnaTs; |
| | | | | | | | dCs; lnaAs; |
| | | | | | | | dAs; lnaAs; |
| | | | | | | | dTs; lnaTs; |
| | | | | | | | dCs; lnaAs; |
| | | | | | | | dTs; lnaCs; |
| | | | | | | | dAs; lnaAs; |
| | | | | | | | dAs; lnaT- |
| | | | | | | | Sup |
| 46 | Oligo13 | FXN-336 | GGGTGGC | Internal | FXN | human | dGs; lnaGs; |
| | | | CCAAAGT | | | | dGs; lnaTs; |
| | | | TCCAGA | | | | dGs; lnaGs; |
| | | | | | | | dCs; lnaCs; |
| | | | | | | | dCs; lnaAs; |
| | | | | | | | dAs; lnaAs; |
| | | | | | | | dGs; lnaTs; |
| | | | | | | | dTs; lnaCs; |
| | | | | | | | dCs; lnaAs; |
| | | | | | | | dGs; lnaA- |
| | | | | | | | Sup |
| 47 | Oligo14 | FXN-337 | TGGTCTC | Internal | FXN | human | dTs; lnaGs; |
| | | | ATCTAGA | | | | dGs; lnaTs; |
| | | | GAGCCT | | | | dCs; lnaTs; |
| | | | | | | | dCs; lnaAs; |
| | | | | | | | dTs; lnaCs; |
| | | | | | | | dTs; lnaAs; |
| | | | | | | | dGs; lnaAs; |
| | | | | | | | dGs; lnaAs; |
| | | | | | | | dGs; lnaCs; |
| | | | | | | | dCs; lnaT- |
| | | | | | | | Sup |
| 48 | Oligo15 | FXN-338 | CTCTGCTA | Internal | FXN | human | dCs; lnaTs; |
| | | | GTCTTTCA | | | | dCs; lnaTs; |
| | | | TAGG | | | | dGs; lnaCs; |
| | | | | | | | dTs; lnaAs; |
| | | | | | | | dGs; lnaTs; |
| | | | | | | | dCs; lnaTs; |
| | | | | | | | dTs; lnaTs; |
| | | | | | | | dCs; lnaAs; |
| | | | | | | | dTs; lnaAs; |
| | | | | | | | dGs; lnaG- |
| | | | | | | | Sup |
| 49 | Oligo16 | FXN-339 | GCTAAAG | Internal | FXN | human | dGs; lnaCs; |
| | | | AGTCCAG | | | | dTs; lnaAs; |
| | | | CGTTTC | | | | dAs; lnaAs; |
| | | | | | | | dGs; lnaAs; |
| | | | | | | | dGs; lnaTs; |
| | | | | | | | dCs; lnaCs; |
| | | | | | | | dAs; lnaGs; |
| | | | | | | | dCs; lnaGs; |
| | | | | | | | dTs; lnaTs; |
| | | | | | | | dTs; lnaC- |
| | | | | | | | Sup |
| 50 | Oligo17 | FXN-340 | GCAAGGT | Internal | FXN | human | dGs; lnaCs; |
| | | | CTTCAAA | | | | dAs; lnaAs; |
| | | | AAACTCT | | | | dGs; lnaGs; |
| | | | | | | | dTs; lnaCs; |
| | | | | | | | dTs; lnaTs; |
| | | | | | | | dCs; lnaAs; |
| | | | | | | | dAs; lnaAs; |
| | | | | | | | dAs; lnaAs; |
| | | | | | | | dAs; lnaCs; |
| | | | | | | | dTs; lnaCs; |
| | | | | | | | dT-Sup |
| 51 | Oligo18 | FXN-341 | CTCAAAC | Internal | FXN | human | dCs; lnaTs; |
| | | | GTGTATG | | | | dCs; lnaAs; |
| | | | GCTTGTCT | | | | dAs; lnaAs; |
| | | | | | | | dCs; lnaGs; |
| | | | | | | | dTs; lnaGs; |
| | | | | | | | dTs; lnaAs; |
| | | | | | | | dTs; lnaGs; |
| | | | | | | | dGs; lnaCs; |
| | | | | | | | dTs; lnaTs; |
| | | | | | | | dGs; lnaTs; |
| | | | | | | | dCs; lnaT-Sup |
| 52 | Oligo19 | FXN-342 | CCCAAAG | Internal | FXN | human | dCs; lnaCs; |
| | | | GAGACAT | | | | dCs; lnaAs; |
| | | | CATAGTC | | | | dAs; lnaAs; |
| | | | | | | | dGs; lnaGs; |
| | | | | | | | dAs; lnaGs; |
| | | | | | | | dAs; lnaCs; |
| | | | | | | | dAs; lnaTs; |
| | | | | | | | dCs; lnaAs; |
| | | | | | | | dTs; lnaAs; |
| | | | | | | | dGs; lnaTs; |
| | | | | | | | dC-Sup |
| 53 | Oligo20 | FXN-343 | CAGTTTG | Internal | FXN | human | dCs; lnaAs; |
| | | | ACAGTTA | | | | dGs; lnaTs; |
| | | | AGACACC | | | | dTs; lnaTs; |
| | | | ACT | | | | dGs; lnaAs; |
| | | | | | | | dCs; lnaAs; |
| | | | | | | | dGs; lnaTs; |
| | | | | | | | dTs; lnaAs; |
| | | | | | | | dAs; lnaGs; |
| | | | | | | | dAs; lnaCs; |
| | | | | | | | dAs; lnaCs; |
| | | | | | | | dCs; lnaAs; |
| | | | | | | | dCs; lnaT- |
| | | | | | | | Sup |
| 54 | Oligo21 | FXN-344 | ATAGGTT | Internal | FXN | human | dAs; lnaTs; |
| | | | CCTAGAT | | | | dAs; lnaGs; |
| | | | CTCCACC | | | | dGs; lnaTs; |
| | | | | | | | dTs; lnaCs; |
| | | | | | | | dCs; lnaTs; |
| | | | | | | | dAs; lnaGs; |
| | | | | | | | dAs; lnaTs; |
| | | | | | | | dCs; lnaTs; |
| | | | | | | | dCs; lnaCs; |
| | | | | | | | dAs; lnaCs; |
| | | | | | | | dC-Sup |
| 55 | Oligo22 | FXN-345 | GGCGTCT | Internal | FXN | human | dGs; lnaGs; |
| | | | GCTTGTT | | | | dCs; lnaGs; |
| | | | GATCAC | | | | dTs; lnaCs; |
| | | | | | | | dTs; lnaGs; |
| | | | | | | | dCs; lnaTs; |
| | | | | | | | dTs; lnaGs; |
| | | | | | | | dTs; lnaTs; |
| | | | | | | | dGs; lnaAs; |
| | | | | | | | dTs; lnaCs; |
| | | | | | | | dAs; lnaC- |
| | | | | | | | Sup |
| 56 | Oligo23 | FXN-346 | AAGATAG | Internal | FXN | human | dAs; lnaAs; |
| | | | CCAGATTT | | | | dGs; lnaAs; |
| | | | GCTTGTTT | | | | dTs; lnaAs; |
| | | | | | | | dGs; lnaCs; |
| | | | | | | | dCs; lnaAs; |
| | | | | | | | dGs; lnaAs; |
| | | | | | | | dTs; lnaTs; |
| | | | | | | | dTs; lnaGs; |
| | | | | | | | dCs; lnaTs; |
| | | | | | | | dTs; lnaGs; |
| | | | | | | | dTs; lnaTs; |
| | | | | | | | dT- |
| | | | | | | | Sup |
| 57 | Oligo24 | FXN-347 | GGTCCAC | Internal | FXN | human | dGs; lnaGs; |
| | | | TACATACC | | | | dTs; lnaCs; |
| | | | TGGATGG | | | | dCs; lnaAs; |
| | | | AG | | | | dCs; lnaTs; |
| | | | | | | | dAs; lnaCs; |
| | | | | | | | dAs; lnaTs; |
| | | | | | | | dAs; lnaCs; |
| | | | | | | | dCs; lnaTs; |
| | | | | | | | dGs; lnaGs; |
| | | | | | | | dAs; lnaTs; |
| | | | | | | | dGs; lnaGs; |
| | | | | | | | dAs; lnaG- |
| | | | | | | | Sup |
| 58 | Oligo25 | FXN-348 | CCCAGTC | Internal | FXN | human | dCs; lnaCs; |
| | | | CAGTCAT | | | | dCs; lnaAs; |
| | | | AACGCTT | | | | dGs; lnaTs; |
| | | | | | | | dCs; lnaCs; |
| | | | | | | | dAs; lnaGs; |
| | | | | | | | dTs; lnaCs; |
| | | | | | | | dAs; lnaTs; |
| | | | | | | | dAs; lnaAs; |
| | | | | | | | dCs; lnaGs; |
| | | | | | | | dCs; lnaTs; |
| | | | | | | | dT-Sup |
| 59 | Oligo26 | FXN-349 | CGTGGGA | Internal | FXN | human | dCs; lnaGs; |
| | | | GTACACC | | | | dTs; lnaGs; |
| | | | CAGTTTTT | | | | dGs; lnaGs; |
| | | | | | | | dAs; lnaGs; |
| | | | | | | | dTs; lnaAs; |
| | | | | | | | dCs; lnaAs; |
| | | | | | | | dCs; lnaCs; |
| | | | | | | | dCs; lnaAs; |
| | | | | | | | dGs; lnaTs; |
| | | | | | | | dTs; lnaTs; |
| | | | | | | | dTs; lnaT-Sup |
| 60 | Oligo27 | FXN-350 | CATGGAG | Internal | FXN | human | dCs; lnaAs; |
| | | | GGACACG | | | | dTs; lnaGs; |
| | | | CCGT | | | | dGs; lnaAs; |
| | | | | | | | dGs; lnaGs; |
| | | | | | | | dGs; lnaAs; |
| | | | | | | | dCs; lnaAs; |
| | | | | | | | dCs; lnaGs; |
| | | | | | | | dCs; lnaCs; |
| | | | | | | | dGs; lnaT- |
| | | | | | | | Sup |
| 61 | Oligo28 | FXN-351 | GTGAGCT | Internal | FXN | human | dGs; lnaTs; |
| | | | CTGCGGC | | | | dGs; lnaAs; |
| | | | CAGCAGCT | | | | dGs; lnaCs; |
| | | | | | | | dTs; lnaCs; |
| | | | | | | | dTs; lnaGs; |
| | | | | | | | dCs; lnaGs; |
| | | | | | | | dGs; lnaCs; |
| | | | | | | | dCs; lnaAs; |
| | | | | | | | dGs; lnaCs; |
| | | | | | | | dAs; lnaGs; |
| | | | | | | | dCs; lnaT- |
| | | | | | | | Sup |
| 62 | Oligo29 | FXN-352 | AGTTTGG | Internal | FXN | human | dAs; lnaGs; |
| | | | TTTTTAAG | | | | dTs; lnaTs; |
| | | | GCTTTA | | | | dTs; lnaGs; |
| | | | | | | | dGs; lnaTs; |
| | | | | | | | dTs; lnaTs; |
| | | | | | | | dTs; lnaTs; |
| | | | | | | | dAs; lnaAs; |
| | | | | | | | dGs; lnaGs; |
| | | | | | | | dCs; lnaTs; |
| | | | | | | | dTs; lnaTs; |
| | | | | | | | dA-Sup |
| 63 | Oligo30 | FXN-353 | TAGGCCA | Internal | FXN | human | dTs; lnaAs; |
| | | | AGGAAGA | | | | dGs; lnaGs; |
| | | | CAAGTCC | | | | dCs; lnaCs; |
| | | | | | | | dAs; lnaAs; |
| | | | | | | | dGs; lnaGs; |
| | | | | | | | dAs; lnaAs; |
| | | | | | | | dGs; lnaAs; |
| | | | | | | | dCs; lnaAs; |
| | | | | | | | dAs; lnaGs; |
| | | | | | | | dTs; lnaCs; |
| | | | | | | | dC-Sup |
| 64 | Oligo31 | FXN-354 | TCAAGCA | Internal | FXN | human | dTs; lnaCs; |
| | | | TCTTTTCC | | | | dAs; lnaAs; |
| | | | GGAA | | | | dGs; lnaCs; |
| | | | | | | | dAs; lnaTs; |
| | | | | | | | dCs; lnaTs; |
| | | | | | | | dTs; lnaTs; |
| | | | | | | | dTs; lnaCs; |
| | | | | | | | dCs; lnaGs; |
| | | | | | | | dGs; lnaAs; |
| | | | | | | | dA- |
| | | | | | | | Sup |
| 65 | Oligo32 | FXN-355 | TCCTTAAA | Internal | FXN | human | dTs; lnaCs; |
| | | | ACGGGGC | | | | dCs; lnaTs; |
| | | | TGGGCA | | | | dTs; lnaAs; |
| | | | | | | | dAs; lnaAs; |
| | | | | | | | dAs; lnaCs; |
| | | | | | | | dGs; lnaGs; |
| | | | | | | | dGs; lnaGs; |
| | | | | | | | dCs; lnaTs; |
| | | | | | | | dGs; lnaGs; |
| | | | | | | | dGs; lnaCs; |
| | | | | | | | dA-Sup |
| 66 | Oligo33 | FXN-356 | TTGGCCT | Internal | FXN | human | dTs; lnaTs; |
| | | | GATAGCT | | | | dGs; lnaGs; |
| | | | TTTAATG | | | | dCs; lnaCs; |
| | | | | | | | dTs; lnaGs; |
| | | | | | | | dAs; lnaTs; |
| | | | | | | | dAs; lnaGs; |
| | | | | | | | dCs; lnaTs; |
| | | | | | | | dTs; lnaTs; |
| | | | | | | | dTs; lnaAs; |
| | | | | | | | dAs; lnaTs; |
| | | | | | | | dG-Sup |
| 67 | Oligo34 | FXN-357 | CCTCAGCT | Internal | FXN | human | dCs; lnaCs; |
| | | | GCATAAT | | | | dTs; lnaCs; |
| | | | GAAGCTG | | | | dAs; lnaGs; |
| | | | GGGTC | | | | dCs; lnaTs; |
| | | | | | | | dGs; lnaCs; |
| | | | | | | | dAs; lnaTs; |
| | | | | | | | dAs; lnaAs; |
| | | | | | | | dTs; lnaGs; |
| | | | | | | | dAs; lnaAs; |
| | | | | | | | dGs; lnaCs; |
| | | | | | | | dTs; lnaGs; |
| | | | | | | | dGs; lnaGs; |
| | | | | | | | dGs; lnaTs; |
| | | | | | | | dC- |
| | | | | | | | Sup |
| 68 | Oligo35 | FXN-358 | AACAACA | Internal | FXN | human | dAs; lnaAs; |
| | | | ACAACAA | | | | dCs; lnaAs; |
| | | | CAAAAAA | | | | dAs; lnaCs; |
| | | | CAGA | | | | dAs; lnaAs; |
| | | | | | | | dCs; lnaAs; |
| | | | | | | | dAs; lnaCs; |
| | | | | | | | dAs; lnaAs; |
| | | | | | | | dCs; lnaAs; |
| | | | | | | | dAs; lnaAs; |
| | | | | | | | dAs; lnaAs; |
| | | | | | | | dAs; lnaCs; |
| | | | | | | | dAs; lnaGs; |
| | | | | | | | dA-Sup |
| 69 | Oligo36 | FXN-359 | CCTCAAA | Internal | FXN | human | dCs; lnaCs; |
| | | | AGCAGGA | | | | dTs; lnaCs; |
| | | | ATAAAAA | | | | dAs; lnaAs; |
| | | | AAATA | | | | dAs; lnaAs; |
| | | | | | | | dGs; lnaCs; |
| | | | | | | | dAs; lnaGs; |
| | | | | | | | dGs; lnaAs; |
| | | | | | | | dAs; lnaTs; |
| | | | | | | | dAs; lnaAs; |
| | | | | | | | dAs; lnaAs; |
| | | | | | | | dAs; lnaAs; |
| | | | | | | | dAs; lnaAs; |
| | | | | | | | dTs; lnaA- |
| | | | | | | | Sup |
| 70 | Oligo37 | FXN-360 | GCTGTGA | Internal | FXN | human | dGs; lnaCs; |
| | | | CACATAG | | | | dTs; lnaGs; |
| | | | CCCAACT | | | | dTs; lnaGs; |
| | | | GT | | | | dAs; lnaCs; |
| | | | | | | | dAs; lnaCs; |
| | | | | | | | dAs; lnaTs; |
| | | | | | | | dAs; lnaGs; |
| | | | | | | | dCs; lnaCs; |
| | | | | | | | dCs; lnaAs; |
| | | | | | | | dAs; lnaCs; |
| | | | | | | | dTs; lnaGs; |
| | | | | | | | dT- |
| | | | | | | | Sup |
| 71 | Oligo38 | FXN-361 | GGAGGCA | Internal | FXN | human | dGs; lnaGs; |
| | | | ACACATTC | | | | dAs; lnaGs; |
| | | | TTTCTACA | | | | dGs; lnaCs; |
| | | | GA | | | | dAs; lnaAs; |
| | | | | | | | dCs; lnaAs; |
| | | | | | | | dCs; lnaAs; |
| | | | | | | | dTs; lnaTs; |
| | | | | | | | dCs; lnaTs; |
| | | | | | | | dTs; lnaTs; |
| | | | | | | | dCs; lnaTs; |
| | | | | | | | dAs; lnaCs; |
| | | | | | | | dAs; lnaGs; |
| | | | | | | | dA-Sup |
| 72 | Oligo39 | FXN-362 | CTATTAAT | Intron | FXN | human | dCs; lnaTs; |
| | | | ATTACTG | | | | dAs; lnaTs; |
| | | | | | | | dTs; lnaAs; |
| | | | | | | | dAs; lnaTs; |
| | | | | | | | dAs; lnaTs; |
| | | | | | | | dTs; lnaAs; |
| | | | | | | | dCs; lnaTs; |
| | | | | | | | dG- |
| | | | | | | | Sup |
| 73 | Oligo40 | FXN-363 | CATTATGT | Intron | FXN | human | dCs; lnaAs; |
| | | | GTATGTAT | | | | dTs; lnaTs; |
| | | | | | | | dAs; lnaTs; |
| | | | | | | | dGs; lnaTs; |
| | | | | | | | dGs; lnaTs; |
| | | | | | | | dAs; lnaTs; |
| | | | | | | | dGs; lnaTs; |
| | | | | | | | dAs; lnaT- |
| | | | | | | | Sup |
| 74 | Oligo41 | FXN-364 | TTTATCTA | Intron | FXN | human | dTs; lnaTs; |
| | | | TGTTATT | | | | dTs; lnaAs; |
| | | | | | | | dTs; lnaCs; |
| | | | | | | | dTs; lnaAs; |
| | | | | | | | dTs; lnaGs; |
| | | | | | | | dTs; lnaTs; |
| | | | | | | | dAs; lnaTs; |
| | | | | | | | dT- |
| | | | | | | | Sup |
| 75 | Oligo42 | FXN-365 | CTAATTTG | Intron | FXN | human | dCs; lnaTs; |
| | | | AAGTTCT | | | | dAs; lnaAs; |
| | | | | | | | dTs; lnaTs; |
| | | | | | | | dTs; lnaGs; |
| | | | | | | | dAs; lnaAs; |
| | | | | | | | dGs; lnaTs; |
| | | | | | | | dTs; lnaCs; |
| | | | | | | | dT- |
| | | | | | | | Sup |
| 76 | Oligo43 | FXN-366 | TTCGAACT | Exon | FXN | human | dTs; lnaTs; |
| | | | TGCGCGG | Spanning | | | dCs; lnaGs; |
| | | | | | | | dAs; lnaAs; |
| | | | | | | | dCs; lnaTs; |
| | | | | | | | dTs; lnaGs; |
| | | | | | | | dCs; lnaGs; |
| | | | | | | | dCs; lnaGs; |
| | | | | | | | dG- |
| | | | | | | | Sup |
| 77 | Oligo44 | FXN-367 | TAGAGAG | Exon | FXN | human | dTs; lnaAs; |
| | | | CCTGGGT | Spanning | | | dGs; lnaAs; |
| | | | | | | | dGs; lnaAs; |
| | | | | | | | dGs; lnaCs; |
| | | | | | | | dCs; lnaTs; |
| | | | | | | | dGs; lnaGs; |
| | | | | | | | dGs; lnaT-Sup |
| 78 | Oligo45 | FXN-368 | ACACCAC | Exon | FXN | human | dAs; lnaCs; |
| | | | TCCCAAAG | Spanning | | | dAs; lnaCs; |
| | | | | | | | dCs; lnaAs; |
| | | | | | | | dCs; lnaTs; |
| | | | | | | | dCs; lnaCs; |
| | | | | | | | dCs; lnaAs; |
| | | | | | | | dAs; lnaAs; |
| | | | | | | | dG- |
| | | | | | | | Sup |
| 79 | Oligo46 | FXN-369 | AGGTCCA | Exon | FXN | human | dAs; lnaGs; |
| | | | CTACATAC | Spanning | | | dGs; lnaTs; |
| | | | | | | | dCs; lnaCs; |
| | | | | | | | dAs; lnaCs; |
| | | | | | | | dTs; lnaAs; |
| | | | | | | | dCs; lnaAs; |
| | | | | | | | dTs; lnaAs; |
| | | | | | | | dC- |
| | | | | | | | Sup |
| 80 | Oligo47 | FXN-370 | CGTTAAC | Exon | FXN | human | dCs; lnaGs; |
| | | | CTGGATGG | Spanning | | | dTs; lnaTs; |
| | | | | | | | dAs; lnaAs; |
| | | | | | | | dCs; lnaCs; |
| | | | | | | | dTs; lnaGs; |
| | | | | | | | dGs; lnaAs; |
| | | | | | | | dTs; lnaGs; |
| | | | | | | | dG- |
| | | | | | | | Sup |
| 81 | Oligo81 | FXN-404 | AAAGCCT | Antisense | FXN | human | dAs; lnaAs; |
| | | | TAAAAACC | | | | dAs; lnaGs; |
| | | | | | | | dCs; lnaCs; |
| | | | | | | | dTs; lnaTs; |
| | | | | | | | dAs; lnaAs; |
| | | | | | | | dAs; lnaAs; |
| | | | | | | | dAs; lnaCs; |
| | | | | | | | dC- |
| | | | | | | | Sup |
| 82 | Oligo82 | FXN-405 | TCAGGCC | Antisense | FXN | human | dTs; lnaCs; |
| | | | AAGACCCC | | | | dAs; lnaGs; |
| | | | | | | | dGs; lnaCs; |
| | | | | | | | dCs; lnaAs; |
| | | | | | | | dAs; lnaGs; |
| | | | | | | | dAs; lnaCs; |
| | | | | | | | dCs; lnaCs; |
| | | | | | | | dC- |
| | | | | | | | Sup |
| 83 | Oligo83 | FXN-406 | CCCAGCTT | Antisense | FXN | human | dCs; lnaCs; |
| | | | CATTATG | | | | dCs; lnaAs; |
| | | | | | | | dGs; lnaCs; |
| | | | | | | | dTs; lnaTs; |
| | | | | | | | dCs; lnaAs; |
| | | | | | | | dTs; lnaTs; |
| | | | | | | | dAs; lnaTs; |
| | | | | | | | dG- |
| | | | | | | | Sup |
| 84 | Oligo84 | FXN-407 | AATGTGT | Antisense | FXN | human | dAs; lnaAs; |
| | | | TGCCTCCT | | | | dTs; lnaGs; |
| | | | | | | | dTs; lnaGs; |
| | | | | | | | dTs; lnaTs; |
| | | | | | | | dGs; lnaCs; |
| | | | | | | | dCs; lnaTs; |
| | | | | | | | dCs; lnaCs; |
| | | | | | | | dT- |
| | | | | | | | Sup |
| 85 | Oligo85 | FXN-408 | AAAAAGC | Antisense | FXN | human | dAs; lnaAs; |
| | | | AAAATAAT | | | | dAs; lnaAs; |
| | | | | | | | dAs; lnaGs; |
| | | | | | | | dCs; lnaAs; |
| | | | | | | | dAs; lnaAs; |
| | | | | | | | dAs; lnaTs; |
| | | | | | | | dAs; lnaAs; |
| | | | | | | | dT- |
| | | | | | | | Sup |
| 86 | Oligo86 | FXN-409 | CCAGGAG | Antisense | FXN | human | dCs; lnaCs; |
| | | | GGAAAATG | | | | dAs; lnaGs; |
| | | | | | | | dGs; lnaAs; |
| | | | | | | | dGs; lnaGs; |
| | | | | | | | dGs; lnaAs; |
| | | | | | | | dAs; lnaAs; |
| | | | | | | | dAs; lnaTs; |
| | | | | | | | dG- |
| | | | | | | | Sup |
| 87 | Oligo87 | FXN-410 | TAAAGGG | Antisense | FXN | human | dTs; lnaAs; |
| | | | TAGCCTAC | | | | dAs; lnaAs; |
| | | | | | | | dGs; lnaGs; |
| | | | | | | | dGs; lnaTs; |
| | | | | | | | dAs; lnaGs; |
| | | | | | | | dCs; lnaCs; |
| | | | | | | | dTs; lnaAs; |
| | | | | | | | dC- |
| | | | | | | | Sup |
| 88 | Oligo88 | FXN-411 | AAAGCAA | Antisense | FXN | human | dAs; lnaAs; |
| | | | CAAGCCTC | | | | dAs; lnaGs; |
| | | | | | | | dCs; lnaAs; |
| | | | | | | | dAs; lnaCs; |
| | | | | | | | dAs; lnaAs; |
| | | | | | | | dGs; lnaCs; |
| | | | | | | | dCs; lnaTs; |
| | | | | | | | dC- |
| | | | | | | | Sup |
| 89 | Oligo89 | FXN-412 | ATAACAT | Antisense | FXN | human | dAs; lnaTs; |
| | | | CATACATG | | | | dAs; lnaAs; |
| | | | | | | | dCs; lnaAs; |
| | | | | | | | dTs; lnaCs; |
| | | | | | | | dAs; lnaTs; |
| | | | | | | | dAs; lnaCs; |
| | | | | | | | dAs; lnaTs; |
| | | | | | | | dG- |
| | | | | | | | Sup |
| 90 | Oligo90 | FXN-413 | GATACTA | Antisense | FXN | human | dGs; lnaAs; |
| | | | TCTTCCTC | | | | dTs; lnaAs; |
| | | | | | | | dCs; lnaTs; |
| | | | | | | | dAs; lnaTs; |
| | | | | | | | dCs; lnaTs; |
| | | | | | | | dTs; lnaCs; |
| | | | | | | | dCs; lnaTs; |
| | | | | | | | dC- |
| | | | | | | | Sup |
| 91 | Oligo91 | FXN-414 | ATGGGGG | Antisense | FXN | human | dAs; lnaTs; |
| | | | ACGGGGCA | | | | dGs; lnaGs; |
| | | | | | | | dGs; lnaGs; |
| | | | | | | | dGs; lnaAs; |
| | | | | | | | dCs; lnaGs; |
| | | | | | | | dGs; lnaGs; |
| | | | | | | | dGs; lnaCs; |
| | | | | | | | dA- |
| | | | | | | | Sup |
| 92 | Oligo92 | FXN-415 | GGTTGAG | Antisense | FXN | human | dGs; lnaGs; |
| | | | ACTGGGTG | | | | dTs; lnaTs; |
| | | | | | | | dGs; lnaAs; |
| | | | | | | | dGs; lnaAs; |
| | | | | | | | dCs; lnaTs; |
| | | | | | | | dGs; lnaGs; |
| | | | | | | | dGs; lnaTs; |
| | | | | | | | dG- |
| | | | | | | | Sup |
| 93 | Oligo93 | FXN-416 | AGACTGA | Antisense | FXN | human | dAs; lnaGs; |
| | | | AGAGGTGC | | | | dAs; lnaCs; |
| | | | | | | | dTs; lnaGs; |
| | | | | | | | dAs; lnaAs; |
| | | | | | | | dGs; lnaAs; |
| | | | | | | | dGs; lnaGs; |
| | | | | | | | dTs; lnaGs; |
| | | | | | | | dC- |
| | | | | | | | Sup |
| 94 | Oligo94 | FXN-417 | CGGGACG | Antisense | FXN | human | dCs; lnaGs; |
| | | | GCTGTGTT | | | | dGs; lnaGs; |
| | | | | | | | dAs; lnaCs; |
| | | | | | | | dGs; lnaGs; |
| | | | | | | | dCs; lnaTs; |
| | | | | | | | dGs; lnaTs; |
| | | | | | | | dGs; lnaTs; |
| | | | | | | | dT- |
| | | | | | | | Sup |
| 95 | Oligo95 | FXN-418 | TCTGTGT | Antisense | FXN | human | dTs; lnaCs; |
| | | | GGGCAGCA | | | | dTs; lnaGs; |
| | | | | | | | dTs; lnaGs; |
| | | | | | | | dTs; lnaGs; |
| | | | | | | | dGs; lnaGs; |
| | | | | | | | dCs; lnaAs; |
| | | | | | | | dGs; lnaCs; |
| | | | | | | | dA- |
| | | | | | | | Sup |
| 96 | Oligo96 | FXN-419 | AAAGCCT | Antisense | FXN | human | lnaAs; lnaAs; |
| | | | TAAAAACC | | | | lnaAs; dGs; |
| | | | | | | | dCs; dCs; |
| | | | | | | | dTs; dTs; |
| | | | | | | | dAs; dAs; |
| | | | | | | | dAs; dAs; |
| | | | | | | | lnaAs; lnaCs; |
| | | | | | | | lnaC- |
| | | | | | | | Sup |
| 97 | Oligo97 | FXN-420 | TCAGGCC | Antisense | FXN | human | lnaTs; lnaCs; |
| | | | AAGACCCC | | | | lnaAs; dGs; |
| | | | | | | | dGs; dCs; |
| | | | | | | | dCs; dAs; |
| | | | | | | | dAs; dGs; |
| | | | | | | | dAs; dCs; |
| | | | | | | | lnaCs; lnaCs; |
| | | | | | | | lnaC- |
| | | | | | | | Sup |
| 98 | Oligo98 | FXN-421 | CCCAGCTT | Antisense | FXN | human | lnaCs; lnaCs; |
| | | | CATTATG | | | | lnaCs; dAs; |
| | | | | | | | dGs; dCs; |
| | | | | | | | dTs; dTs; |
| | | | | | | | dCs; dAs; |
| | | | | | | | dTs; dTs; |
| | | | | | | | lnaAs; lnaTs; |
| | | | | | | | lnaG-Sup |
| 99 | Oligo99 | FXN-422 | AATGTGT | Antisense | FXN | human | lnaAs; lnaAs; |
| | | | TGCCTCCT | | | | lnaTs; dGs; |
| | | | | | | | dTs; dGs; |
| | | | | | | | dTs; dTs; |
| | | | | | | | dGs; dCs; |
| | | | | | | | dCs; dTs; |
| | | | | | | | lnaCs; lnaCs; |
| | | | | | | | lnaT-Sup |
| 100 | Oligo100 | FXN-423 | AAAAAGC | Antisense | FXN | human | lnaAs; lnaAs; |
| | | | AAAATAAT | | | | lnaAs; dAs; |
| | | | | | | | dAs; dGs; |
| | | | | | | | dCs; dAs; |
| | | | | | | | dAs; dAs; |
| | | | | | | | dAs; dTs; |
| | | | | | | | lnaAs; lnaAs; |
| | | | | | | | lnaT- |
| | | | | | | | Sup |
| 101 | Oligo101 | FXN-424 | CCAGGAG | Antisense | FXN | human | lnaCs; lnaCs; |
| | | | GGAAAATG | | | | lnaAs; dGs; |
| | | | | | | | dGs; dAs; |
| | | | | | | | dGs; dGs; |
| | | | | | | | dGs; dAs; |
| | | | | | | | dAs; dAs; |
| | | | | | | | lnaAs; lnaTs; |
| | | | | | | | lnaG- |
| | | | | | | | Sup |
| 102 | Oligo102 | FXN-425 | TAAAGGG | Antisense | FXN | human | lnaTs; lnaAs; |
| | | | TAGCCTAC | | | | lnaAs; dAs; |
| | | | | | | | dGs; dGs; |
| | | | | | | | dGs; dTs; |
| | | | | | | | dAs; dGs; |
| | | | | | | | dCs; dCs; |
| | | | | | | | lnaTs; lnaAs; |
| | | | | | | | lnaC- |
| | | | | | | | Sup |
| 103 | Oligo103 | FXN-426 | AAAGCAA | Antisense | FXN | human | lnaAs; lnaAs; |
| | | | CAAGCCTC | | | | lnaAs; dGs; |
| | | | | | | | dCs; dAs; |
| | | | | | | | dAs; dCs; |
| | | | | | | | dAs; dAs; |
| | | | | | | | dGs; dCs; |
| | | | | | | | lnaCs; lnaTs; |
| | | | | | | | lnaC- |
| | | | | | | | Sup |
| 104 | Oligo104 | FXN-427 | ATAACAT | Antisense | FXN | human | lnaAs; lnaTs; |
| | | | CATACATG | | | | lnaAs; dAs; |
| | | | | | | | dCs; dAs; |
| | | | | | | | dTs; dCs; |
| | | | | | | | dAs; dTs; |
| | | | | | | | dAs; dCs; |
| | | | | | | | lnaAs; lnaTs; |
| | | | | | | | lnaG-Sup |
| 105 | Oligo105 | FXN-428 | GATACTA | Antisense | FXN | human | lnaGs; lnaAs; |
| | | | TCTTCCTC | | | | lnaTs; dAs; |
| | | | | | | | dCs; dTs; |
| | | | | | | | dAs; dTs; |
| | | | | | | | dCs; dTs; |
| | | | | | | | dTs; dCs; |
| | | | | | | | lnaCs; lnaTs; |
| | | | | | | | lnaC-Sup |
| 106 | Oligo106 | FXN-429 | ATGGGGG | Antisense | FXN | human | lnaAs; lnaTs; |
| | | | ACGGGGCA | | | | lnaGs; dGs; |
| | | | | | | | dGs; dGs; |
| | | | | | | | dGs; dAs; |
| | | | | | | | dCs; dGs; |
| | | | | | | | dGs; dGs; |
| | | | | | | | lnaGs; lnaCs; |
| | | | | | | | lnaA- |
| | | | | | | | Sup |
| 107 | Oligo107 | FXN-430 | GGTTGAG | Antisense | FXN | human | lnaGs; lnaGs; |
| | | | ACTGGGTG | | | | lnaTs; dTs; |
| | | | | | | | dGs; dAs; |
| | | | | | | | dGs; dAs; |
| | | | | | | | dCs; dTs; |
| | | | | | | | dGs; dGs; |
| | | | | | | | lnaGs; lnaTs; |
| | | | | | | | lnaG- |
| | | | | | | | Sup |
| 108 | Oligo108 | FXN-431 | AGACTGA | Antisense | FXN | human | lnaAs; lnaGs; |
| | | | AGAGGTGC | | | | lnaAs; dCs; |
| | | | | | | | dTs; dGs; |
| | | | | | | | dAs; dAs; |
| | | | | | | | dGs; dAs; |
| | | | | | | | dGs; dGs; |
| | | | | | | | lnaTs; lnaGs; |
| | | | | | | | lnaC- |
| | | | | | | | Sup |
| 109 | Oligo109 | FXN-432 | CGGGACG | Antisense | FXN | human | lnaCs; lnaGs; |
| | | | GCTGTGTT | | | | lnaGs; dGs; |
| | | | | | | | dAs; dCs; |
| | | | | | | | dGs; dGs; |
| | | | | | | | dCs; dTs; |
| | | | | | | | dGs; dTs; |
| | | | | | | | lnaGs; lnaTs; |
| | | | | | | | lnaT- |
| | | | | | | | Sup |
| 110 | Oligo110 | FXN-433 | TCTGTGT | Antisense | FXN | human | lnaTs; lnaCs; |
| | | | GGGCAGCA | | | | lnaTs; dGs; |
| | | | | | | | dTs; dGs; |
| | | | | | | | dTs; dGs; |
| | | | | | | | dGs; dGs; |
| | | | | | | | dCs; dAs; |
| | | | | | | | lnaGs; lnaCs; |
| | | | | | | | lnaA- |
| | | | | | | | Sup |
| 111 | Oligo111 | FXN-115 | GAAGAAG | Antisense | FXN | human | lnaGs; lnaAs; |
| | | | AAGAAGAA | | | | lnaAs; dGs; |
| | | | | | | | dAs; dAs; |
| | | | | | | | dGs; dAs; |
| | | | | | | | dAs; dGs; |
| | | | | | | | dAs; dAs; |
| | | | | | | | lnaGs; lnaAs; |
| | | | | | | | lnaA- |
| | | | | | | | Sup |
| 112 | Oligo112 | FXN-117 | TTCTTCTT | Antisense | FXN | human | lnaTs; lnaTs; |
| | | | CTTCTTC | | | | lnaCs; dTs; |
| | | | | | | | dTs; dCs; |
| | | | | | | | dTs; dTs; |
| | | | | | | | dCs; dTs; |
| | | | | | | | dTs; dCs; |
| | | | | | | | lnaTs; lnaTs; |
| | | | | | | | lnaC-Sup |

TABLE 5
Oligonucleotide modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |

In Vitro Transfection of Cells with Oligonucleotides

Cells were seeded into each well of 24-well plates at a density of 25,000 cells per 500 uL and transfections were performed with Lipofectamine and the single stranded oligonucleotides. Control wells contained Lipofectamine alone. At time points post-transfection, approximately 200 uL of cell culture supernatants were stored at −80 C for ELISA or Western blot analysis and RNA was harvested from another aliquot of cells and quantitative PCR was carried out as outlined above. The percent induction of target mRNA expression by each oligonucleotide was determined by normalizing mRNA levels in the presence of the oligonucleotide to the mRNA levels in the presence of control (Lipofectamine alone).

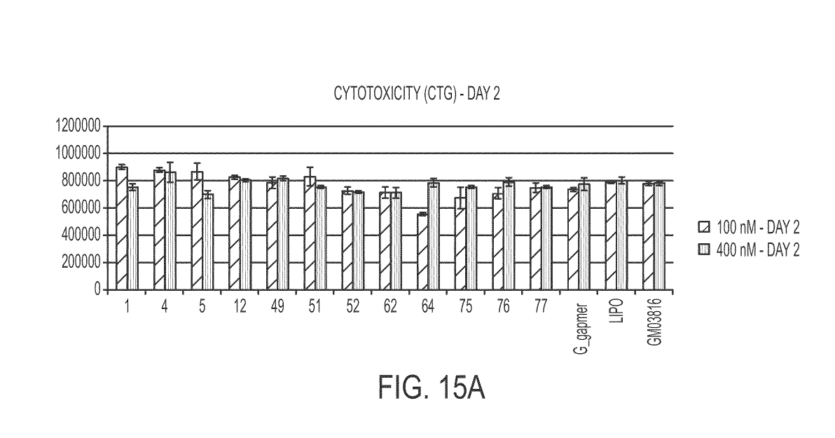

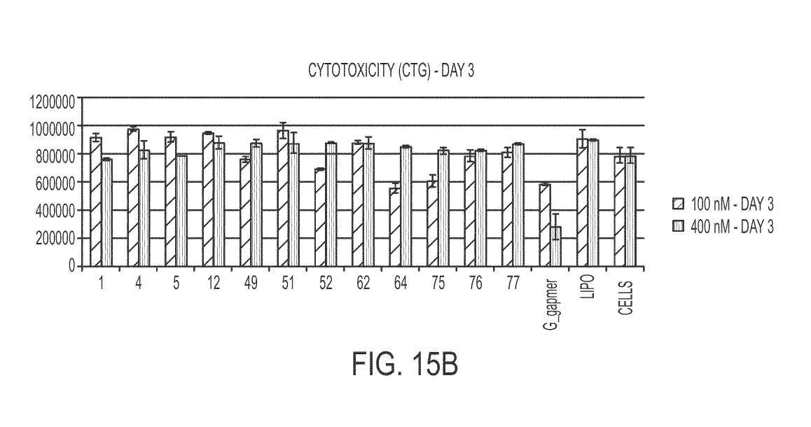

As a control, the oligos were tested for cytotoxic effects. It was determined that cell transfected with oligos did not demonstrate cytotoxicity at either 100 or 400 nM oligo concentrations (FIG. 15).

### Results:

In Vitro Delivery of Single Stranded Oligonucleotides that Target the 5′ and 3′ End of FXN mRNA Upregulated FXN Expression

FXN was chosen as an exemplary target for RNA stabilization because FXN is a housekeeping gene that is challenging to upregulate. Oligonucleotides were designed against the putative 5′ and 3′ ends of FXN mRNA using the methods described above. The 3′ and 5′ oligos were first tested separately and then in combination.

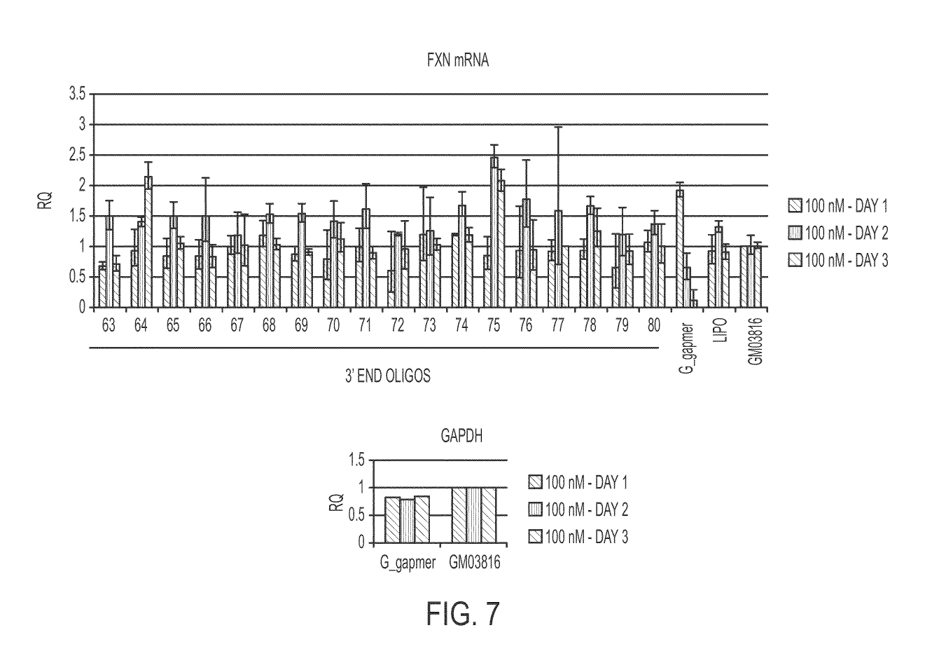

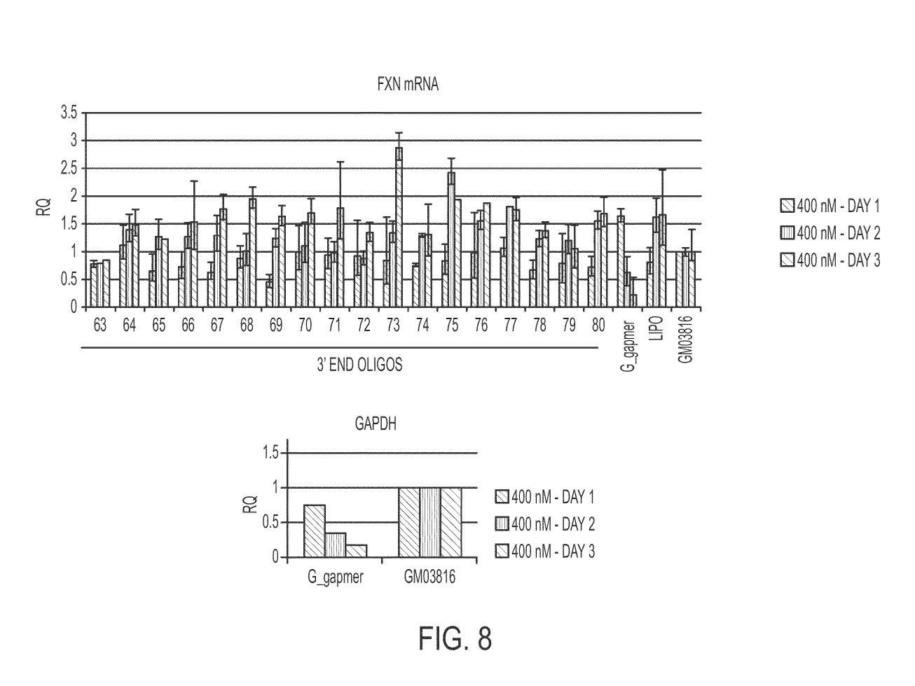

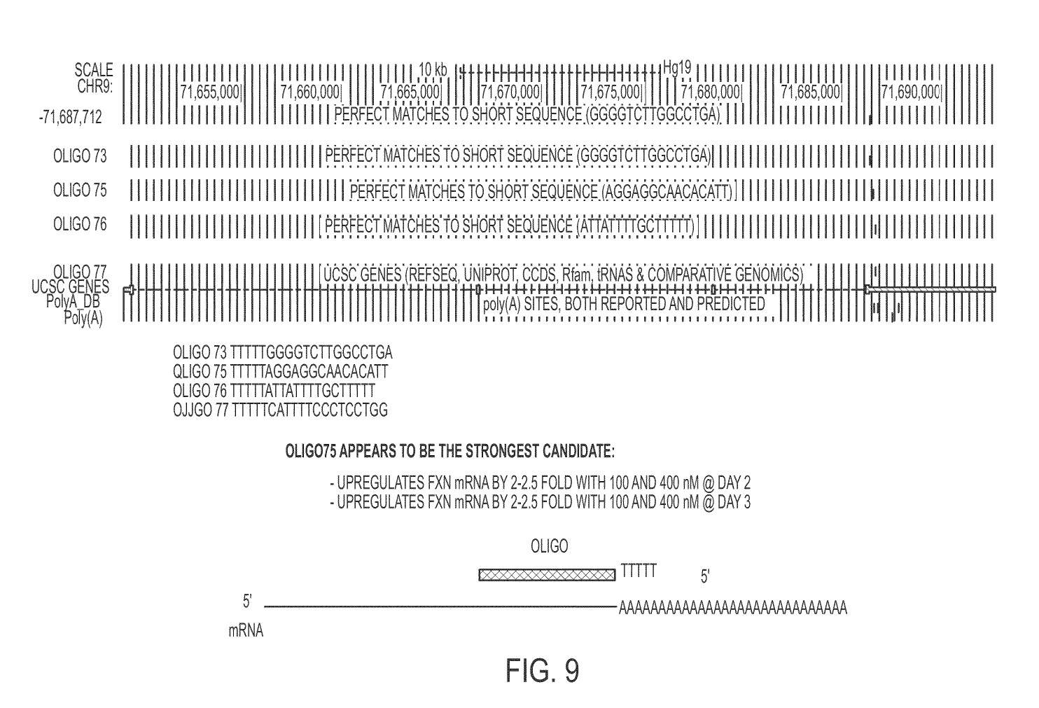

The 3′ and 5′ oligos were initially screened in a cell line from a patient having Friedreich's Ataxia (Cell line GM03816). FIGS. 7 and 8 show the results from transfecting the cell line with FXN 3′ end targeting oligonucleotides, demonstrating that several 3′ oligos were capable of upregulating FXN mRNA. Oligos 73, 75, 76, and 77 were shown to upregulate FXN mRNA to the greatest extent. Upon examination of the sequences of these four oligos, it was determined that oligos 73, 75, 76, and 77 contained poly-T sequences (FIG. 9). It was hypothesized that these oligos bound to the 3′ most end before the poly A tail, thus protecting the 3′ end from degradation. These results demonstrate that oligos designed to target the 3′ end can upregulate FXN expression. These results also suggest that oligos that target the 3′-most end directly adjacent to or overlapping with a poly-A tail can upregulate mRNA levels.

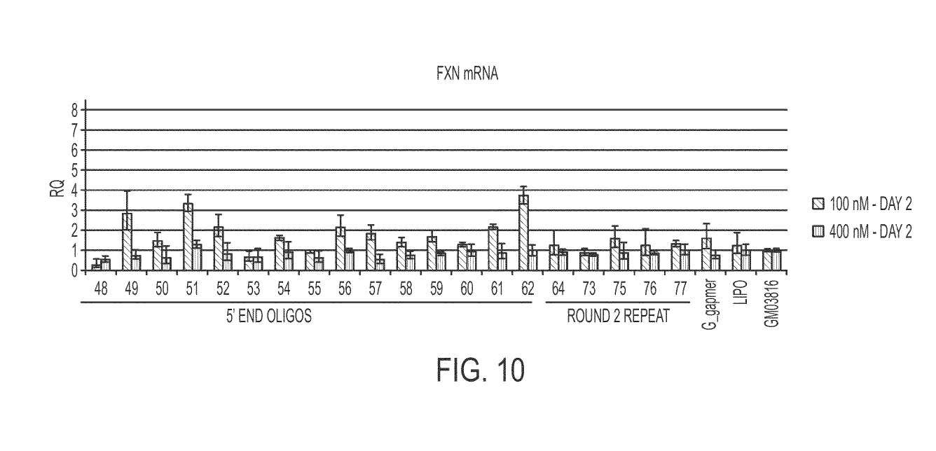

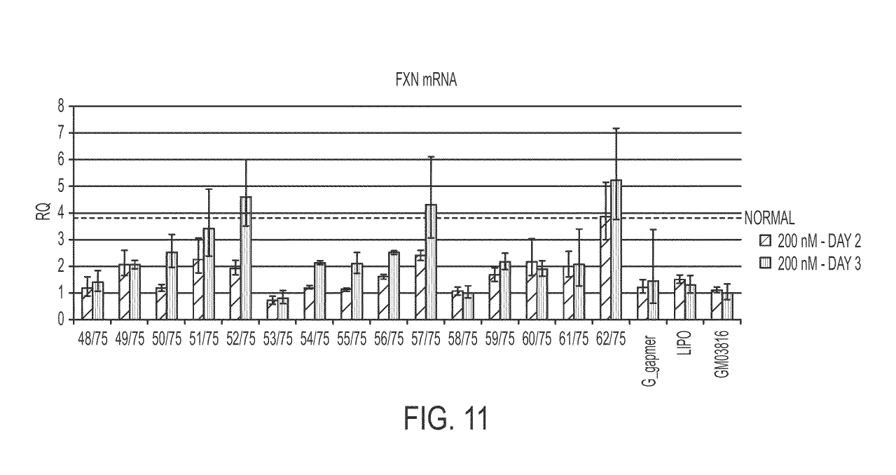

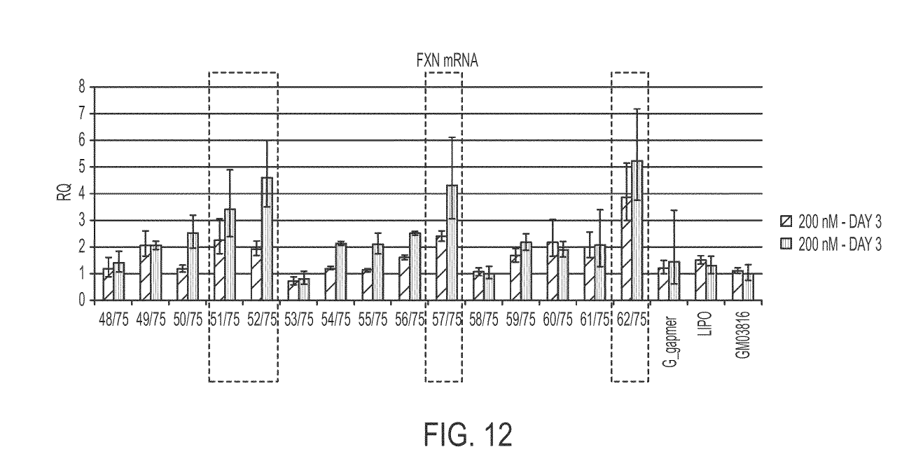

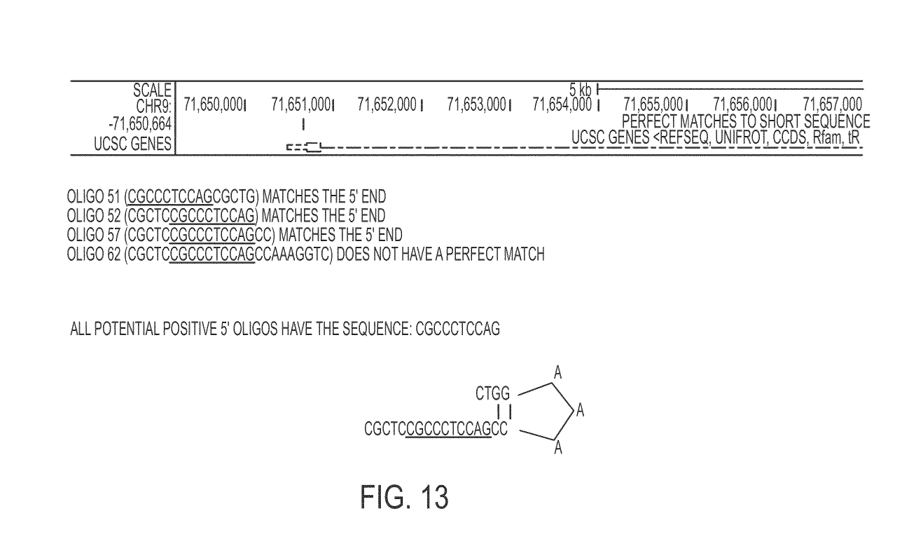

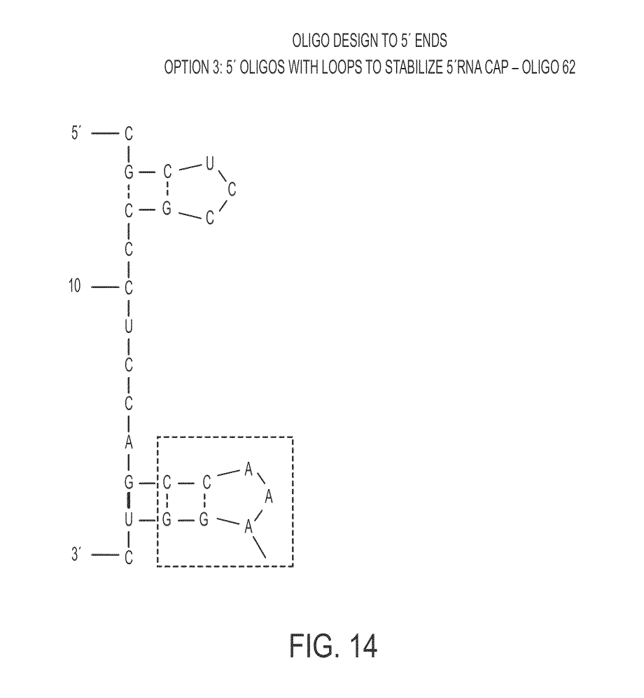

FIG. 10 shows the results from transfecting the GM03816 cell line with FXN 5′ end targeting oligonucleotides, demonstrating that several 5′ oligos are capable of upregulating FXN mRNA expression. FIGS. 11 and 12 show the results of screening FXN 5′ end oligos in combination with FXN 3′ oligo 75 in the GM03816 cell line. The combination of oligos 51 and 75, 52 and 75, 57 and 75, and 62 and 75 showed the highest upregulation of FXN mRNA expression. Upon examination of the sequences of the 5′ oligos, it was determined that oligos 51, 52, 57, and 62 all contained the motif CGCCCTCCAG, which mapped to a putatitive 5′ start site for a FXN mRNA isoform (FIG. 13). It was hypothesized that the oligos bound at the 5′-most end of the FXN mRNA, thus protecting the 5′ end from degradation. Oligo 62 contained a very long overhang sequence beyond the motif, which was hypothesized to form a loop structure that further protected the 5′-end by interacting with the 5′ methylguanosine cap (FIG. 14). These results suggest that targeting of the 5′-most end of an mRNA (which may be adjacent to a 5′ methylguanosine cap) is effective for upregulating mRNA.

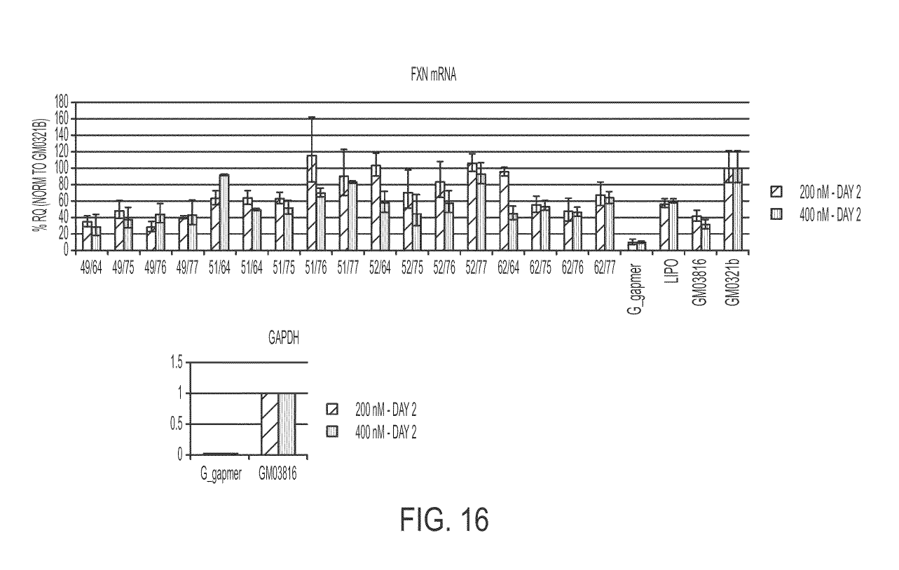

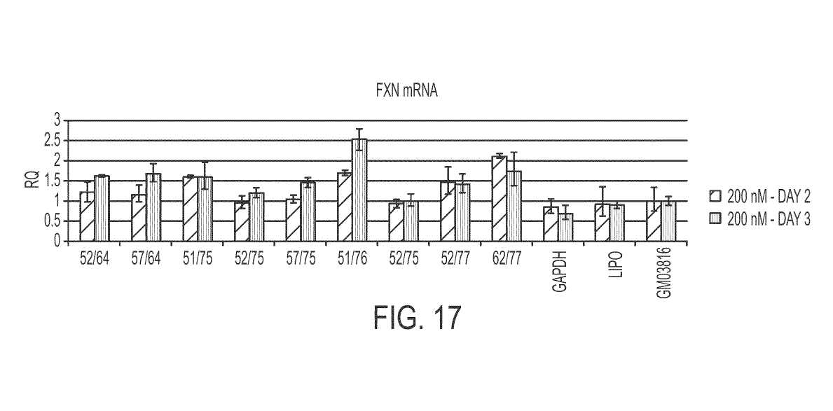

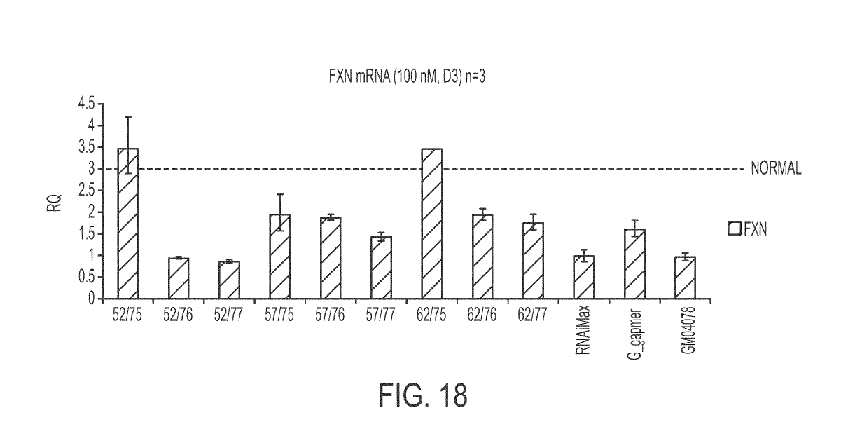

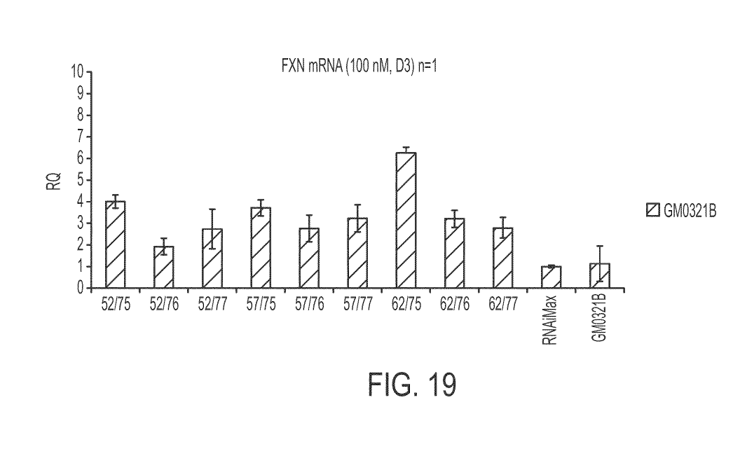

Next, a screening of the combination of positive oligo hits from previous 5′ and 3′ experiments was performed in the GM03816 FRDA patient cell line. It was determined that the FXN mRNA levels for several of the oligo combinations tested approached the levels of FXN mRNA in the GM0321B normal fibroblast cells, indicating that these oligo combinations were capable of upregulating FXN mRNA (FIG. 16). The levels of FXN mRNA at two and three days post transfection were then measured and it was confirmed that an increased steady state FXN mRNA levels was observed at 2 and 3 days post transfection (FIG. 17). The positive hits were then validated and shown to be effective in a second cell line, GM04078 FRDA patient fibroblasts (FIG. 18). Lastly a validation of the hits was performed in a ‘normal’ cell line, GM0321B fibroblasts. It was found that the oligos could upregulate FXN mRNA even in a normal cell line (FIG. 19). Together, these results suggest that combinations of 5′ and 3′ targeting oligos are capable of upregulating FXN expression and that these combinations can be, in some instances, more effective than the use of 5′ or 3′ oligos alone.

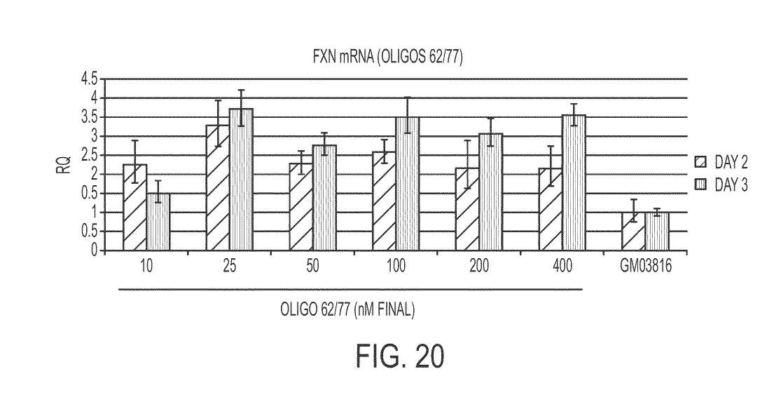

An exemplary 5′ and 3′ oligo combination, oligo 62 and oligo 77, was chosen for further optimization. All concentrations were shown to upregulate FXN in the GM03816 FRDA patient cell line and showed an increased steady-state of FXN mRNA levels at 2-3 days post transfection (FIG. 20). These results suggest that the oligos are effective over a wide range of concentrations, from 10 nM to 400 nM.

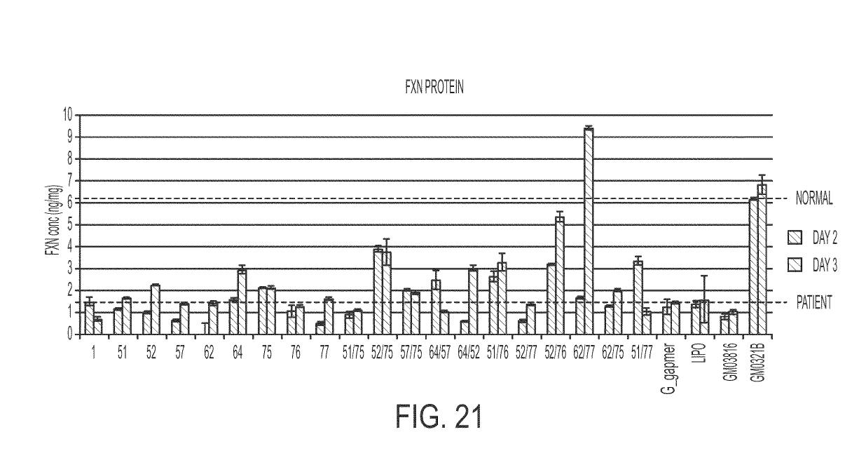

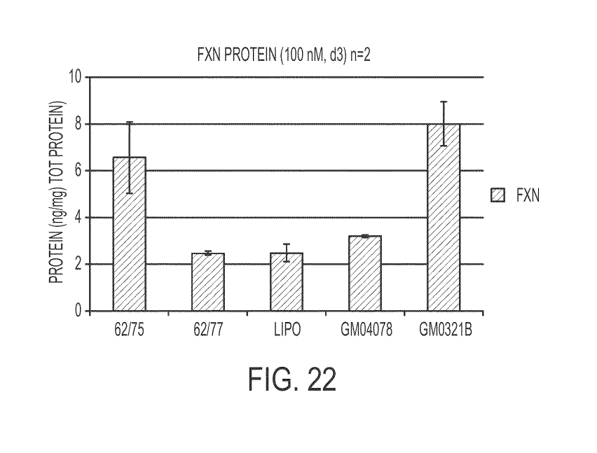

Next the effects of individual oligos and combinations of oligos on protein levels of FXN were investigated. GM03816 FRDA patient fibroblasts were treated with single oligos at 100 nM or two oligos at 200 nM final and the level of FXN protein was measured. Several single oligos and combinations of oligos were shown to upregulate FXN protein expression to some degree. The treatment with the combinations of oligos 52 and 75, oligos 64 and 52, oligos 51 and 76, oligos 52 and 76, oligos 62 and 77, and oligos 62 and 76, caused significant upregulation of FXN protein at day 3 post transfection (FIGS. 21 and 22). These results suggest that 5′ and 3′ targeting oligos are capable of upregulating FXN protein levels.

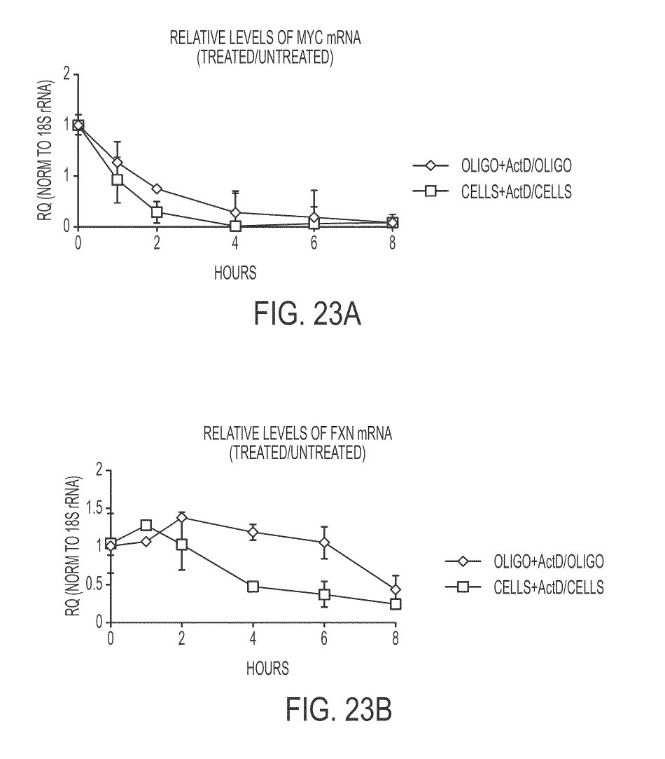

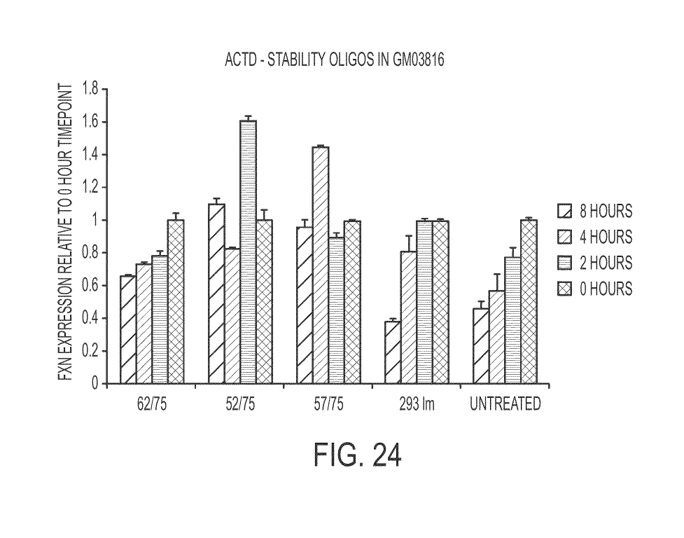

Next, the stability of FXN mRNA in the presence of different oligos was measured. It was hypothesized that the oligos were increasing FXN mRNA stability, rather than increasing the transcription of the FXN mRNA. To test this, cells were transfected with oligos in the presence of the transcription inhibitor Actinomycin D (ActD). The oligo combinations 62 and 75, 52 and 75, and 57 and 75 had higher levels of FXN mRNA in the presence of ActD, indicating that FXN mRNA was more stable in cells treated with the oligo combinations (FIGS. 23 and 24) than untreated cells.

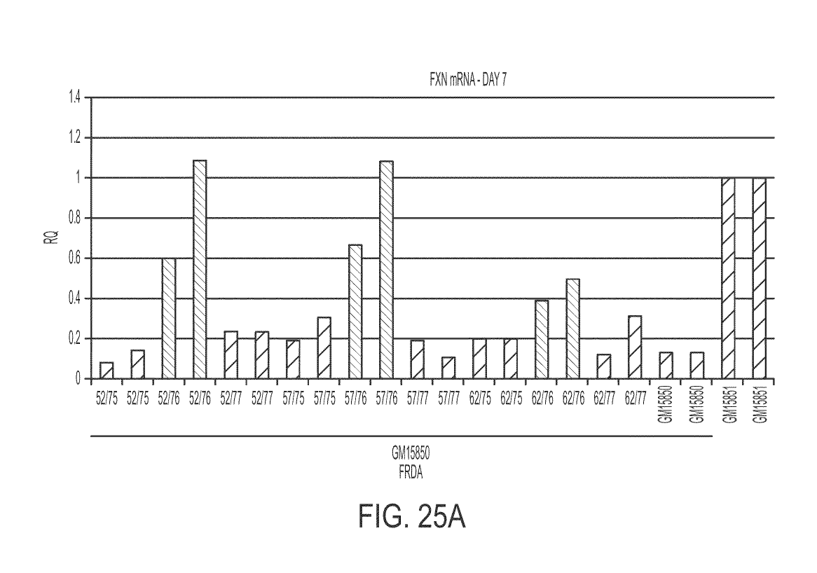

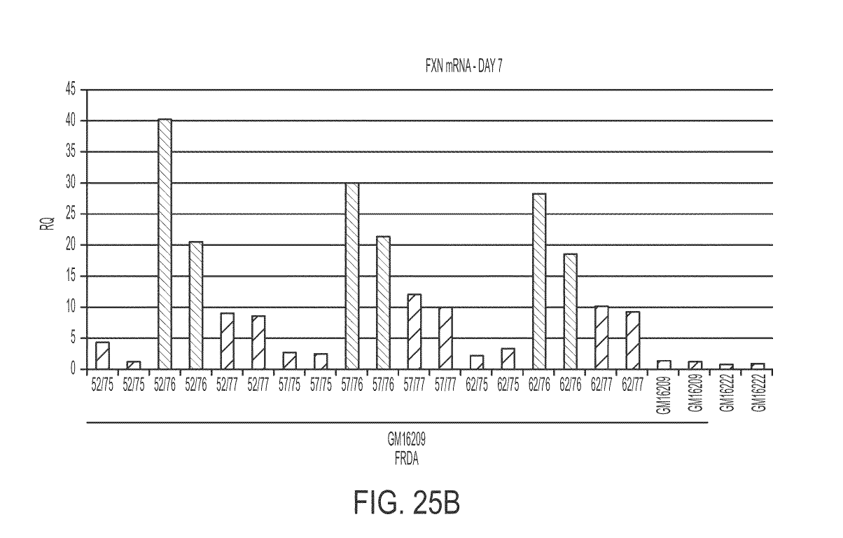

Lastly, several oligo combinations were tested in additional cell lines. One set of cell lines was obtained from a patient with Friedreich's ataxia (cell line GM15850) and from their unaffected sibling (cell line GM15851). The other cell lines were obtained from a patient with Friedreich's ataxia (cell line GM16209) and from their unaffected half-sibling (cell line GM16222). It was found that treatment with the combination of oligos 52 and 76, the combination of oligos 57 and 76, and the combination of oligos 62 and 76 significantly upregulated FXN mRNA levels (FIG. 25). In the GM15850 cell line, the levels of FXN mRNA in cells treated with either oligos 52 and 76 or oligos 57 and 76 approached the levels of the FXN mRNA in cells from the unaffected sibling. These results further indicate the efficacy of 5′ and 3′ end targeting oligonucleotides in upregulating FXN mRNA.

Overall, these results show that 5′ and 3′ end targeting oligos are effective for upregulating mRNA and protein expression and that this upregulation of expression is likely through stabilization of the mRNA.

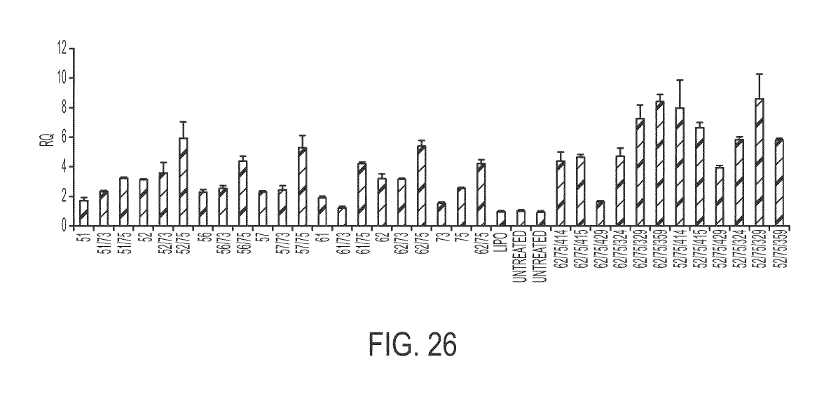

As an additional experiment, the 5′ and 3′ end targeting oligos were further combined with other oligos specific for sequences within the FXN gene (Table 6). The upregulation of the 5′ and 3′ oligos was further enhanced upon addition of subsets of these other oligos, suggesting that providing oligos that target multiple regions of an RNA or gene locus, e.g., a 5′ targeting oligo, a 3′ targeting oligo, and an internal targeting oligo, may be an additional method for upregulating mRNA expression levels (FIG. 26).

TABLE 6
Other targeting FXN

| SEQ | | | | | |
|---|---|---|---|---|---|
| ID | Oligo | | Gene | | Formatted |
| NO | Name | Base Sequence | Name | Organism | Sequence |
| 113 | 324 | CGGCGCCCGAGAG | FXN | human | dCs; lnaGs; |
| | | TCCACAT | | | dGs; lnaCs; |
| | | | | | dGs; lnaCs; |
| | | | | | dCs; lnaCs; |
| | | | | | dGs; lnaAs; |
| | | | | | dGs; lnaAs; |
| | | | | | dGs; lnaTs; |
| | | | | | dCs; lnaCs; |
| | | | | | dAs; lnaCs; |
| | | | | | dAs; lnaT-Sup |
| 114 | 329 | ACGGCGGCCGCAG | FXN | human | dAs; lnaCs; |
| | | AGTGGGG | | | dGs; lnaGs; |
| | | | | | dCs; lnaGs; |
| | | | | | dGs; lnaCs; |
| | | | | | dCs; lnaGs; |
| | | | | | dCs; lnaAs; |
| | | | | | dGs; lnaAs; |
| | | | | | dGs; lnaTs; |
| | | | | | dGs; lnaGs; |
| | | | | | dGs; lnaG-Sup |
| 115 | 359 | CCTCAAAAGCAGGA | FXN | human | dCs; lnaCs; |
| | | ATAAAAAAAATA | | | dTs; lnaCs; |
| | | | | | dAs; lnaAs; |
| | | | | | dAs; lnaAs; |
| | | | | | dGs; lnaCs; |
| | | | | | dAs; lnaGs; |
| | | | | | dGs; lnaAs; |
| | | | | | dAs; lnaTs; |
| | | | | | dAs; lnaAs; |
| | | | | | dAs; lnaAs; |
| | | | | | dAs; lnaAs; |
| | | | | | dAs; lnaAs; |
| | | | | | dTs; lnaA-Sup |
| 116 | 414 | ATGGGGGACGGGG | FXN | human | dAs; lnaTs; |
| | | CA | | | dGs; lnaGs; |
| | | | | | dGs; lnaGs; |
| | | | | | dGs; lnaAs; |
| | | | | | dCs; lnaGs; |
| | | | | | dGs; lnaGs; |
| | | | | | dGs; lnaCs; |
| | | | | | dA-Sup |
| 117 | 415 | GGTTGAGACTGGG | FXN | human | dGs; lnaGs; |
| | | TG | | | dTs; lnaTs; |
| | | | | | dGs; lnaAs; |
| | | | | | dGs; lnaAs; |
| | | | | | dCs; lnaTs; |
| | | | | | dGs; lnaGs; |
| | | | | | dGs; lnaTs; |
| | | | | | dG-Sup |
| 118 | 429 | ATGGGGGACGGGG | FXN | human | dAs; lnaTs; |
| | | CA | | | dGs; lnaGs; |
| | | | | | dGs; lnaGs; |
| | | | | | dGs; lnaAs; |
| | | | | | dCs; lnaGs; |
| | | | | | dGs; lnaGs; |
| | | | | | dGs; lnaCs; |
| | | | | | dA-Sup |

### Example 3

### Further Oligonucleotide Experiments Related to FXN

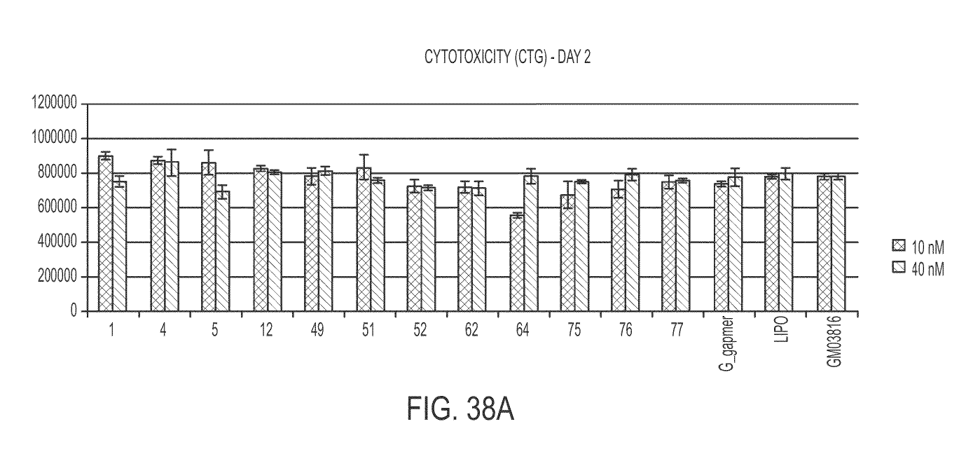

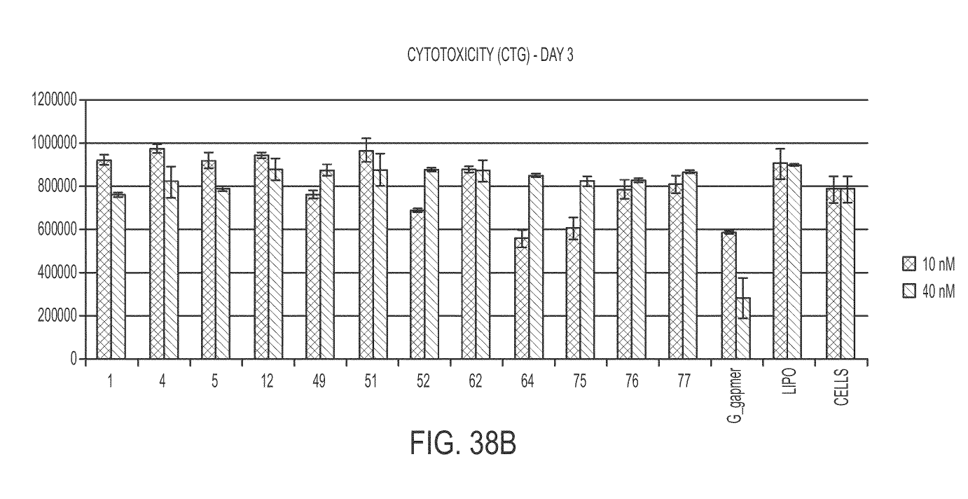

The experiments conducted in Example 3 utilized the same methods as Example 2, except that the oligonucleotide concentrations used were 10 and 40 nm. Transfection with 10 or 40 nM of an oligo was found to not be cytoxic to the cells at day 2 and day 3 post-transfection (FIG. 38).

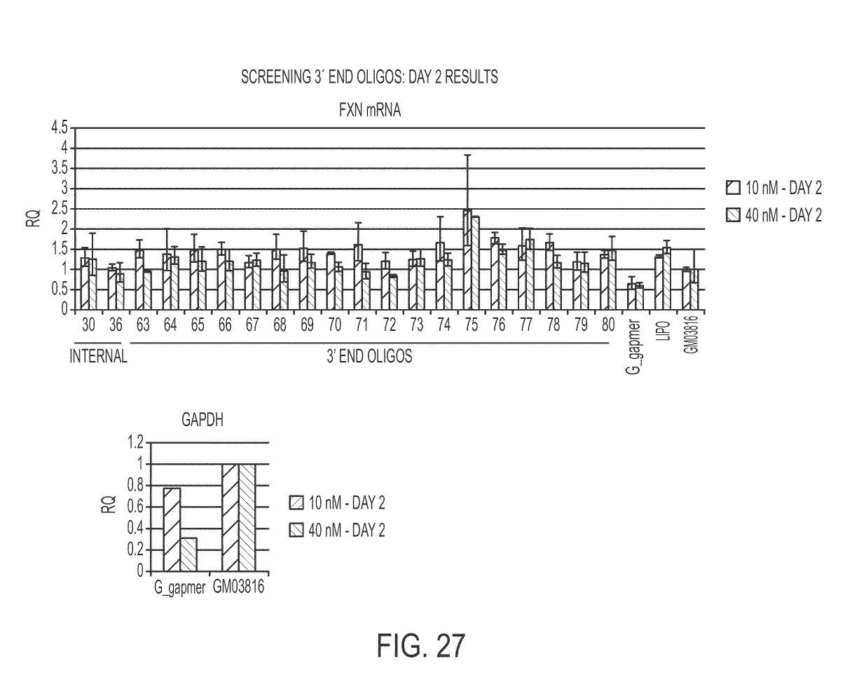

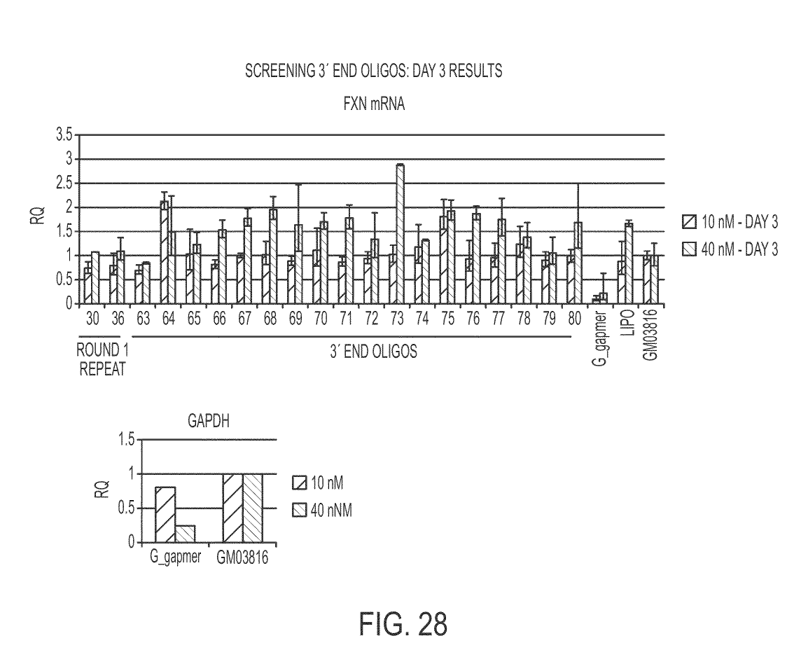

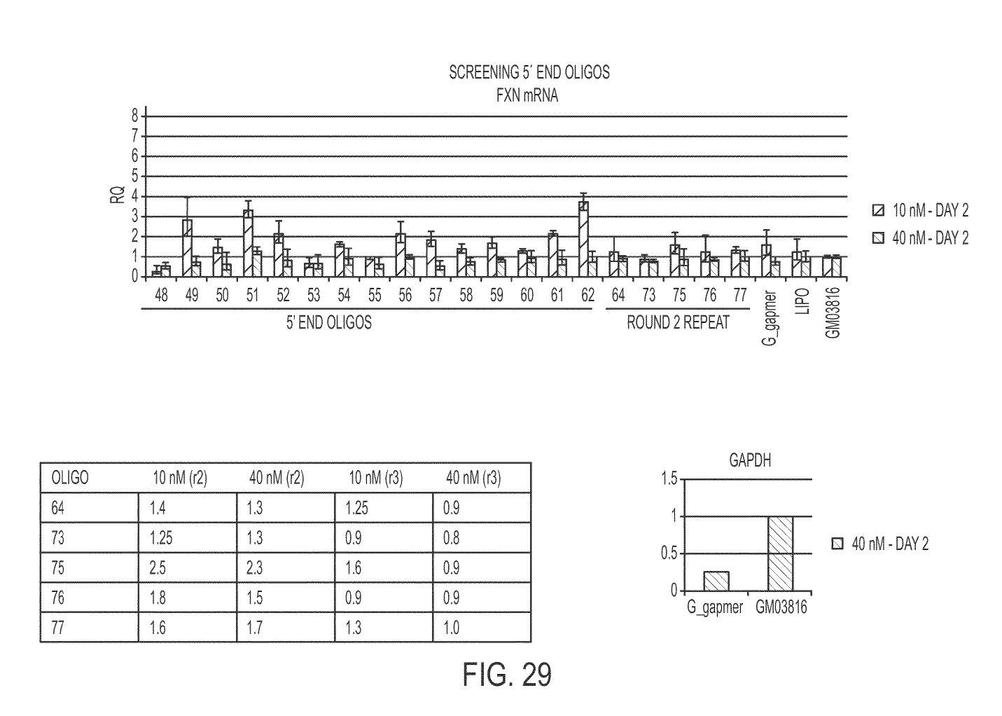

3′ and 5′ end targeting oligos were screened at 10 and 40 nM concentrations and FXN mRNA was measured at 2 and 3 days post-transfection. A subset of oligos were found to be capable of upregulating FXN mRNA at doses of 10 or 40 nM (FIGS. 27-29).

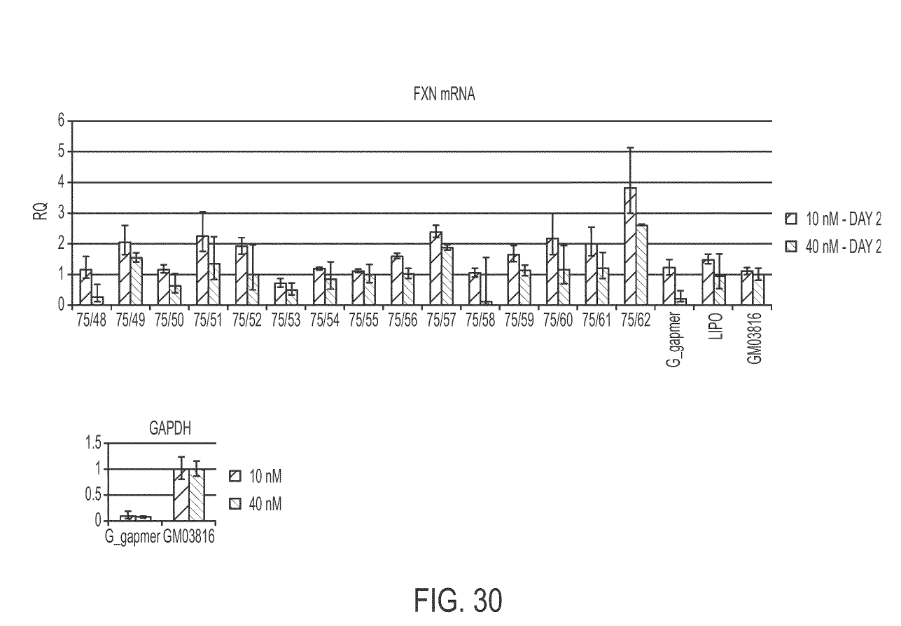

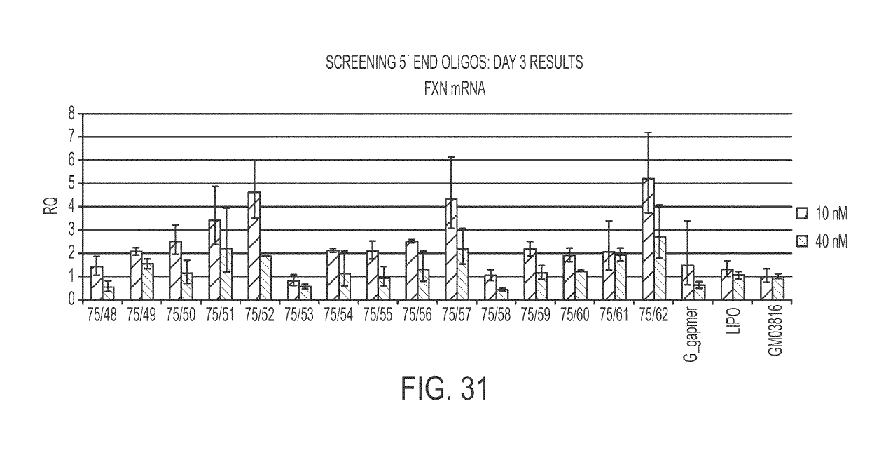

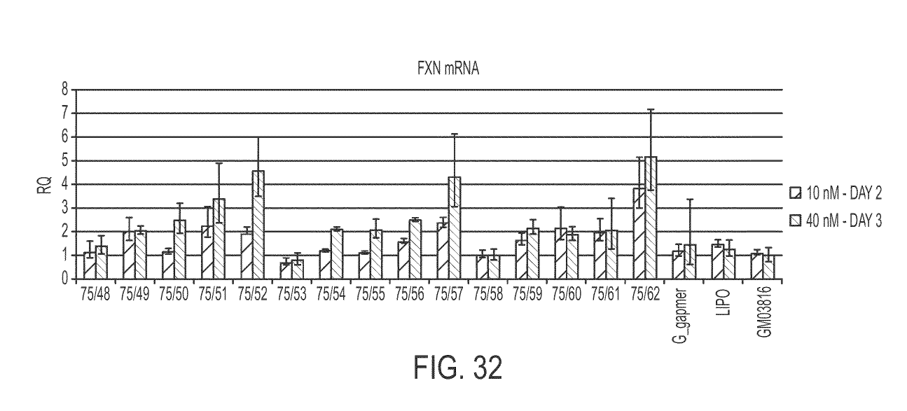

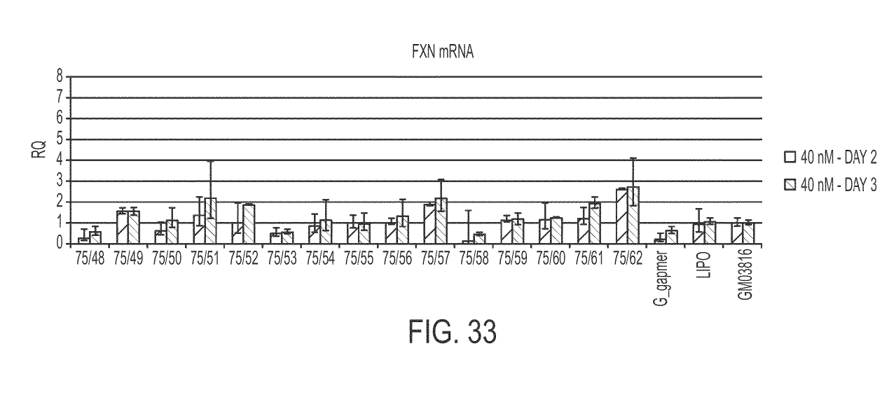

A screening of combinations of 5′ and 3′ end oligos was also performed at 10 and 40 nM concentrations and FXN mRNA was measured at 2 and 3 days post-transfection. A subset of oligo combinations were found to be capable of upregulating FXN mRNA at doses of 10 or 40 nM (FIGS. 30-33).

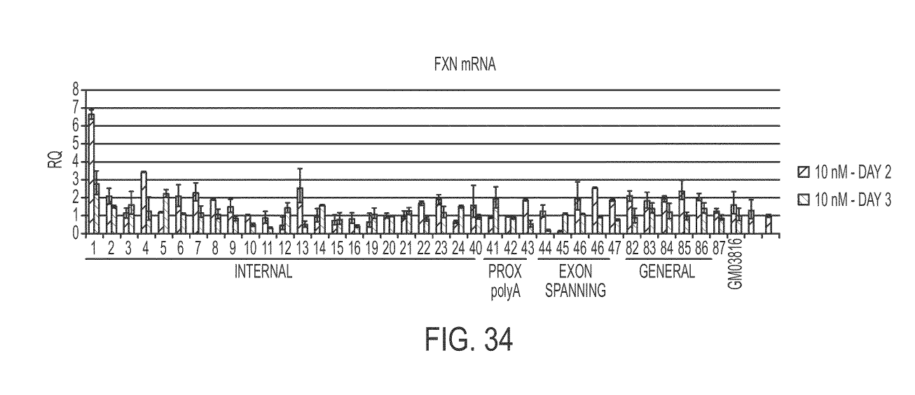

Other oligos that target FXN, e.g., internally, close to a poly-A tail, or spanning an exon, were also found to be capable of upregulating FXN mRNA at doses of 10 or 40 nM (FIG. 34).

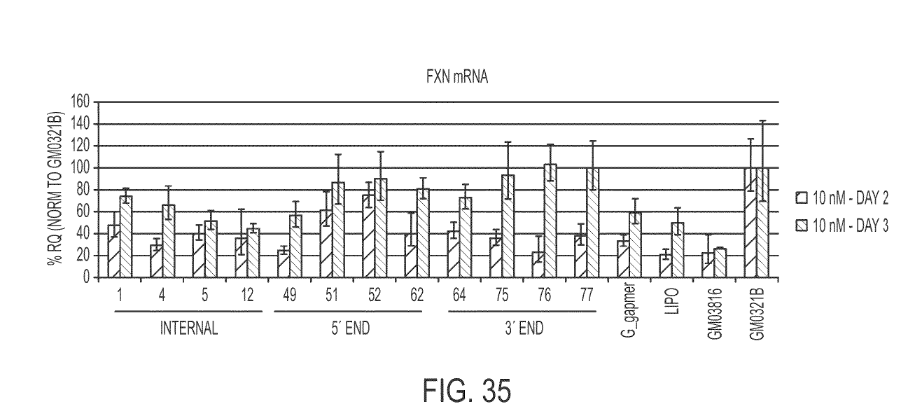

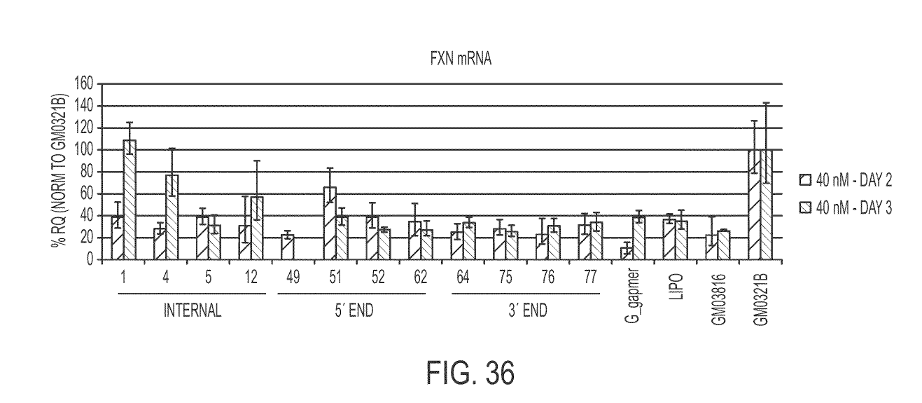

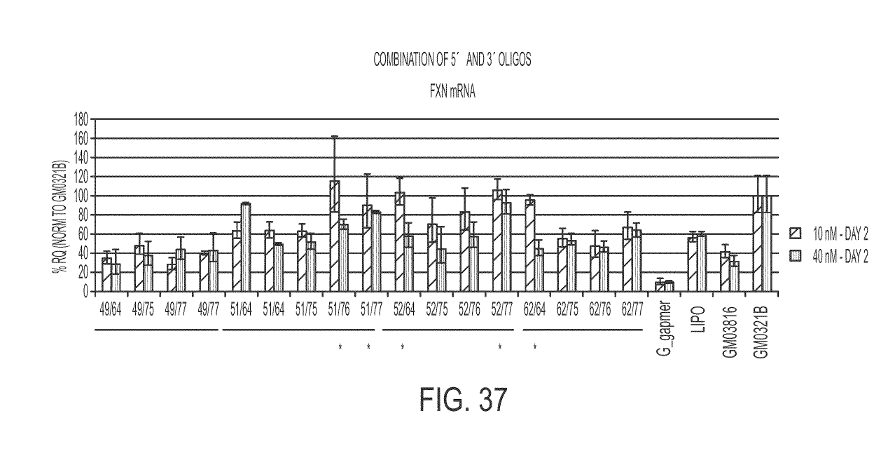

Additional experiments were performed to further demonstrate that FXN mRNA levels can be increased using a single oligonucleotide or combinations of oligonucleotides at 10 and 40 nM concentrations (FIGS. 35-37).

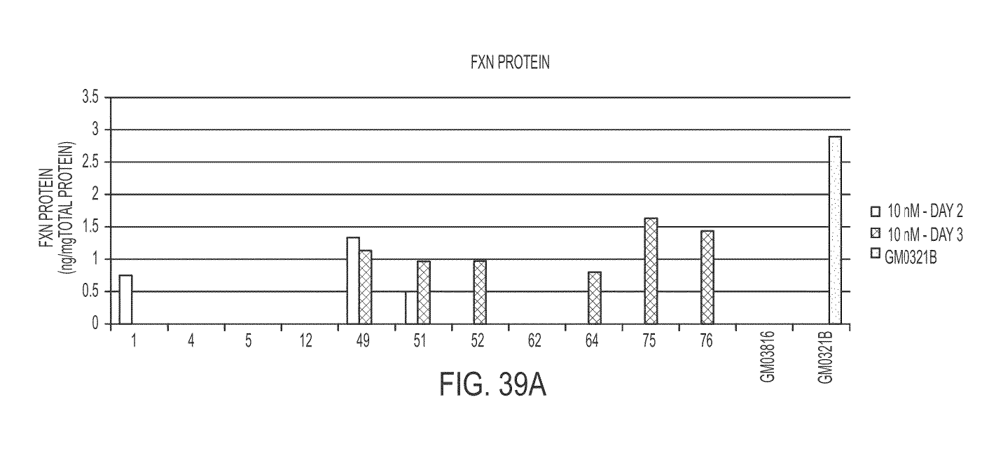

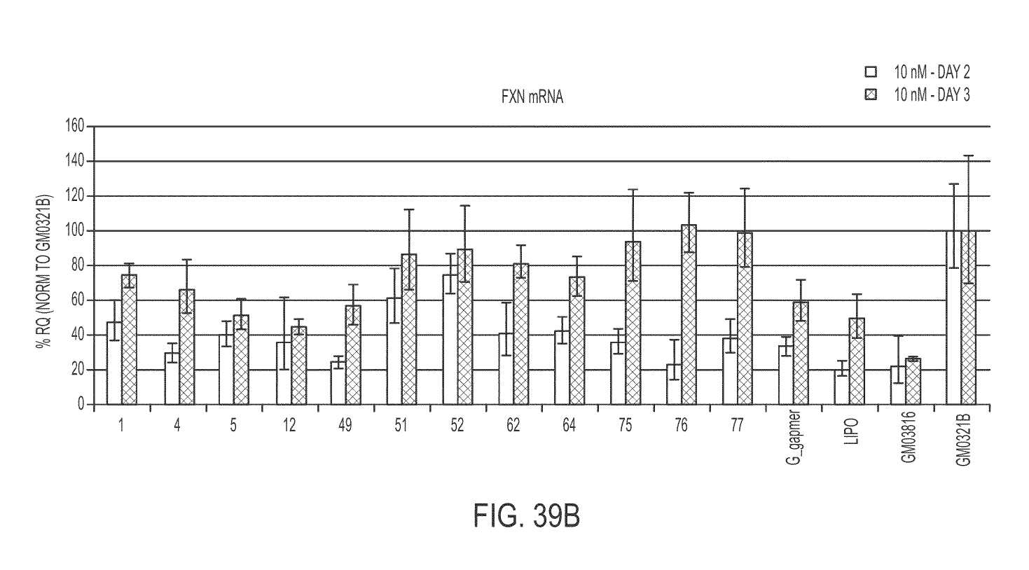

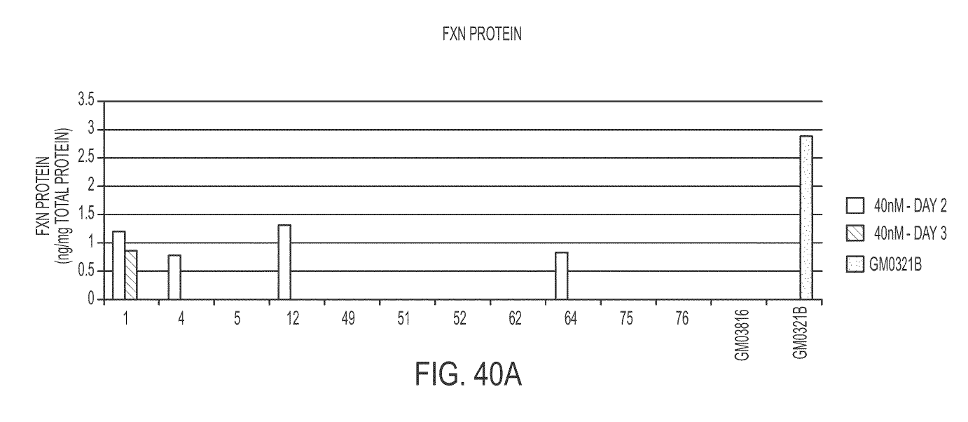

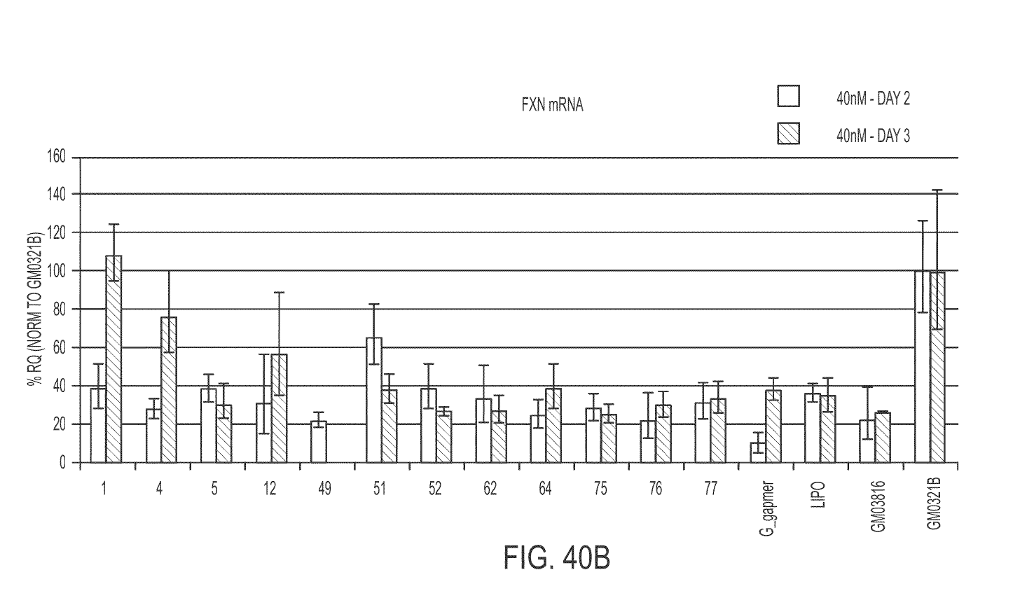

Next, 5′ and 3′ end targeting oligos were tested individually for their capability to upregulate FXN protein levels at 10 and 40 nM concentrations. It was determined that a subset of oligos were capable of upregulating FXN protein levels at 2 and 3 days post-transfection at 10 and 40 nM concentrations (FIGS. 39 and 40). The results indicate that 5′ and 3′ targeting oligos, and combinations thereof, are capable to upregulating FXN mRNA and protein even at concentrations as low as 10 nM.

### Example 4

### Further Oligonucleotides for Increasing mRNA Stability

Several additional oligonucleotides were designed to target the 5′ end of an RNA, the 3′ end of an RNA, or target both the 5′ end and 3′ end of an RNA (“bridging oligos”). These oligos are shown in Table 7.

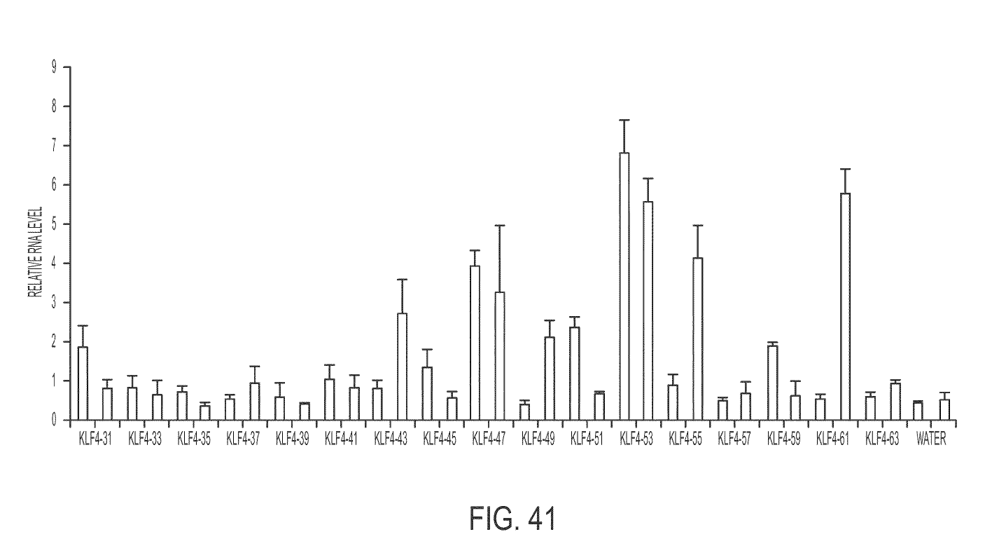

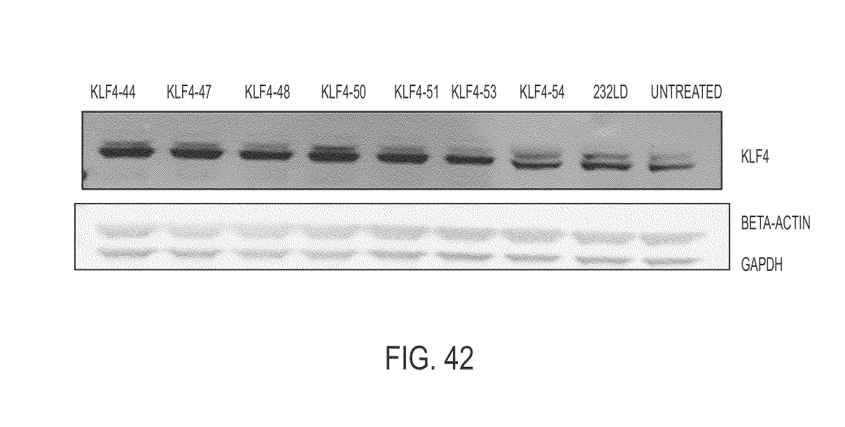

Oligonucleotides specific for KLF4 were tested by treating cells with each oligo. Several KLF4 oligos were able to upregulate KLF4 mRNA levels in the treated cells (FIG. 41). A subset of the KLF4 oligos were also able to upregulate KLF4 protein levels in the treated cells (FIG. 42). These results show that 5′ and 3′ targeting oligos were able to upregulate mRNA and protein levels for KLF4, demonstrating that 5′ and 3′ targeting oligos are generally useful for upregulating expression of an RNA (and also the corresponding protein).

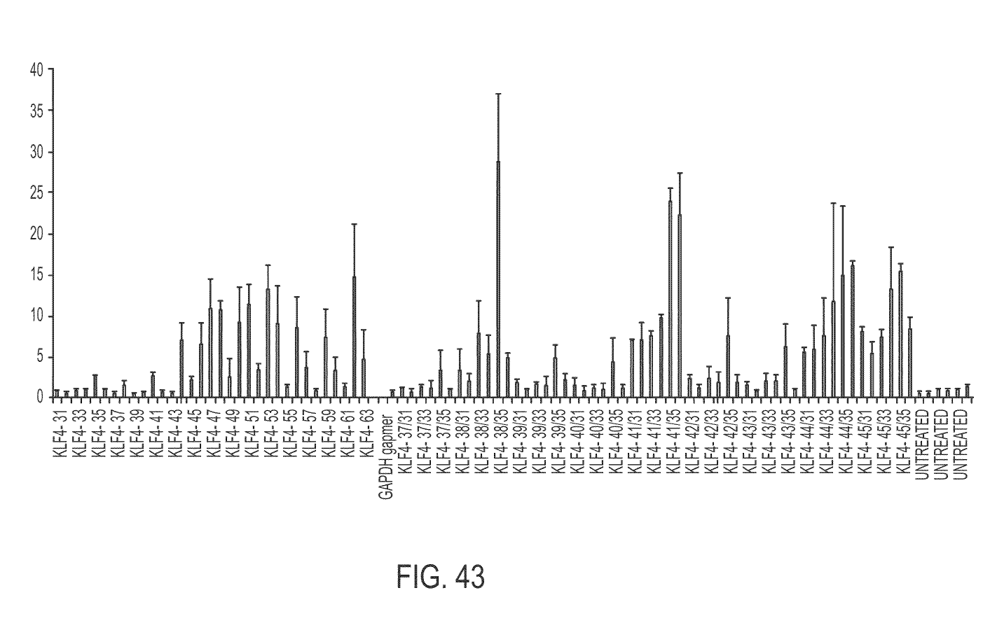

In addition, expression levels of KLF4 mRNA were evaluated in cells treated with KLF4 5′ and 3′ end targeting oligos, including circularized oligonucleotides targeting both 5′ and 3′ ends of KLF4, and individual oligonucleotides targeting 5′ and 3′ ends of KLF4. Results are shown in FIG. 43.

KLF4 5′ and 3′ end oligos were transfected to Hep3B cells at 30 nM concentration using RNAimax. RNA analysis was done with Cells-to-Ct kit (Life Technologies) using KLF4 and ACTIN (housekeeper control) primers purchased from Life Technologies. Western for KLF4 protein was done with KLF4 rabbit (Cell Signaling 4038S).

TABLE 7
Oligonucleotides designed to target 5′ and 3′ ends of RNAs

| SEQ | Oligo | | Gene | Target | | |
|---|---|---|---|---|---|---|
| ID NO | Name | Base Sequence | Name | Region | Organism | Formatted Sequence |
| 119 | FXN-437 | TGACCCAAGGGAGACTT | FXN | 5′ and 3′ | human | dTs; lnaGs; |
| | m02 | TTTGGTTTTTAAGGCTTT | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaCs; |
| | | | | | | dTs; lnaTs; |
| | | | | | | dT-Sup |
| 120 | FXN-438 | TGGCCACTGGCCGCATT | FXN | 5′ and 3′ | human | dTs; lnaGs; |
| | m02 | TTTGGTTTTTAAGGCTTT | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaCs; |
| | | | | | | dTs; lnaTs; |
| | | | | | | dT-Sup |
| 121 | FXN-439 | CGGCGACCCCTGGTGTT | FXN | 5′ and 3′ | human | dCs; lnaGs; |
| | m02 | TTTGGTTTTTAAGGCTTT | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaCs; |
| | | | | | | dTs; lnaTs; |
| | | | | | | dT-Sup |
| 122 | FXN-440 | CGCCCTCCAGCGCTGTT | FXN | 5′ and 3′ | human | dCs; lnaGs; |
| | m02 | TTTGGTTTTTAAGGCTTT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaCs; |
| | | | | | | dTs; lnaTs; |
| | | | | | | dT-Sup |
| 123 | FXN-441 | CGCTCCGCCCTCCAGTTT | FXN | 5′ and 3′ | human | dCs; lnaGs; |
| | m02 | TTGGTTTTTAAGGCTTT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaCs; |
| | | | | | | dTs; lnaTs; |
| | | | | | | dT-Sup |
| 124 | FXN-442 | TGACCCAAGGGAGACTT | FXN | 5′ and 3′ | human | dTs; lnaGs; |
| | m02 | TTTGGGGTCTTGGCCTGA | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dGs; lnaGs; |
| | | | | | | dGs; lnaGs; |
| | | | | | | dTs; lnaCs; |
| | | | | | | dTs; lnaTs; |
| | | | | | | dGs; lnaGs; |
| | | | | | | dCs; lnaCs; |
| | | | | | | dTs; lnaGs; |
| | | | | | | dA-Sup |
| 125 | FXN-443 | TGGCCACTGGCCGCATT | FXN | 5′ and 3′ | human | dTs; lnaGs; |
| | m02 | TTTGGGGTCTTGGCCTGA | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dGs; lnaGs; |
| | | | | | | dGs; lnaGs; |
| | | | | | | dTs; lnaCs; |
| | | | | | | dTs; lnaTs; |
| | | | | | | dGs; lnaGs; |
| | | | | | | dCs; lnaCs; |
| | | | | | | dTs; lnaGs; |
| | | | | | | dA-Sup |
| 126 | FXN-444 | CGGCGACCCCTGGTGTT | FXN | 5′ and 3′ | human | dCs; lnaGs; |
| | m02 | TTTGGGGTCTTGGCCTGA | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dGs; lnaGs; |
| | | | | | | dGs; lnaGs; |
| | | | | | | dTs; lnaCs; |
| | | | | | | dTs; lnaTs; |
| | | | | | | dGs; lnaGs; |
| | | | | | | dCs; lnaCs; |
| | | | | | | dTs; lnaGs; |
| | | | | | | dA-Sup |
| 127 | FXN-445 | CGCCCTCCAGCGCTGTT | FXN | 5′ and 3′ | human | dCs; lnaGs; |
| | m02 | TTTGGGGTCTTGGCCTGA | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dGs; lnaGs; |
| | | | | | | dGs; lnaGs; |
| | | | | | | dTs; lnaCs; |
| | | | | | | dTs; lnaTs; |
| | | | | | | dGs; lnaGs; |
| | | | | | | dCs; lnaCs; |
| | | | | | | dTs; lnaGs; |
| | | | | | | dA-Sup |
| 128 | FXN-446 | CGCTCCGCCCTCCAGTTT | FXN | 5′ and 3′ | human | dCs; lnaGs; |
| | m02 | TTGGGGTCTTGGCCTGA | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dGs; lnaGs; |
| | | | | | | dGs; lnaGs; |
| | | | | | | dTs; lnaCs; |
| | | | | | | dTs; lnaTs; |
| | | | | | | dGs; lnaGs; |
| | | | | | | dCs; lnaCs; |
| | | | | | | dTs; lnaGs; |
| | | | | | | dA-Sup |
| 129 | FXN-447 | TGACCCAAGGGAGACTT | FXN | 5′ and 3′ | human | dTs; lnaGs; |
| | m02 | TTTCATAATGAAGCTGGG | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dCs; lnaAs; |
| | | | | | | dTs; lnaAs; |
| | | | | | | dAs; lnaTs; |
| | | | | | | dGs; lnaAs; |
| | | | | | | dAs; lnaGs; |
| | | | | | | dCs; lnaTs; |
| | | | | | | dGs; lnaGs; |
| | | | | | | dG-Sup |
| 130 | FXN-448 | TGGCCACTGGCCGCATT | FXN | 5′ and 3′ | human | dTs; lnaGs; |
| | m02 | TTTCATAATGAAGCTGGG | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dCs; lnaAs; |
| | | | | | | dTs; lnaAs; |
| | | | | | | dAs; lnaTs; |
| | | | | | | dGs; lnaAs; |
| | | | | | | dAs; lnaGs; |
| | | | | | | dCs; lnaTs; |
| | | | | | | dGs; lnaGs; |
| | | | | | | dG-Sup |
| 131 | FXN-449 | CGGCGACCCCTGGTGTT | FXN | 5′ and 3′ | human | dCs; lnaGs; |
| | m02 | TTTCATAATGAAGCTGGG | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaAs; |
| | | | | | | dAs; lnaTs; |
| | | | | | | dGs; lnaAs; |
| | | | | | | dAs; lnaGs; |
| | | | | | | dCs; lnaTs; |
| | | | | | | dGs; lnaGs; |
| | | | | | | dG-Sup |
| 132 | FXN-450 | CGCCCTCCAGCGCTGTT | FXN | 5′ and 3′ | human | dCs; lnaGs; |
| | m02 | TTTCATAATGAAGCTGGG | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaAs; |
| | | | | | | dAs; lnaTs; |
| | | | | | | dGs; lnaAs; |
| | | | | | | dAs; lnaGs; |
| | | | | | | dCs; lnaTs; |
| | | | | | | dGs; lnaGs; |
| | | | | | | dG-Sup |
| 133 | FXN-451 | CGCTCCGCCCTCCAGTTT | FXN | 5′ and 3′ | human | dCs; lnaGs; |
| | m02 | TTCATAATGAAGCTGGG | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaAs; |
| | | | | | | dAs; lnaTs; |
| | | | | | | dGs; lnaAs; |
| | | | | | | dAs; lnaGs; |
| | | | | | | dCs; lnaTs; |
| | | | | | | dGs; lnaGs; |
| | | | | | | dG-Sup |
| 134 | FXN-452 | TGACCCAAGGGAGACTT | FXN | 5′ and 3′ | human | dTs; lnaGs; |
| | m02 | TTTAGGAGGCAACACATT | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dAs; lnaGs; |
| | | | | | | dGs; lnaAs; |
| | | | | | | dGs; lnaGs; |
| | | | | | | dCs; lnaAs; |
| | | | | | | dAs; lnaCs; |
| | | | | | | dAs; lnaCs; |
| | | | | | | dAs; lnaTs; |
| | | | | | | dT-Sup |
| 135 | FXN-453 | TGGCCACTGGCCGCATT | FXN | 5′ and 3′ | human | dTs; lnaGs; |
| | m02 | TTTAGGAGGCAACACATT | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dAs; lnaGs; |
| | | | | | | dGs; lnaAs; |
| | | | | | | dGs; lnaGs; |
| | | | | | | dCs; lnaAs; |
| | | | | | | dAs; lnaCs; |
| | | | | | | dAs; lnaCs; |
| | | | | | | dAs; lnaTs; |
| | | | | | | dT-Sup |
| 136 | FXN-454 | CGGCGACCCCTGGTGTT | FXN | 5′ and 3′ | human | dCs; lnaGs; |
| | m02 | TTTAGGAGGCAACACATT | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dAs; lnaGs; |
| | | | | | | dGs; lnaAs; |
| | | | | | | dGs; lnaGs; |
| | | | | | | dCs; lnaAs; |
| | | | | | | dAs; lnaCs; |
| | | | | | | dAs; lnaCs; |
| | | | | | | dAs; lnaTs; |
| | | | | | | dT-Sup |
| 137 | FXN-455 | CGCCCTCCAGCGCTGTT | FXN | 5′ and 3′ | human | dCs; lnaGs; |
| | m02 | TTTAGGAGGCAACACATT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dAs; lnaGs; |
| | | | | | | dGs; lnaAs; |
| | | | | | | dGs; lnaGs; |
| | | | | | | dCs; lnaAs; |
| | | | | | | dAs; lnaCs; |
| | | | | | | dAs; lnaCs; |
| | | | | | | dAs; lnaTs; |
| | | | | | | dT-Sup |
| 138 | FXN-456 | CGCTCCGCCCTCCAGTTT | FXN | 5′ and 3′ | human | dCs; lnaGs; |
| | m02 | TTAGGAGGCAACACATT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dAs; lnaGs; |
| | | | | | | dGs; lnaAs; |
| | | | | | | dGs; lnaGs; |
| | | | | | | dCs; lnaAs; |
| | | | | | | dAs; lnaCs; |
| | | | | | | dAs; lnaCs; |
| | | | | | | dAs; lnaTs; |
| | | | | | | dT-Sup |
| 139 | FXN-457 | TGACCCAAGGGAGACTT | FXN | 5′ and 3′ | human | dTs; lnaGs; |
| | m02 | TTTATTATTTTGCTTTTT | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 140 | FXN-458 | TGGCCACTGGCCGCATT | FXN | 5′ and 3′ | human | dTs; lnaGs; |
| | m02 | TTTATTATTTTGCTTTTT | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 141 | FXN-459 | CGGCGACCCCTGGTGTT | FXN | 5′ and 3′ | human | dCs; lnaGs; |
| | m02 | TTTATTATTTTGCTTTTT | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 142 | FXN-460 | CGCCCTCCAGCGCTGTT | FXN | 5′ and 3′ | human | dCs; lnaGs; |
| | m02 | TTTATTATTTTGCTTTTT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 143 | FXN-461 | CGCTCCGCCCTCCAGTTT | FXN | 5′ and 3′ | human | dCs; lnaGs; |
| | m02 | TTATTATTTTGCTTTTT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 144 | FXN-462 | TGACCCAAGGGAGACTT | FXN | 5′ and 3′ | human | dTs; lnaGs; |
| | m02 | TTTCATTTTCCCTCCTGG | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dCs; lnaCs; |
| | | | | | | dCs; lnaTs; |
| | | | | | | dCs; lnaCs; |
| | | | | | | dTs; lnaGs; |
| | | | | | | dG-Sup |
| 145 | FXN-463 | TGGCCACTGGCCGCATT | FXN | 5′ and 3′ | human | dTs; lnaGs; |
| | m02 | TTTCATTTTCCCTCCTGG | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dCs; lnaCs; |
| | | | | | | dCs; lnaTs; |
| | | | | | | dCs; lnaCs; |
| | | | | | | dTs; lnaGs; |
| | | | | | | dG-Sup |
| 146 | FXN-464 | CGGCGACCCCTGGTGTT | FXN | 5′ and 3′ | human | dCs; lnaGs; |
| | m02 | TTTCATTTTCCCTCCTGG | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dCs; lnaCs; |
| | | | | | | dCs; lnaTs; |
| | | | | | | dCs; lnaCs; |
| | | | | | | dTs; lnaGs; |
| | | | | | | dG-Sup |
| 147 | FXN-465 | CGCCCTCCAGCGCTGTT | FXN | 5′ and 3′ | human | dCs; lnaGs; |
| | m02 | TTTCATTTTCCCTCCTGG | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dCs; lnaCs; |
| | | | | | | dCs; lnaTs; |
| | | | | | | dCs; lnaCs; |
| | | | | | | dTs; lnaGs; |
| | | | | | | dG-Sup |
| 148 | FXN-466 | CGCTCCGCCCTCCAGTTT | FXN | 5′ and 3′ | human | dCs; lnaGs; |
| | m02 | TTCATTTTCCCTCCTGG | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dCs; lnaCs; |
| | | | | | | dCs; lnaTs; |
| | | | | | | dCs; lnaCs; |
| | | | | | | dTs; lnaGs; |
| | | | | | | dG-Sup |
| 149 | FXN-467 | TGACCCAAGGGAGACTT | FXN | 5′ and 3′ | human | dTs; lnaGs; |
| | m02 | TTTGTAGGCTACCCTTTA | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dGs; lnaTs; |
| | | | | | | dAs; lnaGs; |
| | | | | | | dGs; lnaCs; |
| | | | | | | dTs; lnaAs; |
| | | | | | | dCs; lnaCs; |
| | | | | | | dCs; lnaTs; |
| | | | | | | dTs; lnaTs; |
| | | | | | | dA-Sup |
| 150 | FXN-468 | TGGCCACTGGCCGCATT | FXN | 5′ and 3′ | human | dTs; lnaGs; |
| | m02 | TTTGTAGGCTACCCTTTA | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dGs; lnaTs; |
| | | | | | | dAs; lnaGs; |
| | | | | | | dGs; lnaCs; |
| | | | | | | dTs; lnaAs; |
| | | | | | | dCs; lnaCs; |
| | | | | | | dCs; lnaTs; |
| | | | | | | dTs; lnaTs; |
| | | | | | | dA-Sup |
| 151 | FXN-469 | CGGCGACCCCTGGTGTT | FXN | 5′ and 3′ | human | dCs; lnaGs; |
| | m02 | TTTGTAGGCTACCCTTTA | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dGs; lnaTs; |
| | | | | | | dAs; lnaGs; |
| | | | | | | dGs; lnaCs; |
| | | | | | | dTs; lnaAs; |
| | | | | | | dCs; lnaCs; |
| | | | | | | dCs; lnaTs; |
| | | | | | | dTs; lnaTs; |
| | | | | | | dA-Sup |
| 152 | FXN-470 | CGCCCTCCAGCGCTGTT | FXN | 5′ and 3′ | human | dCs; lnaGs; |
| | m02 | TTTGTAGGCTACCCTTTA | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dGs; lnaTs; |
| | | | | | | dAs; lnaGs; |
| | | | | | | dGs; lnaCs; |
| | | | | | | dTs; lnaAs; |
| | | | | | | dCs; lnaCs; |
| | | | | | | dCs; lnaTs; |
| | | | | | | dTs; lnaTs; |
| | | | | | | dA-Sup |
| 153 | FXN-471 | CGCTCCGCCCTCCAGTTT | FXN | 5′ and 3′ | human | dCs; lnaGs; |
| | m02 | TTGTAGGCTACCCTTTA | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dGs; lnaTs; |
| | | | | | | dAs; lnaGs; |
| | | | | | | dGs; lnaCs; |
| | | | | | | dTs; lnaAs; |
| | | | | | | dCs; lnaCs; |
| | | | | | | dCs; lnaTs; |
| | | | | | | dTs; lnaTs; |
| | | | | | | dA-Sup |
| 154 | FXN-472 | TGACCCAAGGGAGACTT | FXN | 5′ and 3′ | human | dTs; lnaGs; |
| | m02 | TTTGAGGCTTGTTGCTTT | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dGs; lnaAs; |
| | | | | | | dGs; lnaGs; |
| | | | | | | dCs; lnaTs; |
| | | | | | | dTs; lnaGs; |
| | | | | | | dTs; lnaTs; |
| | | | | | | dGs; lnaCs; |
| | | | | | | dTs; lnaTs; |
| | | | | | | dT-Sup |
| 155 | FXN-473 | TGGCCACTGGCCGCATT | FXN | 5′ and 3′ | human | dTs; lnaGs; |
| | m02 | TTTGAGGCTTGTTGCTTT | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dGs; lnaAs; |
| | | | | | | dGs; lnaGs; |
| | | | | | | dCs; lnaTs; |
| | | | | | | dTs; lnaGs; |
| | | | | | | dTs; lnaTs; |
| | | | | | | dGs; lnaCs; |
| | | | | | | dTs; lnaTs; |
| | | | | | | dT-Sup |
| 156 | FXN-474 | CGGCGACCCCTGGTGTT | FXN | 5′ and 3′ | human | dCs; lnaGs; |
| | m02 | TTTGAGGCTTGTTGCTTT | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dGs; lnaAs; |
| | | | | | | dGs; lnaGs; |
| | | | | | | dCs; lnaTs; |
| | | | | | | dTs; lnaGs; |
| | | | | | | dTs; lnaTs; |
| | | | | | | dGs; lnaCs; |
| | | | | | | dTs; lnaTs; |
| | | | | | | dT-Sup |
| 157 | FXN-475 | CGCCCTCCAGCGCTGTT | FXN | 5′ and 3′ | human | dCs; lnaGs; |
| | m02 | TTTGAGGCTTGTTGCTTT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dGs; lnaAs; |
| | | | | | | dGs; lnaGs; |
| | | | | | | dCs; lnaTs; |
| | | | | | | dTs; lnaGs; |
| | | | | | | dTs; lnaTs; |
| | | | | | | dGs; lnaCs; |
| | | | | | | dTs; lnaTs; |
| | | | | | | dT-Sup |
| 158 | FXN-476 | CGCTCCGCCCTCCAGTTT | FXN | 5′ and 3′ | human | dCs; lnaGs; |
| | m02 | TTGAGGCTTGTTGCTTT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dGs; lnaAs; |
| | | | | | | dGs; lnaGs; |
| | | | | | | dCs; lnaTs; |
| | | | | | | dTs; lnaGs; |
| | | | | | | dTs; lnaTs; |
| | | | | | | dGs; lnaCs; |
| | | | | | | dTs; lnaTs; |
| | | | | | | dT-Sup |
| 159 | FXN-477 | TGACCCAAGGGAGACTT | FXN | 5′ and 3′ | human | dTs; lnaGs; |
| | m02 | TTTCATGTATGATGTTAT | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dCs; lnaAs; |
| | | | | | | dTs; lnaGs; |
| | | | | | | dTs; lnaAs; |
| | | | | | | dTs; lnaGs; |
| | | | | | | dAs; lnaTs; |
| | | | | | | dGs; lnaTs; |
| | | | | | | dTs; lnaAs; |
| | | | | | | dT-Sup |
| 160 | FXN-478 | TGGCCACTGGCCGCATT | FXN | 5′ and 3′ | human | dTs; lnaGs; |
| | m02 | TTTCATGTATGATGTTAT | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dCs; lnaAs; |
| | | | | | | dTs; lnaGs; |
| | | | | | | dTs; lnaAs; |
| | | | | | | dTs; lnaGs; |
| | | | | | | dAs; lnaTs; |
| | | | | | | dGs; lnaTs; |
| | | | | | | dTs; lnaAs; |
| | | | | | | dT-Sup |
| 161 | FXN-479 | CGGCGACCCCTGGTGTT | FXN | 5′ and 3′ | human | dCs; lnaGs; |
| | m02 | TTTCATGTATGATGTTAT | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaGs; |
| | | | | | | dTs; lnaAs; |
| | | | | | | dTs; lnaGs; |
| | | | | | | dAs; lnaTs; |
| | | | | | | dGs; lnaTs; |
| | | | | | | dTs; lnaAs; |
| | | | | | | dT-Sup |
| 162 | FXN-480 | CGCCCTCCAGCGCTGTT | FXN | 5′ and 3′ | human | dCs; lnaGs; |
| | m02 | TTTCATGTATGATGTTAT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaGs; |
| | | | | | | dTs; lnaAs; |
| | | | | | | dTs; lnaGs; |
| | | | | | | dAs; lnaTs; |
| | | | | | | dGs; lnaTs; |
| | | | | | | dTs; lnaAs; |
| | | | | | | dT-Sup |
| 163 | FXN-481 | CGCTCCGCCCTCCAGTTT | FXN | 5′ and 3′ | human | dCs; lnaGs; |
| | m02 | TTCATGTATGATGTTAT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaGs; |
| | | | | | | dTs; lnaAs; |
| | | | | | | dTs; lnaGs; |
| | | | | | | dAs; lnaTs; |
| | | | | | | dGs; lnaTs; |
| | | | | | | dTs; lnaAs; |
| | | | | | | dT-Sup |
| 164 | FXN-482 | CGCCCTCCAGTTTTTGGT | FXN | 5′ and 3′ | human | dCs; lnaGs; |
| | m02 | TTTTAAG | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaGs; |
| | | | | | | dGs; lnaTs; |
| | | | | | | dTs; lnaTs; |
| | | | | | | dTs; lnaTs; |
| | | | | | | dAs; lnaAs; |
| | | | | | | dG- |
| | | | | | | Sup |
| 165 | FXN-483 | CGCCCTCCAGTTTTTGG | FXN | 5′ and 3′ | human | dCs; lnaGs; |
| | m02 | GGTCTTGG | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaGs; |
| | | | | | | dGs; lnaGs; |
| | | | | | | dGs; lnaTs; |
| | | | | | | dCs; lnaTs; |
| | | | | | | dTs; lnaGs; |
| | | | | | | dG-Sup |
| 166 | FXN-484 | CGCCCTCCAGTTTTTCAT | FXN | 5′ and 3′ | human | dCs; lnaGs; |
| | m02 | AATGAAG | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaCs; |
| | | | | | | dAs; lnaTs; |
| | | | | | | dAs; lnaAs; |
| | | | | | | dTs; lnaGs; |
| | | | | | | dAs; lnaAs; |
| | | | | | | dG-Sup |
| 167 | FXN-485 | CGCCCTCCAGTTTTTAG | FXN | 5′ and 3′ | human | dCs; lnaGs; |
| | m02 | GAGGCAAC | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaAs; |
| | | | | | | dGs; lnaGs; |
| | | | | | | dAs; lnaGs; |
| | | | | | | dGs; lnaCs; |
| | | | | | | dAs; lnaAs; |
| | | | | | | dC-Sup |
| 168 | FXN-486 | CGCCCTCCAGTTTTTATT | FXN | 5′ and 3′ | human | dCs; lnaGs; |
| | m02 | ATTTTGC | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaAs; |
| | | | | | | dTs; lnaTs; |
| | | | | | | dAs; lnaTs; |
| | | | | | | dTs; lnaTs; |
| | | | | | | dTs; lnaGs; |
| | | | | | | dC- |
| | | | | | | Sup |
| 169 | FXN-487 | CGCCCTCCAGTTTTTCAT | FXN | 5′ and 3′ | human | dCs; lnaGs; |
| | m02 | TTTCCCT | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaCs; |
| | | | | | | dAs; lnaTs; |
| | | | | | | dTs; lnaTs; |
| | | | | | | dTs; lnaCs; |
| | | | | | | dCs; lnaCs; |
| | | | | | | dT- |
| | | | | | | Sup |
| 170 | FXN-488 | CGCCCTCCAGTTTTTGTA | FXN | 5′ and 3′ | human | dCs; lnaGs; |
| | m02 | GGCTACC | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaGs; |
| | | | | | | dTs; lnaAs; |
| | | | | | | dGs; lnaGs; |
| | | | | | | dCs; lnaTs; |
| | | | | | | dAs; lnaCs; |
| | | | | | | dC- |
| | | | | | | Sup |
| 171 | FXN-489 | CGCCCTCCAGTTTTTGA | FXN | 5′ and 3′ | human | dCs; lnaGs; |
| | m02 | GGCTTGTT | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaGs; |
| | | | | | | dAs; lnaGs; |
| | | | | | | dGs; lnaCs; |
| | | | | | | dTs; lnaTs; |
| | | | | | | dGs; lnaTs; |
| | | | | | | dT- |
| | | | | | | Sup |
| 172 | FXN-490 | CGCCCTCCAGTTTTTCAT | FXN | 5′ and 3′ | human | dCs; lnaGs; |
| | m02 | GTATGAT | | | | dCs; lnaCs; |
| | | | | | | dCs; lnaTs; |
| | | | | | | dCs; lnaCs; |
| | | | | | | dAs; lnaGs; |
| | | | | | | dTs; lnaTs; |
| | | | | | | dTs; lnaTs; |
| | | | | | | dTs; lnaCs; |
| | | | | | | dAs; lnaTs; |
| | | | | | | dGs; lnaTs; |
| | | | | | | dAs; lnaTs; |
| | | | | | | dGs; lnaAs; |
| | | | | | | dT- |
| | | | | | | Sup |
| 173 | FXN-491 | TGACCCAAGGGAGACTT | FXN | 5′ and 3′ | human | dTs; lnaGs; |
| | m02 | TTTTTTTTTT | | | | dAs; lnaCs; |
| | | | | | | dCs; lnaCs; |
| | | | | | | dAs; lnaAs; |
| | | | | | | dGs; lnaGs; |
| | | | | | | dGs; lnaAs; |
| | | | | | | dGs; lnaAs; |
| | | | | | | dCs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dT-Sup |
| 174 | FXN-492 | TGGCCACTGGCCGCATT | FXN | 5′ and 3′ | human | dTs; lnaGs; |
| | m02 | TTTTTTTTTT | | | | dGs; lnaCs; |
| | | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dAs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dT-Sup |
| 175 | FXN-493 | CGGCGACCCCTGGTGTT | FXN | 5′ and 3′ | human | dCs; lnaGs; |
| | m02 | TTTTTTTTTT | | | | dGs; lnaCs; |
| | | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dGs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dT-Sup |
| 176 | FXN-494 | CGCCCTCCAGCGCTGTT | FXN | 5′ and 3′ | human | dCs; lnaGs; |
| | m02 | TTTTTTTTTT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dT-Sup |
| 177 | FXN-495 | CGCTCCGCCCTCCAGTTT | FXN | 5′ and 3′ | human | dCs; lnaGs; |
| | m02 | TTTTTTTTT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dT-Sup |
| 178 | FXN-496 | AAAATAAACAACAAC | FXN | UTR | human | dAs; lnaAs; |
| | m02 | | | | | dAs; lnaAs; |
| | | | | | | dTs; lnaAs; |
| | | | | | | dAs; lnaAs; |
| | | | | | | dCs; lnaAs; |
| | | | | | | dAs; lnaCs; |
| | | | | | | dAs; lnaAs; |
| | | | | | | dC-Sup |
| 179 | FXN-497 | AGGAATAAAAAAAATA | FXN | UTR | human | dAs; lnaGs; |
| | m02 | | | | | dGs; lnaAs; |
| | | | | | | dAs; lnaTs; |
| | | | | | | dAs; lnaAs; |
| | | | | | | dAs; lnaAs; |
| | | | | | | dAs; lnaAs; |
| | | | | | | dAs; lnaAs; |
| | | | | | | dTs; lnaA- |
| | | | | | | Sup |
| 180 | FXN-498 | TCAAAAGCAGGAATA | FXN | UTR | human | dTs; lnaCs; |
| | m02 | | | | | dAs; lnaAs; |
| | | | | | | dAs; lnaAs; |
| | | | | | | dGs; lnaCs; |
| | | | | | | dAs; lnaGs; |
| | | | | | | dGs; lnaAs; |
| | | | | | | dAs; lnaTs; |
| | | | | | | dA-Sup |
| 181 | FXN-499 | ACTGTCCTCAAAAGC | FXN | UTR | human | dAs; lnaCs; |
| | m02 | | | | | dTs; lnaGs; |
| | | | | | | dTs; lnaCs; |
| | | | | | | dCs; lnaTs; |
| | | | | | | dCs; lnaAs; |
| | | | | | | dAs; lnaAs; |
| | | | | | | dAs; lnaGs; |
| | | | | | | dC- |
| | | | | | | Sup |
| 182 | FXN-500 | AGCCCAACTGTCCTC | FXN | UTR | human | dAs; lnaGs; |
| | m02 | | | | | dCs; lnaCs; |
| | | | | | | dCs; lnaAs; |
| | | | | | | dAs; lnaCs; |
| | | | | | | dTs; lnaGs; |
| | | | | | | dTs; lnaCs; |
| | | | | | | dCs; lnaTs; |
| | | | | | | dC- |
| | | | | | | Sup |
| 183 | FXN-501 | TGACACATAGCCCAA | FXN | UTR | human | dTs; lnaGs; |
| | m02 | | | | | dAs; lnaCs; |
| | | | | | | dAs; lnaCs; |
| | | | | | | dAs; lnaTs; |
| | | | | | | dAs; lnaGs; |
| | | | | | | dCs; lnaCs; |
| | | | | | | dCs; lnaAs; |
| | | | | | | dA- |
| | | | | | | Sup |
| 184 | FXN-502 | GAGCTGTGACACATA | FXN | UTR | human | dGs; lnaAs; |
| | m02 | | | | | dGs; lnaCs; |
| | | | | | | dTs; lnaGs; |
| | | | | | | dTs; lnaGs; |
| | | | | | | dAs; lnaCs; |
| | | | | | | dAs; lnaCs; |
| | | | | | | dAs; lnaTs; |
| | | | | | | dA- |
| | | | | | | Sup |
| 185 | FXN-503 | TCTGGGCCTGGGCTG | FXN | UTR/internal | human | dTs; lnaCs; |
| | m02 | | | | | dTs; lnaGs; |
| | | | | | | dGs; lnaGs; |
| | | | | | | dCs; lnaCs; |
| | | | | | | dTs; lnaGs; |
| | | | | | | dGs; lnaGs; |
| | | | | | | dCs; lnaTs; |
| | | | | | | dG-Sup |
| 186 | FXN-504 | GGTGAGGGTCTGGGC | FXN | UTR/internal | human | dGs; lnaGs; |
| | m02 | | | | | dTs; lnaGs; |
| | | | | | | dAs; lnaGs; |
| | | | | | | dGs; lnaGs; |
| | | | | | | dTs; lnaCs; |
| | | | | | | dTs; lnaGs; |
| | | | | | | dGs; lnaGs; |
| | | | | | | dC-Sup |
| 187 | FXN-505 | GGGACCCGGGTGAGG | FXN | UTR/internal | human | dGs; lnaGs; |
| | m02 | | | | | dGs; lnaAs; |
| | | | | | | dCs; lnaCs; |
| | | | | | | dCs; lnaGs; |
| | | | | | | dGs; lnaGs; |
| | | | | | | dTs; lnaGs; |
| | | | | | | dAs; lnaGs; |
| | | | | | | dG-Sup |
| 188 | FXN-506 | CCGGCCGCGGGACCC | FXN | UTR/internal | human | dCs; lnaCs; |
| | m02 | | | | | dGs; lnaGs; |
| | | | | | | dCs; lnaCs; |
| | | | | | | dGs; lnaCs; |
| | | | | | | dGs; lnaGs; |
| | | | | | | dGs; lnaAs; |
| | | | | | | dCs; lnaCs; |
| | | | | | | dC-Sup |
| 189 | FXN-507 | CAACTCTGCCGGCCG | FXN | UTR/internal | human | dCs; lnaAs; |
| | m02 | | | | | dAs; lnaCs; |
| | | | | | | dTs; lnaCs; |
| | | | | | | dTs; lnaGs; |
| | | | | | | dCs; lnaCs; |
| | | | | | | dGs; lnaGs; |
| | | | | | | dCs; lnaCs; |
| | | | | | | dG- |
| | | | | | | Sup |
| 190 | FXN-508 | AGTGGGGCCAACTCT | FXN | UTR/internal | human | dAs; lnaGs; |
| | m02 | | | | | dTs; lnaGs; |
| | | | | | | dGs; lnaGs; |
| | | | | | | dGs; lnaCs; |
| | | | | | | dCs; lnaAs; |
| | | | | | | dAs; lnaCs; |
| | | | | | | dTs; lnaCs; |
| | | | | | | dT- |
| | | | | | | Sup |
| 191 | FXN-509 | GGCCGCAGAGTGGGG | FXN | UTR/internal | human | dGs; lnaGs; |
| | m02 | | | | | dCs; lnaCs; |
| | | | | | | dGs; lnaCs; |
| | | | | | | dAs; lnaGs; |
| | | | | | | dAs; lnaGs; |
| | | | | | | dTs; lnaGs; |
| | | | | | | dGs; lnaGs; |
| | | | | | | dG-Sup |
| 192 | FXN-510 | GCCACGGCGGCCGCA | FXN | UTR/internal | human | dGs; lnaCs; |
| | m02 | | | | | dCs; lnaAs; |
| | | | | | | dCs; lnaGs; |
| | | | | | | dGs; lnaCs; |
| | | | | | | dGs; lnaGs; |
| | | | | | | dCs; lnaCs; |
| | | | | | | dGs; lnaCs; |
| | | | | | | dA-Sup |
| 193 | FXN-511 | GTGCGCAGGCCACGG | FXN | UTR/internal | human | dGs; lnaTs; |
| | m02 | | | | | dGs; lnaCs; |
| | | | | | | dGs; lnaCs; |
| | | | | | | dAs; lnaGs; |
| | | | | | | dGs; lnaCs; |
| | | | | | | dCs; lnaAs; |
| | | | | | | dCs; lnaGs; |
| | | | | | | dG- |
| | | | | | | Sup |
| 194 | FXN-512 | GGGGGACGGGGCAGG | FXN | intron | human | dGs; lnaGs; |
| | m02 | | | | | dGs; lnaGs; |
| | | | | | | dGs; lnaAs; |
| | | | | | | dCs; lnaGs; |
| | | | | | | dGs; lnaGs; |
| | | | | | | dGs; lnaCs; |
| | | | | | | dAs; lnaGs; |
| | | | | | | dG-Sup |
| 195 | FXN-513 | GGGACGGGGCAGGTT | FXN | intron | human | dGs; lnaGs; |
| | m02 | | | | | dGs; lnaAs; |
| | | | | | | dCs; lnaGs; |
| | | | | | | dGs; lnaGs; |
| | | | | | | dGs; lnaCs; |
| | | | | | | dAs; lnaGs; |
| | | | | | | dGs; lnaTs; |
| | | | | | | dT-Sup |
| 196 | FXN-514 | GACGGGGCAGGTTGA | FXN | intron | human | dGs; lnaAs; |
| | m02 | | | | | dCs; lnaGs; |
| | | | | | | dGs; lnaGs; |
| | | | | | | dGs; lnaCs; |
| | | | | | | dAs; lnaGs; |
| | | | | | | dGs; lnaTs; |
| | | | | | | dTs; lnaGs; |
| | | | | | | dA-Sup |
| 197 | FXN-515 | CGGGGCAGGTTGAGA | FXN | intron | human | dCs; lnaGs; |
| | m02 | | | | | dGs; lnaGs; |
| | | | | | | dGs; lnaCs; |
| | | | | | | dAs; lnaGs; |
| | | | | | | dGs; lnaTs; |
| | | | | | | dTs; lnaGs; |
| | | | | | | dAs; lnaGs; |
| | | | | | | dA- |
| | | | | | | Sup |
| 198 | FXN-516 | GGGCAGGTTGAGACT | FXN | intron | human | dGs; lnaGs; |
| | m02 | | | | | dGs; lnaCs; |
| | | | | | | dAs; lnaGs; |
| | | | | | | dGs; lnaTs; |
| | | | | | | dTs; lnaGs; |
| | | | | | | dAs; lnaGs; |
| | | | | | | dAs; lnaCs; |
| | | | | | | dT-Sup |
| 199 | FXN-517 | GCAGGTTGAGACTGG | FXN | intron | human | dGs; lnaCs; |
| | m02 | | | | | dAs; lnaGs; |
| | | | | | | dGs; lnaTs; |
| | | | | | | dTs; lnaGs; |
| | | | | | | dAs; lnaGs; |
| | | | | | | dAs; lnaCs; |
| | | | | | | dTs; lnaGs; |
| | | | | | | dG-Sup |
| 200 | FXN-518 | AGGTTGAGACTGGGT | FXN | intron | human | dAs; lnaGs; |
| | m02 | | | | | dGs; lnaTs; |
| | | | | | | dTs; lnaGs; |
| | | | | | | dAs; lnaGs; |
| | | | | | | dAs; lnaCs; |
| | | | | | | dTs; lnaGs; |
| | | | | | | dGs; lnaGs; |
| | | | | | | dT-Sup |
| 201 | FXN-519 | GGAAAAATTCCAGGA | FXN | Antisense/ | human | dGs; lnaGs; |
| | m02 | | | UTR | | dAs; lnaAs; |
| | | | | | | dAs; lnaAs; |
| | | | | | | dAs; lnaTs; |
| | | | | | | dTs; lnaCs; |
| | | | | | | dCs; lnaAs; |
| | | | | | | dGs; lnaGs; |
| | | | | | | dA-Sup |
| 202 | FXN-520 | AATTCCAGGAGGGAA | FXN | Antisense/ | human | dAs; lnaAs; |
| | m02 | | | UTR | | dTs; lnaTs; |
| | | | | | | dCs; lnaCs; |
| | | | | | | dAs; lnaGs; |
| | | | | | | dGs; lnaAs; |
| | | | | | | dGs; lnaGs; |
| | | | | | | dGs; lnaAs; |
| | | | | | | dA-Sup |
| 203 | FXN-521 | GAGGGAAAATGAATT | FXN | Antisense/ | human | dGs; lnaAs; |
| | m02 | | | UTR | | dGs; lnaGs; |
| | | | | | | dGs; lnaAs; |
| | | | | | | dAs; lnaAs; |
| | | | | | | dAs; lnaTs; |
| | | | | | | dGs; lnaAs; |
| | | | | | | dAs; lnaTs; |
| | | | | | | dT-Sup |
| 204 | FXN-522 | GAAAATGAATTGTCTTC | FXN | Antisense/ | human | dGs; lnaAs; |
| | m02 | | | UTR | | dAs; lnaAs; |
| | | | | | | dAs; lnaTs; |
| | | | | | | dGs; lnaAs; |
| | | | | | | dAs; lnaTs; |
| | | | | | | dTs; lnaGs; |
| | | | | | | dTs; lnaCs; |
| | | | | | | dTs; lnaTs; |
| | | | | | | dC-Sup |
| 205 | FXN-512 | GGGGGACGGGGCAGG | FXN | intron | human | lnaGs; lnaGs; |
| | m08 | | | | | lnaGs; dGs; |
| | | | | | | dGs; dAs; |
| | | | | | | dCs; dGs; |
| | | | | | | dGs; dGs; |
| | | | | | | dGs; dCs; |
| | | | | | | lnaAs; lnaGs; |
| | | | | | | lnaG- |
| | | | | | | Sup |
| 206 | FXN-513 | GGGACGGGGCAGGTT | FXN | intron | human | lnaGs; lnaGs; |
| | m08 | | | | | lnaGs; dAs; |
| | | | | | | dCs; dGs; |
| | | | | | | dGs; dGs; |
| | | | | | | dGs; dCs; |
| | | | | | | dAs; dGs; |
| | | | | | | lnaGs; lnaTs; |
| | | | | | | lnaT- |
| | | | | | | Sup |
| 207 | FXN-514 | GACGGGGCAGGTTGA | FXN | intron | human | lnaGs; lnaAs; |
| | m08 | | | | | lnaCs; dGs; |
| | | | | | | dGs; dGs; |
| | | | | | | dGs; dCs; |
| | | | | | | dAs; dGs; |
| | | | | | | dGs; dTs; |
| | | | | | | lnaTs; lnaGs; |
| | | | | | | lnaA- |
| | | | | | | Sup |
| 208 | FXN-515 | CGGGGCAGGTTGAGA | FXN | intron | human | lnaCs; lnaGs; |
| | m08 | | | | | lnaGs; dGs; |
| | | | | | | dGs; dCs; |
| | | | | | | dAs; dGs; |
| | | | | | | dGs; dTs; |
| | | | | | | dTs; dGs; |
| | | | | | | lnaAs; lnaGs; |
| | | | | | | lnaA- |
| | | | | | | Sup |
| 209 | FXN-516 | GGGCAGGTTGAGACT | FXN | intron | human | lnaGs; lnaGs; |
| | m08 | | | | | lnaGs; dCs; |
| | | | | | | dAs; dGs; |
| | | | | | | dGs; dTs; |
| | | | | | | dTs; dGs; |
| | | | | | | dAs; dGs; |
| | | | | | | lnaAs; lnaCs; |
| | | | | | | lnaT- |
| | | | | | | Sup |
| 210 | FXN-517 | GCAGGTTGAGACTGG | FXN | intron | human | lnaGs; lnaCs; |
| | m08 | | | | | lnaAs; dGs; |
| | | | | | | dGs; dTs; |
| | | | | | | dTs; dGs; |
| | | | | | | dAs; dGs; |
| | | | | | | dAs; dCs; |
| | | | | | | lnaTs; lnaGs; |
| | | | | | | lnaG- |
| | | | | | | Sup |
| 211 | FXN-518 | AGGTTGAGACTGGGT | FXN | intron | human | lnaAs; lnaGs; |
| | m08 | | | | | lnaGs; dTs; |
| | | | | | | dTs; dGs; |
| | | | | | | dAs; dGs; |
| | | | | | | dAs; dCs; |
| | | | | | | dTs; dGs; |
| | | | | | | lnaGs; lnaGs; |
| | | | | | | lnaT- |
| | | | | | | Sup |
| 212 | FXN-519 | GGAAAAATTCCAGGA | FXN | Antisense/ | human | lnaGs; lnaGs; |
| | m08 | | | UTR | | lnaAs; dAs; |
| | | | | | | dAs; dAs; |
| | | | | | | dAs; dTs; |
| | | | | | | dTs; dCs; |
| | | | | | | dCs; dAs; |
| | | | | | | lnaGs; lnaGs; |
| | | | | | | lnaA- |
| | | | | | | Sup |
| 213 | FXN-520 | AATTCCAGGAGGGAA | FXN | Antisense/ | human | lnaAs; lnaAs; |
| | m08 | | | UTR | | lnaTs; dTs; |
| | | | | | | dCs; dCs; |
| | | | | | | dAs; dGs; |
| | | | | | | dGs; dAs; |
| | | | | | | dGs; dGs; |
| | | | | | | lnaGs; lnaAs; |
| | | | | | | lnaA- |
| | | | | | | Sup |
| 214 | FXN-521 | GAGGGAAAATGAATT | FXN | Antisense/ | human | lnaGs; lnaAs; |
| | m08 | | | UTR | | lnaGs; dGs; |
| | | | | | | dGs; dAs; |
| | | | | | | dAs; dAs; |
| | | | | | | dAs; dTs; |
| | | | | | | dGs; dAs; |
| | | | | | | lnaAs; lnaTs; |
| | | | | | | lnaT- |
| | | | | | | Sup |
| 215 | FXN-522 | GAAAATGAATTGTCTTC | FXN | Antisense/ | human | lnaGs; lnaAs; |
| | m08 | | | UTR | | lnaAs; dAs; |
| | | | | | | dAs; dTs; |
| | | | | | | dGs; dAs; |
| | | | | | | dAs; dTs; |
| | | | | | | dTs; dGs; |
| | | | | | | dTs; dCs; |
| | | | | | | lnaTs; lnaTs; |
| | | | | | | lnaC-Sup |
| 216 | EPO-37 | GGTGGTTTCAGTTCT | EPO | 3′ | human | dGs; lnaGs; |
| | m02 | | | | | dTs; lnaGs; |
| | | | | | | dGs; lnaTs; |
| | | | | | | dTs; lnaTs; |
| | | | | | | dCs; lnaAs; |
| | | | | | | dGs; lnaTs; |
| | | | | | | dTs; lnaCs; |
| | | | | | | dT- |
| | | | | | | Sup |
| 217 | EPO-38 | TTTTTGGTGGTTTCAGTT | EPO | 3′ | human | dTs; lnaTs; |
| | m02 | CT | | | | dTs; lnaTs; |
| | | | | | | dTs; lnaGs; |
| | | | | | | dGs; lnaTs; |
| | | | | | | dGs; lnaGs; |
| | | | | | | dTs; lnaTs; |
| | | | | | | dTs; lnaCs; |
| | | | | | | dAs; lnaGs; |
| | | | | | | dTs; lnaTs; |
| | | | | | | dCs; lnaT- |
| | | | | | | Sup |
| 218 | EPO-39 | AGCGTGCTATCTGGG | EPO | 5′ | human | dAs; lnaGs; |
| | m02 | | | | | dCs; lnaGs; |
| | | | | | | dTs; lnaGs; |
| | | | | | | dCs; lnaTs; |
| | | | | | | dAs; lnaTs; |
| | | | | | | dCs; lnaTs; |
| | | | | | | dGs; lnaGs; |
| | | | | | | dG- |
| | | | | | | Sup |
| 219 | EPO-40 | TGGCCCAGGGACTCT | EPO | 5′ | human | dTs; lnaGs; |
| | m02 | | | | | dGs; lnaCs; |
| | | | | | | dCs; lnaCs; |
| | | | | | | dAs; lnaGs; |
| | | | | | | dGs; lnaGs; |
| | | | | | | dAs; lnaCs; |
| | | | | | | dTs; lnaCs; |
| | | | | | | dT- |
| | | | | | | Sup |
| 220 | EPO-41 | TCTGCGGCTCTGGC | EPO | 5′ | human | dTs; lnaCs; |
| | m02 | | | | | dTs; lnaGs; |
| | | | | | | dCs; lnaGs; |
| | | | | | | dGs; lnaCs; |
| | | | | | | dTs; lnaCs; |
| | | | | | | dTs; lnaGs; |
| | | | | | | dGs; lnaC- |
| | | | | | | Sup |
| 221 | EPO-42 | CGGTCCGGCTCTGGG | EPO | 5′ | human | dCs; lnaGs; |
| | m02 | | | | | dGs; lnaTs; |
| | | | | | | dCs; lnaCs; |
| | | | | | | dGs; lnaGs; |
| | | | | | | dCs; lnaTs; |
| | | | | | | dCs; lnaTs; |
| | | | | | | dGs; lnaGs; |
| | | | | | | dG- |
| | | | | | | Sup |
| 222 | EPO-43 | TCATCCCGGGAAGCT | EPO | 5′ | human | dTs; lnaCs; |
| | m02 | | | | | dAs; lnaTs; |
| | | | | | | dCs; lnaCs; |
| | | | | | | dCs; lnaGs; |
| | | | | | | dGs; lnaGs; |
| | | | | | | dAs; lnaAs; |
| | | | | | | dGs; lnaCs; |
| | | | | | | dT-Sup |
| 223 | EPO-44 | CCCCAAGTCCCCGCT | EPO | 5′ | human | dCs; lnaCs; |
| | m02 | | | | | dCs; lnaCs; |
| | | | | | | dAs; lnaAs; |
| | | | | | | dGs; lnaTs; |
| | | | | | | dCs; lnaCs; |
| | | | | | | dCs; lnaCs; |
| | | | | | | dGs; lnaCs; |
| | | | | | | dT- |
| | | | | | | Sup |
| 224 | EPO-45 | CCAACCATGCAAGCA | EPO | 5′ | human | dCs; lnaCs; |
| | m02 | | | | | dAs; lnaAs; |
| | | | | | | dCs; lnaCs; |
| | | | | | | dAs; lnaTs; |
| | | | | | | dGs; lnaCs; |
| | | | | | | dAs; lnaAs; |
| | | | | | | dGs; lnaCs; |
| | | | | | | dA- |
| | | | | | | Sup |
| 225 | EPO-46 | TGGCCCAGGGACTCTTC | EPO | 5′ | human | dTs; lnaGs; |
| | m02 | | | | | dGs; lnaCs; |
| | | | | | | dCs; lnaCs; |
| | | | | | | dAs; lnaGs; |
| | | | | | | dGs; lnaGs; |
| | | | | | | dAs; lnaCs; |
| | | | | | | dTs; lnaCs; |
| | | | | | | dTs; lnaTs; |
| | | | | | | dC-Sup |
| 226 | EPO-47 | CGGTCCGGCTCTGGGTTC | EPO | 5′ | human | dCs; lnaGs; |
| | m02 | | | | | dGs; lnaTs; |
| | | | | | | dCs; lnaCs; |
| | | | | | | dGs; lnaGs; |
| | | | | | | dCs; lnaTs; |
| | | | | | | dCs; lnaTs; |
| | | | | | | dGs; lnaGs; |
| | | | | | | dGs; lnaTs; |
| | | | | | | dTs; lnaC-Sup |
| 227 | EPO-48 | CCAACCATGCAAGCACC | EPO | 5′ | human | dCs; lnaCs; |
| | m02 | | | | | dAs; lnaAs; |
| | | | | | | dCs; lnaCs; |
| | | | | | | dAs; lnaTs; |
| | | | | | | dGs; lnaCs; |
| | | | | | | dAs; lnaAs; |
| | | | | | | dGs; lnaCs; |
| | | | | | | dAs; lnaCs; |
| | | | | | | dC-Sup |
| 228 | EPO-49 | TGGCCCAGGGACTCTCA | EPO | 5′ | human | dTs; lnaGs; |
| | m02 | CAAAGTGAC | | | | dGs; lnaCs; |
| | | | | | | dCs; lnaCs; |
| | | | | | | dAs; lnaGs; |
| | | | | | | dGs; lnaGs; |
| | | | | | | dAs; lnaCs; |
| | | | | | | dTs; lnaCs; |
| | | | | | | dTs; lnaCs; |
| | | | | | | dAs; dCs; |
| | | | | | | dAs; dAs; |
| | | | | | | dAs; dGs; |
| | | | | | | dTs; lnaGs; |
| | | | | | | dAs; lnaC- |
| | | | | | | Sup |
| 229 | EPO-50 | CGGTCCGGCTCTGGGAA | EPO | 5′ | human | dCs; lnaGs; |
| | m02 | GAAACTTTC | | | | dGs; lnaTs; |
| | | | | | | dCs; lnaCs; |
| | | | | | | dGs; lnaGs; |
| | | | | | | dCs; lnaTs; |
| | | | | | | dCs; lnaTs; |
| | | | | | | dGs; lnaGs; |
| | | | | | | dGs; lnaAs; |
| | | | | | | dAs; dGs; |
| | | | | | | dAs; dAs; |
| | | | | | | dAs; dCs; |
| | | | | | | dTs; lnaTs; |
| | | | | | | dTs; lnaC- |
| | | | | | | Sup |
| 230 | EPO-51 | CCAACCATGCAAGCACT | EPO | 5′ | human | dCs; lnaCs; |
| | m02 | CAAAGAGTC | | | | dAs; lnaAs; |
| | | | | | | dCs; lnaCs; |
| | | | | | | dAs; lnaTs; |
| | | | | | | dGs; lnaCs; |
| | | | | | | dAs; lnaAs; |
| | | | | | | dGs; lnaCs; |
| | | | | | | dAs; lnaCs; |
| | | | | | | dTs; dCs; |
| | | | | | | dAs; dAs; |
| | | | | | | dAs; dGs; |
| | | | | | | dAs; lnaGs; |
| | | | | | | dTs; lnaC- |
| | | | | | | Sup |
| 231 | EPO-52 | TGGCCCAGGGACTCTTT | EPO | 5′ and 3′ | human | dTs; lnaGs; |
| | m02 | TTGGTGGTTTCAGTTCT | | | | dGs; lnaCs; |
| | | | | | | dCs; lnaCs; |
| | | | | | | dAs; lnaGs; |
| | | | | | | dGs; lnaGs; |
| | | | | | | dAs; lnaCs; |
| | | | | | | dTs; lnaCs; |
| | | | | | | dTs; lnaTs; |
| | | | | | | dTs; lnaTs; |
| | | | | | | dTs; lnaGs; |
| | | | | | | dGs; lnaTs; |
| | | | | | | dGs; lnaGs; |
| | | | | | | dTs; lnaTs; |
| | | | | | | dTs; lnaCs; |
| | | | | | | dAs; lnaGs; |
| | | | | | | dTs; lnaTs; |
| | | | | | | dCs; lnaT- |
| | | | | | | Sup |
| 232 | EPO-53 | CGGTCCGGCTCTGGGTT | EPO | 5′ and 3′ | human | dCs; lnaGs; |
| | m02 | TTTGGTGGTTTCAGTTCT | | | | dGs; lnaTs; |
| | | | | | | dCs; lnaCs; |
| | | | | | | dGs; lnaGs; |
| | | | | | | dCs; lnaTs; |
| | | | | | | dCs; lnaTs; |
| | | | | | | dGs; lnaGs; |
| | | | | | | dGs; lnaTs; |
| | | | | | | dTs; lnaTs; |
| | | | | | | dTs; lnaTs; |
| | | | | | | dGs; lnaGs; |
| | | | | | | dTs; lnaGs; |
| | | | | | | dGs; lnaTs; |
| | | | | | | dTs; lnaTs; |
| | | | | | | dCs; lnaAs; |
| | | | | | | dGs; lnaTs; |
| | | | | | | dTs; lnaCs; |
| | | | | | | dT-Sup |
| 233 | EPO-54 | CCAACCATGCAAGCATT | EPO | 5′ and 3′ | human | dCs; lnaCs; |
| | m02 | TTTGGTGGTTTCAGTTCT | | | | dAs; lnaAs; |
| | | | | | | dCs; lnaCs; |
| | | | | | | dAs; lnaTs; |
| | | | | | | dGs; lnaCs; |
| | | | | | | dAs; lnaAs; |
| | | | | | | dGs; lnaCs; |
| | | | | | | dAs; lnaTs; |
| | | | | | | dTs; lnaTs; |
| | | | | | | dTs; lnaTs; |
| | | | | | | dGs; lnaGs; |
| | | | | | | dTs; lnaGs; |
| | | | | | | dGs; lnaTs; |
| | | | | | | dTs; lnaTs; |
| | | | | | | dCs; lnaAs; |
| | | | | | | dGs; lnaTs; |
| | | | | | | dTs; lnaCs; |
| | | | | | | dT-Sup |
| 234 | EPO-55 | CAGGGACTCTTTTTGGT | EPO | 5′ and 3′ | human | dCs; lnaAs; |
| | m02 | GGTTTCA | | | | dGs; lnaGs; |
| | | | | | | dGs; lnaAs; |
| | | | | | | dCs; lnaTs; |
| | | | | | | dCs; lnaTs; |
| | | | | | | dTs; lnaTs; |
| | | | | | | dTs; lnaTs; |
| | | | | | | dGs; lnaGs; |
| | | | | | | dTs; lnaGs; |
| | | | | | | dGs; lnaTs; |
| | | | | | | dTs; lnaTs; |
| | | | | | | dCs; lnaA- |
| | | | | | | Sup |
| 235 | EPO-56 | CGGCTCTGGGTTTTTGG | EPO | 5′ and 3′ | human | dCs; lnaGs; |
| | m02 | TGGTTTCA | | | | dGs; lnaCs; |
| | | | | | | dTs; lnaCs; |
| | | | | | | dTs; lnaGs; |
| | | | | | | dGs; lnaGs; |
| | | | | | | dTs; lnaTs; |
| | | | | | | dTs; lnaTs; |
| | | | | | | dTs; lnaGs; |
| | | | | | | dGs; lnaTs; |
| | | | | | | dGs; lnaGs; |
| | | | | | | dTs; lnaTs; |
| | | | | | | dTs; lnaCs; |
| | | | | | | dA-Sup |
| 236 | EPO-57 | CATGCAAGCATTTTTGG | EPO | 5′ and 3′ | human | dCs; lnaAs; |
| | m02 | TGGTTTCA | | | | dTs; lnaGs; |
| | | | | | | dCs; lnaAs; |
| | | | | | | dAs; lnaGs; |
| | | | | | | dCs; lnaAs; |
| | | | | | | dTs; lnaTs; |
| | | | | | | dTs; lnaTs; |
| | | | | | | dTs; lnaGs; |
| | | | | | | dGs; lnaTs; |
| | | | | | | dGs; lnaGs; |
| | | | | | | dTs; lnaTs; |
| | | | | | | dTs; lnaCs; |
| | | | | | | dA-Sup |
| 237 | EPO-58 | TGGCCCAGGGACTCGGT | EPO | 5′ and 3′ | human | dTs; lnaGs; |
| | m02 | GGTTTCAGTTCT | | | | dGs; lnaCs; |
| | | | | | | dCs; lnaCs; |
| | | | | | | dAs; lnaGs; |
| | | | | | | dGs; lnaGs; |
| | | | | | | dAs; lnaCs; |
| | | | | | | dTs; lnaCs; |
| | | | | | | dGs; lnaGs; |
| | | | | | | dTs; lnaGs; |
| | | | | | | dGs; lnaTs; |
| | | | | | | dTs; lnaTs; |
| | | | | | | dCs; lnaAs; |
| | | | | | | dGs; lnaTs; |
| | | | | | | dTs; lnaCs; |
| | | | | | | dT- |
| | | | | | | Sup |
| 238 | EPO-59 | CGGTCCGGCTCTGGTGG | EPO | 5′ and 3′ | human | dCs; lnaGs; |
| | m02 | TGGTTTCAGTTCT | | | | dGs; lnaTs; |
| | | | | | | dCs; lnaCs; |
| | | | | | | dGs; lnaGs; |
| | | | | | | dCs; lnaTs; |
| | | | | | | dCs; lnaTs; |
| | | | | | | dGs; lnaGs; |
| | | | | | | dTs; lnaGs; |
| | | | | | | dGs; lnaTs; |
| | | | | | | dGs; lnaGs; |
| | | | | | | dTs; lnaTs; |
| | | | | | | dTs; lnaCs; |
| | | | | | | dAs; lnaGs; |
| | | | | | | dTs; lnaTs; |
| | | | | | | dCs; lnaT- |
| | | | | | | Sup |
| 239 | EPO-60 | CCAACCATGCAAGCAGG | EPO | 5′ and 3′ | human | dCs; lnaCs; |
| | m02 | TGGTTTCAGTTCT | | | | dAs; lnaAs; |
| | | | | | | dCs; lnaCs; |
| | | | | | | dAs; lnaTs; |
| | | | | | | dGs; lnaCs; |
| | | | | | | dAs; lnaAs; |
| | | | | | | dGs; lnaCs; |
| | | | | | | dAs; lnaGs; |
| | | | | | | dGs; lnaTs; |
| | | | | | | dGs; lnaGs; |
| | | | | | | dTs; lnaTs; |
| | | | | | | dTs; lnaCs; |
| | | | | | | dAs; lnaGs; |
| | | | | | | dTs; lnaTs; |
| | | | | | | dCs; lnaT- |
| | | | | | | Sup |
| 240 | KLF4-31 | TTTTTAGATAAAATATTA | KLF4 | 3′ | human | dTs; lnaTs; |
| | m02 | TA | | | | dTs; lnaTs; |
| | | | | | | dTs; lnaAs; |
| | | | | | | dGs; lnaAs; |
| | | | | | | dTs; lnaAs; |
| | | | | | | dAs; lnaAs; |
| | | | | | | dAs; lnaTs; |
| | | | | | | dAs; lnaTs; |
| | | | | | | dTs; lnaAs; |
| | | | | | | dTs; lnaA- |
| | | | | | | Sup |
| 241 | KLF4-32 | TTTTTATTCAGATAAAATA | KLF4 | 3′ | human | dTs; lnaTs; |
| | m02 | | | | | dTs; lnaTs; |
| | | | | | | dTs; lnaAs; |
| | | | | | | dTs; lnaTs; |
| | | | | | | dCs; lnaAs; |
| | | | | | | dGs; lnaAs; |
| | | | | | | dTs; lnaAs; |
| | | | | | | dAs; lnaAs; |
| | | | | | | dAs; lnaTs; |
| | | | | | | dA- |
| | | | | | | Sup |
| 242 | KLF4-33 | TTTTTGGTTTATTTAAAA | KLF4 | 3′ | human | dTs; lnaTs; |
| | m02 | CT | | | | dTs; lnaTs; |
| | | | | | | dTs; lnaGs; |
| | | | | | | dGs; lnaTs; |
| | | | | | | dTs; lnaTs; |
| | | | | | | dAs; lnaTs; |
| | | | | | | dTs; lnaTs; |
| | | | | | | dAs; lnaAs; |
| | | | | | | dAs; lnaAs; |
| | | | | | | dCs; lnaT- |
| | | | | | | Sup |
| 243 | KLF4-34 | TTTTTAAATTTATATTAC | KLF4 | 3′ | human | dTs; lnaTs; |
| | m02 | AT | | | | dTs; lnaTs; |
| | | | | | | dTs; lnaAs; |
| | | | | | | dAs; lnaAs; |
| | | | | | | dTs; lnaTs; |
| | | | | | | dTs; lnaAs; |
| | | | | | | dTs; lnaAs; |
| | | | | | | dTs; lnaTs; |
| | | | | | | dAs; lnaCs; |
| | | | | | | dAs; lnaT- |
| | | | | | | Sup |
| 244 | KLF4-35 | TTTTTCTTAAATTTATAT | KLF4 | 3′ | human | dTs; lnaTs; |
| | m02 | TA | | | | dTs; lnaTs; |
| | | | | | | dTs; lnaCs; |
| | | | | | | dTs; lnaTs; |
| | | | | | | dAs; lnaAs; |
| | | | | | | dAs; lnaTs; |
| | | | | | | dTs; lnaTs; |
| | | | | | | dAs; lnaTs; |
| | | | | | | dAs; lnaTs; |
| | | | | | | dTs; lnaA- |
| | | | | | | Sup |
| 245 | KLF4-36 | TTTTTCACAAAATGTTCA | KLF4 | 3′ | human | dTs; lnaTs; |
| | m02 | TT | | | | dTs; lnaTs; |
| | | | | | | dTs; lnaCs; |
| | | | | | | dAs; lnaCs; |
| | | | | | | dAs; lnaAs; |
| | | | | | | dAs; lnaAs; |
| | | | | | | dTs; lnaGs; |
| | | | | | | dTs; lnaTs; |
| | | | | | | dCs; lnaAs; |
| | | | | | | dTs; lnaT- |
| | | | | | | Sup |
| 246 | KLF4-37 | CCTCCGCCTTCTCCC | KLF4 | 5′ | human | dCs; lnaCs; |
| | m02 | | | | | dTs; lnaCs; |
| | | | | | | dCs; lnaGs; |
| | | | | | | dCs; lnaCs; |
| | | | | | | dTs; lnaTs; |
| | | | | | | dCs; lnaTs; |
| | | | | | | dCs; lnaCs; |
| | | | | | | dC- |
| | | | | | | Sup |
| 247 | KLF4-38 | TCTGGTCGGGAAACT | KLF4 | 5′ | human | dTs; lnaCs; |
| | m02 | | | | | dTs; lnaGs; |
| | | | | | | dGs; lnaTs; |
| | | | | | | dCs; lnaGs; |
| | | | | | | dGs; lnaGs; |
| | | | | | | dAs; lnaAs; |
| | | | | | | dAs; lnaCs; |
| | | | | | | dT-Sup |
| 248 | KLF4-39 | GCTACAGCCTTTTCC | KLF4 | 5′ | human | dGs; lnaCs; |
| | m02 | | | | | dTs; lnaAs; |
| | | | | | | dCs; lnaAs; |
| | | | | | | dGs; lnaCs; |
| | | | | | | dCs; lnaTs; |
| | | | | | | dTs; lnaTs; |
| | | | | | | dTs; lnaCs; |
| | | | | | | dC- |
| | | | | | | Sup |
| 249 | KLF4-40 | CCTCCGCCTTCTCCCC | KLF4 | 5′ | human | dCs; lnaCs; |
| | m02 | | | | | dTs; lnaCs; |
| | | | | | | dCs; lnaGs; |
| | | | | | | dCs; lnaCs; |
| | | | | | | dTs; lnaTs; |
| | | | | | | dCs; lnaTs; |
| | | | | | | dCs; lnaCs; |
| | | | | | | dCs; lnaC- |
| | | | | | | Sup |
| 250 | KLF4-41 | TCTGGTCGGGAAACTCC | KLF4 | 5′ | human | dTs; lnaCs; |
| | m02 | | | | | dTs; lnaGs; |
| | | | | | | dGs; lnaTs; |
| | | | | | | dCs; lnaGs; |
| | | | | | | dGs; lnaGs; |
| | | | | | | dAs; lnaAs; |
| | | | | | | dAs; lnaCs; |
| | | | | | | dTs; lnaCs; |
| | | | | | | dC-Sup |
| 251 | KLF4-42 | GCTACAGCCTTTTCCC | KLF4 | 5′ | human | dGs; lnaCs; |
| | m02 | | | | | dTs; lnaAs; |
| | | | | | | dCs; lnaAs; |
| | | | | | | dGs; lnaCs; |
| | | | | | | dCs; lnaTs; |
| | | | | | | dTs; lnaTs; |
| | | | | | | dTs; lnaCs; |
| | | | | | | dCs; lnaC- |
| | | | | | | Sup |
| 252 | KLF4-43 | CCTCCGCCTTCTCCCTCT | KLF4 | 5′ | human | dCs; lnaCs; |
| | m02 | TTGATC | | | | dTs; lnaCs; |
| | | | | | | dCs; lnaGs; |
| | | | | | | dCs; lnaCs; |
| | | | | | | dTs; lnaTs; |
| | | | | | | dCs; lnaTs; |
| | | | | | | dCs; lnaCs; |
| | | | | | | dCs; lnaTs; |
| | | | | | | dCs; dTs; |
| | | | | | | dTs; dTs; |
| | | | | | | dGs; lnaAs; |
| | | | | | | dTs; lnaC-Sup |
| 253 | KLF4-44 | TCTGGTCGGGAAACTCA | KLF4 | 5′ | human | dTs; lnaCs; |
| | m02 | ATTATTGTC | | | | dTs; lnaGs; |
| | | | | | | dGs; lnaTs; |
| | | | | | | dCs; lnaGs; |
| | | | | | | dGs; lnaGs; |
| | | | | | | dAs; lnaAs; |
| | | | | | | dAs; lnaCs; |
| | | | | | | dTs; lnaCs; |
| | | | | | | dAs; dAs; |
| | | | | | | dTs; dTs; |
| | | | | | | dAs; dTs; |
| | | | | | | dTs; lnaGs; |
| | | | | | | dTs; lnaC- |
| | | | | | | Sup |
| 254 | KLF4-45 | GCTACAGCCTTTTCCACT | KLF4 | 5′ | human | dGs; lnaCs; |
| | m02 | TTGTTC | | | | dTs; lnaAs; |
| | | | | | | dCs; lnaAs; |
| | | | | | | dGs; lnaCs; |
| | | | | | | dCs; lnaTs; |
| | | | | | | dTs; lnaTs; |
| | | | | | | dTs; lnaCs; |
| | | | | | | dCs; lnaAs; |
| | | | | | | dCs; dTs; |
| | | | | | | dTs; dTs; |
| | | | | | | dGs; lnaTs; |
| | | | | | | dTs; lnaC-Sup |
| 255 | KLF4-46 | CCTCCGCCTTCTCCCTTT | KLF4 | 5′ and 3′ | human | dCs; lnaCs; |
| | m02 | TTAGATAAAATATTATA | | | | dTs; lnaCs; |
| | | | | | | dCs; lnaGs; |
| | | | | | | dCs; lnaCs; |
| | | | | | | dTs; lnaTs; |
| | | | | | | dCs; lnaTs; |
| | | | | | | dCs; lnaCs; |
| | | | | | | dCs; lnaTs; |
| | | | | | | dTs; lnaTs; |
| | | | | | | dTs; lnaTs; |
| | | | | | | dAs; lnaGs; |
| | | | | | | dAs; lnaTs; |
| | | | | | | dAs; lnaAs; |
| | | | | | | dAs; lnaAs; |
| | | | | | | dTs; lnaAs; |
| | | | | | | dTs; lnaTs; |
| | | | | | | dAs; lnaTs; |
| | | | | | | dA-Sup |
| 256 | KLF4-47 | TCTGGTCGGGAAACTTT | KLF4 | 5′ and 3′ | human | dTs; lnaCs; |
| | m02 | TTAGATAAAATATTATA | | | | dTs; lnaGs; |
| | | | | | | dGs; lnaTs; |
| | | | | | | dCs; lnaGs; |
| | | | | | | dGs; lnaGs; |
| | | | | | | dAs; lnaAs; |
| | | | | | | dAs; lnaCs; |
| | | | | | | dTs; lnaTs; |
| | | | | | | dTs; lnaTs; |
| | | | | | | dTs; lnaAs; |
| | | | | | | dGs; lnaAs; |
| | | | | | | dTs; lnaAs; |
| | | | | | | dAs; lnaAs; |
| | | | | | | dAs; lnaTs; |
| | | | | | | dAs; lnaTs; |
| | | | | | | dTs; lnaAs; |
| | | | | | | dTs; lnaA- |
| | | | | | | Sup |
| 257 | KLF4-48 | GCTACAGCCTTTTCCTTT | KLF4 | 5′ and 3′ | human | dGs; lnaCs; |
| | m02 | TTAGATAAAATATTATA | | | | dTs; lnaAs; |
| | | | | | | dCs; lnaAs; |
| | | | | | | dGs; lnaCs; |
| | | | | | | dCs; lnaTs; |
| | | | | | | dTs; lnaTs; |
| | | | | | | dTs; lnaCs; |
| | | | | | | dCs; lnaTs; |
| | | | | | | dTs; lnaTs; |
| | | | | | | dTs; lnaTs; |
| | | | | | | dAs; lnaGs; |
| | | | | | | dAs; lnaTs; |
| | | | | | | dAs; lnaAs; |
| | | | | | | dAs; lnaAs; |
| | | | | | | dTs; lnaAs; |
| | | | | | | dTs; lnaTs; |
| | | | | | | dAs; lnaTs; |
| | | | | | | dA-Sup |
| 258 | KLF4-49 | CCTCCGCCTTCTCCCTTT | KLF4 | 5′ and 3′ | human | dCs; lnaCs; |
| | m02 | TTGGTTTATTTAAAACT | | | | dTs; lnaCs; |
| | | | | | | dCs; lnaGs; |
| | | | | | | dCs; lnaCs; |
| | | | | | | dTs; lnaTs; |
| | | | | | | dCs; lnaTs; |
| | | | | | | dCs; lnaCs; |
| | | | | | | dCs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaAs; |
| | | | | | | dTs; lnaTs; |
| | | | | | | dTs; lnaAs; |
| | | | | | | dAs; lnaAs; |
| | | | | | | dAs; lnaCs; |
| | | | | | | dT-Sup |
| 259 | KLF4-50 | TCTGGTCGGGAAACTTT | KLF4 | 5′ and 3′ | human | dTs; lnaCs; |
| | m02 | TTGGTTTATTTAAAACT | | | | dTs; lnaGs; |
| | | | | | | dGs; lnaTs; |
| | | | | | | dCs; lnaGs; |
| | | | | | | dGs; lnaGs; |
| | | | | | | dAs; lnaAs; |
| | | | | | | dAs; lnaCs; |
| | | | | | | dTs; lnaTs; |
| | | | | | | dTs; lnaTs; |
| | | | | | | dTs; lnaGs; |
| | | | | | | dGs; lnaTs; |
| | | | | | | dTs; lnaTs; |
| | | | | | | dAs; lnaTs; |
| | | | | | | dTs; lnaTs; |
| | | | | | | dAs; lnaAs; |
| | | | | | | dAs; lnaAs; |
| | | | | | | dCs; lnaT- |
| | | | | | | Sup |
| 260 | KLF4-51 | GCTACAGCCTTTTCCTTT | KLF4 | 5′ and 3′ | human | dGs; lnaCs; |
| | m02 | TTGGTTTATTTAAAACT | | | | dTs; lnaAs; |
| | | | | | | dCs; lnaAs; |
| | | | | | | dGs; lnaCs; |
| | | | | | | dCs; lnaTs; |
| | | | | | | dTs; lnaTs; |
| | | | | | | dTs; lnaCs; |
| | | | | | | dCs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaAs; |
| | | | | | | dTs; lnaTs; |
| | | | | | | dTs; lnaAs; |
| | | | | | | dAs; lnaAs; |
| | | | | | | dAs; lnaCs; |
| | | | | | | dT-Sup |
| 261 | KLF4-52 | CCTCCGCCTTCTCCCTTT | KLF4 | 5′ and 3′ | human | dCs; lnaCs; |
| | m02 | TTAAATTTATATTACAT | | | | dTs; lnaCs; |
| | | | | | | dCs; lnaGs; |
| | | | | | | dCs; lnaCs; |
| | | | | | | dTs; lnaTs; |
| | | | | | | dCs; lnaTs; |
| | | | | | | dCs; lnaCs; |
| | | | | | | dCs; lnaTs; |
| | | | | | | dTs; lnaTs; |
| | | | | | | dTs; lnaTs; |
| | | | | | | dAs; lnaAs; |
| | | | | | | dAs; lnaTs; |
| | | | | | | dTs; lnaTs; |
| | | | | | | dAs; lnaTs; |
| | | | | | | dAs; lnaTs; |
| | | | | | | dTs; lnaAs; |
| | | | | | | dCs; lnaAs; |
| | | | | | | dT |
| 262 | KLF4-53 | TCTGGTCGGGAAACTTT | KLF4 | 5′ and 3′ | human | dTs; lnaCs; |
| | m02 | TTAAATTTATATTACAT | | | | dTs; lnaGs; |
| | | | | | | dGs; lnaTs; |
| | | | | | | dCs; lnaGs; |
| | | | | | | dGs; lnaGs; |
| | | | | | | dAs; lnaAs; |
| | | | | | | dAs; lnaCs; |
| | | | | | | dTs; lnaTs; |
| | | | | | | dTs; lnaTs; |
| | | | | | | dTs; lnaAs; |
| | | | | | | dAs; lnaAs; |
| | | | | | | dTs; lnaTs; |
| | | | | | | dTs; lnaAs; |
| | | | | | | dTs; lnaAs; |
| | | | | | | dTs; lnaTs; |
| | | | | | | dAs; lnaCs; |
| | | | | | | dAs; lnaT- |
| | | | | | | Sup |
| 263 | KLF4-54 | GCTACAGCCTTTTCCTTT | KLF4 | 5′ and 3′ | human | dGs; lnaCs; |
| | m02 | TTAAATTTATATTACAT | | | | dTs; lnaAs; |
| | | | | | | dCs; lnaAs; |
| | | | | | | dGs; lnaCs; |
| | | | | | | dCs; lnaTs; |
| | | | | | | dTs; lnaTs; |
| | | | | | | dTs; lnaCs; |
| | | | | | | dCs; lnaTs; |
| | | | | | | dTs; lnaTs; |
| | | | | | | dTs; lnaTs; |
| | | | | | | dAs; lnaAs; |
| | | | | | | dAs; lnaTs; |
| | | | | | | dTs; lnaTs; |
| | | | | | | dAs; lnaTs; |
| | | | | | | dAs; lnaTs; |
| | | | | | | dTs; lnaAs; |
| | | | | | | dCs; lnaAs; |
| | | | | | | dT-Sup |
| 264 | KLF4-55 | GCCTTCTCCCTTTTTAGA | KLF4 | 5′ and 3′ | human | dGs; lnaCs; |
| | m02 | TAAAATA | | | | dCs; lnaTs; |
| | | | | | | dTs; lnaCs; |
| | | | | | | dTs; lnaCs; |
| | | | | | | dCs; lnaCs; |
| | | | | | | dTs; lnaTs; |
| | | | | | | dTs; lnaTs; |
| | | | | | | dTs; lnaAs; |
| | | | | | | dGs; lnaAs; |
| | | | | | | dTs; lnaAs; |
| | | | | | | dAs; lnaAs; |
| | | | | | | dAs; lnaTs; |
| | | | | | | dA- |
| | | | | | | Sup |
| 265 | KLF4-56 | TCGGGAAACTTTTTAGA | KLF4 | 5′ and 3′ | human | dTs; lnaCs; |
| | m02 | TAAAATA | | | | dGs; lnaGs; |
| | | | | | | dGs; lnaAs; |
| | | | | | | dAs; lnaAs; |
| | | | | | | dCs; lnaTs; |
| | | | | | | dTs; lnaTs; |
| | | | | | | dTs; lnaTs; |
| | | | | | | dAs; lnaGs; |
| | | | | | | dAs; lnaTs; |
| | | | | | | dAs; lnaAs; |
| | | | | | | dAs; lnaAs; |
| | | | | | | dTs; lnaA- |
| | | | | | | Sup |
| 266 | KLF4-57 | AGCCTTTTCCTTTTTAGA | KLF4 | 5′ and 3′ | human | dAs; lnaGs; |
| | m02 | TAAAATA | | | | dCs; lnaCs; |
| | | | | | | dTs; lnaTs; |
| | | | | | | dTs; lnaTs; |
| | | | | | | dCs; lnaCs; |
| | | | | | | dTs; lnaTs; |
| | | | | | | dTs; lnaTs; |
| | | | | | | dTs; lnaAs; |
| | | | | | | dGs; lnaAs; |
| | | | | | | dTs; lnaAs; |
| | | | | | | dAs; lnaAs; |
| | | | | | | dAs; lnaTs; |
| | | | | | | dA- |
| | | | | | | Sup |
| 267 | KLF4-58 | GCCTTCTCCCTTTTTGGT | KLF4 | 5′ and 3′ | human | dGs; lnaCs; |
| | m02 | TTATTTA | | | | dCs; lnaTs; |
| | | | | | | dTs; lnaCs; |
| | | | | | | dTs; lnaCs; |
| | | | | | | dCs; lnaCs; |
| | | | | | | dTs; lnaTs; |
| | | | | | | dTs; lnaTs; |
| | | | | | | dTs; lnaGs; |
| | | | | | | dGs; lnaTs; |
| | | | | | | dTs; lnaTs; |
| | | | | | | dAs; lnaTs; |
| | | | | | | dTs; lnaTs; |
| | | | | | | dA- |
| | | | | | | Sup |
| 268 | KLF4-59 | TCGGGAAACTTTTTGGT | KLF4 | 5′ and 3′ | human | dTs; lnaCs; |
| | m02 | TTATTTA | | | | dGs; lnaGs; |
| | | | | | | dGs; lnaAs; |
| | | | | | | dAs; lnaAs; |
| | | | | | | dCs; lnaTs; |
| | | | | | | dTs; lnaTs; |
| | | | | | | dTs; lnaTs; |
| | | | | | | dGs; lnaGs; |
| | | | | | | dTs; lnaTs; |
| | | | | | | dTs; lnaAs; |
| | | | | | | dTs; lnaTs; |
| | | | | | | dTs; lnaA- |
| | | | | | | Sup |
| 269 | KLF4-60 | AGCCTTTTCCTTTTTGGT | KLF4 | 5′ and 3′ | human | dAs; lnaGs; |
| | m02 | TTATTTA | | | | dCs; lnaCs; |
| | | | | | | dTs; lnaTs; |
| | | | | | | dTs; lnaTs; |
| | | | | | | dCs; lnaCs; |
| | | | | | | dTs; lnaTs; |
| | | | | | | dTs; lnaTs; |
| | | | | | | dTs; lnaGs; |
| | | | | | | dGs; lnaTs; |
| | | | | | | dTs; lnaTs; |
| | | | | | | dAs; lnaTs; |
| | | | | | | dTs; lnaTs; |
| | | | | | | dA- |
| | | | | | | Sup |
| 270 | KLF4-61 | GCCTTCTCCCTTTTTAAA | KLF4 | 5′ and 3′ | human | dGs; lnaCs; |
| | m02 | TTTATAT | | | | dCs; lnaTs; |
| | | | | | | dTs; lnaCs; |
| | | | | | | dTs; lnaCs; |
| | | | | | | dCs; lnaCs; |
| | | | | | | dTs; lnaTs; |
| | | | | | | dTs; lnaTs; |
| | | | | | | dTs; lnaAs; |
| | | | | | | dAs; lnaAs; |
| | | | | | | dTs; lnaTs; |
| | | | | | | dTs; lnaAs; |
| | | | | | | dTs; lnaAs; |
| | | | | | | dT- |
| | | | | | | Sup |
| 271 | KLF4-62 | TCGGGAAACTTTTTAAA | KLF4 | 5′ and 3′ | human | dTs; lnaCs; |
| | m02 | TTTATAT | | | | dGs; lnaGs; |
| | | | | | | dGs; lnaAs; |
| | | | | | | dAs; lnaAs; |
| | | | | | | dCs; lnaTs; |
| | | | | | | dTs; lnaTs; |
| | | | | | | dTs; lnaTs; |
| | | | | | | dAs; lnaAs; |
| | | | | | | dAs; lnaTs; |
| | | | | | | dTs; lnaTs; |
| | | | | | | dAs; lnaTs; |
| | | | | | | dAs; lnaT- |
| | | | | | | Sup |
| 272 | KLF4-63 | AGCCTTTTCCTTTTTAAA | KLF4 | 5′ and 3′ | human | dAs; lnaGs; |
| | m02 | TTTATAT | | | | dCs; lnaCs; |
| | | | | | | dTs; lnaTs; |
| | | | | | | dTs; lnaTs; |
| | | | | | | dCs; lnaCs; |
| | | | | | | dTs; lnaTs; |
| | | | | | | dTs; lnaTs; |
| | | | | | | dTs; lnaAs; |
| | | | | | | dAs; lnaAs; |
| | | | | | | dTs; lnaTs; |
| | | | | | | dTs; lnaAs; |
| | | | | | | dTs; lnaAs; |
| | | | | | | dT- |
| | | | | | | Sup |
| 273 | ACTB-01 | AGGTGTGCACTTTTA | ACTB | 3′ | human | dAs; lnaGs; |
| | m02 | | | | | dGs; lnaTs; |
| | | | | | | dGs; lnaTs; |
| | | | | | | dGs; lnaCs; |
| | | | | | | dAs; lnaCs; |
| | | | | | | dTs; lnaTs; |
| | | | | | | dTs; lnaTs; |
| | | | | | | dA- |
| | | | | | | Sup |
| 274 | ACTB-02 | TCATTTTTAAGGTGT | ACTB | 3′ | human | dTs; lnaCs; |
| | m02 | | | | | dAs; lnaTs; |
| | | | | | | dTs; lnaTs; |
| | | | | | | dTs; lnaTs; |
| | | | | | | dAs; lnaAs; |
| | | | | | | dGs; lnaGs; |
| | | | | | | dTs; lnaGs; |
| | | | | | | dT- |
| | | | | | | Sup |
| 275 | ACTB-03 | TTTTTAGGTGTGCACTTT | ACTB | 3′ | human | dTs; lnaTs; |
| | m02 | TA | | | | dTs; lnaTs; |
| | | | | | | dTs; lnaAs; |
| | | | | | | dGs; lnaGs; |
| | | | | | | dTs; lnaGs; |
| | | | | | | dTs; lnaGs; |
| | | | | | | dCs; lnaAs; |
| | | | | | | dCs; lnaTs; |
| | | | | | | dTs; lnaTs; |
| | | | | | | dTs; lnaA- |
| | | | | | | Sup |
| 276 | ACTB-04 | TTTTTCATTTTTAAGGTGT | ACTB | 3′ | human | dTs; lnaTs; |
| | m02 | | | | | dTs; lnaTs; |
| | | | | | | dTs; lnaCs; |
| | | | | | | dAs; lnaTs; |
| | | | | | | dTs; lnaTs; |
| | | | | | | dTs; lnaTs; |
| | | | | | | dAs; lnaAs; |
| | | | | | | dGs; lnaGs; |
| | | | | | | dTs; lnaGs; |
| | | | | | | dT-Sup |
| 277 | ACTB-05 | CGCGGTCTCGGCGGT | ACTB | 5′ | human | dCs; lnaGs; |
| | m02 | | | | | dCs; lnaGs; |
| | | | | | | dGs; lnaTs; |
| | | | | | | dCs; lnaTs; |
| | | | | | | dCs; lnaGs; |
| | | | | | | dGs; lnaCs; |
| | | | | | | dGs; lnaGs; |
| | | | | | | dT-Sup |
| 278 | ACTB-06 | ATCATCCATGGTGAG | ACTB | 5′ | human | dAs; lnaTs; |
| | m02 | | | | | dCs; lnaAs; |
| | | | | | | dTs; lnaCs; |
| | | | | | | dCs; lnaAs; |
| | | | | | | dTs; lnaGs; |
| | | | | | | dGs; lnaTs; |
| | | | | | | dGs; lnaAs; |
| | | | | | | dG- |
| | | | | | | Sup |
| 279 | ACTB-07 | CGCGGTCTCGGCGGTTT | ACTB | 5′ and 3′ | human | dCs; lnaGs; |
| | m02 | TTAGGTGTGCACTTTTA | | | | dCs; lnaGs; |
| | | | | | | dGs; lnaTs; |
| | | | | | | dCs; lnaTs; |
| | | | | | | dCs; lnaGs; |
| | | | | | | dGs; lnaCs; |
| | | | | | | dGs; lnaGs; |
| | | | | | | dTs; lnaTs; |
| | | | | | | dTs; lnaTs; |
| | | | | | | dTs; lnaAs; |
| | | | | | | dGs; lnaGs; |
| | | | | | | dTs; lnaGs; |
| | | | | | | dTs; lnaGs; |
| | | | | | | dCs; lnaAs; |
| | | | | | | dCs; lnaTs; |
| | | | | | | dTs; lnaTs; |
| | | | | | | dTs; lnaA- |
| | | | | | | Sup |
| 280 | ACTB-08 | ATCATCCATGGTGAGTT | ACTB | 5′ and 3′ | human | dAs; lnaTs; |
| | m02 | TTTAGGTGTGCACTTTTA | | | | dCs; lnaAs; |
| | | | | | | dTs; lnaCs; |
| | | | | | | dCs; lnaAs; |
| | | | | | | dTs; lnaGs; |
| | | | | | | dGs; lnaTs; |
| | | | | | | dGs; lnaAs; |
| | | | | | | dGs; lnaTs; |
| | | | | | | dTs; lnaTs; |
| | | | | | | dTs; lnaTs; |
| | | | | | | dAs; lnaGs; |
| | | | | | | dGs; lnaTs; |
| | | | | | | dGs; lnaTs; |
| | | | | | | dGs; lnaCs; |
| | | | | | | dAs; lnaCs; |
| | | | | | | dTs; lnaTs; |
| | | | | | | dTs; lnaTs; |
| | | | | | | dA-Sup |
| 281 | ACTB-09 | CGCGGTCTCGGCGGTTT | ACTB | 5′ and 3′ | human | dCs; lnaGs; |
| | m02 | TTCATTTTTAAGGTGT | | | | dCs; lnaGs; |
| | | | | | | dGs; lnaTs; |
| | | | | | | dCs; lnaTs; |
| | | | | | | dCs; lnaGs; |
| | | | | | | dGs; lnaCs; |
| | | | | | | dGs; lnaGs; |
| | | | | | | dTs; lnaTs; |
| | | | | | | dTs; lnaTs; |
| | | | | | | dTs; lnaCs; |
| | | | | | | dAs; lnaTs; |
| | | | | | | dTs; lnaTs; |
| | | | | | | dTs; lnaTs; |
| | | | | | | dAs; lnaAs; |
| | | | | | | dGs; lnaGs; |
| | | | | | | dTs; lnaGs; |
| | | | | | | dT-Sup |
| 282 | ACTB-10 | ATCATCCATGGTGAGTT | ACTB | 5′ and 3′ | human | dAs; lnaTs; |
| | m02 | TTTCATTTTTAAGGTGT | | | | dCs; lnaAs; |
| | | | | | | dTs; lnaCs; |
| | | | | | | dCs; lnaAs; |
| | | | | | | dTs; lnaGs; |
| | | | | | | dGs; lnaTs; |
| | | | | | | dGs; lnaAs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaTs; |
| | | | | | | dGs; lnaT- |
| | | | | | | Sup |
| 283 | ACTB-11 | TCTCGGCGGTTTTTAGG | ACTB | 5′ and 3′ | human | dTs; lnaCs; |
| | m02 | TGTGCAC | | | | dTs; lnaCs; |
| | | | | | | dGs; lnaGs; |
| | | | | | | dCs; lnaGs; |
| | | | | | | dGs; lnaTs; |
| | | | | | | dTs; lnaTs; |
| | | | | | | dTs; lnaTs; |
| | | | | | | dAs; lnaGs; |
| | | | | | | dGs; lnaTs; |
| | | | | | | dGs; lnaTs; |
| | | | | | | dGs; lnaCs; |
| | | | | | | dAs; lnaC- |
| | | | | | | Sup |
| 284 | ACTB-12 | CCATGGTGAGTTTTTAG | ACTB | 5′ and 3′ | human | dCs; lnaCs; |
| | m02 | GTGTGCAC | | | | dAs; lnaTs; |
| | | | | | | dGs; lnaGs; |
| | | | | | | dTs; lnaGs; |
| | | | | | | dAs; lnaGs; |
| | | | | | | dTs; lnaTs; |
| | | | | | | dTs; lnaTs; |
| | | | | | | dTs; lnaAs; |
| | | | | | | dGs; lnaGs; |
| | | | | | | dTs; lnaGs; |
| | | | | | | dTs; lnaGs; |
| | | | | | | dCs; lnaAs; |
| | | | | | | dC-Sup |
| 285 | ACTB-13 | TCTCGGCGGTTTTTCATT | ACTB | 5′ and 3′ | human | dTs; lnaCs; |
| | m02 | TTTAA | | | | dTs; lnaCs; |
| | | | | | | dGs; lnaGs; |
| | | | | | | dCs; lnaGs; |
| | | | | | | dGs; lnaTs; |
| | | | | | | dTs; lnaTs; |
| | | | | | | dTs; lnaTs; |
| | | | | | | dCs; lnaAs; |
| | | | | | | dTs; lnaTs; |
| | | | | | | dTs; lnaTs; |
| | | | | | | dTs; lnaAs; |
| | | | | | | dA-Sup |
| 286 | ACTB-14 | CCATGGTGAGTTTTTCA | ACTB | 5′ and 3′ | human | dCs; lnaCs; |
| | m02 | TTTTTAA | | | | dAs; lnaTs; |
| | | | | | | dGs; lnaGs; |
| | | | | | | dTs; lnaGs; |
| | | | | | | dAs; lnaGs; |
| | | | | | | dTs; lnaTs; |
| | | | | | | dTs; lnaTs; |
| | | | | | | dTs; lnaCs; |
| | | | | | | dAs; lnaTs; |
| | | | | | | dTs; lnaTs; |
| | | | | | | dTs; lnaTs; |
| | | | | | | dAs; lnaA- |
| | | | | | | Sup |
| 287 | ACTB-15 | CGCGGTCTCGGCGGTA | ACTB | 5′ and 3′ | human | dCs; lnaGs; |
| | m02 | GGTGTGCACTTTTA | | | | dCs; lnaGs; |
| | | | | | | dGs; lnaTs; |
| | | | | | | dCs; lnaTs; |
| | | | | | | dCs; lnaGs; |
| | | | | | | dGs; lnaCs; |
| | | | | | | dGs; lnaGs; |
| | | | | | | dTs; lnaAs; |
| | | | | | | dGs; lnaGs; |
| | | | | | | dTs; lnaGs; |
| | | | | | | dTs; lnaGs; |
| | | | | | | dCs; lnaAs; |
| | | | | | | dCs; lnaTs; |
| | | | | | | dTs; lnaTs; |
| | | | | | | dTs; lnaA- |
| | | | | | | Sup |
| 288 | ACTB-16 | ATCATCCATGGTGAGAG | ACTB | 5′ and 3′ | human | dAs; lnaTs; |
| | m02 | GTGTGCACTTTTA | | | | dCs; lnaAs; |
| | | | | | | dTs; lnaCs; |
| | | | | | | dCs; lnaAs; |
| | | | | | | dTs; lnaGs; |
| | | | | | | dGs; lnaTs; |
| | | | | | | dGs; lnaAs; |
| | | | | | | dGs; lnaAs; |
| | | | | | | dGs; lnaGs; |
| | | | | | | dTs; lnaGs; |
| | | | | | | dTs; lnaGs; |
| | | | | | | dCs; lnaAs; |
| | | | | | | dCs; lnaTs; |
| | | | | | | dTs; lnaTs; |
| | | | | | | dTs; lnaA- |
| | | | | | | Sup |
| 289 | ACTB-17 | CGCGGTCTCGGCGGTTC | ACTB | 5′ and 3′ | human | dCs; lnaGs; |
| | m02 | ATTTTTAAGGTGT | | | | dCs; lnaGs; |
| | | | | | | dGs; lnaTs; |
| | | | | | | dCs; lnaTs; |
| | | | | | | dCs; lnaGs; |
| | | | | | | dGs; lnaCs; |
| | | | | | | dGs; lnaGs; |
| | | | | | | dTs; lnaTs; |
| | | | | | | dCs; lnaAs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaTs; |
| | | | | | | dGs; lnaT- |
| | | | | | | Sup |
| 290 | ACTB-18 | ATCATCCATGGTGAGTC | ACTB | 5′ and 3′ | human | dAs; lnaTs; |
| | m02 | ATTTTTAAGGTGT | | | | dCs; lnaAs; |
| | | | | | | dTs; lnaCs; |
| | | | | | | dCs; lnaAs; |
| | | | | | | dTs; lnaGs; |
| | | | | | | dGs; lnaTs; |
| | | | | | | dGs; lnaAs; |
| | | | | | | dGs; lnaTs; |
| | | | | | | dCs; lnaAs; |
| | | | | | | dTs; lnaTs; |
| | | | | | | dTs; lnaTs; |
| | | | | | | dTs; lnaAs; |
| | | | | | | dAs; lnaGs; |
| | | | | | | dGs; lnaTs; |
| | | | | | | dGs; lnaT- |
| | | | | | | Sup |
| 291 | UTRN- | TGGAGCCGAGCGCTG | UTRN | 5′ | human | dTs; lnaGs; |
| | 192 m02 | | | | | dGs; lnaAs; |
| | | | | | | dGs; lnaCs; |
| | | | | | | dCs; lnaGs; |
| | | | | | | dAs; lnaGs; |
| | | | | | | dCs; lnaGs; |
| | | | | | | dCs; lnaTs; |
| | | | | | | dG- |
| | | | | | | Sup |
| 292 | UTRN- | GGGCCTGCCCCTTTG | UTRN | 5′ | human | dGs; lnaGs; |
| | 193 m02 | | | | | dGs; lnaCs; |
| | | | | | | dCs; lnaTs; |
| | | | | | | dGs; lnaCs; |
| | | | | | | dCs; lnaCs; |
| | | | | | | dCs; lnaTs; |
| | | | | | | dTs; lnaTs; |
| | | | | | | dG- |
| | | | | | | Sup |
| 293 | UTRN- | CCCCAAGTCACCTGA | UTRN | 5′ | human | dCs; lnaCs; |
| | 194 m02 | | | | | dCs; lnaCs; |
| | | | | | | dAs; lnaAs; |
| | | | | | | dGs; lnaTs; |
| | | | | | | dCs; lnaAs; |
| | | | | | | dCs; lnaCs; |
| | | | | | | dTs; lnaGs; |
| | | | | | | dA- |
| | | | | | | Sup |
| 294 | UTRN- | GACATCAATACCTAA | UTRN | 5′ | human | dGs; lnaAs; |
| | 195 m02 | | | | | dCs; lnaAs; |
| | | | | | | dTs; lnaCs; |
| | | | | | | dAs; lnaAs; |
| | | | | | | dTs; lnaAs; |
| | | | | | | dCs; lnaCs; |
| | | | | | | dTs; lnaAs; |
| | | | | | | dA- |
| | | | | | | Sup |
| 295 | UTRN- | AAACTTTACCAAGTC | UTRN | 5′ | human | dAs; lnaAs; |
| | 196 m02 | | | | | dAs; lnaCs; |
| | | | | | | dTs; lnaTs; |
| | | | | | | dTs; lnaAs; |
| | | | | | | dCs; lnaCs; |
| | | | | | | dAs; lnaAs; |
| | | | | | | dGs; lnaTs; |
| | | | | | | dC- |
| | | | | | | Sup |
| 296 | UTRN- | TGGAGCCGAGCGCTGCC | UTRN | 5′ | human | dTs; lnaGs; |
| | 197 m02 | | | | | dGs; lnaAs; |
| | | | | | | dGs; lnaCs; |
| | | | | | | dCs; lnaGs; |
| | | | | | | dAs; lnaGs; |
| | | | | | | dCs; lnaGs; |
| | | | | | | dCs; lnaTs; |
| | | | | | | dGs; lnaCs; |
| | | | | | | dC-Sup |
| 297 | UTRN- | GGGCCTGCCCCTTTGCC | UTRN | 5′ | human | dGs; lnaGs; |
| | 198 m02 | | | | | dGs; lnaCs; |
| | | | | | | dCs; lnaTs; |
| | | | | | | dGs; lnaCs; |
| | | | | | | dCs; lnaCs; |
| | | | | | | dCs; lnaTs; |
| | | | | | | dTs; lnaTs; |
| | | | | | | dGs; lnaCs; |
| | | | | | | dC-Sup |
| 298 | UTRN- | CCCCAAGTCACCTGACC | UTRN | 5′ | human | dCs; lnaCs; |
| | 199 m02 | | | | | dCs; lnaCs; |
| | | | | | | dAs; lnaAs; |
| | | | | | | dGs; lnaTs; |
| | | | | | | dCs; lnaAs; |
| | | | | | | dCs; lnaCs; |
| | | | | | | dTs; lnaGs; |
| | | | | | | dAs; lnaCs; |
| | | | | | | dC-Sup |
| 299 | UTRN- | GACATCAATACCTAACC | UTRN | 5′ | human | dGs; lnaAs; |
| | 200 m02 | | | | | dCs; lnaAs; |
| | | | | | | dTs; lnaCs; |
| | | | | | | dAs; lnaAs; |
| | | | | | | dTs; lnaAs; |
| | | | | | | dCs; lnaCs; |
| | | | | | | dTs; lnaAs; |
| | | | | | | dAs; lnaCs; |
| | | | | | | dC-Sup |
| 300 | UTRN- | AAACTTTACCAAGTCCC | UTRN | 5′ | human | dAs; lnaAs; |
| | 201 m02 | | | | | dAs; lnaCs; |
| | | | | | | dTs; lnaTs; |
| | | | | | | dTs; lnaAs; |
| | | | | | | dCs; lnaCs; |
| | | | | | | dAs; lnaAs; |
| | | | | | | dGs; lnaTs; |
| | | | | | | dCs; lnaCs; |
| | | | | | | dC-Sup |
| 301 | UTRN- | TGGAGCCGAGCGCTGG | UTRN | 5′ | human | dTs; lnaGs; |
| | 202 | GAAACCAC | | | | dGs; lnaAs; |
| | m1000 | | | | | dGs; lnaCs; |
| | | | | | | dCs; lnaGs; |
| | | | | | | dAs; lnaGs; |
| | | | | | | dCs; lnaGs; |
| | | | | | | dCs; lnaTs; |
| | | | | | | dGs; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 302 | UTRN- | GGGCCTGCCCCTTTGGG | UTRN | 5′ | human | dGs; lnaGs; |
| | 203 | AAACCAC | | | | dGs; lnaCs; |
| | m1000 | | | | | dCs; lnaTs; |
| | | | | | | dGs; lnaCs; |
| | | | | | | dCs; lnaCs; |
| | | | | | | dCs; lnaTs; |
| | | | | | | dTs; lnaTs; |
| | | | | | | dGs; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 303 | UTRN- | CCCCAAGTCACCTGAGG | UTRN | 5′ | human | dCs; lnaCs; |
| | 204 | AAACCAC | | | | dCs; lnaCs; |
| | m1000 | | | | | dAs; lnaAs; |
| | | | | | | dGs; lnaTs; |
| | | | | | | dCs; lnaAs; |
| | | | | | | dCs; lnaCs; |
| | | | | | | dTs; lnaGs; |
| | | | | | | dAs; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 304 | UTRN- | GACATCAATACCTAAGG | UTRN | 5′ | human | dGs; lnaAs; |
| | 205 | AAACCAC | | | | dCs; lnaAs; |
| | m1000 | | | | | dTs; lnaCs; |
| | | | | | | dAs; lnaAs; |
| | | | | | | dTs; lnaAs; |
| | | | | | | dCs; lnaCs; |
| | | | | | | dTs; lnaAs; |
| | | | | | | dAs; lnaGs; |
| | | | | | | dGs; dAs; |
| | | | | | | dAs; dAs; |
| | | | | | | dCs; lnaCs; |
| | | | | | | dAs; lnaC-Sup |
| 305 | UTRN- | AAACTTTACCAAGTCGG | UTRN | 5′ | human | dAs; lnaAs; |
| | 206 | AAACCAC | | | | dAs; lnaCs; |
| | m1000 | | | | | dTs; lnaTs; |
| | | | | | | dTs; lnaAs; |
| | | | | | | dCs; lnaCs; |
| | | | | | | dAs; lnaAs; |
| | | | | | | dGs; lnaTs; |
| | | | | | | dCs; lnaGs; |
| | | | | | | dGs; dAs; |
| | | | | | | dAs; dAs; |
| | | | | | | dCs; lnaCs; |
| | | | | | | dAs; lnaC-Sup |
| 306 | UTRN- | ACTGCAATATATTTC | UTRN | 3′ | human | dAs; lnaCs; |
| | 207 m02 | | | | | dTs; lnaGs; |
| | | | | | | dCs; lnaAs; |
| | | | | | | dAs; lnaTs; |
| | | | | | | dAs; lnaTs; |
| | | | | | | dAs; lnaTs; |
| | | | | | | dTs; lnaTs; |
| | | | | | | dC- |
| | | | | | | Sup |
| 307 | UTRN- | GTGTTAAAATTACTT | UTRN | 3′ | human | dGs; lnaTs; |
| | 208 m02 | | | | | dGs; lnaTs; |
| | | | | | | dTs; lnaAs; |
| | | | | | | dAs; lnaAs; |
| | | | | | | dAs; lnaTs; |
| | | | | | | dTs; lnaAs; |
| | | | | | | dCs; lnaTs; |
| | | | | | | dT- |
| | | | | | | Sup |
| 308 | UTRN- | TTTTTACTGCAATATATT | UTRN | 3′ | human | dTs; lnaTs; |
| | 209 m02 | TC | | | | dTs; lnaTs; |
| | | | | | | dTs; lnaAs; |
| | | | | | | dCs; lnaTs; |
| | | | | | | dGs; lnaCs; |
| | | | | | | dAs; lnaAs; |
| | | | | | | dTs; lnaAs; |
| | | | | | | dTs; lnaAs; |
| | | | | | | dTs; lnaTs; |
| | | | | | | dTs; lnaC- |
| | | | | | | Sup |
| 309 | UTRN- | TTTTTGTGTTAAAATTAC | UTRN | 3′ | human | dTs; lnaTs; |
| | 210 m02 | TT | | | | dTs; lnaTs; |
| | | | | | | dTs; lnaGs; |
| | | | | | | dTs; lnaGs; |
| | | | | | | dTs; lnaTs; |
| | | | | | | dAs; lnaAs; |
| | | | | | | dAs; lnaAs; |
| | | | | | | dTs; lnaTs; |
| | | | | | | dAs; lnaCs; |
| | | | | | | dTs; lnaT- |
| | | | | | | Sup |
| 310 | UTRN- | CCGAGCGCTGTTTTTAC | UTRN | 5′ and 3′ | human | dCs; lnaCs; |
| | 211 m02 | TGCAATAT | | | | dGs; lnaAs; |
| | | | | | | dGs; lnaCs; |
| | | | | | | dGs; lnaCs; |
| | | | | | | dTs; lnaGs; |
| | | | | | | dTs; lnaTs; |
| | | | | | | dTs; lnaTs; |
| | | | | | | dTs; lnaAs; |
| | | | | | | dCs; lnaTs; |
| | | | | | | dGs; lnaCs; |
| | | | | | | dAs; lnaAs; |
| | | | | | | dTs; lnaAs; |
| | | | | | | dT-Sup |
| 311 | UTRN- | TGCCCCTTTGTTTTTACT | UTRN | 5′ and 3′ | human | dTs; lnaGs; |
| | 212 m02 | GCAATAT | | | | dCs; lnaCs; |
| | | | | | | dCs; lnaCs; |
| | | | | | | dTs; lnaTs; |
| | | | | | | dTs; lnaGs; |
| | | | | | | dTs; lnaTs; |
| | | | | | | dTs; lnaTs; |
| | | | | | | dTs; lnaAs; |
| | | | | | | dCs; lnaTs; |
| | | | | | | dGs; lnaCs; |
| | | | | | | dAs; lnaAs; |
| | | | | | | dTs; lnaAs; |
| | | | | | | dT- |
| | | | | | | Sup |
| 312 | UTRN- | AGTCACCTGATTTTTACT | UTRN | 5′ and 3′ | human | dAs; lnaGs; |
| | 213 m02 | GCAATAT | | | | dTs; lnaCs; |
| | | | | | | dAs; lnaCs; |
| | | | | | | dCs; lnaTs; |
| | | | | | | dGs; lnaAs; |
| | | | | | | dTs; lnaTs; |
| | | | | | | dTs; lnaTs; |
| | | | | | | dTs; lnaAs; |
| | | | | | | dCs; lnaTs; |
| | | | | | | dGs; lnaCs; |
| | | | | | | dAs; lnaAs; |
| | | | | | | dTs; lnaAs; |
| | | | | | | dT- |
| | | | | | | Sup |
| 313 | UTRN- | CAATACCTAATTTTTACT | UTRN | 5′ and 3′ | human | dCs; lnaAs; |
| | 214 m02 | GCAATAT | | | | dAs; lnaTs; |
| | | | | | | dAs; lnaCs; |
| | | | | | | dCs; lnaTs; |
| | | | | | | dAs; lnaAs; |
| | | | | | | dTs; lnaTs; |
| | | | | | | dTs; lnaTs; |
| | | | | | | dTs; lnaAs; |
| | | | | | | dCs; lnaTs; |
| | | | | | | dGs; lnaCs; |
| | | | | | | dAs; lnaAs; |
| | | | | | | dTs; lnaAs; |
| | | | | | | dT- |
| | | | | | | Sup |
| 314 | UTRN- | TTACCAAGTCTTTTTACT | UTRN | 5′ and 3′ | human | dTs; lnaTs; |
| | 215 m02 | GCAATAT | | | | dAs; lnaCs; |
| | | | | | | dCs; lnaAs; |
| | | | | | | dAs; lnaGs; |
| | | | | | | dTs; lnaCs; |
| | | | | | | dTs; lnaTs; |
| | | | | | | dTs; lnaTs; |
| | | | | | | dTs; lnaAs; |
| | | | | | | dCs; lnaTs; |
| | | | | | | dGs; lnaCs; |
| | | | | | | dAs; lnaAs; |
| | | | | | | dTs; lnaAs; |
| | | | | | | dT-Sup |
| 315 | UTRN- | CCGAGCGCTGTTTTTGT | UTRN | 5′ and 3′ | human | dCs; lnaCs; |
| | 216 m02 | GTTAAAAT | | | | dGs; lnaAs; |
| | | | | | | dGs; lnaCs; |
| | | | | | | dGs; lnaCs; |
| | | | | | | dTs; lnaGs; |
| | | | | | | dTs; lnaTs; |
| | | | | | | dTs; lnaTs; |
| | | | | | | dTs; lnaGs; |
| | | | | | | dTs; lnaGs; |
| | | | | | | dTs; lnaTs; |
| | | | | | | dAs; lnaAs; |
| | | | | | | dAs; lnaAs; |
| | | | | | | dT-Sup |
| 316 | UTRN- | TGCCCCTTTGTTTTTGTG | UTRN | 5′ and 3′ | human | dTs; lnaGs; |
| | 217 m02 | TTAAAAT | | | | dCs; lnaCs; |
| | | | | | | dCs; lnaCs; |
| | | | | | | dTs; lnaTs; |
| | | | | | | dTs; lnaGs; |
| | | | | | | dTs; lnaTs; |
| | | | | | | dTs; lnaTs; |
| | | | | | | dTs; lnaGs; |
| | | | | | | dTs; lnaGs; |
| | | | | | | dTs; lnaTs; |
| | | | | | | dAs; lnaAs; |
| | | | | | | dAs; lnaAs; |
| | | | | | | dT- |
| | | | | | | Sup |
| 317 | UTRN- | AGTCACCTGATTTTTGT | UTRN | 5′ and 3′ | human | dAs; lnaGs; |
| | 218 m02 | GTTAAAAT | | | | dTs; lnaCs; |
| | | | | | | dAs; lnaCs; |
| | | | | | | dCs; lnaTs; |
| | | | | | | dGs; lnaAs; |
| | | | | | | dTs; lnaTs; |
| | | | | | | dTs; lnaTs; |
| | | | | | | dTs; lnaGs; |
| | | | | | | dTs; lnaGs; |
| | | | | | | dTs; lnaTs; |
| | | | | | | dAs; lnaAs; |
| | | | | | | dAs; lnaAs; |
| | | | | | | dT- |
| | | | | | | Sup |
| 318 | UTRN- | CAATACCTAATTTTTGTG | UTRN | 5′ and 3′ | human | dCs; lnaAs; |
| | 219 m02 | TTAAAAT | | | | dAs; lnaTs; |
| | | | | | | dAs; lnaCs; |
| | | | | | | dCs; lnaTs; |
| | | | | | | dAs; lnaAs; |
| | | | | | | dTs; lnaTs; |
| | | | | | | dTs; lnaTs; |
| | | | | | | dTs; lnaGs; |
| | | | | | | dTs; lnaGs; |
| | | | | | | dTs; lnaTs; |
| | | | | | | dAs; lnaAs; |
| | | | | | | dAs; lnaAs; |
| | | | | | | dT- |
| | | | | | | Sup |
| 319 | UTRN- | TTACCAAGTCTTTTTGTG | UTRN | 5′ and 3′ | human | dTs; lnaTs; |
| | 220 m02 | TTAAAAT | | | | dAs; lnaCs; |
| | | | | | | dCs; lnaAs; |
| | | | | | | dAs; lnaGs; |
| | | | | | | dTs; lnaCs; |
| | | | | | | dTs; lnaTs; |
| | | | | | | dTs; lnaTs; |
| | | | | | | dTs; lnaGs; |
| | | | | | | dTs; lnaGs; |
| | | | | | | dTs; lnaTs; |
| | | | | | | dAs; lnaAs; |
| | | | | | | dAs; lnaAs; |
| | | | | | | dT-Sup |
| 320 | HBF-XXX | TGTCTGTAGCTCCAG | HBF | 5′ | human | dTs; lnaGs; |
| | m02 | | | | | dTs; lnaCs; |
| | | | | | | dTs; lnaG; |
| | | | | | | dTs; lnaA; |
| | | | | | | dGs; lnaC; |
| | | | | | | dTs; lnaC; |
| | | | | | | dCs; lnaA; |
| | | | | | | dGs-Sup |
| 321 | HBF-XXX | TAGCTCCAGTGAGGC | HBF | 5′ | human | dTs; lnaAs; |
| | m02 | | | | | dGs; lnaCs; |
| | | | | | | dTs; lnaCs; |
| | | | | | | dCs; lnaAs; |
| | | | | | | dGs; lnaTs; |
| | | | | | | dGs; lnaAs; |
| | | | | | | dGs; lnaGs; |
| | | | | | | dC-Sup |
| 322 | HBF-XXX | TTTCTTCTCCCACCA | HBF | 5′ | human | dTs; lnaTs; |
| | m02 | | | | | dTs; lnaCs; |
| | | | | | | dTs; lnaTs; |
| | | | | | | dCs; lnaTs; |
| | | | | | | dCs; lnaCs; |
| | | | | | | dCs; lnaAs; |
| | | | | | | dCs; lnaCs; |
| | | | | | | dA-Sup |
| 323 | HBF-XXX | TGTCTGTAGCTCCAGCC | HBF | 5′ | human | dTs; lnaGs; |
| | m02 | | | | | dTs; lnaCs; |
| | | | | | | dTs; lnaG; |
| | | | | | | dTs; lnaA; |
| | | | | | | dGs; lnaC; |
| | | | | | | dTs; lnaC; |
| | | | | | | dCs; lnaA; |
| | | | | | | dGs; lnaCs; |
| | | | | | | dC-Sup |
| 324 | HBF-XXX | TAGCTCCAGTGAGGC | HBF | 5′ | human | dTs; lnaAs; |
| | m02 | CC | | | | dGs; lnaCs; |
| | | | | | | dTs; lnaCs; |
| | | | | | | dCs; lnaAs; |
| | | | | | | dGs; lnaTs; |
| | | | | | | dGs; lnaAs; |
| | | | | | | dGs; lnaGs; |
| | | | | | | dC; lnaCs; |
| | | | | | | dC-Sup |
| 325 | HBF-XXX | TTTCTTCTCCCACCACC | HBF | 5′ | human | dTs; lnaTs; |
| | m02 | | | | | dTs; lnaCs; |
| | | | | | | dTs; lnaTs; |
| | | | | | | dCs; lnaTs; |
| | | | | | | dCs; lnaCs; |
| | | | | | | dCs; lnaAs; |
| | | | | | | dCs; lnaCs; |
| | | | | | | dA; lnaCs; |
| | | | | | | dC-Sup |
| 326 | HBF-XXX | TGTCTGTAGCTCCAG | HBF | 5′ | human | dTs; lnaGs; |
| | m03 | GGAAACCAC | | | | dTs; lnaCs; |
| | | | | | | dTs; lnaG; |
| | | | | | | dTs; lnaA; |
| | | | | | | dGs; lnaC; |
| | | | | | | dTs; lnaC; |
| | | | | | | dCs; lnaA; |
| | | | | | | dGs; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 327 | HBF-XXX | TAGCTCCAGTGAGGC | HBF | 5′ | human | dTs; lnaAs; |
| | m04 | GGAAACCAC | | | | dGs; lnaCs; |
| | | | | | | dTs; lnaCs; |
| | | | | | | dCs; lnaAs; |
| | | | | | | dGs; lnaTs; |
| | | | | | | dGs; lnaAs; |
| | | | | | | dGs; lnaGs; |
| | | | | | | dC; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 328 | HBF-XXX | TTTCTTCTCCCACCAG | HBF | 5′ | human | dTs; lnaTs; |
| | m05 | GAAACCAC | | | | dTs; lnaCs; |
| | | | | | | dTs; lnaTs; |
| | | | | | | dCs; lnaTs; |
| | | | | | | dCs; lnaCs; |
| | | | | | | dCs; lnaAs; |
| | | | | | | dCs; lnaCs; |
| | | | | | | dA; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 329 | HBF-XXX | TTTTTGTGTGATCTCT | HBF | 3′ | human | dTs; lnaTs; |
| | m06 | TAGC | | | | dTs; lnaTs; |
| | | | | | | dTs; lnaGs; |
| | | | | | | dATs; lnaGs; |
| | | | | | | dTs; lnaGs; |
| | | | | | | dAs; lnaTs; |
| | | | | | | dCs; lnaTs; |
| | | | | | | dCs; lnaTs; |
| | | | | | | dTs; lnaAs; |
| | | | | | | dGs; lnaC- |
| | | | | | | Sup |
| 330 | HBF-XXX | TTTTTGTGATCTCTTA | HBF | 3′ | human | dTs; lnaTs; |
| | m07 | GCAG | | | | dTs; lnaTs; |
| | | | | | | dTs; lnaGs; |
| | | | | | | dTs; lnaGs; |
| | | | | | | dAs; lnaTs; |
| | | | | | | dCs; lnaTs; |
| | | | | | | dCs; lnaTs; |
| | | | | | | dTs; lnaAs; |
| | | | | | | dGs; lnaCs; |
| | | | | | | dAs; lnaG- |
| | | | | | | Sup |
| 331 | HBF-XXX | TTTTTTGATCTCTTAG | HBF | 3′ | human | dTs; lnaTs; |
| | m08 | CAGA | | | | dTs; lnaTs; |
| | | | | | | dTs; lnaTs; |
| | | | | | | dGs; lnaAs; |
| | | | | | | dTs; lnaCs; |
| | | | | | | dTs; lnaCs; |
| | | | | | | dTs; lnaTs; |
| | | | | | | dAs; lnaGs; |
| | | | | | | dCs; lnaAs; |
| | | | | | | dGs; lnaA-Sup |
| 332 | SMN- | ATTTCTCTCAATCCT | SMN | 5′ | human | dAs; lnaTs; |
| | XXX | | | | | dTs; lnaTs; |
| | m02 | | | | | dCs; lnaT; |
| | | | | | | dCs; lnaT; |
| | | | | | | dCs; lnaA; |
| | | | | | | dAs; lnaT; |
| | | | | | | dCs; lnaC; |
| | | | | | | dTs-Sup |
| 333 | SMN- | GGCGTGTATATTTTT | SMN | 5′ | human | dGs; lnaGs; |
| | XXX | | | | | dCs; lnaGs; |
| | m03 | | | | | dTs; lnaGs; |
| | | | | | | dTs; lnaAs; |
| | | | | | | dTs; lnaAs; |
| | | | | | | dTs; lnaTs; |
| | | | | | | dTs; lnaTs; |
| | | | | | | dT-Sup |
| 334 | SMN- | GGTTATCGCCCTCCC | SMN | 5′ | human | dGs; lnaGs; |
| | XXX | | | | | dTs; lnaTs; |
| | m04 | | | | | dAs; lnaTs; |
| | | | | | | dCs; lnaGs; |
| | | | | | | dCs; lnaCs; |
| | | | | | | dCs; lnaTs; |
| | | | | | | dCs; lnaCs; |
| | | | | | | dC-Sup |
| 335 | SMN- | ACGACTTCCGCCGCC | SMN | 5′ | human | dAs; lnaCs; |
| | XXX | | | | | dGs; lnaAs; |
| | m05 | | | | | dCs; lnaTs; |
| | | | | | | dTs; lnaCs; |
| | | | | | | dCs; lnaGs; |
| | | | | | | dCs; lnaCs; |
| | | | | | | dGs; lnaCs; |
| | | | | | | dC-Sup |
| 336 | SMN- | ATTTCTCTCAATCCTCC | SMN | 5′ | human | dAs; lnaTs; |
| | XXX | | | | | dTs; lnaTs; |
| | m06 | | | | | dCs; lnaT; |
| | | | | | | dCs; lnaT; |
| | | | | | | dCs; lnaA; |
| | | | | | | dAs; lnaT; |
| | | | | | | dCs; lnaC; |
| | | | | | | dTs; lnaCs; |
| | | | | | | dC-Sup |
| 337 | SMN- | GGCGTGTATATTTTTCC | SMN | 5′ | human | dGs; lnaGs; |
| | XXX | | | | | dCs; lnaGs; |
| | m07 | | | | | dTs; lnaGs; |
| | | | | | | dTs; lnaAs; |
| | | | | | | dTs; lnaAs; |
| | | | | | | dTs; lnaTs; |
| | | | | | | dTs; lnaTs; |
| | | | | | | dT; lnaCs; |
| | | | | | | dC-Sup |
| 338 | SMN- | GGTTATCGCCCTCCCCC | SMN | 5′ | human | dGs; lnaGs; |
| | XXX | | | | | dTs; lnaTs; |
| | m08 | | | | | dAs; lnaTs; |
| | | | | | | dCs; lnaGs; |
| | | | | | | dCs; lnaCs; |
| | | | | | | dCs; lnaTs; |
| | | | | | | dCs; lnaCs; |
| | | | | | | dC; lnaCs; |
| | | | | | | dC-Sup |
| 339 | SMN- | ACGACTTCCGCCGCCCC | SMN | 5′ | human | dAs; lnaCs; |
| | XXX | | | | | dGs; lnaAs; |
| | m09 | | | | | dCs; lnaTs; |
| | | | | | | dTs; lnaCs; |
| | | | | | | dCs; lnaGs; |
| | | | | | | dCs; lnaCs; |
| | | | | | | dGs; lnaCs; |
| | | | | | | dC; lnaCs; |
| | | | | | | dC-Sup |
| 340 | SMN- | ATTTCTCTCAATCCTG | SMN | 5′ | human | dAs; lnaTs; |
| | XXX | GAAACCAC | | | | dTs; lnaTs; |
| | m10 | | | | | dCs; lnaT; |
| | | | | | | dCs; lnaT; |
| | | | | | | dCs; lnaA; |
| | | | | | | dAs; lnaT; |
| | | | | | | dCs; lnaC; |
| | | | | | | dTs; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 341 | SMN- | GGCGTGTATATTTTTG | SMN | 5′ | human | dGs; lnaGs; |
| | XXX | GAAACCAC | | | | dCs; lnaGs; |
| | m11 | | | | | dTs; lnaGs; |
| | | | | | | dTs; lnaAs; |
| | | | | | | dTs; lnaAs; |
| | | | | | | dTs; lnaTs; |
| | | | | | | dTs; lnaTs; |
| | | | | | | dT; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 342 | SMN- | GGTTATCGCCCTCCCG | SMN | 5′ | human | dGs; lnaGs; |
| | XXX | GAAACCAC | | | | dTs; lnaTs; |
| | m12 | | | | | dAs; lnaTs; |
| | | | | | | dCs; lnaGs; |
| | | | | | | dCs; lnaCs; |
| | | | | | | dCs; lnaTs; |
| | | | | | | dCs; lnaCs; |
| | | | | | | dC; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 343 | SMN- | ACGACTTCCGCCGCC | SMN | 5′ | human | dAs; lnaCs; |
| | XXX | GGAAACCAC | | | | dGs; lnaAs; |
| | m13 | | | | | dCs; lnaTs; |
| | | | | | | dTs; lnaCs; |
| | | | | | | dCs; lnaGs; |
| | | | | | | dCs; lnaCs; |
| | | | | | | dGs; lnaCs; |
| | | | | | | dC; lnaGs; |
| | | | | | | dGs; dAs; |
| | | | | | | dAs; dAs; |
| | | | | | | dCs; lnaCs; |
| | | | | | | dAs; lnaC- |
| | | | | | | Sup |
| 344 | SMN- | TTTTTTAATTTTTTTTT | SMN | 3′ | human | dTs; lnaTs; |
| | XXX | AAA | | | | dTs; lnaTs; |
| | m14 | | | | | dTs; lnaTs; |
| | | | | | | dAs; lnaAs; |
| | | | | | | dTs; lnaTs; |
| | | | | | | dTs; lnaTs; |
| | | | | | | dTs; lnaTs; |
| | | | | | | dTs; lnaTs; |
| | | | | | | dTs; lnaAs; |
| | | | | | | dAs; lnaA-Sup |
| 345 | SMN- | TTTTTATATGCAAAAA | SMN | 3′ | human | dTs; lnaTs; |
| | XXX | AGAA | | | | dTs; lnaTs; |
| | m15 | | | | | dTs; lnaAs; |
| | | | | | | dTs; lnaAs; |
| | | | | | | dTs; lnaGs; |
| | | | | | | dCs; lnaAs; |
| | | | | | | dAs; lnaAs; |
| | | | | | | dAs; lnaAs; |
| | | | | | | dAs; lnaGs; |
| | | | | | | dAs; lnaA-Sup |
| 346 | SMN- | TTTTTCAAAATATGGG | SMN | 3′ | human | dTs; lnaTs; |
| | XXX | CCAA | | | | dTs; lnaTs; |
| | m16 | | | | | dTs; lnaCs; |
| | | | | | | dAs; lnaAs; |
| | | | | | | dAs; lnaAs; |
| | | | | | | dTs; lnaAs; |
| | | | | | | dTs; lnaGs; |
| | | | | | | dGs; lnaGs; |
| | | | | | | dCs; lnaCs; |
| | | | | | | dAs; lnaA-Sup |

### Example 5

### Further Oligonucleotides for Increasing RNA Stability

Table 8 provides exemplary oligonucleotides for targeting the 5′ and 3′ ends of noncoding RNAs HOTAIR and ANRIL.

TABLE 8
Oligos targeting non-coding RNAs

| | | | | Target | | |
|---|---|---|---|---|---|---|
| SEQ | Oligo | | Gene | Region (5′ | | Formatted |
| ID NO | Name | Base Sequence | Name | or 3′ End) | Organism | Sequence |
| 347 | HOTAIR-1 | TTCACCACATGTAAA | HOTAIR | 3′ | Human | dTs; lnaTs; |
| | | | | | | dCs; lnaAs; |
| | | | | | | dCs; lnaCs; |
| | | | | | | dAs; lnaCs; |
| | | | | | | dAs; lnaTs; |
| | | | | | | dGs; lnaTs; |
| | | | | | | dAs; lnaAs; |
| | | | | | | dA-Sup |
| 348 | HOTAIR-2 | TTTTTTCACCACATGTAAA | HOTAIR | 3′ | Human | dTs; lnaTs; |
| | | | | | | dTs; lnaTs; |
| | | | | | | dTs; lnaTs; |
| | | | | | | dCs; lnaAs; |
| | | | | | | dCs; lnaCs; |
| | | | | | | dAs; lnaCs; |
| | | | | | | dAs; lnaTs; |
| | | | | | | dGs; lnaTs; |
| | | | | | | dAs; lnaAs; |
| | | | | | | dA- |
| | | | | | | Sup |
| 349 | HOTAIR-3 | AAATCAGGGCAGAATGT | HOTAIR | 5′ | Human | dAs; lnaAs; |
| | | | | | | dAs; lnaTs; |
| | | | | | | dCs; lnaAs; |
| | | | | | | dGs; lnaGs; |
| | | | | | | dGs; lnaCs; |
| | | | | | | dAs; lnaGs; |
| | | | | | | dAs; lnaAs; |
| | | | | | | dTs; lnaGs; |
| | | | | | | dT-Sup |
| 350 | HOTAIR-4 | AAATCAGGGCAGAATG | HOTAIR | 5′ | Human | dAs; lnaAs; |
| | | TCC | | | | dAs; lnaTs; |
| | | | | | | dCs; lnaAs; |
| | | | | | | dGs; lnaGs; |
| | | | | | | dGs; lnaCs; |
| | | | | | | dAs; lnaGs; |
| | | | | | | dAs; lnaAs; |
| | | | | | | dTs; lnaGs; |
| | | | | | | dTs; lnaCs; |
| | | | | | | dC- |
| | | | | | | Sup |
| 351 | HOTAIR-5 | AAATCAGGGCAGAATG | HOTAIR | 5′ | Human | dAs; lnaAs; |
| | | TCCAAAGGTC | | | | dAs; lnaTs; |
| | | | | | | dCs; lnaAs; |
| | | | | | | dGs; lnaGs; |
| | | | | | | dGs; lnaCs; |
| | | | | | | dAs; lnaGs; |
| | | | | | | dAs; lnaAs; |
| | | | | | | dTs; lnaGs; |
| | | | | | | dTs; lnaCs; |
| | | | | | | dCs; lnaAs; |
| | | | | | | dAs; lnaAs; |
| | | | | | | dGs; lnaGs; |
| | | | | | | dTs; dC- |
| | | | | | | Sup |
| 352 | HOTAIR-6 | AAATCAGGGCAGAATG | HOTAIR | 5′ and 3′ | Human | dAs; lnaAs; |
| | | TTTTTTTCACCACATGTA | | | | dAs; lnaTs; |
| | | AA | | | | dCs; lnaAs; |
| | | | | | | dGs; lnaGs; |
| | | | | | | dGs; lnaCs; |
| | | | | | | dAs; lnaGs; |
| | | | | | | dAs; lnaAs; |
| | | | | | | dTs; lnaGs; |
| | | | | | | dTs; lnaTs; |
| | | | | | | dTs; lnaTs; |
| | | | | | | dTs; lnaTs; |
| | | | | | | dTs; lnaCs; |
| | | | | | | dAs; lnaCs; |
| | | | | | | dCs; lnaAs; |
| | | | | | | dCs; lnaAs; |
| | | | | | | dTs; lnaGs; |
| | | | | | | dTs; lnaAs; |
| | | | | | | dAs; dA- |
| | | | | | | Sup |
| 353 | ANRIL-1 | TTATTGTCTGAGCCC | ANRIL | 3′ | Human | dTs; lnaTs; |
| | | | | | | dAs; lnaTs; |
| | | | | | | dTs; lnaGs; |
| | | | | | | dTs; lnaCs; |
| | | | | | | dTs; lnaGs; |
| | | | | | | dAs; lnaGs; |
| | | | | | | dCs; lnaCs; |
| | | | | | | dC-Sup |
| 354 | ANRIL-2 | TTTTTATTGTCTGAGCCC | ANRIL | 3′ | Human | dTs; lnaTs; |
| | | | | | | dTs; lnaTs; |
| | | | | | | dTs; lnaAs; |
| | | | | | | dTs; lnaTs; |
| | | | | | | dGs; lnaTs; |
| | | | | | | dCs; lnaTs; |
| | | | | | | dGs; lnaAs; |
| | | | | | | dGs; lnaCs; |
| | | | | | | dCs; dC-Sup |
| 355 | ANRIL-3 | TCAGGTGACGGATGT | ANRIL | 5′ | Human | dTs; lnaCs; |
| | | | | | | dAs; lnaGs; |
| | | | | | | dGs; lnaTs; |
| | | | | | | dGs; lnaAs; |
| | | | | | | dCs; lnaGs; |
| | | | | | | dGs; lnaAs; |
| | | | | | | dTs; lnaGs; |
| | | | | | | dT-Sup |
| 356 | ANRIL-4 | TCAGGTGACGGATGTCC | ANRIL | 5′ | Human | dTs; lnaCs; |
| | | | | | | dAs; lnaGs; |
| | | | | | | dGs; lnaTs; |
| | | | | | | dGs; lnaAs; |
| | | | | | | dCs; lnaGs; |
| | | | | | | dGs; lnaAs; |
| | | | | | | dTs; lnaGs; |
| | | | | | | dTs; lnaCs; |
| | | | | | | dC-Sup |
| 357 | ANRIL-5 | TCAGGTGACGGATGTCC | ANRIL | 5′ | Human | dTs; lnaCs; |
| | | AAAGGTC | | | | dAs; lnaGs; |
| | | | | | | dGs; lnaTs; |
| | | | | | | dGs; lnaAs; |
| | | | | | | dCs; lnaGs; |
| | | | | | | dGs; lnaAs; |
| | | | | | | dTs; lnaGs; |
| | | | | | | dTs; lnaCs; |
| | | | | | | dCs; lnaAs; |
| | | | | | | dAs; lnaAs; |
| | | | | | | dGs; lnaGs; |
| | | | | | | dTs; dC-Sup |
| 358 | ANRIL-6 | TCAGGTGACGGATGTTT | ANRIL | 5′ and 3′ | Human | dTs; lnaCs; |
| | | TTTATTGTCTGAGCCC | | | | dAs; lnaGs; |
| | | | | | | dGs; lnaTs; |
| | | | | | | dGs; lnaAs; |
| | | | | | | dCs; lnaGs; |
| | | | | | | dGs; lnaAs; |
| | | | | | | dTs; lnaGs; |
| | | | | | | dTs; lnaTs; |
| | | | | | | dTs; lnaTs; |
| | | | | | | dTs; lnaTs; |
| | | | | | | dAs; lnaTs; |
| | | | | | | dTs; lnaGs; |
| | | | | | | dTs; lnaCs; |
| | | | | | | dTs; lnaGs; |
| | | | | | | dAs; lnaGs; |
| | | | | | | dCs; lnaCs; |
| | | | | | | dC-Sup |

### Example 6

### Other Stability Oligos

Table 9 provides further exemplary RNA stability oligos for multiple human and mouse genes.

| SEQ | Oligo | | | Target | | Formatted |
|---|---|---|---|---|---|---|
| ID NO | Name | Base Sequence | Gene Name | Region | Organism | Sequence |
| 359 | FOXP3- | TGTGGGGAGCTCGGC | FOXP3 | 3′ | human | dTs; lnaGs; |
| | 61 m02 | | | | | dTs; lnaGs; |
| | | | | | | dGs; lnaGs; |
| | | | | | | dGs; lnaAs; |
| | | | | | | dGs; lnaCs; |
| | | | | | | dTs; lnaCs; |
| | | | | | | dGs; lnaGs; |
| | | | | | | dC-Sup |
| 360 | FOXP3- | GGGGAGCTCGGCTGC | FOXP3 | 3′ | human | dGs; lnaGs; |
| | 62 m02 | | | | | dGs; lnaGs; |
| | | | | | | dAs; lnaGs; |
| | | | | | | dCs; lnaTs; |
| | | | | | | dCs; lnaGs; |
| | | | | | | dGs; lnaCs; |
| | | | | | | dTs; lnaGs; |
| | | | | | | dC-Sup |
| 361 | FOXP3- | TTTTTGTGGGGAGCTC | FOXP3 | 3′ | human | dTs; lnaTs; |
| | 63 m02 | GGC | | | | dTs; lnaTs; |
| | | | | | | dTs; lnaGs; |
| | | | | | | dTs; lnaGs; |
| | | | | | | dGs; lnaGs; |
| | | | | | | dGs; lnaAs; |
| | | | | | | dGs; lnaCs; |
| | | | | | | dTs; lnaCs; |
| | | | | | | dGs; lnaGs; |
| | | | | | | dC-Sup |
| 362 | FOXP3- | TTTTGGGGAGCTCGGC | FOXP3 | 3′ | human | dTs; lnaTs; |
| | 64 m02 | TGC | | | | dTs; lnaTs; |
| | | | | | | dGs; lnaGs; |
| | | | | | | dGs; lnaGs; |
| | | | | | | dAs; lnaGs; |
| | | | | | | dCs; lnaTs; |
| | | | | | | dCs; lnaGs; |
| | | | | | | dGs; lnaCs; |
| | | | | | | dTs; lnaGs; |
| | | | | | | dC-Sup |
| 363 | FOXP3- | TTGTCCAAGGGCAGG | FOXP3 | 5′ | human | dTs; lnaTs; |
| | 65 m02 | | | | | dGs; lnaTs; |
| | | | | | | dCs; lnaCs; |
| | | | | | | dAs; lnaAs; |
| | | | | | | dGs; lnaGs; |
| | | | | | | dGs; lnaCs; |
| | | | | | | dAs; lnaGs; |
| | | | | | | dG-Sup |
| 364 | FOXP3- | TCGATGAGTGTGTGC | FOXP3 | 5′ | human | dTs; lnaCs; |
| | 66 m02 | | | | | dGs; lnaAs; |
| | | | | | | dTs; lnaGs; |
| | | | | | | dAs; lnaGs; |
| | | | | | | dTs; lnaGs; |
| | | | | | | dTs; lnaGs; |
| | | | | | | dTs; lnaGs; |
| | | | | | | dC-Sup |
| 365 | FOXP3- | AGAAGAAAAACCACG | FOXP3 | 5′ | human | dAs; lnaGs; |
| | 67 m02 | | | | | dAs; lnaAs; |
| | | | | | | dGs; lnaAs; |
| | | | | | | dAs; lnaAs; |
| | | | | | | dAs; lnaAs; |
| | | | | | | dCs; lnaCs; |
| | | | | | | dAs; lnaCs; |
| | | | | | | dG-Sup |
| 366 | FOXP3- | AATATGATTTCTTCC | FOXP3 | 5′ | human | dAs; lnaAs; |
| | 68 m02 | | | | | dTs; lnaAs; |
| | | | | | | dTs; lnaGs; |
| | | | | | | dAs; lnaTs; |
| | | | | | | dTs; lnaTs; |
| | | | | | | dCs; lnaTs; |
| | | | | | | dTs; lnaCs; |
| | | | | | | dC-Sup |
| 367 | FOXP3- | GAGATGGGGGACATG | FOXP3 | 5′ | human | dGs; lnaAs; |
| | 69 m02 | | | | | dGs; lnaAs; |
| | | | | | | dTs; lnaGs; |
| | | | | | | dGs; lnaGs; |
| | | | | | | dGs; lnaGs; |
| | | | | | | dAs; lnaCs; |
| | | | | | | dAs; lnaTs; |
| | | | | | | dG-Sup |
| 368 | PTEN- | TTCAGTTTATTCAAG | PTEN | 3′ | human | dTs; lnaTs; |
| | 101 m02 | | | | | dCs; lnaAs; |
| | | | | | | dGs; lnaTs; |
| | | | | | | dTs; lnaTs; |
| | | | | | | dAs; lnaTs; |
| | | | | | | dTs; lnaCs; |
| | | | | | | dAs; lnaAs; |
| | | | | | | dG-Sup |
| 369 | PTEN- | CTGTCTCCACTTTTT | PTEN | 3′ | human | dCs; lnaTs; |
| | 102 m02 | | | | | dGs; lnaTs; |
| | | | | | | dCs; lnaTs; |
| | | | | | | dCs; lnaCs; |
| | | | | | | dAs; lnaCs; |
| | | | | | | dTs; lnaTs; |
| | | | | | | dTs; lnaTs; |
| | | | | | | dT-Sup |
| 370 | PTEN- | TGGAATAAAACGGG | PTEN | 3′ | human | dTs; lnaGs; |
| | 103 m02 | | | | | dGs; lnaAs; |
| | | | | | | dAs; lnaTs; |
| | | | | | | dAs; lnaAs; |
| | | | | | | dAs; lnaAs; |
| | | | | | | dCs; lnaGs; |
| | | | | | | dGs; lnaG- |
| | | | | | | Sup |
| 371 | PTEN- | ACAATTGAGAAAACA | PTEN | 3′ | human | dAs; lnaCs; |
| | 104 m02 | | | | | dAs; lnaAs; |
| | | | | | | dTs; lnaTs; |
| | | | | | | dGs; lnaAs; |
| | | | | | | dGs; lnaAs; |
| | | | | | | dAs; lnaAs; |
| | | | | | | dAs; lnaCs; |
| | | | | | | dA-Sup |
| 372 | PTEN- | CAGTTTTAAGTGGAG | PTEN | 3′ | human | dCs; lnaAs; |
| | 105 m02 | | | | | dGs; lnaTs; |
| | | | | | | dTs; lnaTs; |
| | | | | | | dTs; lnaAs; |
| | | | | | | dAs; lnaGs; |
| | | | | | | dTs; lnaGs; |
| | | | | | | dGs; lnaAs; |
| | | | | | | dG-Sup |
| 373 | PTEN- | TGACAAGAATGAGAC | PTEN | 3′ | human | dTs; lnaGs; |
| | 106 m02 | | | | | dAs; lnaCs; |
| | | | | | | dAs; lnaAs; |
| | | | | | | dGs; lnaAs; |
| | | | | | | dAs; lnaTs; |
| | | | | | | dGs; lnaAs; |
| | | | | | | dGs; lnaAs; |
| | | | | | | dC-Sup |
| 374 | PTEN- | CCGGGCGAGGGGAGG | PTEN | 5′ | human | dCs; lnaCs; |
| | 107 m02 | | | | | dGs; lnaGs; |
| | | | | | | dGs; lnaCs; |
| | | | | | | dGs; lnaAs; |
| | | | | | | dGs; lnaGs; |
| | | | | | | dGs; lnaGs; |
| | | | | | | dAs; lnaGs; |
| | | | | | | dG-Sup |
| 375 | PTEN- | CCGCCGGCCTGCCCG | PTEN | 5′ | human | dCs; lnaCs; |
| | 108 m02 | | | | | dGs; lnaCs; |
| | | | | | | dCs; lnaGs; |
| | | | | | | dGs; lnaCs; |
| | | | | | | dCs; lnaTs; |
| | | | | | | dGs; lnaCs; |
| | | | | | | dCs; lnaCs; |
| | | | | | | dG-Sup |
| 376 | PTEN- | CGAGCGCGTATCCTG | PTEN | 5′ | human | dCs; lnaGs; |
| | 109 m02 | | | | | dAs; lnaGs; |
| | | | | | | dCs; lnaGs; |
| | | | | | | dCs; lnaGs; |
| | | | | | | dTs; lnaAs; |
| | | | | | | dTs; lnaCs; |
| | | | | | | dCs; lnaTs; |
| | | | | | | dG-Sup |
| 377 | PTEN- | CTGCTTCTCCTCAGC | PTEN | 5′ | human | dCs; lnaTs; |
| | 110 m02 | | | | | dGs; lnaCs; |
| | | | | | | dTs; lnaTs; |
| | | | | | | dCs; lnaTs; |
| | | | | | | dCs; lnaCs; |
| | | | | | | dTs; lnaCs; |
| | | | | | | dAs; lnaGs; |
| | | | | | | dC-Sup |
| 378 | PTEN- | TTTTCAGTTTATTCAAG | PTEN | 3′ | human | dTs; lnaTs; |
| | 111 m02 | | | | | dTs; lnaTs; |
| | | | | | | dCs; lnaAs; |
| | | | | | | dGs; lnaTs; |
| | | | | | | dTs; lnaTs; |
| | | | | | | dAs; lnaTs; |
| | | | | | | dTs; lnaCs; |
| | | | | | | dAs; lnaAs; |
| | | | | | | dG-Sup |
| 379 | PTEN- | TTTTCTGTCTCCACTTTTT | PTEN | 3′ | human | dTs; lnaTs; |
| | 112 m02 | | | | | dTs; lnaTs; |
| | | | | | | dCs; lnaTs; |
| | | | | | | dGs; lnaTs; |
| | | | | | | dCs; lnaTs; |
| | | | | | | dCs; lnaCs; |
| | | | | | | dAs; lnaCs; |
| | | | | | | dTs; lnaTs; |
| | | | | | | dTs; lnaTs; |
| | | | | | | dT-Sup |
| 380 | PTEN- | TTTTTGGAATAAAACG | PTEN | 3′ | human | dTs; lnaTs; |
| | 113 m02 | GG | | | | dTs; lnaTs; |
| | | | | | | dTs; lnaGs; |
| | | | | | | dGs; lnaAs; |
| | | | | | | dAs; lnaTs; |
| | | | | | | dAs; lnaAs; |
| | | | | | | dAs; lnaAs; |
| | | | | | | dCs; lnaGs; |
| | | | | | | dGs; lnaG- |
| | | | | | | Sup |
| 381 | PTEN- | TTTTACAATTGAGAAAA | PTEN | 3′ | human | dTs; lnaTs; |
| | 114 m02 | CA | | | | dTs; lnaTs; |
| | | | | | | dAs; lnaCs; |
| | | | | | | dAs; lnaAs; |
| | | | | | | dTs; lnaTs; |
| | | | | | | dGs; lnaAs; |
| | | | | | | dGs; lnaAs; |
| | | | | | | dAs; lnaAs; |
| | | | | | | dAs; lnaCs; |
| | | | | | | dA-Sup |
| 382 | PTEN- | TTTTCAGTTTTAAGTGG | PTEN | 3′ | human | dTs; lnaTs; |
| | 115 m02 | AG | | | | dTs; lnaTs; |
| | | | | | | dCs; lnaAs; |
| | | | | | | dGs; lnaTs; |
| | | | | | | dTs; lnaTs; |
| | | | | | | dTs; lnaAs; |
| | | | | | | dAs; lnaGs; |
| | | | | | | dTs; lnaGs; |
| | | | | | | dGs; lnaAs; |
| | | | | | | dG-Sup |
| 383 | PTEN- | TTTTTGACAAGAATGA | PTEN | 3′ | human | dTs; lnaTs; |
| | 116 m02 | GAC | | | | dTs; lnaTs; |
| | | | | | | dTs; lnaGs; |
| | | | | | | dAs; lnaCs; |
| | | | | | | dAs; lnaAs; |
| | | | | | | dGs; lnaAs; |
| | | | | | | dAs; lnaTs; |
| | | | | | | dGs; lnaAs; |
| | | | | | | dGs; lnaAs; |
| | | | | | | dC-Sup |
| 384 | NFE2L2- | AACAGTCATAATAAT | NFE2L2 | 3′ | human | dAs; lnaAs; |
| | 01 m02 | | | | | dCs; lnaAs; |
| | | | | | | dGs; lnaTs; |
| | | | | | | dCs; lnaAs; |
| | | | | | | dTs; lnaAs; |
| | | | | | | dAs; lnaTs; |
| | | | | | | dAs; lnaAs; |
| | | | | | | dT-Sup |
| 385 | NFE2L2- | TAATTTAACAGTCAT | NFE2L2 | 3′ | human | dTs; lnaAs; |
| | 02 m02 | | | | | dAs; lnaTs; |
| | | | | | | dTs; lnaTs; |
| | | | | | | dAs; lnaAs; |
| | | | | | | dCs; lnaAs; |
| | | | | | | dGs; lnaTs; |
| | | | | | | dCs; lnaAs; |
| | | | | | | dT-Sup |
| 386 | NFE2L2- | GCACGCTATAAAGCA | NFE2L2 | 5′ | human | dGs; lnaCs; |
| | 03 m02 | | | | | dAs; lnaCs; |
| | | | | | | dGs; lnaCs; |
| | | | | | | dTs; lnaAs; |
| | | | | | | dTs; lnaAs; |
| | | | | | | dAs; lnaAs; |
| | | | | | | dGs; lnaCs; |
| | | | | | | dA-Sup |
| 387 | NFE2L2- | CCCGGGGCTGGGCTT | NFE2L2 | 5′ | human | dCs; lnaCs; |
| | 04 m02 | | | | | dCs; lnaGs; |
| | | | | | | dGs; lnaGs; |
| | | | | | | dGs; lnaCs; |
| | | | | | | dTs; lnaGs; |
| | | | | | | dGs; lnaGs; |
| | | | | | | dCs; lnaTs; |
| | | | | | | dT-Sup |
| 388 | NFE2L2- | CCCCGCTCCGCCTCC | NFE2L2 | 5′ | human | dCs; lnaCs; |
| | 05 m02 | | | | | dCs; lnaCs; |
| | | | | | | dGs; lnaCs; |
| | | | | | | dTs; lnaCs; |
| | | | | | | dCs; lnaGs; |
| | | | | | | dCs; lnaCs; |
| | | | | | | dTs; lnaCs; |
| | | | | | | dC-Sup |
| 389 | NFE2L2- | GCGCCTCCCTGATTT | NFE2L2 | 5′ | human | dGs; lnaCs; |
| | 06 m02 | | | | | dGs; lnaCs; |
| | | | | | | dCs; lnaTs; |
| | | | | | | dCs; lnaCs; |
| | | | | | | dCs; lnaTs; |
| | | | | | | dGs; lnaAs; |
| | | | | | | dTs; lnaTs; |
| | | | | | | dT-Sup |
| 390 | NFE2L2- | TCGCCGCGGTGGCTG | NFE2L2 | 5′ | human | dTs; lnaCs; |
| | 07 m02 | | | | | dGs; lnaCs; |
| | | | | | | dCs; lnaGs; |
| | | | | | | dCs; lnaGs; |
| | | | | | | dGs; lnaTs; |
| | | | | | | dGs; lnaGs; |
| | | | | | | dCs; lnaTs; |
| | | | | | | dG-Sup |
| 391 | NFE2L2- | CAGCGAATGGTCGCG | NFE2L2 | 5′ | human | dCs; lnaAs; |
| | 08 m02 | | | | | dGs; lnaCs; |
| | | | | | | dGs; lnaAs; |
| | | | | | | dAs; lnaTs; |
| | | | | | | dGs; lnaGs; |
| | | | | | | dTs; lnaCs; |
| | | | | | | dGs; lnaCs; |
| | | | | | | dG-Sup |
| 392 | NFE2L2- | TTTTTAACAGTCATAAT | NFE2L2 | 3′ | human | dTs; lnaTs; |
| | 09 m02 | AAT | | | | dTs; lnaTs; |
| | | | | | | dTs; lnaAs; |
| | | | | | | dAs; lnaCs; |
| | | | | | | dAs; lnaGs; |
| | | | | | | dTs; lnaCs; |
| | | | | | | dAs; lnaTs; |
| | | | | | | dAs; lnaAs; |
| | | | | | | dTs; lnaAs; |
| | | | | | | dAs; lnaT-Sup |
| 393 | NFE2L2- | TTTTTAATTTAACAGTC | NFE2L2 | 3′ | human | dTs; lnaTs; |
| | 10 m02 | AT | | | | dTs; lnaTs; |
| | | | | | | dTs; lnaAs; |
| | | | | | | dAs; lnaTs; |
| | | | | | | dTs; lnaTs; |
| | | | | | | dAs; lnaAs; |
| | | | | | | dCs; lnaAs; |
| | | | | | | dGs; lnaTs; |
| | | | | | | dCs; lnaAs; |
| | | | | | | dT-Sup |
| 394 | ATP2A2- | GCGGCGGCTGCTCTA | ATP2A2 | 5′ | human | dGs; lnaCs; |
| | 56 m02 | | | | | dGs; lnaGs; |
| | | | | | | dCs; lnaGs; |
| | | | | | | dGs; lnaCs; |
| | | | | | | dTs; lnaGs; |
| | | | | | | dCs; lnaTs; |
| | | | | | | dCs; lnaTs; |
| | | | | | | dA-Sup |
| 395 | ATP2A2- | TTATCGGCCGCTGCC | ATP2A2 | 5′ | human | dTs; lnaTs; |
| | 34 m02 | | | | | dAs; lnaTs; |
| | | | | | | dCs; lnaGs; |
| | | | | | | dGs; lnaCs; |
| | | | | | | dCs; lnaGs; |
| | | | | | | dCs; lnaTs; |
| | | | | | | dGs; lnaCs; |
| | | | | | | dC-Sup |
| 396 | ATP2A2- | GCGTCGGGGACGGCT | ATP2A2 | 5′ | human | dGs; lnaCs; |
| | 57 m02 | | | | | dGs; lnaTs; |
| | | | | | | dCs; lnaGs; |
| | | | | | | dGs; lnaGs; |
| | | | | | | dGs; lnaAs; |
| | | | | | | dCs; lnaGs; |
| | | | | | | dGs; lnaCs; |
| | | | | | | dT-Sup |
| 397 | ATP2A2- | GCGGAGGAAACTGCG | ATP2A2 | 5′ | human | dGs; lnaCs; |
| | 58 m02 | | | | | dGs; lnaGs; |
| | | | | | | dAs; lnaGs; |
| | | | | | | dGs; lnaAs; |
| | | | | | | dAs; lnaAs; |
| | | | | | | dCs; lnaTs; |
| | | | | | | dGs; lnaCs; |
| | | | | | | dG-Sup |
| 398 | ATP2A2- | GCCGCACGCCCGACA | ATP2A2 | 5′ | human | dGs; lnaCs; |
| | 59 m02 | | | | | dCs; lnaGs; |
| | | | | | | dCs; lnaAs; |
| | | | | | | dCs; lnaGs; |
| | | | | | | dCs; lnaCs; |
| | | | | | | dCs; lnaGs; |
| | | | | | | dAs; lnaCs; |
| | | | | | | dA-Sup |
| 399 | ATP2A2- | CCTGACCCACCCTCC | ATP2A2 | 5′ | human | dCs; lnaCs; |
| | 60 m02 | | | | | dTs; lnaGs; |
| | | | | | | dAs; lnaCs; |
| | | | | | | dCs; lnaCs; |
| | | | | | | dAs; lnaCs; |
| | | | | | | dCs; lnaCs; |
| | | | | | | dTs; lnaCs; |
| | | | | | | dC-Sup |
| 400 | ATP2A2- | AGGGCAGGCCGCGGC | ATP2A2 | 5′ | human | dAs; lnaGs; |
| | 61 m02 | | | | | dGs; lnaGs; |
| | | | | | | dCs; lnaAs; |
| | | | | | | dGs; lnaGs; |
| | | | | | | dCs; lnaCs; |
| | | | | | | dGs; lnaCs; |
| | | | | | | dGs; lnaGs; |
| | | | | | | dC-Sup |
| 401 | ATP2A2- | CTGAATCACCCCGCG | ATP2A2 | 5′ | human | dCs; lnaTs; |
| | 62 m02 | | | | | dGs; lnaAs; |
| | | | | | | dAs; lnaTs; |
| | | | | | | dCs; lnaAs; |
| | | | | | | dCs; lnaCs; |
| | | | | | | dCs; lnaCs; |
| | | | | | | dGs; lnaCs; |
| | | | | | | dG-Sup |
| 402 | ATP2A2- | GGCCCCGAGCTCCGC | ATP2A2 | 5′ | human | dGs; lnaGs; |
| | 63 m02 | | | | | dCs; lnaCs; |
| | | | | | | dCs; lnaCs; |
| | | | | | | dGs; lnaAs; |
| | | | | | | dGs; lnaCs; |
| | | | | | | dTs; lnaCs; |
| | | | | | | dCs; lnaGs; |
| | | | | | | dC-Sup |
| 403 | ATP2A2- | GCGGCTGCTCTAATA | ATP2A2 | 5′ | human | dGs; lnaCs; |
| | 64 m02 | | | | | dGs; lnaGs; |
| | | | | | | dCs; lnaTs; |
| | | | | | | dGs; lnaCs; |
| | | | | | | dTs; lnaCs; |
| | | | | | | dTs; lnaAs; |
| | | | | | | dAs; lnaTs; |
| | | | | | | dA-Sup |
| 404 | ATP2A2- | CGCCGCGGCATGTGG | ATP2A2 | 5′ | human | dCs; lnaGs; |
| | 65 m02 | | | | | dCs; lnaCs; |
| | | | | | | dGs; lnaCs; |
| | | | | | | dGs; lnaGs; |
| | | | | | | dCs; lnaAs; |
| | | | | | | dTs; lnaGs; |
| | | | | | | dTs; lnaGs; |
| | | | | | | dG-Sup |
| 405 | ATP2A2- | CCCTCCTCCTCTTGC | ATP2A2 | 5′ | human | dCs; lnaCs; |
| | 66 m02 | | | | | dCs; lnaTs; |
| | | | | | | dCs; lnaCs; |
| | | | | | | dTs; lnaCs; |
| | | | | | | dCs; lnaTs; |
| | | | | | | dCs; lnaTs; |
| | | | | | | dTs; lnaGs; |
| | | | | | | dC-Sup |
| 406 | ATP2A2- | GGCCGCGGGCTCGTG | ATP2A2 | 5′ | human | dGs; lnaGs; |
| | 67 m02 | | | | | dCs; lnaCs; |
| | | | | | | dGs; lnaCs; |
| | | | | | | dGs; lnaGs; |
| | | | | | | dGs; lnaCs; |
| | | | | | | dTs; lnaCs; |
| | | | | | | dGs; lnaTs; |
| | | | | | | dG-Sup |
| 407 | ATP2A2- | GTTATTTTTCTCTGT | ATP2A2 | 3′ | human | dGs; lnaTs; |
| | 68 m02 | | | | | dTs; lnaAs; |
| | | | | | | dTs; lnaTs; |
| | | | | | | dTs; lnaTs; |
| | | | | | | dTs; lnaCs; |
| | | | | | | dTs; lnaCs; |
| | | | | | | dTs; lnaGs; |
| | | | | | | dT-Sup |
| 408 | ATP2A2- | ATTTAAAATGTTTTA | ATP2A2 | 3′ | human | dAs; lnaTs; |
| | 69 m02 | | | | | dTs; lnaTs; |
| | | | | | | dAs; lnaAs; |
| | | | | | | dAs; lnaAs; |
| | | | | | | dTs; lnaGs; |
| | | | | | | dTs; lnaTs; |
| | | | | | | dTs; lnaTs; |
| | | | | | | dA-Sup |
| 409 | ATP2A2- | TCTCTGTCCATTTAA | ATP2A2 | 3′ | human | dTs; lnaCs; |
| | 70 m02 | | | | | dTs; lnaCs; |
| | | | | | | dTs; lnaGs; |
| | | | | | | dTs; lnaCs; |
| | | | | | | dCs; lnaAs; |
| | | | | | | dTs; lnaTs; |
| | | | | | | dTs; lnaAs; |
| | | | | | | dA-Sup |
| 410 | ATP2A2- | TCATTTGGTCATGTG | ATP2A2 | 3′ | human | dTs; lnaCs; |
| | 71 m02 | | | | | dAs; lnaTs; |
| | | | | | | dTs; lnaTs; |
| | | | | | | dGs; lnaGs; |
| | | | | | | dTs; lnaCs; |
| | | | | | | dAs; lnaTs; |
| | | | | | | dGs; lnaTs; |
| | | | | | | dG-Sup |
| 411 | ATP2A2- | TAGTTCTCTGTACAT | ATP2A2 | 3′ | human | dTs; lnaAs; |
| | 72 m02 | | | | | dGs; lnaTs; |
| | | | | | | dTs; lnaCs; |
| | | | | | | dTs; lnaCs; |
| | | | | | | dTs; lnaGs; |
| | | | | | | dTs; lnaAs; |
| | | | | | | dCs; lnaAs; |
| | | | | | | dT-Sup |
| 412 | ATP2A2- | TCTGCTGGCTCAACT | ATP2A2 | 3′ | human | dTs; lnaCs; |
| | 73 m02 | | | | | dTs; lnaGs; |
| | | | | | | dCs; lnaTs; |
| | | | | | | dGs; lnaGs; |
| | | | | | | dCs; lnaTs; |
| | | | | | | dCs; lnaAs; |
| | | | | | | dAs; lnaCs; |
| | | | | | | dT-Sup |
| 413 | ATP2A2- | ATCATAGAATAGATT | ATP2A2 | 3′ | human | dAs; lnaTs; |
| | 74 m02 | | | | | dCs; lnaAs; |
| | | | | | | dTs; lnaAs; |
| | | | | | | dGs; lnaAs; |
| | | | | | | dAs; lnaTs; |
| | | | | | | dAs; lnaGs; |
| | | | | | | dAs; lnaTs; |
| | | | | | | dT-Sup |
| 414 | ATP2A2- | TTATCATAGAATAGA | ATP2A2 | 3′ | human | dTs; lnaTs; |
| | 75 m02 | | | | | dAs; lnaTs; |
| | | | | | | dCs; lnaAs; |
| | | | | | | dTs; lnaAs; |
| | | | | | | dGs; lnaAs; |
| | | | | | | dAs; lnaTs; |
| | | | | | | dAs; lnaGs; |
| | | | | | | dA-Sup |
| 415 | ATP2A2- | AATTGACATTTAGCA | ATP2A2 | 3′ | human | dAs; lnaAs; |
| | 76 m02 | | | | | dTs; lnaTs; |
| | | | | | | dGs; lnaAs; |
| | | | | | | dCs; lnaAs; |
| | | | | | | dTs; lnaTs; |
| | | | | | | dTs; lnaAs; |
| | | | | | | dGs; lnaCs; |
| | | | | | | dA-Sup |
| 416 | ATP2A2- | GACATTTAGCATTTT | ATP2A2 | 3′ | human | dGs; lnaAs; |
| | 77 m02 | | | | | dCs; lnaAs; |
| | | | | | | dTs; lnaTs; |
| | | | | | | dTs; lnaAs; |
| | | | | | | dGs; lnaCs; |
| | | | | | | dAs; lnaTs; |
| | | | | | | dTs; lnaTs; |
| | | | | | | dT-Sup |
| 417 | ATP2A2- | TTAACCATTCAACAC | ATP2A2 | 3′ | human | dTs; lnaTs; |
| | 78 m02 | | | | | dAs; lnaAs; |
| | | | | | | dCs; lnaCs; |
| | | | | | | dAs; lnaTs; |
| | | | | | | dTs; lnaCs; |
| | | | | | | dAs; lnaAs; |
| | | | | | | dCs; lnaAs; |
| | | | | | | dC-Sup |
| 418 | mKLF4- | CTTGGCCGGGGAACT | KLF4 | 5′ | mouse | dCs; lnaTs; |
| | 01 m02 | | | | | dTs; lnaGs; |
| | | | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dT-Sup |
| 419 | mKLF4- | GCCGGGGAACTGCCG | KLF4 | 5′ | mouse | dGs; lnaCs; |
| | 02 m02 | | | | | dCs; lnaGs; |
| | | | | | | dGs; lnaGs; |
| | | | | | | dGs; lnaAs; |
| | | | | | | dAs; lnaCs; |
| | | | | | | dTs; lnaGs; |
| | | | | | | dCs; lnaCs; |
| | | | | | | dG-Sup |
| 420 | mKLF4- | CGCCCGGAGCCGCGC | KLF4 | 5′ | mouse | dCs; lnaGs; |
| | 03 m02 | | | | | dCs; lnaCs; |
| | | | | | | dCs; lnaGs; |
| | | | | | | dGs; lnaAs; |
| | | | | | | dGs; lnaCs; |
| | | | | | | dCs; lnaGs; |
| | | | | | | dCs; lnaGs; |
| | | | | | | dC-Sup |
| 421 | mKLF4- | CTTGGCCGGGGAAC | KLF4 | 5′ | mouse | dCs; lnaTs; |
| | 04 m02 | TCC | | | | dTs; lnaGs; |
| | | | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dTs; lnaCs; |
| | | | | | | dC-Sup |
| 422 | mKLF4- | GCCGGGGAACTGCC | KLF4 | 5′ | mouse | dGs; lnaCs; |
| | 05 m02 | GC | | | | dCs; lnaGs; |
| | | | | | | dGs; lnaGs; |
| | | | | | | dGs; lnaAs; |
| | | | | | | dAs; lnaCs; |
| | | | | | | dTs; lnaGs; |
| | | | | | | dCs; lnaCs; |
| | | | | | | dGs; lnaC- |
| | | | | | | Sup |
| 423 | mKLF4- | CGCCCGGAGCCGCG | KLF4 | 5′ | mouse | dCs; lnaGs; |
| | 06 m02 | CC | | | | dCs; lnaCs; |
| | | | | | | dCs; lnaGs; |
| | | | | | | dGs; lnaAs; |
| | | | | | | dGs; lnaCs; |
| | | | | | | dCs; lnaGs; |
| | | | | | | dCs; lnaGs; |
| | | | | | | dCs; lnaC- |
| | | | | | | Sup |
| 424 | mKLF4- | CTTGGCCGGGGAAC | KLF4 | 5′ and | mouse | dCs; lnaTs; |
| | 07 m02 | TATAAAATTC | | 3′ | | dTs; lnaGs; |
| | | | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dTs; lnaAs; |
| | | | | | | dTs; dAs; |
| | | | | | | dAs; dAs; |
| | | | | | | dAs; lnaTs; |
| | | | | | | dTs; lnaC- |
| | | | | | | Sup |
| 425 | mKLF4- | CTTGGCCGGGGAAC | KLF4 | 5′ and | mouse | dCs; lnaTs; |
| | 08 m02 | TTTTTGTCGTTCAGAT | | 3′ | | dTs; lnaGs; |
| | | AAAA | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dTs; lnaTs; |
| | | | | | | dTs; lnaTs; |
| | | | | | | dTs; lnaGs; |
| | | | | | | dTs; lnaCs; |
| | | | | | | dGs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dAs; lnaA-Sup |
| 426 | mKLF4- | CTTGGCCGGGGAAC | KLF4 | 5′ and | mouse | dCs; lnaTs; |
| | 09 m02 | TTTTTCAGATAAAAT | | 3′ | | dTs; lnaGs; |
| | | ATT | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dTs; lnaTs; |
| | | | | | | dTs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dAs; lnaAs; |
| | | | | | | dTs; lnaAs; |
| | | | | | | dTs; lnaT- |
| | | | | | | Sup |
| 427 | mKLF4- | CTTGGCCGGGGAAC | KLF4 | 5′ and | mouse | dCs; lnaTs; |
| | 10 m02 | TGTCGTTCAGATAAAA | | 3′ | | dTs; lnaGs; |
| | | | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dTs; lnaGs; |
| | | | | | | dTs; lnaCs; |
| | | | | | | dGs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dAs; lnaA-Sup |
| 428 | mKLF4- | CTTGGCCGGGGAAC | KLF4 | 5′ and | mouse | dCs; lnaTs; |
| | 11 m02 | TTTCAGATAAAATATT | | 3′ | | dTs; lnaGs; |
| | | | | | | dGs; lnaCs; |
| | | | | | | dCs; lnaGs; |
| | | | | | | dGs; lnaGs; |
| | | | | | | dGs; lnaAs; |
| | | | | | | dAs; lnaCs; |
| | | | | | | dTs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dAs; lnaAs; |
| | | | | | | dTs; lnaAs; |
| | | | | | | dTs; lnaT- |
| | | | | | | Sup |
| 429 | mKLF4- | CCGGGGAACTTTTTG | KLF4 | 5′ and | mouse | dCs; lnaCs; |
| | 12 m02 | TCGTTCAGA | | 3′ | | dGs; lnaGs; |
| | | | | | | dGs; lnaGs; |
| | | | | | | dAs; lnaAs; |
| | | | | | | dCs; lnaTs; |
| | | | | | | dTs; lnaTs; |
| | | | | | | dTs; lnaTs; |
| | | | | | | dGs; lnaTs; |
| | | | | | | dCs; lnaGs; |
| | | | | | | dTs; lnaTs; |
| | | | | | | dCs; lnaAs; |
| | | | | | | dGs; lnaA-Sup |
| 430 | mKLF4- | CGGGGAACTTTTTCA | KLF4 | 5′ and | mouse | dCs; lnaGs; |
| | 13 m02 | GATAAA | | 3′ | | dGs; lnaGs; |
| | | | | | | dGs; lnaAs; |
| | | | | | | dAs; lnaCs; |
| | | | | | | dTs; lnaTs; |
| | | | | | | dTs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dA-Sup |
| 431 | mKLF4- | CGGGGAACTGTCGTT | KLF4 | 5′ and | mouse | dCs; lnaGs; |
| | 14 m02 | CAGA | | 3′ | | dGs; lnaGs; |
| | | | | | | dGs; lnaAs; |
| | | | | | | dAs; lnaCs; |
| | | | | | | dTs; lnaGs; |
| | | | | | | dTs; lnaCs; |
| | | | | | | dGs; lnaTs; |
| | | | | | | dTs; lnaCs; |
| | | | | | | dAs; lnaGs; |
| | | | | | | dA- |
| | | | | | | Sup |
| 432 | mKLF4- | CCGGGGAACTTTCAG | KLF4 | 5′ and | mouse | dCs; lnaCs; |
| | 15 m02 | ATAAA | | 3′ | | dGs; lnaGs; |
| | | | | | | dGs; lnaGs; |
| | | | | | | dAs; lnaAs; |
| | | | | | | dCs; lnaTs; |
| | | | | | | dTs; lnaTs; |
| | | | | | | dCs; lnaAs; |
| | | | | | | dGs; lnaAs; |
| | | | | | | dTs; lnaAs; |
| | | | | | | dAs; lnaA- |
| | | | | | | Sup |
| 433 | mKLF4- | GTCGTTCAGATAAAA | KLF4 | 3′ | mouse | dGs; lnaTs; |
| | 16 m02 | | | | | dCs; lnaGs; |
| | | | | | | dTs; lnaTs; |
| | | | | | | dCs; lnaAs; |
| | | | | | | dGs; lnaAs; |
| | | | | | | dTs; lnaAs; |
| | | | | | | dAs; lnaAs; |
| | | | | | | dA-Sup |
| 434 | mKLF4- | TTCAGATAAAATATT | KLF4 | 3′ | mouse | dTs; lnaTs; |
| | 17 m02 | | | | | dCs; lnaAs; |
| | | | | | | dGs; lnaAs; |
| | | | | | | dTs; lnaAs; |
| | | | | | | dAs; lnaAs; |
| | | | | | | dAs; lnaTs; |
| | | | | | | dAs; lnaTs; |
| | | | | | | dT-Sup |
| 435 | mKLF4- | TTTTTGTCGTTCAGAT | KLF4 | 3′ | mouse | dTs; lnaTs; |
| | 18 m02 | AAAA | | | | dTs; lnaTs; |
| | | | | | | dTs; lnaGs; |
| | | | | | | dTs; lnaCs; |
| | | | | | | dGs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dAs; lnaA- |
| | | | | | | Sup |
| 436 | mKLF4- | TTTTTCAGATAAAAT | KLF4 | 3′ | mouse | dTs; lnaTs; |
| | 19 m02 | ATT | | | | dTs; lnaTs; |
| | | | | | | dTs; lnaCs; |
| | | | | | | dAs; lnaGs; |
| | | | | | | dAs; lnaTs; |
| | | | | | | dAs; lnaAs; |
| | | | | | | dAs; lnaAs; |
| | | | | | | dTs; lnaAs; |
| | | | | | | dTs; lnaT-Sup |
| 437 | mFXN- | CTCCGCGGCCGCTCC | FXN | 5′ | mouse | dCs; lnaTs; |
| | 01 m02 | | | | | dCs; lnaCs; |
| | | | | | | dGs; lnaCs; |
| | | | | | | dGs; lnaGs; |
| | | | | | | dCs; lnaCs; |
| | | | | | | dGs; lnaCs; |
| | | | | | | dTs; lnaCs; |
| | | | | | | dC-Sup |
| 438 | mFXN- | GCCCACATGCTACTC | FXN | 5′ | mouse | dGs; lnaCs; |
| | 02 m02 | | | | | dCs; lnaCs; |
| | | | | | | dAs; lnaCs; |
| | | | | | | dAs; lnaTs; |
| | | | | | | dGs; lnaCs; |
| | | | | | | dTs; lnaAs; |
| | | | | | | dCs; lnaTs; |
| | | | | | | dC-Sup |
| 439 | mFXN- | TCCGAACGCCCACAT | FXN | 5′ | mouse | dTs; lnaCs; |
| | 03 m02 | | | | | dCs; lnaGs; |
| | | | | | | dAs; lnaAs; |
| | | | | | | dCs; lnaGs; |
| | | | | | | dCs; lnaCs; |
| | | | | | | dCs; lnaAs; |
| | | | | | | dCs; lnaAs; |
| | | | | | | dT-Sup |
| 440 | mFXN- | CGAGGACTCGGTGGT | FXN | 5′ | mouse | dCs; lnaGs; |
| | 04 m02 | | | | | dAs; lnaGs; |
| | | | | | | dGs; lnaAs; |
| | | | | | | dCs; lnaTs; |
| | | | | | | dCs; lnaGs; |
| | | | | | | dGs; lnaTs; |
| | | | | | | dGs; lnaGs; |
| | | | | | | dT-Sup |
| 441 | mFXN- | CCAGCTCCGCGGCCG | FXN | 5′ | mouse | dCs; lnaCs; |
| | 05 m02 | | | | | dAs; lnaGs; |
| | | | | | | dCs; lnaTs; |
| | | | | | | dCs; lnaCs; |
| | | | | | | dGs; lnaCs; |
| | | | | | | dGs; lnaGs; |
| | | | | | | dCs; lnaCs; |
| | | | | | | dG-Sup |
| 442 | mFXN- | CTCCGCGGCCGCTCCC | FXN | 5′ | mouse | dCs; lnaTs; |
| | 06 m02 | | | | | dCs; lnaCs; |
| | | | | | | dGs; lnaCs; |
| | | | | | | dGs; lnaGs; |
| | | | | | | dCs; lnaCs; |
| | | | | | | dGs; lnaCs; |
| | | | | | | dTs; lnaCs; |
| | | | | | | dCs; lnaC- |
| | | | | | | Sup |
| 443 | mFXN- | GCCCACATGCTACTCC | FXN | 5′ | mouse | dGs; lnaCs; |
| | 07 m02 | | | | | dCs; lnaCs; |
| | | | | | | dAs; lnaCs; |
| | | | | | | dAs; lnaTs; |
| | | | | | | dGs; lnaCs; |
| | | | | | | dTs; lnaAs; |
| | | | | | | dCs; lnaTs; |
| | | | | | | dCs; lnaC- |
| | | | | | | Sup |
| 444 | mFXN- | CTCCGCGGCCGCTCC | FXN | 5′ | mouse | dCs; lnaTs; |
| | 08 m02 | TCAAAGATC | | | | dCs; lnaCs; |
| | | | | | | dGs; lnaCs; |
| | | | | | | dGs; lnaGs; |
| | | | | | | dCs; lnaCs; |
| | | | | | | dGs; lnaCs; |
| | | | | | | dTs; lnaCs; |
| | | | | | | dCs; lnaTs; |
| | | | | | | dCs; dAs; |
| | | | | | | dAs; dAs; |
| | | | | | | dGs; lnaAs; |
| | | | | | | dTs; lnaC- |
| | | | | | | Sup |
| 445 | mFXN- | GCCCACATGCTACTC | FXN | 5′ | mouse | dGs; lnaCs; |
| | 09 m02 | CCAAAGGTC | | | | dCs; lnaCs; |
| | | | | | | dAs; lnaCs; |
| | | | | | | dAs; lnaTs; |
| | | | | | | dGs; lnaCs; |
| | | | | | | dTs; lnaAs; |
| | | | | | | dCs; lnaTs; |
| | | | | | | dCs; lnaCs; |
| | | | | | | dCs; dAs; |
| | | | | | | dAs; dAs; |
| | | | | | | dGs; lnaGs; |
| | | | | | | dTs; lnaC- |
| | | | | | | Sup |
| 446 | mFXN- | CTCCGCGGCCGCTCC | FXN | 5′ and | mouse | dCs; lnaTs; |
| | 10 m02 | TTTTTGGGAGGGAAC | | 3′ | | dCs; lnaCs; |
| | | ACACT | | | | dGs; lnaCs; |
| | | | | | | dGs; lnaGs; |
| | | | | | | dCs; lnaCs; |
| | | | | | | dGs; lnaCs; |
| | | | | | | dTs; lnaCs; |
| | | | | | | dCs; lnaTs; |
| | | | | | | dTs; lnaTs; |
| | | | | | | dTs; lnaTs; |
| | | | | | | dGs; lnaGs; |
| | | | | | | dGs; lnaAs; |
| | | | | | | dGs; lnaGs; |
| | | | | | | dGs; lnaAs; |
| | | | | | | dAs; lnaCs; |
| | | | | | | dAs; lnaCs; |
| | | | | | | dAs; lnaCs; |
| | | | | | | dT- |
| | | | | | | Sup |
| 447 | mFXN- | GCCCACATGCTACTC | FXN | 5′ and | mouse | dGs; lnaCs; |
| | 11 m02 | TTTTTGGGAGGGAAC | | 3′ | | dCs; lnaCs; |
| | | ACACT | | | | dAs; lnaCs; |
| | | | | | | dAs; lnaTs; |
| | | | | | | dGs; lnaCs; |
| | | | | | | dTs; lnaAs; |
| | | | | | | dCs; lnaTs; |
| | | | | | | dCs; lnaTs; |
| | | | | | | dTs; lnaTs; |
| | | | | | | dTs; lnaTs; |
| | | | | | | dGs; lnaGs; |
| | | | | | | dGs; lnaAs; |
| | | | | | | dGs; lnaGs; |
| | | | | | | dGs; lnaAs; |
| | | | | | | dAs; lnaCs; |
| | | | | | | dAs; lnaCs; |
| | | | | | | dAs; lnaCs; |
| | | | | | | dT- |
| | | | | | | Sup |
| 448 | mFXN- | CTCCGCGGCCGCTCC | FXN | 5′ and | mouse | dCs; lnaTs; |
| | 12 m02 | GGGAGGGAACACACT | | 3′ | | dCs; lnaCs; |
| | | | | | | dGs; lnaCs; |
| | | | | | | dGs; lnaGs; |
| | | | | | | dCs; lnaCs; |
| | | | | | | dGs; lnaCs; |
| | | | | | | dTs; lnaCs; |
| | | | | | | dCs; lnaGs; |
| | | | | | | dGs; lnaGs; |
| | | | | | | dAs; lnaGs; |
| | | | | | | dGs; lnaGs; |
| | | | | | | dAs; lnaAs; |
| | | | | | | dCs; lnaAs; |
| | | | | | | dCs; lnaAs; |
| | | | | | | dCs; lnaT-Sup |
| 449 | mFXN- | GCCCACATGCTACTC | FXN | 5′ and | mouse | dGs; lnaCs; |
| | 13 m02 | GGGAGGGAACACACT | | 3′ | | dCs; lnaCs; |
| | | | | | | dAs; lnaCs; |
| | | | | | | dAs; lnaTs; |
| | | | | | | dGs; lnaCs; |
| | | | | | | dTs; lnaAs; |
| | | | | | | dCs; lnaTs; |
| | | | | | | dCs; lnaGs; |
| | | | | | | dGs; lnaGs; |
| | | | | | | dAs; lnaGs; |
| | | | | | | dGs; lnaGs; |
| | | | | | | dAs; lnaAs; |
| | | | | | | dCs; lnaAs; |
| | | | | | | dCs; lnaAs; |
| | | | | | | dCs; lnaT- |
| | | | | | | Sup |
| 450 | mFXN- | CGGCCGCTCCGGGA | FXN | 5′ and | mouse | dCs; lnaGs; |
| | 14 m02 | GGGAAC | | 3′ | | dGs; lnaCs; |
| | | | | | | dCs; lnaGs; |
| | | | | | | dCs; lnaTs; |
| | | | | | | dCs; lnaCs; |
| | | | | | | dGs; lnaGs; |
| | | | | | | dGs; lnaAs; |
| | | | | | | dGs; lnaGs; |
| | | | | | | dGs; lnaAs; |
| | | | | | | dAs; lnaC- |
| | | | | | | Sup |
| 451 | mFXN- | CATGCTACTCGGGAG | FXN | 5′ and | mouse | dCs; lnaAs; |
| | 15 m02 | GGAAC | | 3′ | | dTs; lnaGs; |
| | | | | | | dCs; lnaTs; |
| | | | | | | dAs; lnaCs; |
| | | | | | | dTs; lnaCs; |
| | | | | | | dGs; lnaGs; |
| | | | | | | dGs; lnaAs; |
| | | | | | | dGs; lnaGs; |
| | | | | | | dGs; lnaAs; |
| | | | | | | dAs; lnaC- |
| | | | | | | Sup |
| 452 | mFXN- | GGGAGGGAACACACT | FXN | 3′ | mouse | dGs; lnaGs; |
| | 16 m02 | | | | | dGs; lnaAs; |
| | | | | | | dGs; lnaGs; |
| | | | | | | dGs; lnaAs; |
| | | | | | | dAs; lnaCs; |
| | | | | | | dAs; lnaCs; |
| | | | | | | dAs; lnaCs; |
| | | | | | | dT- |
| | | | | | | Sup |
| 453 | mFXN- | GGGGTCTTCACCTGA | FXN | 3′ | mouse | dGs; lnaGs; |
| | 17 m02 | | | | | dGs; lnaGs; |
| | | | | | | dTs; lnaCs; |
| | | | | | | dTs; lnaTs; |
| | | | | | | dCs; lnaAs; |
| | | | | | | dCs; lnaCs; |
| | | | | | | dTs; lnaGs; |
| | | | | | | dA-Sup |
| 454 | mFXN- | GGCTGTTATATCATG | FXN | 3′ | mouse | dGs; lnaGs; |
| | 18 m02 | | | | | dCs; lnaTs; |
| | | | | | | dGs; lnaTs; |
| | | | | | | dTs; lnaAs; |
| | | | | | | dTs; lnaAs; |
| | | | | | | dTs; lnaCs; |
| | | | | | | dAs; lnaTs; |
| | | | | | | dG-Sup |
| 455 | mFXN- | GGCATTTTAAGATGG | FXN | 3′ | mouse | dGs; lnaGs; |
| | 19 m02 | | | | | dCs; lnaAs; |
| | | | | | | dTs; lnaTs; |
| | | | | | | dTs; lnaTs; |
| | | | | | | dAs; lnaAs; |
| | | | | | | dGs; lnaAs; |
| | | | | | | dTs; lnaGs; |
| | | | | | | dG-Sup |
| 456 | mFXN- | TTTTTGGGAGGGAAC | FXN | 3′ | mouse | dTs; lnaTs; |
| | 20 m02 | ACACT | | | | dTs; lnaTs; |
| | | | | | | dTs; lnaGs; |
| | | | | | | dGs; lnaGs; |
| | | | | | | dAs; lnaGs; |
| | | | | | | dGs; lnaGs; |
| | | | | | | dAs; lnaAs; |
| | | | | | | dCs; lnaAs; |
| | | | | | | dCs; lnaAs; |
| | | | | | | dCs; lnaT- |
| | | | | | | Sup |
| 457 | mFXN- | TTTTTGGCTGTTATAT | FXN | 3′ | mouse | dTs; lnaTs; |
| | 21 m02 | CATG | | | | dTs; lnaTs; |
| | | | | | | dTs; lnaGs; |
| | | | | | | dGs; lnaCs; |
| | | | | | | dTs; lnaGs; |
| | | | | | | dTs; lnaTs; |
| | | | | | | dAs; lnaTs; |
| | | | | | | dAs; lnaTs; |
| | | | | | | dCs; lnaAs; |
| | | | | | | dTs; lnaG- |
| | | | | | | Sup |

### Example 7

### PTEN and KLF4 Oligos

### Methods

Protein measurements: Hepal-6 and GM04078 cells were plated at 150000 cells per well. The cells were transfected with PTEN or KLF4 oligos using Lipofectamine 2000. 30 nM of each PTEN oligo was used for transfection. If two oligos were combined in an experiment, then 30 nM of each PTEN oligo was used for transfection. 50 nM of each KLF4 oligo was used for transfection. If two oligos were combined in an experiment, then 50 nM of each PTEN oligo was used for transfection. Lysate was harvested from the cells at 1 or 2 days after transfection for PTEN oligos or 3 days after transfection for KLF4 oligos. The antibodies used for detection were Cell Signaling KLF4 4038 and Cell Signaling PTEN 9552.

RNA measurements: Hepal-6 and GM04078 were plated at 4000 cells per well. The cells were transfected with the oligos using Lipofectamine 2000. 30 nM of each PTEN oligo was used for transfection. If two oligos were combined in an experiment, then 30 nM of each PTEN oligo was used for transfection. 50 nM of each KLF4 oligo was used for transfection. If two oligos were combined in an experiment, then 50 nM of each PTEN oligo was used for transfection. RNA was extracted from lysate collected 3 days post-transfection. Cells-to-Ct (Life Technologies) procedure was used to analyze RNA levels following manufacturer's protocol. Taqman® probes used were from Life Technologies:

KLF4 Mm00516104_m1

PTEN Hs02621230_s1

Actin Hs01060665_g1

Gapdh Hs02758991_g1

Actinomycin D treatment: Actinomycin D (Life Technologies) was added to cell culture media at 10 microgram/ml concentration and incubated. RNA isolation was done using Trizol (Sigma) following manufacturer's instructions. KLF4 probes were purchased from Life Technologies.

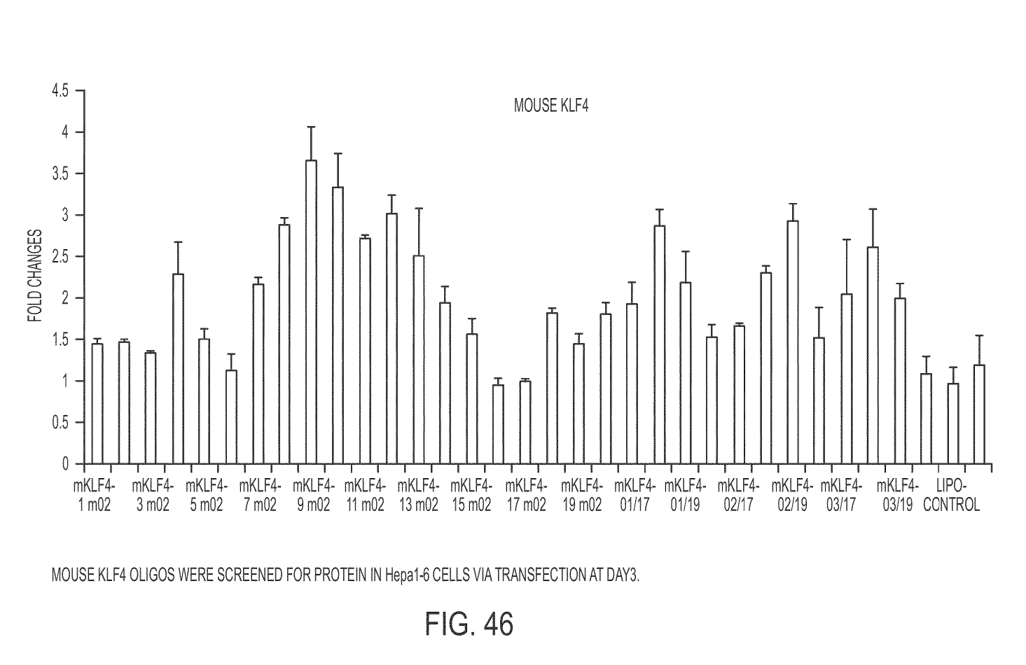

Oligo sequences tested: The oligos tested in FIGS. 44-48 correspond to the same oligo sequences provided in Table 9. For example, PTEN 101 in FIG. 44A is the same as PTEN-101 in Table 9, mKLF4-1 m02 in FIG. 46 is the same as mKLF4-1 m02 in Table 9, etc.

### Results

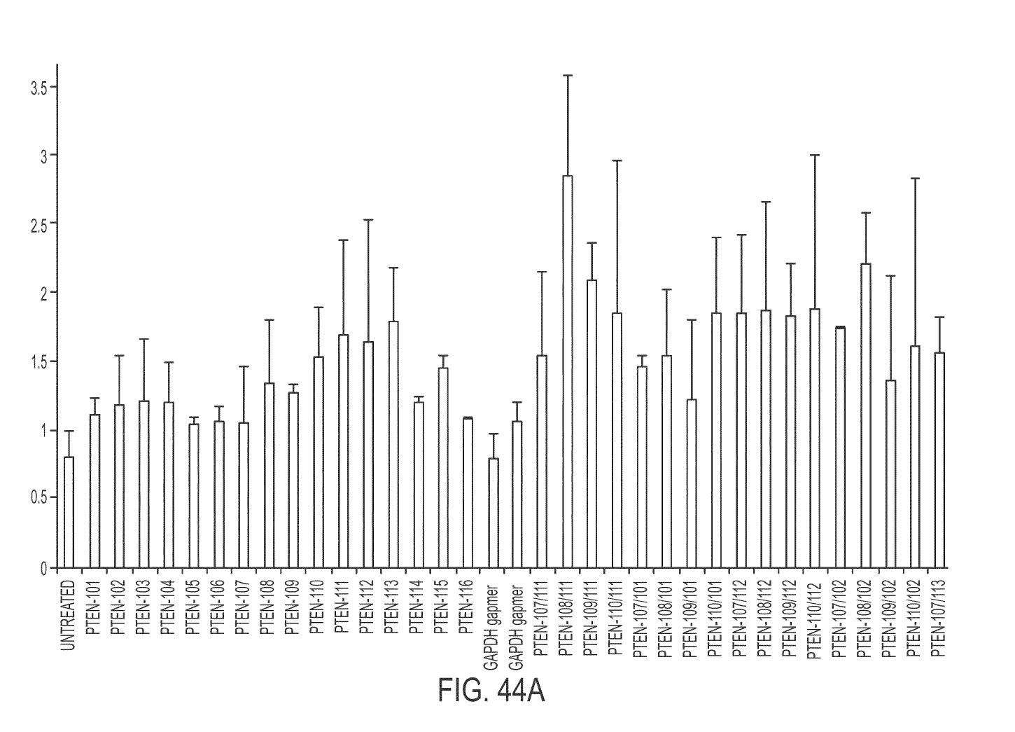

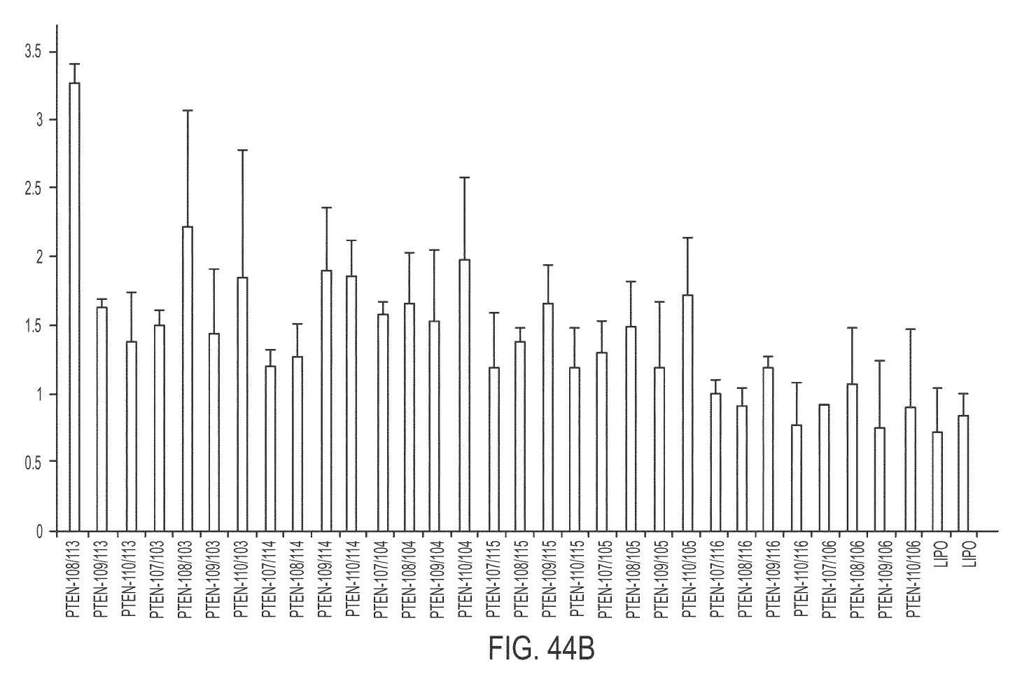

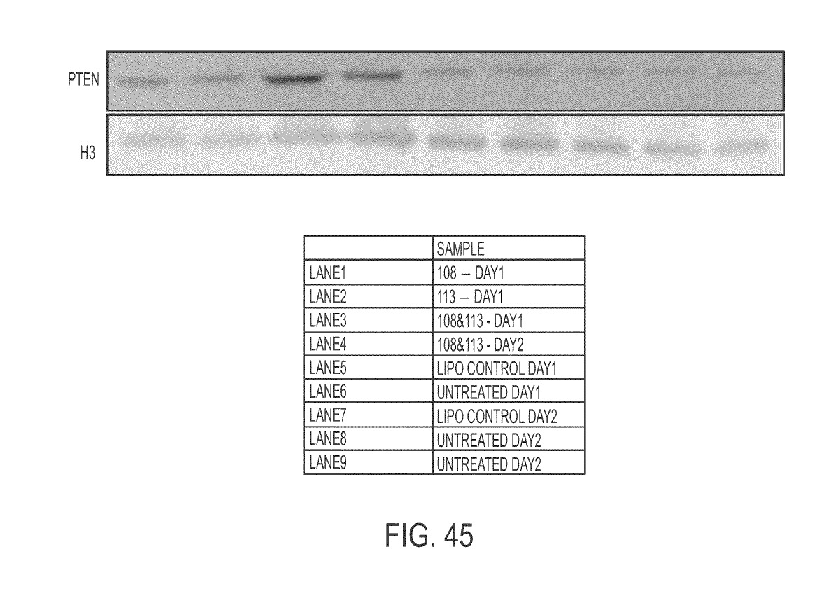

Oligonucleotides specific for PTEN were tested by treating cells with each oligo. Several PTEN oligos were able to upregulate PTEN mRNA levels in the treated cells (FIGS. 44A and 44B). PTEN oligos 108 and 113, when combined, were also able to upregulate PTEN protein levels in the treated cells more than either oligo used separately (FIG. 45).

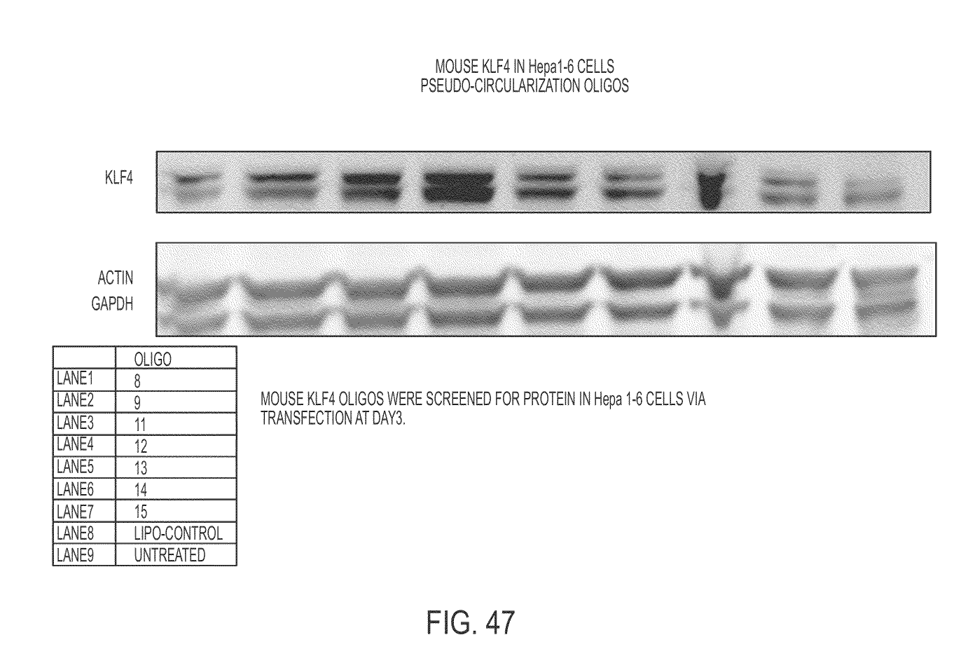

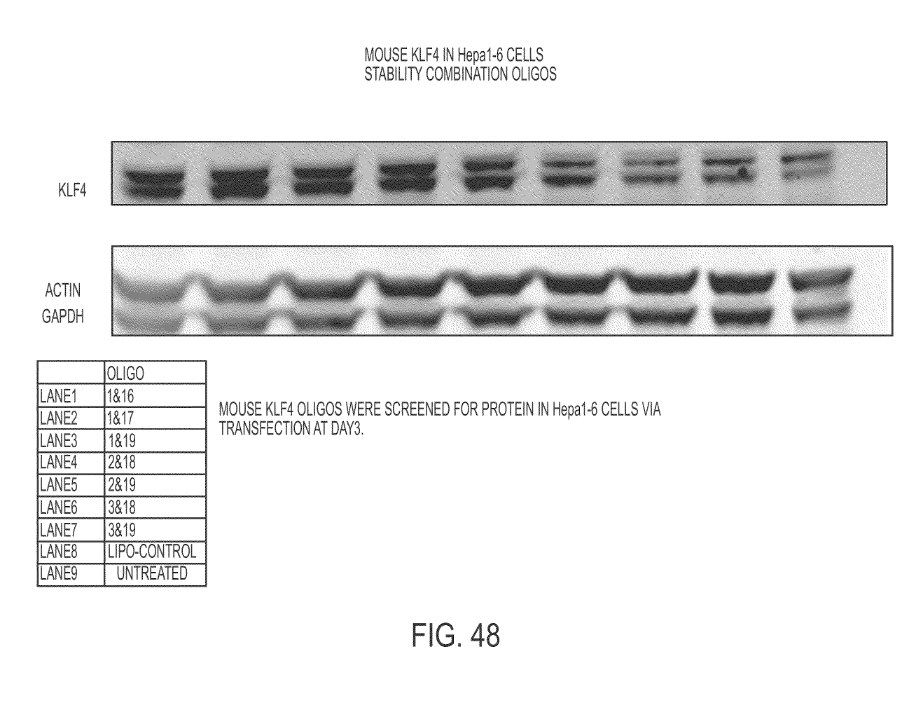

Oligonucleotides specific for KLF4 were tested by treating cells with each oligo. Several KLF4 oligos were able to upregulate KLF4 mRNA levels in the treated cells (FIG. 46). Several KLF4 oligos, used alone or in combination, were also able to upregulate KLF4 protein levels in the treated cells (FIGS. 47 and 48).

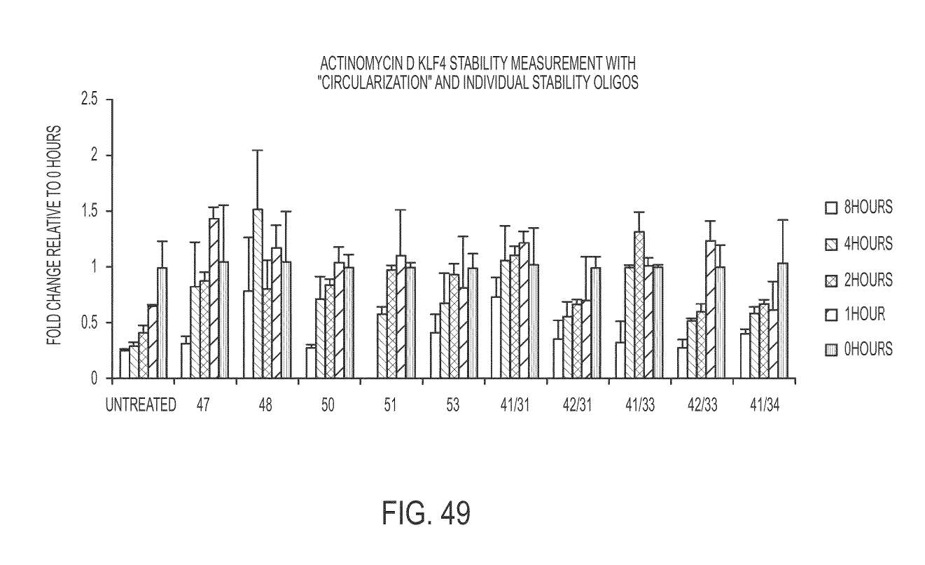

In another experiment, cells were treated with actinomycin D and a circularization or other type of stability oligo and the stability of KLF4 was measured. It was found that the RNA stability increase level (˜2 hours vs. ˜4-8 hours) was comparable between “circularization” and individual 5′/3′ end oligos, showing that both types of oligos were effective (FIG. 49).

These results demonstrate that both mRNA and protein levels can be upregulated using oligos that are capable of increasing RNA stability.

### Example 8

### Increased mRNA Stability in a Gene with a Long mRNA Half-Life

### Methods

RNA measurements: RNA analysis, cDNA synthesis and QRT-PCR was done with Life Technologies Cells-to-Ct kit and StepOne Plus instrument. ACTB oligos were transfected to Hep3B cells at 30 nM concentration using RNAimax (Life Technologies). For combinations, each oligo were transfected at 30 nM concentration. RNA analysis was done with Cells-to-Ct kit (Life Technologies) using ACTIN (Hs01060665_g1) and GAPDH (Hs02758991_g1, housekeeper control) primers purchased from Life Technologies.

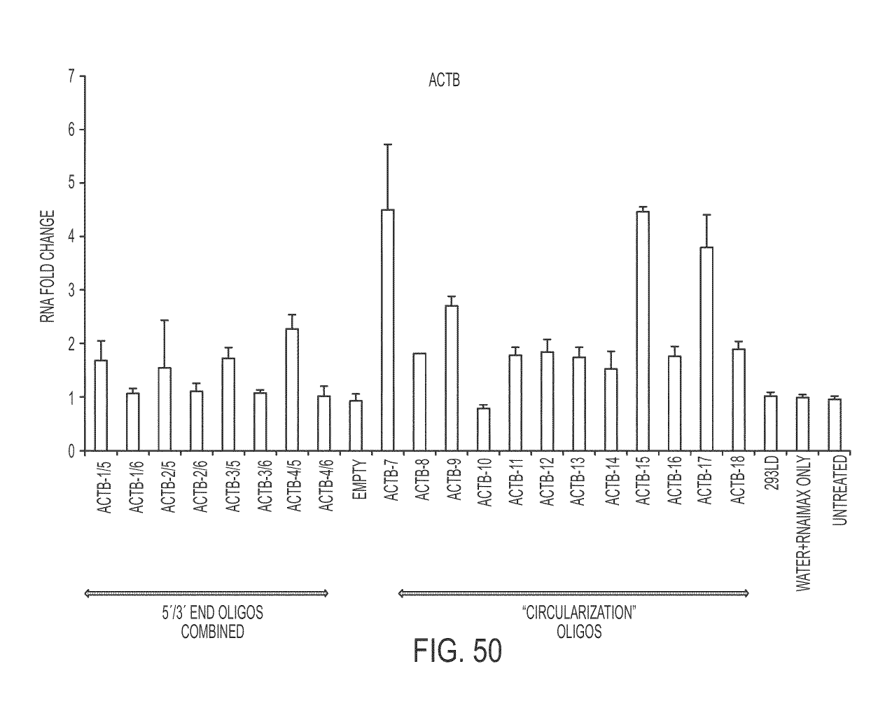

Oligo sequences tested: The oligos tested in FIG. 50 correspond to the same oligo sequences provided in Table 7. For example, ACTB-8 in FIG. 50 is the same as ACTB-8 in Table 7, ACTB-9 in FIG. 50 is the same as ACTB-9 in Table 7, etc.

### Results

Actin-beta is a housekeeper gene that has highly stable mRNA. Oligonucleotides specific for Actin-Beta mRNA were tested by treating cells with each oligo or a combination thereof. Several oligos, both 5′ and 3′ targeting, as well as circularization oligos, were able to upregulate actin-beta mRNA levels (FIG. 50). These data show that stability oligos can improve the stability of even already-highly-stable mRNA.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### Example 9

### Further 5′ and 3′ End Targeting Oligonucleotides

Table 10 provides further exemplary RNA 5′ and 3′ end targeting oligos for multiple human and mouse genes.

TABLE 10
Oligonucleotides designed to target 5′ and 3′ ends of RNAs

| SEQ | Oligo | | Gene | | | Formatted |
|---|---|---|---|---|---|---|
| ID NO | Name | Base Sequence | Name | | Organism | Sequence |
| | | | | Target Region | | |
| 459 | FXN-654 | TGTCTCATTTGGAGA | FXN | 3′ | human | dTs; lnaGs; |
| | m02 | | | | | dTs; lnaCs; |
| | | | | | | dTs; lnaCs; |
| | | | | | | dAs; lnaTs; |
| | | | | | | dTs; lnaTs; |
| | | | | | | dGs; lnaGs; |
| | | | | | | dAs; lnaGs; |
| | | | | | | dA- |
| | | | | | | Sup |
| 460 | FXN-655 | ATAATGAAGCTGGG | FXN | 3′ | human | dAs; lnaTs; |
| | m02 | | | | | dAs; lnaAs; |
| | | | | | | dTs; lnaGs; |
| | | | | | | dAs; lnaAs; |
| | | | | | | dGs; lnaCs; |
| | | | | | | dTs; lnaGs; |
| | | | | | | dGs; lnaG-Sup |
| 461 | FXN-656 | TTTTCCCTCCTGGAA | FXN | 3′ | human | dTs; lnaTs; |
| | m02 | | | | | dTs; lnaTs; |
| | | | | | | dCs; lnaCs; |
| | | | | | | dCs; lnaTs; |
| | | | | | | dCs; lnaCs; |
| | | | | | | dTs; lnaGs; |
| | | | | | | dGs; lnaAs; |
| | | | | | | dA- |
| | | | | | | Sup |
| 462 | FXN-657 | TGCATAATGAAGCTG | FXN | 3′ | human | dTs; lnaGs; |
| | m02 | | | | | dCs; lnaAs; |
| | | | | | | dTs; lnaAs; |
| | | | | | | dAs; lnaTs; |
| | | | | | | dGs; lnaAs; |
| | | | | | | dAs; lnaGs; |
| | | | | | | dCs; lnaTs; |
| | | | | | | dG- |
| | | | | | | Sup |
| 463 | FXN-658 | AAATCCTTCAAAGAA | FXN | 3′ | human | dAs; lnaAs; |
| | m02 | | | | | dAs; lnaTs; |
| | | | | | | dCs; lnaCs; |
| | | | | | | dTs; lnaTs; |
| | | | | | | dCs; lnaAs; |
| | | | | | | dAs; lnaAs; |
| | | | | | | dGs; lnaAs; |
| | | | | | | dA- |
| | | | | | | Sup |
| 464 | FXN-659 | TTGGAAGATTTTTTG | FXN | 3′ | human | dTs; lnaTs; |
| | m02 | | | | | dGs; lnaGs; |
| | | | | | | dAs; lnaAs; |
| | | | | | | dGs; lnaAs; |
| | | | | | | dTs; lnaTs; |
| | | | | | | dTs; lnaTs; |
| | | | | | | dTs; lnaTs; |
| | | | | | | dG- |
| | | | | | | Sup |
| 465 | FXN-660 | GCATTCTTGTAGCAG | FXN | 3′ | human | dGs; lnaCs; |
| | m02 | | | | | dAs; lnaTs; |
| | | | | | | dTs; lnaCs; |
| | | | | | | dTs; lnaTs; |
| | | | | | | dGs; lnaTs; |
| | | | | | | dAs; lnaGs; |
| | | | | | | dCs; lnaAs; |
| | | | | | | dG- |
| | | | | | | Sup |
| 466 | FXN-557 | ACAACAAAAAACAGA | FXN | 3′ | human | dAs; lnaCs; |
| | m02 | | | | | dAs; lnaAs; |
| | | | | | | dCs; lnaAs; |
| | | | | | | dAs; lnaAs; |
| | | | | | | dAs; lnaAs; |
| | | | | | | dAs; lnaCs; |
| | | | | | | dAs; lnaGs; |
| | | | | | | dA- |
| | | | | | | Sup |
| 467 | FXN-662 | TGAAGCTGGGGTCTT | FXN | 3′ | human | dTs; lnaGs; |
| | m02 | | | | | dAs; lnaAs; |
| | | | | | | dGs; lnaCs; |
| | | | | | | dTs; lnaGs; |
| | | | | | | dGs; lnaGs; |
| | | | | | | dGs; lnaTs; |
| | | | | | | dCs; lnaTs; |
| | | | | | | dT- |
| | | | | | | Sup |
| 468 | FXN-663 | CCTGAAAACATTTGT | FXN | 3′ | human | dCs; lnaCs; |
| | m02 | | | | | dTs; lnaGs; |
| | | | | | | dAs; lnaAs; |
| | | | | | | dAs; lnaAs; |
| | | | | | | dCs; lnaAs; |
| | | | | | | dTs; lnaTs; |
| | | | | | | dTs; lnaGs; |
| | | | | | | dT- |
| | | | | | | Sup |
| 469 | FXN-664 | TTCATTTTCCCTCCT | FXN | 3′ | human | dTs; lnaTs; |
| | m02 | | | | | dCs; lnaAs; |
| | | | | | | dTs; lnaTs; |
| | | | | | | dTs; lnaTs; |
| | | | | | | dCs; lnaCs; |
| | | | | | | dCs; lnaTs; |
| | | | | | | dCs; lnaCs; |
| | | | | | | dT- |
| | | | | | | Sup |
| 470 | FXN-665 | TTATTATTATTATAT | FXN | 3′ | human | dTs; lnaTs; |
| | m02 | | | | | dAs; lnaTs; |
| | | | | | | dTs; lnaAs; |
| | | | | | | dTs; lnaTs; |
| | | | | | | dAs; lnaTs; |
| | | | | | | dTs; lnaAs; |
| | | | | | | dTs; lnaAs; |
| | | | | | | dT- |
| | | | | | | Sup |
| 471 | FXN-666 | TAACTTTGCATGAAT | FXN | 3′ | human | dTs; lnaAs; |
| | m02 | | | | | dAs; lnaCs; |
| | | | | | | dTs; lnaTs; |
| | | | | | | dTs; lnaGs; |
| | | | | | | dCs; lnaAs; |
| | | | | | | dTs; lnaGs; |
| | | | | | | dAs; lnaAs; |
| | | | | | | dT- |
| | | | | | | Sup |
| 472 | FXN-667 | ATACAAACATGTATG | FXN | 3′ | human | dAs; lnaTs; |
| | m02 | | | | | dAs; lnaCs; |
| | | | | | | dAs; lnaAs; |
| | | | | | | dAs; lnaCs; |
| | | | | | | dAs; lnaTs; |
| | | | | | | dGs; lnaTs; |
| | | | | | | dAs; lnaTs; |
| | | | | | | dG- |
| | | | | | | Sup |
| 473 | FXN-668 | ATTGTAAACCTATAA | FXN | 3′ | human | dAs; lnaTs; |
| | m02 | | | | | dTs; lnaGs; |
| | | | | | | dTs; lnaAs; |
| | | | | | | dAs; lnaAs; |
| | | | | | | dCs; lnaCs; |
| | | | | | | dTs; lnaAs; |
| | | | | | | dTs; lnaAs; |
| | | | | | | dA- |
| | | | | | | Sup |
| 474 | FXN-669 | TGGAGTTGGGGTTAT | FXN | 3′ | human | dTs; lnaGs; |
| | m02 | | | | | dGs; lnaAs; |
| | | | | | | dGs; lnaTs; |
| | | | | | | dTs; lnaGs; |
| | | | | | | dGs; lnaGs; |
| | | | | | | dGs; lnaTs; |
| | | | | | | dTs; lnaAs; |
| | | | | | | dT- |
| | | | | | | Sup |
| 475 | FXN-670 | GTTGGGGTTATTTAG | FXN | 3′ | human | dGs; lnaTs; |
| | m02 | | | | | dTs; lnaGs; |
| | | | | | | dGs; lnaGs; |
| | | | | | | dGs; lnaTs; |
| | | | | | | dTs; lnaAs; |
| | | | | | | dTs; lnaTs; |
| | | | | | | dTs; lnaAs; |
| | | | | | | dG- |
| | | | | | | Sup |
| 476 | FXN-671 | CTCCGCCCTCCAG | FXN | 5′ | human | dCs; lnaTs; |
| | m02 | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dG-Sup |
| 477 | FXN-672 | CCGCCCTCCAG | FXN | 5′ | human | dCs; lnaCs; |
| | m02 | | | | | dGs; lnaCs; |
| | | | | | | dCs; lnaCs; |
| | | | | | | dTs; lnaCs; |
| | | | | | | dCs; lnaAs; |
| | | | | | | dG- |
| | | | | | | Sup |
| 478 | FXN-673 | GCCCTCCAG | FXN | 5′ | human | dGs; lnaCs; |
| | m02 | | | | | dCs; lnaCs; |
| | | | | | | dTs; lnaCs; |
| | | | | | | dCs; lnaAs; |
| | | | | | | dG-Sup |
| 479 | FXN-674 | CCCGCTCCGCCCTCC | FXN | 5′ | human | dCs; lnaCs; |
| | m02 | | | | | dCs; lnaGs; |
| | | | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dC- |
| | | | | | | Sup |
| 480 | FXN-675 | CGCTCCGCCCTCC | FXN | 5′ | human | dCs; lnaGs; |
| | m02 | | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dC-Sup |
| 481 | FXN-676 | CTCCGCCCTCC | FXN | 5′ | human | dCs; lnaTs; |
| | m02 | | | | | dCs; lnaCs; |
| | | | | | | dGs; lnaCs; |
| | | | | | | dCs; lnaCs; |
| | | | | | | dTs; lnaCs; |
| | | | | | | dC- |
| | | | | | | Sup |
| 482 | FXN-677 | CCGCCCTCC | FXN | 5′ | human | dCs; lnaCs; |
| | m02 | | | | | dGs; lnaCs; |
| | | | | | | dCs; lnaCs; |
| | | | | | | dTs; lnaCs; |
| | | | | | | dC-Sup |
| 483 | FXN-678 | GCCACTGGCCGCA | FXN | 5′ | human | dGs; lnaCs; |
| | m02 | | | | | dCs; lnaAs; |
| | | | | | | dCs; lnaTs; |
| | | | | | | dGs; lnaGs; |
| | | | | | | dCs; lnaCs; |
| | | | | | | dGs; lnaCs; |
| | | | | | | dA-Sup |
| 484 | FXN-679 | CACTGGCCGCA | FXN | 5′ | human | dCs; lnaAs; |
| | m02 | | | | | dCs; lnaTs; |
| | | | | | | dGs; lnaGs; |
| | | | | | | dCs; lnaCs; |
| | | | | | | dGs; lnaCs; |
| | | | | | | dA- |
| | | | | | | Sup |
| 485 | FXN-680 | GCGACCCCTGGTG | FXN | 5′ | human | dGs; lnaCs; |
| | m02 | | | | | dGs; lnaAs; |
| | | | | | | dCs; lnaCs; |
| | | | | | | dCs; lnaCs; |
| | | | | | | dTs; lnaGs; |
| | | | | | | dGs; lnaTs; |
| | | | | | | dG-Sup |
| 486 | FXN-681 | GACCCCTGGTG | FXN | 5′ | human | dGs; lnaAs; |
| | m02 | | | | | dCs; lnaCs; |
| | | | | | | dCs; lnaCs; |
| | | | | | | dTs; lnaGs; |
| | | | | | | dGs; lnaTs; |
| | | | | | | dG- |
| | | | | | | Sup |
| 487 | FXN-682 | CTGGCCGCAGGCA | FXN | 5′ | human | dCs; lnaTs; |
| | m02 | | | | | dGs; lnaGs; |
| | | | | | | dCs; lnaCs; |
| | | | | | | dGs; lnaCs; |
| | | | | | | dAs; lnaGs; |
| | | | | | | dGs; lnaCs; |
| | | | | | | dA-Sup |
| 488 | FXN-683 | GGCCACTGGCCGC | FXN | 5′ | human | dGs; lnaGs; |
| | m02 | | | | | dCs; lnaCs; |
| | | | | | | dAs; lnaCs; |
| | | | | | | dTs; lnaGs; |
| | | | | | | dGs; lnaCs; |
| | | | | | | dCs; lnaGs; |
| | | | | | | dC-Sup |
| 489 | FXN-684 | CTGGTGGCCACTG | FXN | 5′ | human | dCs; lnaTs; |
| | m02 | | | | | dGs; lnaGs; |
| | | | | | | dTs; lnaGs; |
| | | | | | | dGs; lnaCs; |
| | | | | | | dCs; lnaAs; |
| | | | | | | dCs; lnaTs; |
| | | | | | | dG-Sup |
| 490 | FXN-685 | GACCCCTGGTGGC | FXN | 5′ | human | dGs; lnaAs; |
| | m02 | | | | | dCs; lnaCs; |
| | | | | | | dCs; lnaCs; |
| | | | | | | dTs; lnaGs; |
| | | | | | | dGs; lnaTs; |
| | | | | | | dGs; lnaGs; |
| | | | | | | dC-Sup |
| 491 | FXN-686 | GCGGCGACCCCTG | FXN | 5′ | human | dGs; lnaCs; |
| | m02 | | | | | dGs; lnaGs; |
| | | | | | | dCs; lnaGs; |
| | | | | | | dAs; lnaCs; |
| | | | | | | dCs; lnaCs; |
| | | | | | | dCs; lnaTs; |
| | | | | | | dG-Sup |
| 492 | FXN-687 | GTGCTGCGGCGAC | FXN | 5′ | human | dGs; lnaTs; |
| | m02 | | | | | dGs; lnaCs; |
| | | | | | | dTs; lnaGs; |
| | | | | | | dCs; lnaGs; |
| | | | | | | dGs; lnaCs; |
| | | | | | | dGs; lnaAs; |
| | | | | | | dC-Sup |
| 493 | FXN-688 | GCTGGGTGCTGCG | FXN | 5′ | human | dGs; lnaCs; |
| | m02 | | | | | dTs; lnaGs; |
| | | | | | | dGs; lnaGs; |
| | | | | | | dTs; lnaGs; |
| | | | | | | dCs; lnaTs; |
| | | | | | | dGs; lnaCs; |
| | | | | | | dG-Sup |
| 494 | FXN-689 | CCAGCGCTGGGTG | FXN | 5′ | human | dCs; lnaCs; |
| | m02 | | | | | dAs; lnaGs; |
| | | | | | | dCs; lnaGs; |
| | | | | | | dCs; lnaTs; |
| | | | | | | dGs; lnaGs; |
| | | | | | | dGs; lnaTs; |
| | | | | | | dG-Sup |
| 495 | FXN-690 | GCCCTCCAGCGCT | FXN | 5′ | human | dGs; lnaCs; |
| | m02 | | | | | dCs; lnaCs; |
| | | | | | | dTs; lnaCs; |
| | | | | | | dCs; lnaAs; |
| | | | | | | dGs; lnaCs; |
| | | | | | | dGs; lnaCs; |
| | | | | | | dT-Sup |
| 496 | FXN-691 | CGCCCGCTCCGCC | FXN | 5′ | human | dCs; lnaGs; |
| | m02 | | | | | dCs; lnaCs; |
| | | | | | | dCs; lnaGs; |
| | | | | | | dCs; lnaTs; |
| | | | | | | dCs; lnaCs; |
| | | | | | | dGs; lnaCs; |
| | | | | | | dC-Sup |
| 497 | FXN-460 | CGCCCTCCAGCGCTGTT | FXN | 5′ and 3′ | human | dCs; lnaGs; |
| | m1000 | TTTATTATTTTGCTTTTT | | | | dCs; lnaCs; |
| | | | | | | dCs; lnaTs; |
| | | | | | | dCs; lnaCs; |
| | | | | | | dAs; lnaGs; |
| | | | | | | dCs; lnaGs; |
| | | | | | | dCs; lnaTs; |
| | | | | | | dGs; dT; |
| | | | | | | dT; dT; |
| | | | | | | dT; dT; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 498 | FXN-461 | CGCTCCGCCCTCCAGTTT | FXN | 5′ and 3′ | human | dCs; lnaGs; |
| | m1000 | TTATTATTTTGCTTTTT | | | | dCs; lnaTs; |
| | | | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; dT; |
| | | | | | | dT; dT; |
| | | | | | | dT; dT; |
| | | | | | | dAs; lnaTs; |
| | | | | | | dTs; lnaAs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 499 | FXN-523 | CAAGTCCAGTTTGGTTT | FXN | 3′ | human | lnaCs; omeAs; |
| | m01 | | | | | lnaAs; omeGs; |
| | | | | | | lnaTs; omeCs; |
| | | | | | | lnaCs; omeAs; |
| | | | | | | lnaGs; omeUs; |
| | | | | | | lnaTs; omeUs; |
| | | | | | | lnaGs; omeGs; |
| | | | | | | lnaTs; omeUs; |
| | | | | | | lnaT- |
| | | | | | | Sup |
| 500 | FXN-524 | GAATAGGCCAAGGAAGA | FXN | 3′ | human | lnaGs; omeAs; |
| | m01 | | | | | lnaAs; omeUs; |
| | | | | | | lnaAs; omeGs; |
| | | | | | | lnaGs; omeCs; |
| | | | | | | lnaCs; omeAs; |
| | | | | | | lnaAs; omeGs; |
| | | | | | | lnaGs; omeAs; |
| | | | | | | lnaAs; omeGs; |
| | | | | | | lnaA- |
| | | | | | | Sup |
| 501 | FXN-525 | ATCAAGCATCTTTTCCG | FXN | 3′ | human | lnaAs; omeUs; |
| | m01 | | | | | lnaCs; omeAs; |
| | | | | | | lnaAs; omeGs; |
| | | | | | | lnaCs; omeAs; |
| | | | | | | lnaTs; omeCs; |
| | | | | | | lnaTs; omeUs; |
| | | | | | | lnaTs; omeUs; |
| | | | | | | lnaCs; omeCs; |
| | | | | | | lnaG- |
| | | | | | | Sup |
| 502 | FXN-526 | TTAAAACGGGGCTGGGC | FXN | 3′ | human | lnaTs; omeUs; |
| | m01 | | | | | lnaAs; omeAs; |
| | | | | | | lnaAs; omeAs; |
| | | | | | | lnaCs; omeGs; |
| | | | | | | lnaGs; deaGs; |
| | | | | | | lnaGs; omeCs; |
| | | | | | | lnaTs; omeGs; |
| | | | | | | lnaGs; omeGs; |
| | | | | | | lnaC- |
| | | | | | | Sup |
| 503 | FXN-527 | GATAGCTTTTAATGTCC | FXN | 3′ | human | lnaGs; omeAs; |
| | m01 | | | | | lnaTs; omeAs; |
| | | | | | | lnaGs; omeCs; |
| | | | | | | lnaTs; omeUs; |
| | | | | | | lnaTs; omeUs; |
| | | | | | | lnaAs; omeAs; |
| | | | | | | lnaTs; omeGs; |
| | | | | | | lnaTs; omeCs; |
| | | | | | | lnaC- |
| | | | | | | Sup |
| 504 | FXN-528 | AGCTGGGGTCTTGGCCT | FXN | 3′ | human | lnaAs; omeGs; |
| | m01 | | | | | lnaCs; omeUs; |
| | | | | | | lnaGs; deaGs; |
| | | | | | | lnaGs; omeGs; |
| | | | | | | lnaTs; omeCs; |
| | | | | | | lnaTs; omeUs; |
| | | | | | | lnaGs; omeGs; |
| | | | | | | lnaCs; omeCs; |
| | | | | | | lnaT- |
| | | | | | | Sup |
| 505 | FXN-529 | CCTCAGCTGCATAATGA | FXN | 3′ | human | lnaCs; omeCs; |
| | m01 | | | | | lnaTs; omeCs; |
| | | | | | | lnaAs; omeGs; |
| | | | | | | lnaCs; omeUs; |
| | | | | | | lnaGs; omeCs; |
| | | | | | | lnaAs; omeUs; |
| | | | | | | lnaAs; omeAs; |
| | | | | | | lnaTs; omeGs; |
| | | | | | | lnaA- |
| | | | | | | Sup |
| 506 | FXN-530 | CAACAACAAAAAACAGA | FXN | 3′ | human | lnaCs; omeAs; |
| | m01 | | | | | lnaAs; omeCs; |
| | | | | | | lnaAs; omeAs; |
| | | | | | | lnaCs; omeAs; |
| | | | | | | lnaAs; omeAs; |
| | | | | | | lnaAs; omeAs; |
| | | | | | | lnaAs; omeCs; |
| | | | | | | lnaAs; omeGs; |
| | | | | | | lnaA- |
| | | | | | | Sup |
| 507 | FXN-531 | AAAAAAATAAACAACAA | FXN | 3′ | human | lnaAs; omeAs; |
| | m01 | | | | | lnaAs; omeAs; |
| | | | | | | lnaAs; omeAs; |
| | | | | | | lnaAs; omeUs; |
| | | | | | | lnaAs; omeAs; |
| | | | | | | lnaAs; omeCs; |
| | | | | | | lnaAs; omeAs; |
| | | | | | | lnaCs; omeAs; |
| | | | | | | lnaA- |
| | | | | | | Sup |
| 508 | FXN-532 | CCTCAAAAGCAGGAATA | FXN | 3′ | human | lnaCs; omeCs; |
| | m01 | | | | | lnaTs; omeCs; |
| | | | | | | lnaAs; omeAs; |
| | | | | | | lnaAs; omeAs; |
| | | | | | | lnaGs; omeCs; |
| | | | | | | lnaAs; omeGs; |
| | | | | | | lnaGs; omeAs; |
| | | | | | | lnaAs; omeUs; |
| | | | | | | lnaA- |
| | | | | | | Sup |
| 509 | FXN-533 | ACACATAGCCCAACTGT | FXN | 3′ | human | lnaAs; omeCs; |
| | m01 | | | | | lnaAs; omeCs; |
| | | | | | | lnaAs; omeUs; |
| | | | | | | lnaAs; omeGs; |
| | | | | | | lnaCs; omeCs; |
| | | | | | | lnaCs; omeAs; |
| | | | | | | lnaAs; omeCs; |
| | | | | | | lnaTs; omeGs; |
| | | | | | | lnaT- |
| | | | | | | Sup |
| 510 | FXN-534 | CTTTCTACAGAGCTGTG | FXN | 3′ | human | lnaCs; omeUs; |
| | m01 | | | | | lnaTs; omeUs; |
| | | | | | | lnaCs; omeUs; |
| | | | | | | lnaAs; omeCs; |
| | | | | | | lnaAs; omeGs; |
| | | | | | | lnaAs; omeGs; |
| | | | | | | lnaCs; omeUs; |
| | | | | | | lnaGs; omeUs; |
| | | | | | | lnaG- |
| | | | | | | Sup |
| 511 | FXN-535 | GTAGGAGGCAACACATT | FXN | 3′ | human | lnaGs; omeUs; |
| | m01 | | | | | lnaAs; omeGs; |
| | | | | | | lnaGs; omeAs; |
| | | | | | | lnaGs; omeGs; |
| | | | | | | lnaCs; omeAs; |
| | | | | | | lnaAs; omeCs; |
| | | | | | | lnaAs; omeCs; |
| | | | | | | lnaAs; omeUs; |
| | | | | | | lnaT- |
| | | | | | | Sup |
| 512 | FXN-536 | CAGAACTTGGGGGCAAG | FXN | 3′ | human | lnaCs; omeAs; |
| | m01 | | | | | lnaGs; omeAs; |
| | | | | | | lnaAs; omeCs; |
| | | | | | | lnaTs; omeUs; |
| | | | | | | lnaGs; deaGs; |
| | | | | | | lnaGs; deaGs; |
| | | | | | | lnaGs; omeCs; |
| | | | | | | lnaAs; omeAs; |
| | | | | | | lnaG- |
| | | | | | | Sup |
| 513 | FXN-537 | CCATAGAAATTAAAAAT | FXN | 3′ | human | lnaCs; omeCs; |
| | m01 | | | | | lnaAs; omeUs; |
| | | | | | | lnaAs; omeGs; |
| | | | | | | lnaAs; omeAs; |
| | | | | | | lnaAs; omeUs; |
| | | | | | | lnaTs; omeAs; |
| | | | | | | lnaAs; omeAs; |
| | | | | | | lnaAs; omeAs; |
| | | | | | | lnaT- |
| | | | | | | Sup |
| 514 | FXN-538 | ACAATCCAAAAAATCTT | FXN | 3′ | human | lnaAs; omeCs; |
| | m01 | | | | | lnaAs; omeAs; |
| | | | | | | lnaTs; omeCs; |
| | | | | | | lnaCs; omeAs; |
| | | | | | | lnaAs; omeAs; |
| | | | | | | lnaAs; omeAs; |
| | | | | | | lnaAs; omeUs; |
| | | | | | | lnaCs; omeUs; |
| | | | | | | lnaT- |
| | | | | | | Sup |
| 515 | FXN-539 | GTGAGGGAGGAAATCCG | FXN | 3′ | human | lnaGs; omeUs; |
| | m01 | | | | | lnaGs; omeAs; |
| | | | | | | lnaGs; omeGs; |
| | | | | | | lnaGs; omeAs; |
| | | | | | | lnaGs; omeGs; |
| | | | | | | lnaAs; omeAs; |
| | | | | | | lnaAs; omeUs; |
| | | | | | | lnaCs; omeCs; |
| | | | | | | lnaG- |
| | | | | | | Sup |
| 516 | FXN-540 | AAGATAAGGGGTATCAT | FXN | 3′ | human | lnaAs; omeAs; |
| | m01 | | | | | lnaGs; omeAs; |
| | | | | | | lnaTs; omeAs; |
| | | | | | | lnaAs; omeGs; |
| | | | | | | lnaGs; omeGs; |
| | | | | | | lnaGs; omeUs; |
| | | | | | | lnaAs; omeUs; |
| | | | | | | lnaCs; omeAs; |
| | | | | | | lnaT- |
| | | | | | | Sup |
| 517 | FXN-541 | GGCATAAGACATTATAA | FXN | 3′ | human | lnaGs; omeGs; |
| | m01 | | | | | lnaCs; omeAs; |
| | | | | | | lnaTs; omeAs; |
| | | | | | | lnaAs; omeGs; |
| | | | | | | lnaAs; omeCs; |
| | | | | | | lnaAs; omeUs; |
| | | | | | | lnaTs; omeAs; |
| | | | | | | lnaTs; omeAs; |
| | | | | | | lnaA- |
| | | | | | | Sup |
| 518 | FXN-542 | TGTTATATTCAGGTATA | FXN | 3′ | human | lnaTs; omeGs; |
| | m01 | | | | | lnaTs; omeUs; |
| | | | | | | lnaAs; omeUs; |
| | | | | | | lnaAs; omeUs; |
| | | | | | | lnaTs; omeCs; |
| | | | | | | lnaAs; omeGs; |
| | | | | | | lnaGs; omeUs; |
| | | | | | | lnaAs; omeUs; |
| | | | | | | lnaA- |
| | | | | | | Sup |
| 519 | FXN-543 | TTTGCTTTTTTAAAGGT | FXN | 3′ | human | lnaTs; omeUs; |
| | m01 | | | | | lnaTs; omeGs; |
| | | | | | | lnaCs; omeUs; |
| | | | | | | lnaTs; omeUs; |
| | | | | | | lnaTs; omeUs; |
| | | | | | | lnaTs; omeAs; |
| | | | | | | lnaAs; omeAs; |
| | | | | | | lnaGs; omeGs; |
| | | | | | | lnaT- |
| | | | | | | Sup |
| 520 | FXN-544 | TTTTTCCTTCTTATTAT | FXN | 3′ | human | lnaTs; omeUs; |
| | m01 | | | | | lnaTs; omeUs; |
| | | | | | | lnaTs; omeCs; |
| | | | | | | lnaCs; omeUs; |
| | | | | | | lnaTs; omeCs; |
| | | | | | | lnaTs; omeUs; |
| | | | | | | lnaAs; omeUs; |
| | | | | | | lnaTs; omeAs; |
| | | | | | | lnaT- |
| | | | | | | Sup |
| 521 | FXN-545 | CATTTTCCCTCCTGGAA | FXN | 3′ | human | lnaCs; omeAs; |
| | m01 | | | | | lnaTs; omeUs; |
| | | | | | | lnaTs; omeUs; |
| | | | | | | lnaCs; omeCs; |
| | | | | | | lnaCs; omeUs; |
| | | | | | | lnaCs; omeCs; |
| | | | | | | lnaTs; omeGs; |
| | | | | | | lnaGs; omeAs; |
| | | | | | | lnaA- |
| | | | | | | Sup |
| 522 | FXN-546 | GAAGAGTGAAGACAATT | FXN | 3′ | human | lnaGs; omeAs; |
| | m01 | | | | | lnaAs; omeGs; |
| | | | | | | lnaAs; omeGs; |
| | | | | | | lnaTs; omeGs; |
| | | | | | | lnaAs; omeAs; |
| | | | | | | lnaGs; omeAs; |
| | | | | | | lnaCs; omeAs; |
| | | | | | | lnaAs; omeUs; |
| | | | | | | lnaT- |
| | | | | | | Sup |
| 523 | FXN-547 | TAAATCCTTCAAAGAAT | FXN | 3′ | human | lnaTs; omeAs; |
| | m01 | | | | | lnaAs; omeAs; |
| | | | | | | lnaTs; omeCs; |
| | | | | | | lnaCs; omeUs; |
| | | | | | | lnaTs; omeCs; |
| | | | | | | lnaAs; omeAs; |
| | | | | | | lnaAs; omeGs; |
| | | | | | | lnaAs; omeAs; |
| | | | | | | lnaT- |
| | | | | | | Sup |
| 524 | FXN-548 | TCATGTACTTCTTGCAG | FXN | 3′ | human | lnaTs; omeCs; |
| | m01 | | | | | lnaAs; omeUs; |
| | | | | | | lnaGs; omeUs; |
| | | | | | | lnaAs; omeCs; |
| | | | | | | lnaTs; omeUs; |
| | | | | | | lnaCs; omeUs; |
| | | | | | | lnaTs; omeGs; |
| | | | | | | lnaCs; omeAs; |
| | | | | | | lnaG- |
| | | | | | | Sup |
| 525 | FXN-549 | GGTTGACCAGCTGCTCT | FXN | 3′ | human | lnaGs; omeGs; |
| | m01 | | | | | lnaTs; omeUs; |
| | | | | | | lnaGs; omeAs; |
| | | | | | | lnaCs; omeCs; |
| | | | | | | lnaAs; omeGs; |
| | | | | | | lnaCs; omeUs; |
| | | | | | | lnaGs; omeCs; |
| | | | | | | lnaTs; omeCs; |
| | | | | | | lnaT- |
| | | | | | | Sup |
| 526 | FXN-550 | AGATAGAACAGTGAGCA | FXN | 3′ | human | lnaAs; omeGs; |
| | m01 | | | | | lnaAs; omeUs; |
| | | | | | | lnaAs; omeGs; |
| | | | | | | lnaAs; omeAs; |
| | | | | | | lnaCs; omeAs; |
| | | | | | | lnaGs; omeUs; |
| | | | | | | lnaGs; omeAs; |
| | | | | | | lnaGs; omeCs; |
| | | | | | | lnaA- |
| | | | | | | Sup |
| 527 | FXN-551 | TAATGTGTCTCATTTGG | FXN | 3′ | human | lnaTs; omeAs; |
| | m01 | | | | | lnaAs; omeUs; |
| | | | | | | lnaGs; omeUs; |
| | | | | | | lnaGs; omeUs; |
| | | | | | | lnaCs; omeUs; |
| | | | | | | lnaCs; omeAs; |
| | | | | | | lnaTs; omeUs; |
| | | | | | | lnaTs; omeGs; |
| | | | | | | lnaG- |
| | | | | | | Sup |
| 528 | FXN-552 | ATTTGTAGGCTACCCTT | FXN | 3′ | human | lnaAs; omeUs; |
| | m01 | | | | | lnaTs; omeUs; |
| | | | | | | lnaGs; omeUs; |
| | | | | | | lnaAs; omeGs; |
| | | | | | | lnaGs; omeCs; |
| | | | | | | lnaTs; omeAs; |
| | | | | | | lnaCs; omeCs; |
| | | | | | | lnaCs; omeUs; |
| | | | | | | lnaT- |
| | | | | | | Sup |
| 529 | FXN-553 | GAAAGAAGCCTGAAAAC | FXN | 3′ | human | lnaGs; omeAs; |
| | m01 | | | | | lnaAs; omeAs; |
| | | | | | | lnaGs; omeAs; |
| | | | | | | lnaAs; omeGs; |
| | | | | | | lnaCs; omeCs; |
| | | | | | | lnaTs; omeGs; |
| | | | | | | lnaAs; omeAs; |
| | | | | | | lnaAs; omeAs; |
| | | | | | | lnaC- |
| | | | | | | Sup |
| 530 | FXN-554 | AGAAGTGCTTACACTTT | FXN | 3′ | human | lnaAs; omeGs; |
| | m01 | | | | | lnaAs; omeAs; |
| | | | | | | lnaGs; omeUs; |
| | | | | | | lnaGs; omeCs; |
| | | | | | | lnaTs; omeUs; |
| | | | | | | lnaAs; omeCs; |
| | | | | | | lnaAs; omeCs; |
| | | | | | | lnaTs; omeUs; |
| | | | | | | lnaT- |
| | | | | | | Sup |
| 531 | FXN-555 | TCAATGCTAAAGAGCTC | FXN | 3′ | human | lnaTs; omeCs; |
| | m01 | | | | | lnaAs; omeAs; |
| | | | | | | lnaTs; omeGs; |
| | | | | | | lnaCs; omeUs; |
| | | | | | | lnaAs; omeAs; |
| | | | | | | lnaAs; omeGs; |
| | | | | | | lnaAs; omeGs; |
| | | | | | | lnaCs; omeUs; |
| | | | | | | lnaC- |
| | | | | | | Sup |
| 532 | Apoa1_mus- | AGTCTGGGTGTCC | Apoa1 | 5′ | mouse | lnaAs; dGs; |
| | 01 | | | | | lnaTs; dCs; |
| | m12 | | | | | lnaTs; dGs; |
| | | | | | | lnaGs; dGs; |
| | | | | | | lnaTs; dGs; |
| | | | | | | lnaTs; dCs; |
| | | | | | | lnaC- |
| | | | | | | Sup |
| 533 | Apoa1_mus- | CCGACAGTCTGGG | Apoa1 | 5′ | mouse | lnaCs; dCs; |
| | 02 | | | | | lnaGs; dAs; |
| | m12 | | | | | lnaCs; dAs; |
| | | | | | | lnaGs; dTs; |
| | | | | | | lnaCs; dTs; |
| | | | | | | lnaGs; dGs; |
| | | | | | | lnaG- |
| | | | | | | Sup |
| 534 | Apoa1_mus- | CTCCGACAGTCTG | Apoa1 | 5′ | mouse | lnaCs; dTs; |
| | 03 | | | | | lnaCs; dCs; |
| | m12 | | | | | lnaGs; dAs; |
| | | | | | | lnaCs; dAs; |
| | | | | | | lnaGs; dTs; |
| | | | | | | lnaCs; dTs; |
| | | | | | | lnaG- |
| | | | | | | Sup |
| 535 | Apoa1_mus- | GACAGTCTGGGTG | Apoa1 | 5′ | mouse | lnaGs; dAs; |
| | 04 | | | | | lnaCs; dAs; |
| | m12 | | | | | lnaGs; dTs; |
| | | | | | | lnaCs; dTs; |
| | | | | | | lnaGs; dGs; |
| | | | | | | lnaGs; dTs; |
| | | | | | | lnaG- |
| | | | | | | Sup |
| 536 | Apoa1_mus- | CAGTCTGGGTG | Apoa1 | 5′ | mouse | lnaCs; dAs; |
| | 05 | | | | | lnaGs; dTs; |
| | m12 | | | | | lnaCs; dTs; |
| | | | | | | lnaGs; dGs; |
| | | | | | | lnaGs; dTs; |
| | | | | | | lnaG-Sup |
| 537 | Apoa1_mus- | CTCAGCCTGGCCCTG | Apoa1 | 5′ | mouse | lnaCs; dTs; |
| | 06 | | | | | lnaCs; dAs; |
| | m12 | | | | | lnaGs; dCs; |
| | | | | | | lnaCs; dTs; |
| | | | | | | lnaGs; dGs; |
| | | | | | | lnaCs; dCs; |
| | | | | | | lnaCs; dTs; |
| | | | | | | lnaG-Sup |
| 538 | Apoa1_mus- | AGTTCAAGGATCAGC | Apoa1 | 5′ | mouse | lnaAs; dGs; |
| | 07 | | | | | lnaTs; dTs; |
| | m12 | | | | | lnaCs; dAs; |
| | | | | | | lnaAs; dGs; |
| | | | | | | lnaGs; dAs; |
| | | | | | | lnaTs; dCs; |
| | | | | | | lnaAs; dGs; |
| | | | | | | lnaC-Sup |
| 539 | Apoa1_mus- | GCTCTCCGACAGTCT | Apoa1 | 5′ | mouse | lnaGs; dCs; |
| | 08 | | | | | lnaTs; dCs; |
| | m12 | | | | | lnaTs; dCs; |
| | | | | | | lnaCs; dGs; |
| | | | | | | lnaAs; dCs; |
| | | | | | | lnaAs; dGs; |
| | | | | | | lnaTs; dCs; |
| | | | | | | lnaT-Sup |
| 540 | Apoa1_mus- | TCTCCGACAGTCT | Apoa1 | 5′ | mouse | lnaTs; dCs; |
| | 09 | | | | | lnaTs; dCs; |
| | m12 | | | | | lnaCs; dGs; |
| | | | | | | lnaAs; dCs; |
| | | | | | | lnaAs; dGs; |
| | | | | | | lnaTs; dCs; |
| | | | | | | lnaT- |
| | | | | | | Sup |
| 541 | Apoa1_mus- | TCCGACAGTCT | Apoa1 | 5′ | mouse | lnaTs; dCs; |
| | 10 | | | | | lnaCs; dGs; |
| | m12 | | | | | lnaAs; dCs; |
| | | | | | | lnaAs; dGs; |
| | | | | | | lnaTs; dCs; |
| | | | | | | lnaT-Sup |
| 542 | Apoa1_mus- | CGGAGCTCTCCGACA | Apoa1 | 5′ | mouse | lnaCs; dGs; |
| | 11 | | | | | lnaGs; dAs; |
| | m12 | | | | | lnaGs; dCs; |
| | | | | | | lnaTs; dCs; |
| | | | | | | lnaTs; dCs; |
| | | | | | | lnaCs; dGs; |
| | | | | | | lnaAs; dCs; |
| | | | | | | lnaA-Sup |
| 543 | Apoa1_mus- | GAGCTCTCCGACA | Apoa1 | 5′ | mouse | lnaGs; dAs; |
| | 12 | | | | | lnaGs; dCs; |
| | m12 | | | | | lnaTs; dCs; |
| | | | | | | lnaTs; dCs; |
| | | | | | | lnaCs; dGs; |
| | | | | | | lnaAs; dCs; |
| | | | | | | lnaA- |
| | | | | | | Sup |
| 544 | Apoa1_mus- | GCTCTCCGACA | Apoa1 | 5′ | mouse | lnaGs; dCs; |
| | 13 | | | | | lnaTs; dCs; |
| | m12 | | | | | lnaTs; dCs; |
| | | | | | | lnaCs; dGs; |
| | | | | | | lnaAs; dCs; |
| | | | | | | lnaA- |
| | | | | | | Sup |
| 545 | Apoa1_mus- | CTATTCCATTTTGGA | Apoa1 | 3′ | mouse | lnaCs; dTs; |
| | 14 | | | | | lnaAs; dTs; |
| | m12 | | | | | lnaTs; dCs; |
| | | | | | | lnaCs; dAs; |
| | | | | | | lnaTs; dTs; |
| | | | | | | lnaTs; dTs; |
| | | | | | | lnaGs; dGs; |
| | | | | | | lnaA-Sup |
| 546 | Apoa1_mus- | CTATTCCATTTTG | Apoa1 | 3′ | mouse | lnaCs; dTs; |
| | 15 | | | | | lnaAs; dTs; |
| | m12 | | | | | lnaTs; dCs; |
| | | | | | | lnaCs; dAs; |
| | | | | | | lnaTs; dTs; |
| | | | | | | lnaTs; dTs; |
| | | | | | | lnaG- |
| | | | | | | Sup |
| 547 | Apoa1_mus- | ATTCCATTTTGGAAA | Apoa1 | 3′ | mouse | lnaAs; dTs; |
| | 16 | | | | | lnaTs; dCs; |
| | m12 | | | | | lnaCs; dAs; |
| | | | | | | lnaTs; dTs; |
| | | | | | | lnaTs; dTs; |
| | | | | | | lnaGs; dGs; |
| | | | | | | lnaAs; dAs; |
| | | | | | | lnaA-Sup |
| 548 | Apoa1_mus- | CCATTTTGGAAAGGT | Apoa1 | 3′ | mouse | lnaCs; dCs; |
| | 17 | | | | | lnaAs; dTs; |
| | m12 | | | | | lnaTs; dTs; |
| | | | | | | lnaTs; dGs; |
| | | | | | | lnaGs; dAs; |
| | | | | | | lnaAs; dAs; |
| | | | | | | lnaGs; dGs; |
| | | | | | | lnaT-Sup |
| 549 | Apoa1_mus- | CCATTTTGGAAAG | Apoa1 | 3′ | mouse | lnaCs; dCs; |
| | 18 | | | | | lnaAs; dTs; |
| | m12 | | | | | lnaTs; dTs; |
| | | | | | | lnaTs; dGs; |
| | | | | | | lnaGs; dAs; |
| | | | | | | lnaAs; dAs; |
| | | | | | | lnaG- |
| | | | | | | Sup |
| 550 | Apoa1_mus- | CATTTTGGAAAGGTT | Apoa1 | 3′ | mouse | lnaCs; dAs; |
| | 19 | | | | | lnaTs; dTs; |
| | m12 | | | | | lnaTs; dTs; |
| | | | | | | lnaGs; dGs; |
| | | | | | | lnaAs; dAs; |
| | | | | | | lnaAs; dGs; |
| | | | | | | lnaGs; dTs; |
| | | | | | | lnaT-Sup |
| 551 | Apoa1_mus- | CATTTTGGAAAGG | Apoa1 | 3′ | mouse | lnaCs; dAs; |
| | 20 | | | | | lnaTs; dTs; |
| | m12 | | | | | lnaTs; dTs; |
| | | | | | | lnaGs; dGs; |
| | | | | | | lnaAs; dAs; |
| | | | | | | lnaAs; dGs; |
| | | | | | | lnaG- |
| | | | | | | Sup |
| 552 | Apoa1_mus- | GGAAAGGTTTATTGT | Apoa1 | 3′ | mouse | lnaGs; dGs; |
| | 21 | | | | | lnaAs; dAs; |
| | m12 | | | | | lnaAs; dGs; |
| | | | | | | lnaGs; dTs; |
| | | | | | | lnaTs; dTs; |
| | | | | | | lnaAs; dTs; |
| | | | | | | lnaTs; dGs; |
| | | | | | | lnaT-Sup |
| 553 | Apoa1_mus- | TCCGACAGTCTCCATT | Apoa1 | 5′ and 3′ | mouse | lnaTs; dCs; |
| | 22 | TTGGAA | | | | dCs; lnaGs; |
| | m22 | | | | | dAs; dCs; |
| | | | | | | lnaAs; dGs; |
| | | | | | | dTs; lnaCs; |
| | | | | | | dTs; dCs; |
| | | | | | | lnaCs; dAs; |
| | | | | | | dTs; lnaTs; |
| | | | | | | dTs; dTs; |
| | | | | | | lnaGs; dGs; |
| | | | | | | dAs; lnaA- |
| | | | | | | Sup |
| 554 | Apoa1_mus- | GCTCTCCGACACCATT | Apoa1 | 5′ and 3′ | mouse | lnaGs; dCs; |
| | 23 | TTGGAA | | | | dTs; lnaCs; |
| | m22 | | | | | dTs; dCs; |
| | | | | | | lnaCs; dGs; |
| | | | | | | dAs; lnaCs; |
| | | | | | | dAs; dCs; |
| | | | | | | lnaCs; dAs; |
| | | | | | | dTs; lnaTs; |
| | | | | | | dTs; dTs; |
| | | | | | | lnaGs; dGs; |
| | | | | | | dAs; lnaA- |
| | | | | | | Sup |
| 555 | Apoa1_mus- | TCCGACAGTCTCATTT | Apoa1 | 5′ and 3′ | mouse | lnaTs; dCs; |
| | 24 | TGGAAA | | | | dCs; lnaGs; |
| | m22 | | | | | dAs; dCs; |
| | | | | | | lnaAs; dGs; |
| | | | | | | dTs; lnaCs; |
| | | | | | | dTs; dCs; |
| | | | | | | lnaAs; dTs; |
| | | | | | | dTs; lnaTs; |
| | | | | | | dTs; dGs; |
| | | | | | | lnaGs; dAs; |
| | | | | | | dAs; lnaA- |
| | | | | | | Sup |
| 556 | Apoa1_mus- | GCTCTCCGACACATTT | Apoa1 | 5′ and 3′ | mouse | lnaGs; dCs; |
| | 25 | TGGAAA | | | | dTs; lnaCs; |
| | m22 | | | | | dTs; dCs; |
| | | | | | | lnaCs; dGs; |
| | | | | | | dAs; lnaCs; |
| | | | | | | dAs; dCs; |
| | | | | | | lnaAs; dTs; |
| | | | | | | dTs; lnaTs; |
| | | | | | | dTs; dGs; |
| | | | | | | lnaGs; dAs; |
| | | | | | | dAs; lnaA- |
| | | | | | | Sup |
| 557 | FXN-761 | CCTCAAAAGCAGGAA | FXN | 3′ | human | lnaCs; omeCs; |
| | m01 | | | | | lnaTs; omeCs; |
| | | | | | | lnaAs; omeAs; |
| | | | | | | lnaAs; omeAs; |
| | | | | | | lnaGs; omeCs; |
| | | | | | | lnaAs; omeGs; |
| | | | | | | lnaGs; omeAs; |
| | | | | | | lnaA- |
| | | | | | | Sup |
| 558 | FXN-762 | CCTCAAAAGCAGG | FXN | 3′ | human | lnaCs; omeCs; |
| | m01 | | | | | lnaTs; omeCs; |
| | | | | | | lnaAs; omeAs; |
| | | | | | | lnaAs; omeAs; |
| | | | | | | lnaGs; omeCs; |
| | | | | | | lnaAs; omeGs; |
| | | | | | | lnaG- |
| | | | | | | Sup |
| 559 | FXN-763 | CCTCAAAAGCA | FXN | 3′ | human | lnaCs; omeCs; |
| | m01 | | | | | lnaTs; omeCs; |
| | | | | | | lnaAs; omeAs; |
| | | | | | | lnaAs; omeAs; |
| | | | | | | lnaGs; omeCs; |
| | | | | | | lnaA- |
| | | | | | | Sup |
| 560 | FXN-764 | TCAAAAGCAGGAA | FXN | 3′ | human | lnaTs; omeCs; |
| | m01 | | | | | lnaAs; omeAs; |
| | | | | | | lnaAs; omeAs; |
| | | | | | | lnaGs; omeCs; |
| | | | | | | lnaAs; omeGs; |
| | | | | | | lnaGs; omeAs; |
| | | | | | | lnaA- |
| | | | | | | Sup |
| 561 | FXN-765 | CAAAAGCAGGA | FXN | 3′ | human | lnaCs; omeAs; |
| | m01 | | | | | lnaAs; omeAs; |
| | | | | | | lnaAs; omeGs; |
| | | | | | | lnaCs; omeAs; |
| | | | | | | lnaGs; omeGs; |
| | | | | | | lnaA-Sup |
| 562 | FXN-766 | CCGCCCTCCAGCCTCA | FXN | 5′ and 3′ | human | lnaCs; omeCs; |
| | m01 | AAAGCAGGAAT | | | | lnaGs; omeCs; |
| | | | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaGs; omeGs; |
| | | | | | | lnaAs; omeAs; |
| | | | | | | lnaT-Sup |
| 563 | FXN-767 | CCGCCCTCCAGCCTCA | FXN | 5′ and 3′ | human | lnaCs; omeCs; |
| | m01 | AAAGCAGGA | | | | lnaGs; omeCs; |
| | | | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaGs; omeGs; |
| | | | | | | lnaA- |
| | | | | | | Sup |
| 564 | FXN-768 | CCGCCCTCCAGCCTCA | FXN | 5′ and 3′ | human | lnaCs; omeCs; |
| | m01 | AAAGCAG | | | | lnaGs; omeCs; |
| | | | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaG- |
| | | | | | | Sup |
| 565 | FXN-769 | CCGCCCTCCAGCCTCA | FXN | 5′ and 3′ | human | lnaCs; omeCs; |
| | m01 | AAAGC | | | | lnaGs; omeCs; |
| | | | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaC- |
| | | | | | | Sup |
| 566 | FXN-770 | GCCCTCCAGCCTCAAA | FXN | 5′ and 3′ | human | lnaGs; omeCs; |
| | m01 | AGCAGGAAT | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaGs; omeGs; |
| | | | | | | lnaAs; omeAs; |
| | | | | | | lnaT- |
| | | | | | | Sup |
| 567 | FXN-771 | GCCCTCCAGCCTCAAA | FXN | 5′ and 3′ | human | lnaGs; omeCs; |
| | m01 | AGCAGGA | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaGs; omeGs; |
| | | | | | | lnaA- |
| | | | | | | Sup |
| 568 | FXN-772 | GCCCTCCAGCCTCAAA | FXN | 5′ and 3′ | human | lnaGs; omeCs; |
| | m01 | AGCAG | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaG- |
| | | | | | | Sup |
| 569 | FXN-773 | GCCCTCCAGCCTCAAA | FXN | 5′ and 3′ | human | lnaGs; omeCs; |
| | m01 | AGC | | | | lnaCs; omeCs; |
| | | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaAs; omeGs; |
| | | | | | | lnaC-Sup |
| 570 | FXN-774 | CCCTCCAGCCTCAAAAG | FXN | 5′ and 3′ | human | lnaCs; omeCs; |
| | m01 | | | | | lnaCs; omeTs; |
| | | | | | | lnaCs; omeCs; |
| | | | | | | lnaAs; omeGs; |
| | | | | | | lnaCs; omeCs; |
| | | | | | | lnaTs; omeCs; |
| | | | | | | lnaAs; omeAs; |
| | | | | | | lnaAs; omeAs; |
| | | | | | | lnaG-Sup |
| 571 | FXN-776 | CCTCCAGCCTCAAAA | FXN | 5′ and 3′ | human | lnaCs; omeCs; |
| | m01 | | | | | lnaTs; omeCs; |
| | | | | | | lnaCs; omeAs; |
| | | | | | | lnaGs; omeCs; |
| | | | | | | lnaCs; omeTs; |
| | | | | | | lnaCs; omeAs; |
| | | | | | | lnaAs; omeAs; |
| | | | | | | lnaA- |
| | | | | | | Sup |
| 572 | FXN-777 | GCCCTCCAGTCAAAA | FXN | 5′ and 3′ | human | lnaGs; omeCs; |
| | m01 | GCAGGA | | | | lnaCs; omeCs; |
| | | | | | | lnaTs; omeCs; |
| | | | | | | lnaCs; omeAs; |
| | | | | | | lnaGs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaGs; omeGs; |
| | | | | | | lnaA- |
| | | | | | | Sup |
| 573 | FXN-778 | GCCCTCCAGCAAAAG | FXN | 5′ and 3′ | human | lnaGs; omeCs; |
| | m01 | CAGG | | | | lnaCs; omeCs; |
| | | | | | | lnaTs; omeCs; |
| | | | | | | lnaCs; omeAs; |
| | | | | | | lnaGs; omeCs; |
| | | | | | | lnaAs; omeAs; |
| | | | | | | lnaAs; omeAs; |
| | | | | | | lnaGs; omeCs; |
| | | | | | | lnaAs; omeGs; |
| | | | | | | lnaG-Sup |
| 574 | FXN-779 | CCGCCCTCCAGTCAAA | FXN | 5′ and 3′ | human | lnaCs; omeCs; |
| | m01 | AGCAGGA | | | | lnaGs; omeCs; |
| | | | | | | lnaCs; omeCs; |
| | | | | | | lnaTs; omeCs; |
| | | | | | | lnaCs; omeAs; |
| | | | | | | lnaGs; omeTs; |
| | | | | | | lnaCs; omeAs; |
| | | | | | | lnaAs; omeAs; |
| | | | | | | lnaAs; omeGs; |
| | | | | | | lnaCs; omeAs; |
| | | | | | | lnaGs; omeGs; |
| | | | | | | lnaA- |
| | | | | | | Sup |
| 575 | FXN-780 | CCGCCCTCCAGCAAA | FXN | 5′ and 3′ | human | lnaCs; omeCs; |
| | m01 | AGCAGG | | | | lnaGs; omeCs; |
| | | | | | | lnaCs; omeCs; |
| | | | | | | lnaTs; omeCs; |
| | | | | | | lnaCs; omeAs; |
| | | | | | | lnaGs; omeCs; |
| | | | | | | lnaAs; omeAs; |
| | | | | | | lnaAs; omeAs; |
| | | | | | | lnaGs; omeCs; |
| | | | | | | lnaAs; omeGs; |
| | | | | | | lnaG-Sup |
| 576 | FXN-671 | CTCCGCCCTCCAG | FXN | 5′ | human | lnaCs; omeTs; |
| | m01 | | | | | lnaCs; omeCs; |
| | | | | | | lnaGs; omeCs; |
| | | | | | | lnaCs; omeCs; |
| | | | | | | lnaTs; omeCs; |
| | | | | | | lnaCs; omeAs; |
| | | | | | | lnaG- |
| | | | | | | Sup |
| 577 | FXN-672 | CCGCCCTCCAG | FXN | 5′ | human | lnaCs; omeCs; |
| | m01 | | | | | lnaGs; omeCs; |
| | | | | | | lnaCs; omeCs; |
| | | | | | | lnaTs; omeCs; |
| | | | | | | lnaCs; omeAs; |
| | | | | | | lnaG-Sup |
| 578 | FXN-673 | GCCCTCCAG | FXN | 5′ | human | lnaGs; omeCs; |
| | m01 | | | | | lnaCs; omeCs; |
| | | | | | | lnaTs; omeCs; |
| | | | | | | lnaCs; omeAs; |
| | | | | | | lnaG-Sup |
| 579 | FXN-674 | CCCGCTCCGCCCTCC | FXN | 5′ | human | lnaCs; omeCs; |
| | m01 | | | | | lnaCs; omeGs; |
| | | | | | | lnaCs; omeTs; |
| | | | | | | lnaCs; omeCs; |
| | | | | | | lnaGs; omeCs; |
| | | | | | | lnaCs; omeCs; |
| | | | | | | lnaTs; omeCs; |
| | | | | | | lnaC- |
| | | | | | | Sup |
| 580 | FXN-675 | CGCTCCGCCCTCC | FXN | 5′ | human | lnaCs; omeGs; |
| | m01 | | | | | lnaCs; omeTs; |
| | | | | | | lnaCs; omeCs; |
| | | | | | | lnaGs; omeCs; |
| | | | | | | lnaCs; omeCs; |
| | | | | | | lnaTs; omeCs; |
| | | | | | | lnaC- |
| | | | | | | Sup |
| 581 | FXN-676 | CTCCGCCCTCC | FXN | 5′ | human | lnaCs; omeTs; |
| | m01 | | | | | lnaCs; omeCs; |
| | | | | | | lnaGs; omeCs; |
| | | | | | | lnaCs; omeCs; |
| | | | | | | lnaTs; omeCs; |
| | | | | | | lnaC- |
| | | | | | | Sup |
| 582 | FXN-677 | CCGCCCTCC | FXN | 5′ | human | lnaCs; omeCs; |
| | m01 | | | | | lnaGs; omeCs; |
| | | | | | | lnaCs; omeCs; |
| | | | | | | lnaTs; omeCs; |
| | | | | | | lnaC-Sup |
| | | | | Targeting | | |
| | | | | Region | | |
| 583 | CD247- | GCCTTTGAGAAAGCA | CD247 | 5′ | human | dGs; lnaCs; |
| | 90 m02 | | | | | dCs; lnaTs; |
| | | | | | | dTs; lnaTs; |
| | | | | | | dGs; lnaAs; |
| | | | | | | dGs; lnaAs; |
| | | | | | | dAs; lnaAs; |
| | | | | | | dGs; lnaCs; |
| | | | | | | dA-Sup |
| 584 | CD247- | GACTGTGGGGCCTTT | CD247 | 5′ | human | dGs; lnaAs; |
| | 91 m02 | | | | | dCs; lnaTs; |
| | | | | | | dGs; lnaTs; |
| | | | | | | dGs; lnaGs; |
| | | | | | | dGs; lnaGs; |
| | | | | | | dCs; lnaCs; |
| | | | | | | dTs; lnaTs; |
| | | | | | | dT-Sup |
| 585 | CD247- | AGGAAGTGGAGGACT | CD247 | 5′ | human | dAs; lnaGs; |
| | 92 m02 | | | | | dGs; lnaAs; |
| | | | | | | dAs; lnaGs; |
| | | | | | | dTs; lnaGs; |
| | | | | | | dGs; lnaAs; |
| | | | | | | dGs; lnaGs; |
| | | | | | | dAs; lnaCs; |
| | | | | | | dT-Sup |
| 586 | CD247- | TGCATTTTCACTGAA | CD247 | 3′ | human | dTs; lnaGs; |
| | 93 m02 | | | | | dCs; lnaAs; |
| | | | | | | dTs; lnaTs; |
| | | | | | | dTs; lnaTs; |
| | | | | | | dCs; lnaAs; |
| | | | | | | dCs; lnaTs; |
| | | | | | | dGs; lnaAs; |
| | | | | | | dA-Sup |
| 587 | CD247- | CATTTTCACTGAAGC | CD247 | 3′ | human | dCs; lnaAs; |
| | 94 m02 | | | | | dTs; lnaTs; |
| | | | | | | dTs; lnaTs; |
| | | | | | | dCs; lnaAs; |
| | | | | | | dCs; lnaTs; |
| | | | | | | dGs; lnaAs; |
| | | | | | | dAs; lnaGs; |
| | | | | | | dC-Sup |
| 588 | CD247- | ACTGAAGCATTTATT | CD247 | 3′ | human | dAs; lnaCs; |
| | 95 m02 | | | | | dTs; lnaGs; |
| | | | | | | dAs; lnaAs; |
| | | | | | | dGs; lnaCs; |
| | | | | | | dAs; lnaTs; |
| | | | | | | dTs; lnaTs; |
| | | | | | | dAs; lnaTs; |
| | | | | | | dT-Sup |
| 589 | CFTR-84 | CACACAAATGTATGG | CFTR | 3′ | human | dCs; lnaAs; |
| | m02 | | | | | dCs; lnaAs; |
| | | | | | | dCs; lnaAs; |
| | | | | | | dAs; lnaAs; |
| | | | | | | dTs; lnaGs; |
| | | | | | | dTs; lnaAs; |
| | | | | | | dTs; lnaGs; |
| | | | | | | dG-Sup |
| 590 | CFTR-85 | GGATTTTATTGACAA | CFTR | 3′ | human | dGs; lnaGs; |
| | m02 | | | | | dAs; lnaTs; |
| | | | | | | dTs; lnaTs; |
| | | | | | | dTs; lnaAs; |
| | | | | | | dTs; lnaTs; |
| | | | | | | dGs; lnaAs; |
| | | | | | | dCs; lnaAs; |
| | | | | | | dA-Sup |
| 591 | CFTR-86 | AAAACAACAAAGTTT | CFTR | 3′ | human | dAs; lnaAs; |
| | m02 | | | | | dAs; lnaAs; |
| | | | | | | dCs; lnaAs; |
| | | | | | | dAs; lnaCs; |
| | | | | | | dAs; lnaAs; |
| | | | | | | dAs; lnaGs; |
| | | | | | | dTs; lnaTs; |
| | | | | | | dT-Sup |
| 592 | CFTR-87 | AGTGCCATAAAAAGT | CFTR | 3′ | human | dAs; lnaGs; |
| | m02 | | | | | dTs; lnaGs; |
| | | | | | | dCs; lnaCs; |
| | | | | | | dAs; lnaTs; |
| | | | | | | dAs; lnaAs; |
| | | | | | | dAs; lnaAs; |
| | | | | | | dAs; lnaGs; |
| | | | | | | dT-Sup |
| 593 | CFTR-88 | TCAAATATAAAAATT | CFTR | 3′ | human | dTs; lnaCs; |
| | m02 | | | | | dAs; lnaAs; |
| | | | | | | dAs; lnaTs; |
| | | | | | | dAs; lnaTs; |
| | | | | | | dAs; lnaAs; |
| | | | | | | dAs; lnaAs; |
| | | | | | | dAs; lnaTs; |
| | | | | | | dT-Sup |
| 594 | CFTR-89 | TTCCCCCCACCCACC | CFTR | 3′ | human | dTs; lnaTs; |
| | m02 | | | | | dCs; lnaCs; |
| | | | | | | dCs; lnaCs; |
| | | | | | | dCs; lnaCs; |
| | | | | | | dAs; lnaCs; |
| | | | | | | dCs; lnaCs; |
| | | | | | | dAs; lnaCs; |
| | | | | | | dC-Sup |
| 595 | CFTR-90 | CATTTGCTTCCAATT | CFTR | 5′ | human | dCs; lnaAs; |
| | m02 | | | | | dTs; lnaTs; |
| | | | | | | dTs; lnaGs; |
| | | | | | | dCs; lnaTs; |
| | | | | | | dTs; lnaCs; |
| | | | | | | dCs; lnaAs; |
| | | | | | | dAs; lnaTs; |
| | | | | | | dT-Sup |
| 596 | CFTR-91 | GCTCAACCCTTTTTC | CFTR | 5′ | human | dGs; lnaCs; |
| | m02 | | | | | dTs; lnaCs; |
| | | | | | | dAs; lnaAs; |
| | | | | | | dCs; lnaCs; |
| | | | | | | dCs; lnaTs; |
| | | | | | | dTs; lnaTs; |
| | | | | | | dTs; lnaTs; |
| | | | | | | dC-Sup |
| 597 | CFTR-92 | AGACCTACTACTCTG | CFTR | 5′ | human | dAs; lnaGs; |
| | m02 | | | | | dAs; lnaCs; |
| | | | | | | dCs; lnaTs; |
| | | | | | | dAs; lnaCs; |
| | | | | | | dTs; lnaAs; |
| | | | | | | dCs; lnaTs; |
| | | | | | | dCs; lnaTs; |
| | | | | | | dG-Sup |
| 598 | FMR1- | CCCTCCACCGGAAGT | FMR1 | 5′ | human | dCs; lnaCs; |
| | 58 m02 | | | | | dCs; lnaTs; |
| | | | | | | dCs; lnaCs; |
| | | | | | | dAs; lnaCs; |
| | | | | | | dCs; lnaGs; |
| | | | | | | dGs; lnaAs; |
| | | | | | | dAs; lnaGs; |
| | | | | | | dT-Sup |
| 599 | FMR1- | GCCCGCGCTCGCCGT | FMR1 | 5′ | human | dGs; lnaCs; |
| | 59 m02 | | | | | dCs; lnaCs; |
| | | | | | | dGs; lnaCs; |
| | | | | | | dGs; lnaCs; |
| | | | | | | dTs; lnaCs; |
| | | | | | | dGs; lnaCs; |
| | | | | | | dCs; lnaGs; |
| | | | | | | dT-Sup |
| 600 | FMR1- | ACGCCCCCTGGCAGC | FMR1 | 5′ | human | dAs; lnaCs; |
| | 60 m02 | | | | | dGs; lnaCs; |
| | | | | | | dCs; lnaCs; |
| | | | | | | dCs; lnaCs; |
| | | | | | | dTs; lnaGs; |
| | | | | | | dGs; lnaCs; |
| | | | | | | dAs; lnaGs; |
| | | | | | | dC-Sup |
| 601 | FMR1- | GCTCAGCCCCTCGGC | FMR1 | 5′ | human | dGs; lnaCs; |
| | 61 m02 | | | | | dTs; lnaCs; |
| | | | | | | dAs; lnaGs; |
| | | | | | | dCs; lnaCs; |
| | | | | | | dCs; lnaCs; |
| | | | | | | dTs; lnaCs; |
| | | | | | | dGs; lnaGs; |
| | | | | | | dC-Sup |
| 602 | FMR1- | AGCAGAGGAAGATCA | FMR1 | 3′ | human | dAs; lnaGs; |
| | 62 m02 | | | | | dCs; lnaAs; |
| | | | | | | dGs; lnaAs; |
| | | | | | | dGs; lnaGs; |
| | | | | | | dAs; lnaAs; |
| | | | | | | dGs; lnaAs; |
| | | | | | | dTs; lnaCs; |
| | | | | | | dA-Sup |
| 603 | FMR1- | CAGAGGAAGATCAAA | FMR1 | 3′ | human | dCs; lnaAs; |
| | 63 m02 | | | | | dGs; lnaAs; |
| | | | | | | dGs; lnaGs; |
| | | | | | | dAs; lnaAs; |
| | | | | | | dGs; lnaAs; |
| | | | | | | dTs; lnaCs; |
| | | | | | | dAs; lnaAs; |
| | | | | | | dA-Sup |
| 604 | FMR1- | CAGATTTTTGAAACT | FMR1 | 3′ | human | dCs; lnaAs; |
| | 64 m02 | | | | | dGs; lnaAs; |
| | | | | | | dTs; lnaTs; |
| | | | | | | dTs; lnaTs; |
| | | | | | | dTs; lnaGs; |
| | | | | | | dAs; lnaAs; |
| | | | | | | dAs; lnaCs; |
| | | | | | | dT-Sup |
| 605 | FMR1- | CAGACTAATTTTTTG | FMR1 | 3′ | human | dCs; lnaAs; |
| | 65 m02 | | | | | dGs; lnaAs; |
| | | | | | | dCs; lnaTs; |
| | | | | | | dAs; lnaAs; |
| | | | | | | dTs; lnaTs; |
| | | | | | | dTs; lnaTs; |
| | | | | | | dTs; lnaTs; |
| | | | | | | dG-Sup |
| 606 | FMR1- | TTTTTGCTTTTTCAT | FMR1 | 3′ | human | dTs; lnaTs; |
| | 66 m02 | | | | | dTs; lnaTs; |
| | | | | | | dTs; lnaGs; |
| | | | | | | dCs; lnaTs; |
| | | | | | | dTs; lnaTs; |
| | | | | | | dTs; lnaTs; |
| | | | | | | dCs; lnaAs; |
| | | | | | | dT-Sup |
| 607 | FMR1- | AATTTTTTGCTTTTT | FMR1 | 3′ | human | dAs; lnaAs; |
| | 67 m02 | | | | | dTs; lnaTs; |
| | | | | | | dTs; lnaTs; |
| | | | | | | dTs; lnaTs; |
| | | | | | | dGs; lnaCs; |
| | | | | | | dTs; lnaTs; |
| | | | | | | dTs; lnaTs; |
| | | | | | | dT-Sup |
| 608 | FMR1- | ATGTTTGGCAATACT | FMR1 | 3′ | human | dAs; lnaTs; |
| | 68 m02 | | | | | dGs; lnaTs; |
| | | | | | | dTs; lnaTs; |
| | | | | | | dGs; lnaGs; |
| | | | | | | dCs; lnaAs; |
| | | | | | | dAs; lnaTs; |
| | | | | | | dAs; lnaCs; |
| | | | | | | dT-Sup |
| 609 | FMR1- | TTGGCAATACTTTTT | FMR1 | 3′ | human | dTs; lnaTs; |
| | 69 m02 | | | | | dGs; lnaGs; |
| | | | | | | dCs; lnaAs; |
| | | | | | | dAs; lnaTs; |
| | | | | | | dAs; lnaCs; |
| | | | | | | dTs; lnaTs; |
| | | | | | | dTs; lnaTs; |
| | | | | | | dT-Sup |
| 610 | LAMA1- | GCTGCCCTGGCCCCG | LAMA1 | 5′ | human | dGs; lnaCs; |
| | 105 | | | | | dTs; lnaGs; |
| | m02 | | | | | dCs; lnaCs; |
| | | | | | | dCs; lnaTs; |
| | | | | | | dGs; lnaGs; |
| | | | | | | dCs; lnaCs; |
| | | | | | | dCs; lnaCs; |
| | | | | | | dG-Sup |
| 611 | LAMA1- | CGGACACACCCCTCG | LAMA1 | 5′ | human | dCs; lnaGs; |
| | 106 | | | | | dGs; lnaAs; |
| | m02 | | | | | dCs; lnaAs; |
| | | | | | | dCs; lnaAs; |
| | | | | | | dCs; lnaCs; |
| | | | | | | dCs; lnaCs; |
| | | | | | | dTs; lnaCs; |
| | | | | | | dG-Sup |
| 612 | LAMA1- | ACGGGACGCGAGTCC | LAMA1 | 5′ | human | dAs; lnaCs; |
| | 107 | | | | | dGs; lnaGs; |
| | m02 | | | | | dGs; lnaAs; |
| | | | | | | dCs; lnaGs; |
| | | | | | | dCs; lnaGs; |
| | | | | | | dAs; lnaGs; |
| | | | | | | dTs; lnaCs; |
| | | | | | | dC-Sup |
| 613 | LAMA1- | GTCTGGGGAGAAAGC | LAMA1 | 5′ | human | dGs; lnaTs; |
| | 108 | | | | | dCs; lnaTs; |
| | m02 | | | | | dGs; lnaGs; |
| | | | | | | dGs; lnaGs; |
| | | | | | | dAs; lnaGs; |
| | | | | | | dAs; lnaAs; |
| | | | | | | dAs; lnaGs; |
| | | | | | | dC-Sup |
| 614 | LAMA1- | CCACTCGGTGGGTCT | LAMA1 | 5′ | human | dCs; lnaCs; |
| | 109 | | | | | dAs; lnaCs; |
| | m02 | | | | | dTs; lnaCs; |
| | | | | | | dGs; lnaGs; |
| | | | | | | dTs; lnaGs; |
| | | | | | | dGs; lnaGs; |
| | | | | | | dTs; lnaCs; |
| | | | | | | dT-Sup |
| 615 | LAMA1- | TGATCTGTTATCATC | LAMA1 | 5′ | human | dTs; lnaGs; |
| | 110 | | | | | dAs; lnaTs; |
| | m02 | | | | | dCs; lnaTs; |
| | | | | | | dGs; lnaTs; |
| | | | | | | dTs; lnaAs; |
| | | | | | | dTs; lnaCs; |
| | | | | | | dAs; lnaTs; |
| | | | | | | dC-Sup |
| 616 | LAMA1- | CTGTTATCATCTGTA | LAMA1 | 3′ | human | dCs; lnaTs; |
| | 111 | | | | | dGs; lnaTs; |
| | m02 | | | | | dTs; lnaAs; |
| | | | | | | dTs; lnaCs; |
| | | | | | | dAs; lnaTs; |
| | | | | | | dCs; lnaTs; |
| | | | | | | dGs; lnaTs; |
| | | | | | | dA-Sup |
| 617 | LAMA1- | GTGTATAAAGATTTT | LAMA1 | 3′ | human | dGs; lnaTs; |
| | 112 | | | | | dGs; lnaTs; |
| | m02 | | | | | dAs; lnaTs; |
| | | | | | | dAs; lnaAs; |
| | | | | | | dAs; lnaGs; |
| | | | | | | dAs; lnaTs; |
| | | | | | | dTs; lnaTs; |
| | | | | | | dT-Sup |
| 618 | LAMA1- | CAATTTACATTTTAG | LAMA1 | 3′ | human | dCs; lnaAs; |
| | 113 | | | | | dAs; lnaTs; |
| | m02 | | | | | dTs; lnaTs; |
| | | | | | | dAs; lnaCs; |
| | | | | | | dAs; lnaTs; |
| | | | | | | dTs; lnaTs; |
| | | | | | | dTs; lnaAs; |
| | | | | | | dG-Sup |
| 619 | LAMA1- | TACATTTTAGACCAT | LAMA1 | 3′ | human | dTs; lnaAs; |
| | 114 | | | | | dCs; lnaAs; |
| | m02 | | | | | dTs; lnaTs; |
| | | | | | | dTs; lnaTs; |
| | | | | | | dAs; lnaGs; |
| | | | | | | dAs; lnaCs; |
| | | | | | | dCs; lnaAs; |
| | | | | | | dT-Sup |
| 620 | MBNL1- | TGCTATAAGATGTAA | MBNL1 | 5′ | human | dTs; lnaGs; |
| | 73 m02 | | | | | dCs; lnaTs; |
| | | | | | | dAs; lnaTs; |
| | | | | | | dAs; lnaAs; |
| | | | | | | dGs; lnaAs; |
| | | | | | | dTs; lnaGs; |
| | | | | | | dTs; lnaAs; |
| | | | | | | dA-Sup |
| 621 | MBNL1- | AAGGAAGCCGGCAAG | MBNL1 | 5′ | human | dAs; lnaAs; |
| | 74 m02 | | | | | dGs; lnaGs; |
| | | | | | | dAs; lnaAs; |
| | | | | | | dGs; lnaCs; |
| | | | | | | dCs; lnaGs; |
| | | | | | | dGs; lnaCs; |
| | | | | | | dAs; lnaAs; |
| | | | | | | dG-Sup |
| 622 | MBNL1- | CGCCACAACTCATTC | MBNL1 | 5′ | human | dCs; lnaGs; |
| | 75 m02 | | | | | dCs; lnaCs; |
| | | | | | | dAs; lnaCs; |
| | | | | | | dAs; lnaAs; |
| | | | | | | dCs; lnaTs; |
| | | | | | | dCs; lnaAs; |
| | | | | | | dTs; lnaTs; |
| | | | | | | dC-Sup |
| 623 | MBNL1- | ATGGGAGCATTGTGG | MBNL1 | 5′ | human | dAs; lnaTs; |
| | 76 m02 | | | | | dGs; lnaGs; |
| | | | | | | dGs; lnaAs; |
| | | | | | | dGs; lnaCs; |
| | | | | | | dAs; lnaTs; |
| | | | | | | dTs; lnaGs; |
| | | | | | | dTs; lnaGs; |
| | | | | | | dG-Sup |
| 624 | MBNL1- | CGCCCGCCCAGCCCC | MBNL1 | 5′ | human | dCs; lnaGs; |
| | 77 m02 | | | | | dCs; lnaCs; |
| | | | | | | dCs; lnaGs; |
| | | | | | | dCs; lnaCs; |
| | | | | | | dCs; lnaAs; |
| | | | | | | dGs; lnaCs; |
| | | | | | | dCs; lnaCs; |
| | | | | | | dC-Sup |
| 625 | MBNL1- | CCCCTCCCCCGCCCG | MBNL1 | 5′ | human | dCs; lnaCs; |
| | 78 m02 | | | | | dCs; lnaCs; |
| | | | | | | dTs; lnaCs; |
| | | | | | | dCs; lnaCs; |
| | | | | | | dCs; lnaCs; |
| | | | | | | dGs; lnaCs; |
| | | | | | | dCs; lnaCs; |
| | | | | | | dG-Sup |
| 626 | MBNL1- | CTTCCGCTGCTGCTG | MBNL1 | 5′ | human | dCs; lnaTs; |
| | 79 m02 | | | | | dTs; lnaCs; |
| | | | | | | dCs; lnaGs; |
| | | | | | | dCs; lnaTs; |
| | | | | | | dGs; lnaCs; |
| | | | | | | dTs; lnaGs; |
| | | | | | | dCs; lnaTs; |
| | | | | | | dG-Sup |
| 627 | MBNL1- | CTTCTTAGTACCAAC | MBNL1 | 5′ | human | dCs; lnaTs; |
| | 80 m02 | | | | | dTs; lnaCs; |
| | | | | | | dTs; lnaTs; |
| | | | | | | dAs; lnaGs; |
| | | | | | | dTs; lnaAs; |
| | | | | | | dCs; lnaCs; |
| | | | | | | dAs; lnaAs; |
| | | | | | | dC-Sup |
| 628 | MBNL1- | TTTAGAGCAAAATCG | MBNL1 | 5′ | human | dTs; lnaTs; |
| | 81 m02 | | | | | dTs; lnaAs; |
| | | | | | | dGs; lnaAs; |
| | | | | | | dGs; lnaCs; |
| | | | | | | dAs; lnaAs; |
| | | | | | | dAs; lnaAs; |
| | | | | | | dTs; lnaCs; |
| | | | | | | dG-Sup |
| 629 | MBNL1- | GGTAGTTAAATGTTT | MBNL1 | 5′ | human | dGs; lnaGs; |
| | 82 m02 | | | | | dTs; lnaAs; |
| | | | | | | dGs; lnaTs; |
| | | | | | | dTs; lnaAs; |
| | | | | | | dAs; lnaAs; |
| | | | | | | dTs; lnaGs; |
| | | | | | | dTs; lnaTs; |
| | | | | | | dT-Sup |
| 630 | MBNL1- | TACTTAAGAAAGAGA | MBNL1 | 3′ | human | dTs; lnaAs; |
| | 83 m02 | | | | | dCs; lnaTs; |
| | | | | | | dTs; lnaAs; |
| | | | | | | dAs; lnaGs; |
| | | | | | | dAs; lnaAs; |
| | | | | | | dAs; lnaGs; |
| | | | | | | dAs; lnaGs; |
| | | | | | | dA-Sup |
| 631 | MBNL1- | TATACTTAAGAAAGA | MBNL1 | 3′ | human | dTs; lnaAs; |
| | 84 m02 | | | | | dTs; lnaAs; |
| | | | | | | dCs; lnaTs; |
| | | | | | | dTs; lnaAs; |
| | | | | | | dAs; lnaGs; |
| | | | | | | dAs; lnaAs; |
| | | | | | | dAs; lnaGs; |
| | | | | | | dA-Sup |
| 632 | MECP2- | CGCCGCCGACGCCGG | MECP2 | 5′ | human | dCs; lnaGs; |
| | 61 m02 | | | | | dCs; lnaCs; |
| | | | | | | dGs; lnaCs; |
| | | | | | | dCs; lnaGs; |
| | | | | | | dAs; lnaCs; |
| | | | | | | dGs; lnaCs; |
| | | | | | | dCs; lnaGs; |
| | | | | | | dG-Sup |
| 633 | MECP2- | CTCTCTCCGAGAGGA | MECP2 | 5′ | human | dCs; lnaTs; |
| | 62 m02 | | | | | dCs; lnaTs; |
| | | | | | | dCs; lnaTs; |
| | | | | | | dCs; lnaCs; |
| | | | | | | dGs; lnaAs; |
| | | | | | | dGs; lnaAs; |
| | | | | | | dGs; lnaGs; |
| | | | | | | dA-Sup |
| 634 | MECP2- | CGCCCCGCCCTCTTG | MECP2 | 5′ | human | dCs; lnaGs; |
| | 63 m02 | | | | | dCs; lnaCs; |
| | | | | | | dCs; lnaCs; |
| | | | | | | dGs; lnaCs; |
| | | | | | | dCs; lnaCs; |
| | | | | | | dTs; lnaCs; |
| | | | | | | dTs; lnaTs; |
| | | | | | | dG-Sup |
| 635 | MECP2- | CCGCGCGCTGCTGCA | MECP2 | 5′ | human | dCs; lnaCs; |
| | 64 m02 | | | | | dGs; lnaCs; |
| | | | | | | dGs; lnaCs; |
| | | | | | | dGs; lnaCs; |
| | | | | | | dTs; lnaGs; |
| | | | | | | dCs; lnaTs; |
| | | | | | | dGs; lnaCs; |
| | | | | | | dA-Sup |
| 636 | MECP2- | CACTTTCACAGAGAG | MECP2 | 3′ | human | dCs; lnaAs; |
| | 65 m02 | | | | | dCs; lnaTs; |
| | | | | | | dTs; lnaTs; |
| | | | | | | dCs; lnaAs; |
| | | | | | | dCs; lnaAs; |
| | | | | | | dGs; lnaAs; |
| | | | | | | dGs; lnaAs; |
| | | | | | | dG-Sup |
| 637 | MECP2- | CTTTCACATGTATTAA | MECP2 | 3′ | human | dCs; lnaTs; |
| | 66 m02 | | | | | dTs; lnaTs; |
| | | | | | | dCs; lnaAs; |
| | | | | | | dCs; lnaAs; |
| | | | | | | dTs; lnaGs; |
| | | | | | | dTs; lnaAs; |
| | | | | | | dTs; lnaTs; |
| | | | | | | dAs; dA-Sup |
| 638 | MECP2- | ATGTATTAAAAAACT | MECP2 | 3′ | human | dAs; lnaTs; |
| | 67 m02 | | | | | dGs; lnaTs; |
| | | | | | | dAs; lnaTs; |
| | | | | | | dTs; lnaAs; |
| | | | | | | dAs; lnaAs; |
| | | | | | | dAs; lnaAs; |
| | | | | | | dAs; lnaCs; |
| | | | | | | dT-Sup |
| 639 | MECP2- | GACATTTTTATGTAA | MECP2 | 3′ | human | dGs; lnaAs; |
| | 68 m02 | | | | | dCs; lnaAs; |
| | | | | | | dTs; lnaTs; |
| | | | | | | dTs; lnaTs; |
| | | | | | | dTs; lnaAs; |
| | | | | | | dTs; lnaGs; |
| | | | | | | dTs; lnaAs; |
| | | | | | | dA-Sup |
| 640 | MECP2- | CATTTTTATGTAAAT | MECP2 | 3′ | human | dCs; lnaAs; |
| | 69 m02 | | | | | dTs; lnaTs; |
| | | | | | | dTs; lnaTs; |
| | | | | | | dTs; lnaAs; |
| | | | | | | dTs; lnaGs; |
| | | | | | | dTs; lnaAs; |
| | | | | | | dAs; lnaAs; |
| | | | | | | dT-Sup |
| 641 | MECP2- | AAATTTATAAGGCAA | MECP2 | 3′ | human | dAs; lnaAs; |
| | 70 m02 | | | | | dAs; lnaTs; |
| | | | | | | dTs; lnaTs; |
| | | | | | | dAs; lnaTs; |
| | | | | | | dAs; lnaAs; |
| | | | | | | dGs; lnaGs; |
| | | | | | | dCs; lnaAs; |
| | | | | | | dA-Sup |
| 642 | MECP2- | AGGCAAACTCTTTAT | MECP2 | 3′ | human | dAs; lnaGs; |
| | 71 m02 | | | | | dGs; lnaCs; |
| | | | | | | dAs; lnaAs; |
| | | | | | | dAs; lnaCs; |
| | | | | | | dTs; lnaCs; |
| | | | | | | dTs; lnaTs; |
| | | | | | | dTs; lnaAs; |
| | | | | | | dT-Sup |
| 643 | MECP2- | GTCTCTGGAACAATT | MECP2 | 3′ | human | dGs; lnaTs; |
| | 72 m02 | | | | | dCs; lnaTs; |
| | | | | | | dCs; lnaTs; |
| | | | | | | dGs; lnaGs; |
| | | | | | | dAs; lnaAs; |
| | | | | | | dCs; lnaAs; |
| | | | | | | dAs; lnaTs; |
| | | | | | | dT-Sup |
| 644 | MECP2- | CAGTTCAAACACAGA | MECP2 | 3′ | human | dCs; lnaAs; |
| | 73 m02 | | | | | dGs; lnaTs; |
| | | | | | | dTs; lnaCs; |
| | | | | | | dAs; lnaAs; |
| | | | | | | dAs; lnaCs; |
| | | | | | | dAs; lnaCs; |
| | | | | | | dAs; lnaGs; |
| | | | | | | dA-Sup |
| 645 | MECP2- | CAAACACAGAAGAGA | MECP2 | 3′ | human | dCs; lnaAs; |
| | 74 m02 | | | | | dAs; lnaAs; |
| | | | | | | dCs; lnaAs; |
| | | | | | | dCs; lnaAs; |
| | | | | | | dGs; lnaAs; |
| | | | | | | dAs; lnaGs; |
| | | | | | | dAs; lnaGs; |
| | | | | | | dA-Sup |
| 646 | MECP2- | AACACAGAAGAGATT | MECP2 | 3′ | human | dAs; lnaAs; |
| | 75 m02 | | | | | dCs; lnaAs; |
| | | | | | | dCs; lnaAs; |
| | | | | | | dGs; lnaAs; |
| | | | | | | dAs; lnaGs; |
| | | | | | | dAs; lnaGs; |
| | | | | | | dAs; lnaTs; |
| | | | | | | dT-Sup |
| 647 | MECP2- | GGGGGAGAAGAAAGG | MECP2 | 3′ | human | dGs; lnaGs; |
| | 76 m02 | | | | | dGs; lnaGs; |
| | | | | | | dGs; lnaAs; |
| | | | | | | dGs; lnaAs; |
| | | | | | | dAs; lnaGs; |
| | | | | | | dAs; lnaAs; |
| | | | | | | dAs; lnaGs; |
| | | | | | | dG-Sup |
| 648 | MECP2- | TCGTTTTTTTTTCTT | MECP2 | 3′ | human | dTs; lnaCs; |
| | 77 m02 | | | | | dGs; lnaTs; |
| | | | | | | dTs; lnaTs; |
| | | | | | | dTs; lnaTs; |
| | | | | | | dTs; lnaTs; |
| | | | | | | dTs; lnaTs; |
| | | | | | | dCs; lnaTs; |
| | | | | | | dT-Sup |
| 649 | MECP2- | CTTTTTTTTCTTTTT | MECP2 | 3′ | human | dCs; lnaTs; |
| | 78 m02 | | | | | dTs; lnaTs; |
| | | | | | | dTs; lnaTs; |
| | | | | | | dTs; lnaTs; |
| | | | | | | dTs; lnaCs; |
| | | | | | | dTs; lnaTs; |
| | | | | | | dTs; lnaTs; |
| | | | | | | dT-Sup |
| 650 | MECP2- | CCTATGCTATGGTTA | MECP2 | 3′ | human | dCs; lnaCs; |
| | 79 m02 | | | | | dTs; lnaAs; |
| | | | | | | dTs; lnaGs; |
| | | | | | | dCs; lnaTs; |
| | | | | | | dAs; lnaTs; |
| | | | | | | dGs; lnaGs; |
| | | | | | | dTs; lnaTs; |
| | | | | | | dA-Sup |
| 651 | MECP2- | AGTTTACTGAAAGAA | MECP2 | 3′ | human | dAs; lnaGs; |
| | 80 m02 | | | | | dTs; lnaTs; |
| | | | | | | dTs; lnaAs; |
| | | | | | | dCs; lnaTs; |
| | | | | | | dGs; lnaAs; |
| | | | | | | dAs; lnaAs; |
| | | | | | | dGs; lnaAs; |
| | | | | | | dA-Sup |
| 652 | MECP2- | ACTGAAAGAAAAAAA | MECP2 | 3′ | human | dAs; lnaCs; |
| | 81 m02 | | | | | dTs; lnaGs; |
| | | | | | | dAs; lnaAs; |
| | | | | | | dAs; lnaGs; |
| | | | | | | dAs; lnaAs; |
| | | | | | | dAs; lnaAs; |
| | | | | | | dAs; lnaAs; |
| | | | | | | dA-Sup |
| 653 | MERTK- | CCTTATTCATATTTT | MERTK | 3′ | human | dCs; lnaCs; |
| | 66 m02 | | | | | dTs; lnaTs; |
| | | | | | | dAs; lnaTs; |
| | | | | | | dTs; lnaCs; |
| | | | | | | dAs; lnaTs; |
| | | | | | | dAs; lnaTs; |
| | | | | | | dTs; lnaTs; |
| | | | | | | dT-Sup |
| 654 | MERTK- | CTTCCTTATTCATAT | MERTK | 3′ | human | dCs; lnaTs; |
| | 67 m02 | | | | | dTs; lnaCs; |
| | | | | | | dCs; lnaTs; |
| | | | | | | dTs; lnaAs; |
| | | | | | | dTs; lnaTs; |
| | | | | | | dCs; lnaAs; |
| | | | | | | dTs; lnaAs; |
| | | | | | | dT-Sup |
| 655 | MERTK- | CAATCCTTCAATATT | MERTK | 3′ | human | dCs; lnaAs; |
| | 68 m02 | | | | | dAs; lnaTs; |
| | | | | | | dCs; lnaCs; |
| | | | | | | dTs; lnaTs; |
| | | | | | | dCs; lnaAs; |
| | | | | | | dAs; lnaTs; |
| | | | | | | dAs; lnaTs; |
| | | | | | | dT-Sup |
| 656 | MERTK- | GGCATTTCATTTTAC | MERTK | 3′ | human | dGs; lnaGs; |
| | 69 m02 | | | | | dCs; lnaAs; |
| | | | | | | dTs; lnaTs; |
| | | | | | | dTs; lnaCs; |
| | | | | | | dAs; lnaTs; |
| | | | | | | dTs; lnaTs; |
| | | | | | | dTs; lnaAs; |
| | | | | | | dC-Sup |
| 657 | MERTK- | CATTTTACAAATATT | MERTK | 3′ | human | dCs; lnaAs; |
| | 70 m02 | | | | | dTs; lnaTs; |
| | | | | | | dTs; lnaTs; |
| | | | | | | dAs; lnaCs; |
| | | | | | | dAs; lnaAs; |
| | | | | | | dAs; lnaTs; |
| | | | | | | dAs; lnaTs; |
| | | | | | | dT-Sup |
| 658 | MERTK- | GAAATGAAATAAGTA | MERTK | 3′ | human | dGs; lnaAs; |
| | 71 m02 | | | | | dAs; lnaAs; |
| | | | | | | dTs; lnaGs; |
| | | | | | | dAs; lnaAs; |
| | | | | | | dAs; lnaTs; |
| | | | | | | dAs; lnaAs; |
| | | | | | | dGs; lnaTs; |
| | | | | | | dA-Sup |
| 659 | MERTK- | AGATATGCAAGATAA | MERTK | 3′ | human | dAs; lnaGs; |
| | 72 m02 | | | | | dAs; lnaTs; |
| | | | | | | dAs; lnaTs; |
| | | | | | | dGs; lnaCs; |
| | | | | | | dAs; lnaAs; |
| | | | | | | dGs; lnaAs; |
| | | | | | | dTs; lnaAs; |
| | | | | | | dA-Sup |
| 660 | MERTK- | GCGGGCCCAGCAGGT | MERTK | 5′ | human | dGs; lnaCs; |
| | 73 m02 | | | | | dGs; lnaGs; |
| | | | | | | dGs; lnaCs; |
| | | | | | | dCs; lnaCs; |
| | | | | | | dAs; lnaGs; |
| | | | | | | dCs; lnaAs; |
| | | | | | | dGs; lnaGs; |
| | | | | | | dT-Sup |
| 661 | MERTK- | CAGTGAGTGCCGAGT | MERTK | 5′ | human | dCs; lnaAs; |
| | 74 m02 | | | | | dGs; lnaTs; |
| | | | | | | dGs; lnaAs; |
| | | | | | | dGs; lnaTs; |
| | | | | | | dGs; lnaCs; |
| | | | | | | dCs; lnaGs; |
| | | | | | | dAs; lnaGs; |
| | | | | | | dT-Sup |
| 662 | MERTK- | GCCCGGGCAGTGAGT | MERTK | 5′ | human | dGs; lnaCs; |
| | 75 m02 | | | | | dCs; lnaCs; |
| | | | | | | dGs; lnaGs; |
| | | | | | | dGs; lnaCs; |
| | | | | | | dAs; lnaGs; |
| | | | | | | dTs; lnaGs; |
| | | | | | | dAs; lnaGs; |
| | | | | | | dT-Sup |
| 663 | MERTK- | TGTCCGGGCGGCCCG | MERTK | 5′ | human | dTs; lnaGs; |
| | 76 m02 | | | | | dTs; lnaCs; |
| | | | | | | dCs; lnaGs; |
| | | | | | | dGs; lnaGs; |
| | | | | | | dCs; lnaGs; |
| | | | | | | dGs; lnaCs; |
| | | | | | | dCs; lnaCs; |
| | | | | | | dG-Sup |
| 664 | SSPN-47 | CGCGCGTGTGCGAGT | SSPN | 5′ | human | dCs; lnaGs; |
| | m02 | | | | | dCs; lnaGs; |
| | | | | | | dCs; lnaGs; |
| | | | | | | dTs; lnaGs; |
| | | | | | | dTs; lnaGs; |
| | | | | | | dCs; lnaGs; |
| | | | | | | dAs; lnaGs; |
| | | | | | | dT-Sup |
| 665 | SSPN-48 | CTTCAGACAGGCTGC | SSPN | 5′ | human | dCs; lnaTs; |
| | m02 | | | | | dTs; lnaCs; |
| | | | | | | dAs; lnaGs; |
| | | | | | | dAs; lnaCs; |
| | | | | | | dAs; lnaGs; |
| | | | | | | dGs; lnaCs; |
| | | | | | | dTs; lnaGs; |
| | | | | | | dC-Sup |
| 666 | SSPN-49 | ACCTCTGCACTTCAG | SSPN | 5′ | human | dAs; lnaCs; |
| | m02 | | | | | dCs; lnaTs; |
| | | | | | | dCs; lnaTs; |
| | | | | | | dGs; lnaCs; |
| | | | | | | dAs; lnaCs; |
| | | | | | | dTs; lnaTs; |
| | | | | | | dCs; lnaAs; |
| | | | | | | dG-Sup |
| 667 | SSPN-50 | CGGCGCGGGTCCCTT | SSPN | 5′ | human | dCs; lnaGs; |
| | m02 | | | | | dGs; lnaCs; |
| | | | | | | dGs; lnaCs; |
| | | | | | | dGs; lnaGs; |
| | | | | | | dGs; lnaTs; |
| | | | | | | dCs; lnaCs; |
| | | | | | | dCs; lnaTs; |
| | | | | | | dT-Sup |
| 668 | SSPN-51 | TGGTATTCGAATTAT | SSPN | 5′ | human | dTs; lnaGs; |
| | m02 | | | | | dGs; lnaTs; |
| | | | | | | dAs; lnaTs; |
| | | | | | | dTs; lnaCs; |
| | | | | | | dGs; lnaAs; |
| | | | | | | dAs; lnaTs; |
| | | | | | | dTs; lnaAs; |
| | | | | | | dT-Sup |
| 669 | SSPN-52 | CGGCCTGCCCTGGTA | SSPN | 5′ | human | dCs; lnaGs; |
| | m02 | | | | | dGs; lnaCs; |
| | | | | | | dCs; lnaTs; |
| | | | | | | dGs; lnaCs; |
| | | | | | | dCs; lnaCs; |
| | | | | | | dTs; lnaGs; |
| | | | | | | dGs; lnaTs; |
| | | | | | | dA-Sup |
| 670 | SSPN-53 | TCAGAGATTATGAAA | SSPN | 3′ | human | dTs; lnaCs; |
| | m02 | | | | | dAs; lnaGs; |
| | | | | | | dAs; lnaGs; |
| | | | | | | dAs; lnaTs; |
| | | | | | | dTs; lnaAs; |
| | | | | | | dTs; lnaGs; |
| | | | | | | dAs; lnaAs; |
| | | | | | | dA-Sup |
| 671 | SSPN-54 | TGTTTTCAGAGATTA | SSPN | 3′ | human | dTs; lnaGs; |
| | m02 | | | | | dTs; lnaTs; |
| | | | | | | dTs; lnaTs; |
| | | | | | | dCs; lnaAs; |
| | | | | | | dGs; lnaAs; |
| | | | | | | dGs; lnaAs; |
| | | | | | | dTs; lnaTs; |
| | | | | | | dA-Sup |
| 672 | SSPN-55 | CATGTAGAAATGCTT | SSPN | 3′ | human | dCs; lnaAs; |
| | m02 | | | | | dTs; lnaGs; |
| | | | | | | dTs; lnaAs; |
| | | | | | | dGs; lnaAs; |
| | | | | | | dAs; lnaAs; |
| | | | | | | dTs; lnaGs; |
| | | | | | | dCs; lnaTs; |
| | | | | | | dT-Sup |
| 673 | SSPN-56 | AAACATGTAGAAATG | SSPN | 3′ | human | dAs; lnaAs; |
| | m02 | | | | | dAs; lnaCs; |
| | | | | | | dAs; lnaTs; |
| | | | | | | dGs; lnaTs; |
| | | | | | | dAs; lnaGs; |
| | | | | | | dAs; lnaAs; |
| | | | | | | dAs; lnaTs; |
| | | | | | | dG-Sup |
| 674 | SSPN-57 | TTGATACCATTTATG | SSPN | 3′ | human | dTs; lnaTs; |
| | m02 | | | | | dGs; lnaAs; |
| | | | | | | dTs; lnaAs; |
| | | | | | | dCs; lnaCs; |
| | | | | | | dAs; lnaTs; |
| | | | | | | dTs; lnaTs; |
| | | | | | | dAs; lnaTs; |
| | | | | | | dG-Sup |
| 675 | SSPN-58 | GAACTCAATTATTAT | SSPN | 3′ | human | dGs; lnaAs; |
| | m02 | | | | | dAs; lnaCs; |
| | | | | | | dTs; lnaCs; |
| | | | | | | dAs; lnaAs; |
| | | | | | | dTs; lnaTs; |
| | | | | | | dAs; lnaTs; |
| | | | | | | dTs; lnaAs; |
| | | | | | | dT-Sup |
| 676 | UTRN- | AAAACGACTCCACAA | UTRN | 5′ | human | dAs; lnaAs; |
| | 972 | | | | | dAs; lnaAs; |
| | m02 | | | | | dCs; lnaGs; |
| | | | | | | dAs; lnaCs; |
| | | | | | | dTs; lnaCs; |
| | | | | | | dCs; lnaAs; |
| | | | | | | dCs; lnaAs; |
| | | | | | | dA-Sup |
| 677 | UTRN- | CTCCGAGGAAAAACG | UTRN | 5′ | human | dCs; lnaTs; |
| | 312 | | | | | dCs; lnaCs; |
| | m02 | | | | | dGs; lnaAs; |
| | | | | | | dGs; lnaGs; |
| | | | | | | dAs; lnaAs; |
| | | | | | | dAs; lnaAs; |
| | | | | | | dAs; lnaCs; |
| | | | | | | dG-Sup |
| 678 | UTRN- | GCTCCGAGGAAAAAC | UTRN | 5′ | human | dGs; lnaCs; |
| | 313 | | | | | dTs; lnaCs; |
| | m02 | | | | | dCs; lnaGs; |
| | | | | | | dAs; lnaGs; |
| | | | | | | dGs; lnaAs; |
| | | | | | | dAs; lnaAs; |
| | | | | | | dAs; lnaAs; |
| | | | | | | dC-Sup |
| 679 | UTRN- | CTCGGCGGGAGAAAG | UTRN | 5′ | human | dCs; lnaTs; |
| | 975 | | | | | dCs; lnaGs; |
| | m02 | | | | | dGs; lnaCs; |
| | | | | | | dGs; lnaGs; |
| | | | | | | dGs; lnaAs; |
| | | | | | | dGs; lnaAs; |
| | | | | | | dAs; lnaAs; |
| | | | | | | dG-Sup |
| 680 | UTRN- | GAACCGAAATTTT | UTRN | 5′ | human | dGs; lnaAs; |
| | 976 | | | | | dAs; lnaCs; |
| | m02 | | | | | dCs; lnaGs; |
| | | | | | | dAs; lnaAs; |
| | | | | | | dAs; lnaTs; |
| | | | | | | dTs; lnaTs; |
| | | | | | | dT-Sup |
| 681 | UTRN- | GAGAAGGGTGCAGAT | UTRN | 5′ | human | dGs; lnaAs; |
| | 977 | | | | | dGs; lnaAs; |
| | m02 | | | | | dAs; lnaGs; |
| | | | | | | dGs; lnaGs; |
| | | | | | | dTs; lnaGs; |
| | | | | | | dCs; lnaAs; |
| | | | | | | dGs; lnaAs; |
| | | | | | | dT-Sup |
| 682 | UTRN- | CTCTCCAGATGAGAA | UTRN | 5′ | human | dCs; lnaTs; |
| | 978 | | | | | dCs; lnaTs; |
| | m02 | | | | | dCs; lnaCs; |
| | | | | | | dAs; lnaGs; |
| | | | | | | dAs; lnaTs; |
| | | | | | | dGs; lnaAs; |
| | | | | | | dGs; lnaAs; |
| | | | | | | dA-Sup |
| 683 | UTRN- | CAGGGGTCCGCTCTC | UTRN | 5′ | human | dCs; lnaAs; |
| | 979 | | | | | dGs; lnaGs; |
| | m02 | | | | | dGs; lnaGs; |
| | | | | | | dTs; lnaCs; |
| | | | | | | dCs; lnaGs; |
| | | | | | | dCs; lnaTs; |
| | | | | | | dCs; lnaTs; |
| | | | | | | dC-Sup |
| 684 | UTRN- | TCCGGGCAGCCAGGG | UTRN | 5′ | human | dTs; lnaCs; |
| | 980 | | | | | dCs; lnaGs; |
| | m02 | | | | | dGs; lnaGs; |
| | | | | | | dCs; lnaAs; |
| | | | | | | dGs; lnaCs; |
| | | | | | | dCs; lnaAs; |
| | | | | | | dGs; lnaGs; |
| | | | | | | dG-Sup |
| 685 | UTRN- | GGGGCTCGCCTCCGG | UTRN | 5′ | human | dGs; lnaGs; |
| | 981 | | | | | dGs; lnaGs; |
| | m02 | | | | | dCs; lnaTs; |
| | | | | | | dCs; lnaGs; |
| | | | | | | dCs; lnaCs; |
| | | | | | | dTs; lnaCs; |
| | | | | | | dCs; lnaGs; |
| | | | | | | dG-Sup |
| 686 | UTRN- | CCCCCGGGAAGGGGC | UTRN | 5′ | human | dCs; lnaCs; |
| | 982 | | | | | dCs; lnaCs; |
| | m02 | | | | | dCs; lnaGs; |
| | | | | | | dGs; lnaGs; |
| | | | | | | dAs; lnaAs; |
| | | | | | | dGs; lnaGs; |
| | | | | | | dGs; lnaGs; |
| | | | | | | dC-Sup |
| 687 | UTRN- | CCCACCCCCCGGGAA | UTRN | 5′ | human | dCs; lnaCs; |
| | 983 | | | | | dCs; lnaAs; |
| | m02 | | | | | dCs; lnaCs; |
| | | | | | | dCs; lnaCs; |
| | | | | | | dCs; lnaCs; |
| | | | | | | dGs; lnaGs; |
| | | | | | | dGs; lnaAs; |
| | | | | | | dA-Sup |
| 688 | UTRN- | GCGTTGCCGCCCCCAC | UTRN | 5′ | human | dGs; lnaCs; |
| | 984 | | | | | dGs; lnaTs; |
| | m02 | | | | | dTs; lnaGs; |
| | | | | | | dCs; lnaCs; |
| | | | | | | dGs; lnaCs; |
| | | | | | | dCs; lnaCs; |
| | | | | | | dCs; lnaCs; |
| | | | | | | dAs; dC-Sup |
| 689 | UTRN- | GCTGGGTCGCGCGTT | UTRN | 5′ | human | dGs; lnaCs; |
| | 985 | | | | | dTs; lnaGs; |
| | m02 | | | | | dGs; lnaGs; |
| | | | | | | dTs; lnaCs; |
| | | | | | | dGs; lnaCs; |
| | | | | | | dGs; lnaCs; |
| | | | | | | dGs; lnaTs; |
| | | | | | | dT-Sup |
| 690 | UTRN- | GCGCAGGACCGCTGG | UTRN | 5′ | human | dGs; lnaCs; |
| | 986 | | | | | dGs; lnaCs; |
| | m02 | | | | | dAs; lnaGs; |
| | | | | | | dGs; lnaAs; |
| | | | | | | dCs; lnaCs; |
| | | | | | | dGs; lnaCs; |
| | | | | | | dTs; lnaGs; |
| | | | | | | dG-Sup |
| 691 | UTRN- | AGGAGGGAGGGTGGG | UTRN | 5′ | human | dAs; lnaGs; |
| | 987 | | | | | dGs; lnaAs; |
| | m02 | | | | | dGs; lnaGs; |
| | | | | | | dGs; lnaAs; |
| | | | | | | dGs; lnaGs; |
| | | | | | | dGs; lnaTs; |
| | | | | | | dGs; lnaGs; |
| | | | | | | dG-Sup |
| 692 | UTRN- | CGCTGGAGGCGGAGG | UTRN | 5′ | human | dCs; lnaGs; |
| | 988 | | | | | dCs; lnaTs; |
| | m02 | | | | | dGs; lnaGs; |
| | | | | | | dAs; lnaGs; |
| | | | | | | dGs; lnaCs; |
| | | | | | | dGs; lnaGs; |
| | | | | | | dAs; lnaGs; |
| | | | | | | dG-Sup |
| 693 | UTRN- | TGGAGCCGAGCGCTG | UTRN | 5′ | human | dTs; lnaGs; |
| | 192 | | | | | dGs; lnaAs; |
| | m02 | | | | | dGs; lnaCs; |
| | | | | | | dCs; lnaGs; |
| | | | | | | dAs; lnaGs; |
| | | | | | | dCs; lnaGs; |
| | | | | | | dCs; lnaTs; |
| | | | | | | dG-Sup |
| 694 | UTRN- | CTGCCCCTTTGTTGG | UTRN | 5′ | human | dCs; lnaTs; |
| | 303 | | | | | dGs; lnaCs; |
| | m02 | | | | | dCs; lnaCs; |
| | | | | | | dCs; lnaTs; |
| | | | | | | dTs; lnaTs; |
| | | | | | | dGs; lnaTs; |
| | | | | | | dTs; lnaGs; |
| | | | | | | dG-Sup |
| 695 | UTRN- | CTCCCCGCTGCGGGC | UTRN | 5′ | human | dCs; lnaTs; |
| | 991 | | | | | dCs; lnaCs; |
| | m02 | | | | | dCs; lnaCs; |
| | | | | | | dGs; lnaCs; |
| | | | | | | dTs; lnaGs; |
| | | | | | | dCs; lnaGs; |
| | | | | | | dGs; lnaGs; |
| | | | | | | dC-Sup |
| 696 | UTRN- | CGGCTCCTCCTCCTC | UTRN | 5′ | human | dCs; lnaGs; |
| | 992 | | | | | dGs; lnaCs; |
| | m02 | | | | | dTs; lnaCs; |
| | | | | | | dCs; lnaTs; |
| | | | | | | dCs; lnaCs; |
| | | | | | | dTs; lnaCs; |
| | | | | | | dCs; lnaTs; |
| | | | | | | dC-Sup |
| 697 | UTRN- | GGCTCGCTCCTTCGG | UTRN | 5′ | human | dGs; lnaGs; |
| | 993 | | | | | dCs; lnaTs; |
| | m02 | | | | | dCs; lnaGs; |
| | | | | | | dCs; lnaTs; |
| | | | | | | dCs; lnaCs; |
| | | | | | | dTs; lnaTs; |
| | | | | | | dCs; lnaGs; |
| | | | | | | dG-Sup |
| 698 | UTRN- | TTTGTGCGCGAGAGA | UTRN | 5′ | human | dTs; lnaTs; |
| | 994 | | | | | dTs; lnaGs; |
| | m02 | | | | | dTs; lnaGs; |
| | | | | | | dCs; lnaGs; |
| | | | | | | dCs; lnaGs; |
| | | | | | | dAs; lnaGs; |
| | | | | | | dAs; lnaGs; |
| | | | | | | dA-Sup |
| 699 | UTRN- | ACGACTCCACAACTT | UTRN | 5′ | human | dAs; lnaCs; |
| | 995 | | | | | dGs; lnaAs; |
| | m02 | | | | | dCs; lnaTs; |
| | | | | | | dCs; lnaCs; |
| | | | | | | dAs; lnaCs; |
| | | | | | | dAs; lnaAs; |
| | | | | | | dCs; lnaTs; |
| | | | | | | dT-Sup |
| 700 | UTRN- | GCCCGCTTCCCTGCT | UTRN | 5′ | human | dGs; lnaCs; |
| | 997 | | | | | dCs; lnaCs; |
| | m02 | | | | | dGs; lnaCs; |
| | | | | | | dTs; lnaTs; |
| | | | | | | dCs; lnaCs; |
| | | | | | | dCs; lnaTs; |
| | | | | | | dGs; lnaCs; |
| | | | | | | dT-Sup |
| 701 | UTRN- | CGGCCGGCTGCTGCT | UTRN | 5′ | human | dCs; lnaGs; |
| | 662 | | | | | dGs; lnaCs; |
| | m02 | | | | | dCs; lnaGs; |
| | | | | | | dGs; lnaCs; |
| | | | | | | dTs; lnaGs; |
| | | | | | | dCs; lnaTs; |
| | | | | | | dGs; lnaCs; |
| | | | | | | dT-Sup |
| 702 | UTRN- | GCGGGAGAAAGCCCG | UTRN | 5′ | human | dGs; lnaCs; |
| | 999 | | | | | dGs; lnaGs; |
| | m02 | | | | | dGs; lnaAs; |
| | | | | | | dGs; lnaAs; |
| | | | | | | dAs; lnaAs; |
| | | | | | | dGs; lnaCs; |
| | | | | | | dCs; lnaCs; |
| | | | | | | dG-Sup |
| 703 | UTRN- | CCTCCTCGCCCCTCG | UTRN | 5′ | human | dCs; lnaCs; |
| | 1000 | | | | | dTs; lnaCs; |
| | m02 | | | | | dCs; lnaTs; |
| | | | | | | dCs; lnaGs; |
| | | | | | | dCs; lnaCs; |
| | | | | | | dCs; lnaCs; |
| | | | | | | dTs; lnaCs; |
| | | | | | | dG-Sup |
| 704 | UTRN- | AGAGGCTCCTCCTCG | UTRN | 5′ | human | dAs; lnaGs; |
| | 1001 | | | | | dAs; lnaGs; |
| | m02 | | | | | dGs; lnaCs; |
| | | | | | | dTs; lnaCs; |
| | | | | | | dCs; lnaTs; |
| | | | | | | dCs; lnaCs; |
| | | | | | | dTs; lnaCs; |
| | | | | | | dG-Sup |
| 705 | UTRN- | TCGGCTTCTGGAGCC | UTRN | 5′ | human | dTs; lnaCs; |
| | 1002 | | | | | dGs; lnaGs; |
| | m02 | | | | | dCs; lnaTs; |
| | | | | | | dTs; lnaCs; |
| | | | | | | dTs; lnaGs; |
| | | | | | | dGs; lnaAs; |
| | | | | | | dGs; lnaCs; |
| | | | | | | dC-Sup |
| 706 | UTRN- | CCGTGATTCCCCAAT | UTRN | 5′ | human | dCs; lnaCs; |
| | 1003 | | | | | dGs; lnaTs; |
| | m02 | | | | | dGs; lnaAs; |
| | | | | | | dTs; lnaTs; |
| | | | | | | dCs; lnaCs; |
| | | | | | | dCs; lnaCs; |
| | | | | | | dAs; lnaAs; |
| | | | | | | dT-Sup |
| 707 | UTRN- | AGGGGGGCGCCGCTC | UTRN | 5′ | human | dAs; lnaGs; |
| | 1004 | | | | | dGs; lnaGs; |
| | m02 | | | | | dGs; lnaGs; |
| | | | | | | dGs; lnaCs; |
| | | | | | | dGs; lnaCs; |
| | | | | | | dCs; lnaGs; |
| | | | | | | dCs; lnaTs; |
| | | | | | | dC-Sup |
| 708 | UTRN- | AAATGACCCAAAAGA | UTRN | 5′ | human | dAs; lnaAs; |
| | 323 | | | | | dAs; lnaTs; |
| | m02 | | | | | dGs; lnaAs; |
| | | | | | | dCs; lnaCs; |
| | | | | | | dCs; lnaAs; |
| | | | | | | dAs; lnaAs; |
| | | | | | | dAs; lnaGs; |
| | | | | | | dA-Sup |
| 709 | UTRN- | GTTTTCCGTTTGCAG | UTRN | 5′ | human | dGs; lnaTs; |
| | 328 | | | | | dTs; lnaTs; |
| | m02 | | | | | dTs; lnaCs; |
| | | | | | | dCs; lnaGs; |
| | | | | | | dTs; lnaTs; |
| | | | | | | dTs; lnaGs; |
| | | | | | | dCs; lnaAs; |
| | | | | | | dG-Sup |
| 710 | UTRN- | CCAAACGCTACAGAG | UTRN | 5′ | human | dCs; lnaCs; |
| | 334 | | | | | dAs; lnaAs; |
| | m02 | | | | | dAs; lnaCs; |
| | | | | | | dGs; lnaCs; |
| | | | | | | dTs; lnaAs; |
| | | | | | | dCs; lnaAs; |
| | | | | | | dGs; lnaAs; |
| | | | | | | dG-Sup |
| 711 | UTRN- | CAGGCACCAACTTTG | UTRN | 5′ | human | dCs; lnaAs; |
| | 1008 | | | | | dGs; lnaGs; |
| | m02 | | | | | dCs; lnaAs; |
| | | | | | | dCs; lnaCs; |
| | | | | | | dAs; lnaAs; |
| | | | | | | dCs; lnaTs; |
| | | | | | | dTs; lnaTs; |
| | | | | | | dG-Sup |
| 712 | UTRN- | CCTGGAAGGGGCGCG | UTRN | 5′ | human | dCs; lnaCs; |
| | 1009 | | | | | dTs; lnaGs; |
| | m02 | | | | | dGs; lnaAs; |
| | | | | | | dAs; lnaGs; |
| | | | | | | dGs; lnaGs; |
| | | | | | | dGs; lnaCs; |
| | | | | | | dGs; lnaCs; |
| | | | | | | dG-Sup |
| 713 | UTRN- | CAGTCAAAGCGCAAA | UTRN | 5′ | human | dCs; lnaAs; |
| | 345 | | | | | dGs; lnaTs; |
| | m02 | | | | | dCs; lnaAs; |
| | | | | | | dAs; lnaAs; |
| | | | | | | dGs; lnaCs; |
| | | | | | | dGs; lnaCs; |
| | | | | | | dAs; lnaAs; |
| | | | | | | dA-Sup |
| 714 | UTRN- | CCAAAAACAAAACAG | UTRN | 5′ | human | dCs; lnaCs; |
| | 1011 | | | | | dAs; lnaAs; |
| | m02 | | | | | dAs; lnaAs; |
| | | | | | | dAs; lnaCs; |
| | | | | | | dAs; lnaAs; |
| | | | | | | dAs; lnaAs; |
| | | | | | | dCs; lnaAs; |
| | | | | | | dG-Sup |
| 715 | UTRN- | TTCCGCCAAAAACAA | UTRN | 5′ | human | dTs; lnaTs; |
| | 674 | | | | | dCs; lnaCs; |
| | m02 | | | | | dGs; lnaCs; |
| | | | | | | dCs; lnaAs; |
| | | | | | | dAs; lnaAs; |
| | | | | | | dAs; lnaAs; |
| | | | | | | dCs; lnaAs; |
| | | | | | | dA-Sup |
| 716 | UTRN- | GGAGGAGGGAGGGTG | UTRN | 5′ | human | dGs; lnaGs; |
| | 1013 | | | | | dAs; lnaGs; |
| | m02 | | | | | dGs; lnaAs; |
| | | | | | | dGs; lnaGs; |
| | | | | | | dGs; lnaAs; |
| | | | | | | dGs; lnaGs; |
| | | | | | | dGs; lnaTs; |
| | | | | | | dG-Sup |
| 717 | UTRN- | CGAGCGCTGGAGGCG | UTRN | 5′ | human | dCs; lnaGs; |
| | 1014 | | | | | dAs; lnaGs; |
| | m02 | | | | | dCs; lnaGs; |
| | | | | | | dCs; lnaTs; |
| | | | | | | dGs; lnaGs; |
| | | | | | | dAs; lnaGs; |
| | | | | | | dGs; lnaCs; |
| | | | | | | dG-Sup |
| 718 | UTRN- | CCTGCCCCTTTGTTG | UTRN | 5′ | human | dCs; lnaCs; |
| | 1015 | | | | | dTs; lnaGs; |
| | m02 | | | | | dCs; lnaCs; |
| | | | | | | dCs; lnaCs; |
| | | | | | | dTs; lnaTs; |
| | | | | | | dTs; lnaGs; |
| | | | | | | dTs; lnaTs; |
| | | | | | | dG-Sup |
| 719 | UTRN- | GGCGGCTCCTCCTCC | UTRN | 5′ | human | dGs; lnaGs; |
| | 1016 | | | | | dCs; lnaGs; |
| | m02 | | | | | dGs; lnaCs; |
| | | | | | | dTs; lnaCs; |
| | | | | | | dCs; lnaTs; |
| | | | | | | dCs; lnaCs; |
| | | | | | | dTs; lnaCs; |
| | | | | | | dC-Sup |

### Example 10

### Further Data for FXN Oligos

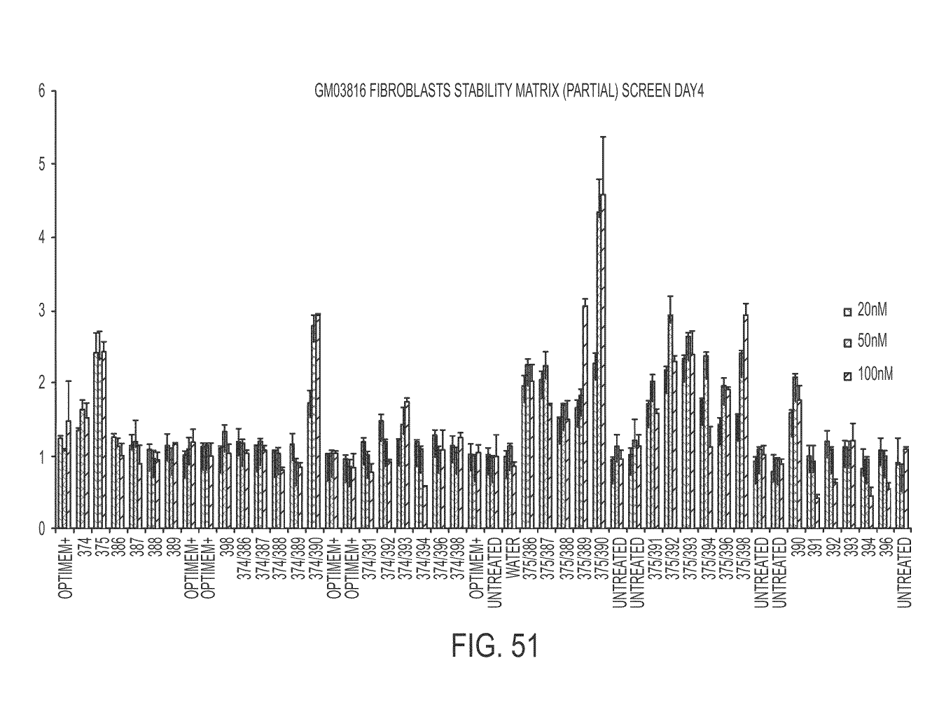

Using FXN-374 and FXN-375 as 5′ oligos, all 3′ oligos available in Table 3 were screened for RNA upregulation of human FXN in GM03816 cells via transfection at 20 nM, 50 nM and 100 nM concentrations (FIG. 51). Concentrations were total oligo concentrations (e.g. 20 nM means 10 nM for each oligo). In general, cell treated with the oligo combinations that included the 375 oligo had upregulation of human FXN compared to untreated cells. The 375 and 390 combination gave a dose responsive upregulation of human FXN at the highest levels (FIG. 51).

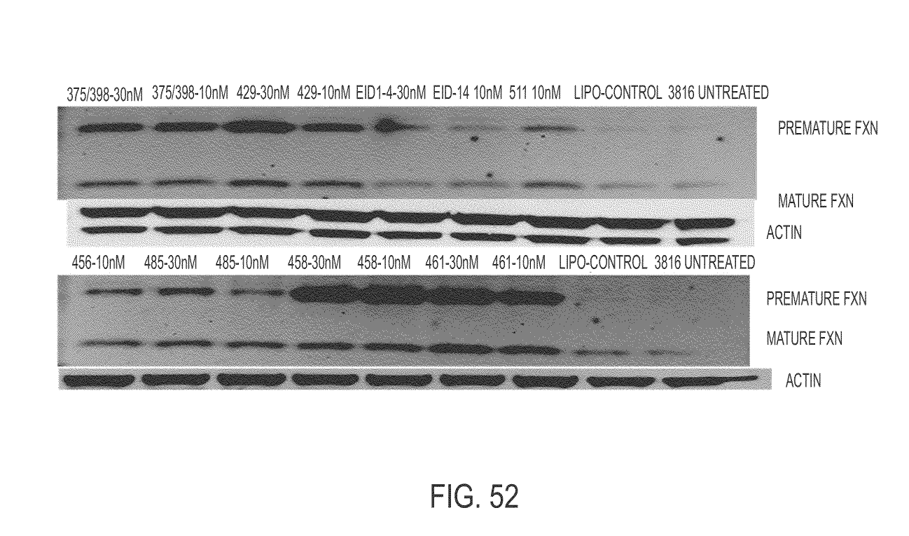

Various FXN oligos from Table 3, Table 6, Table 7 and Table 10 were transfected to the GM03816 cell lines (FXN-375/FXN-398 combo at 10 or 30 nM, FXN-429 at 10 or 30 nM, 511 at 10 nM, FXN-456 at 10 nM, FXN-485 at 10 nM or 30 nM, FXN-458 at 10 nM, FXN-461 m02 at 10 or 30 nM). Abcam ab48281 antibody was used to measure premature and mature FXN protein levels. Oligos 456, 458, 485 and 461 are pseudo-circularization oligos. Oligo 461 is a pseudo-circularization oligo that contains the sequences of the 375 (5′) and 390 (3′) oligo. Actin was used as the loading control (Cell signaling, 8457). Levels of premature and mature FXN, in general, were upregulated in all oligo-treated cells (FIG. 52). Premature and mature FXN were dramatically upregulated in a dose responsive manner by FXN-458 and FXN-461 (FIG. 52).

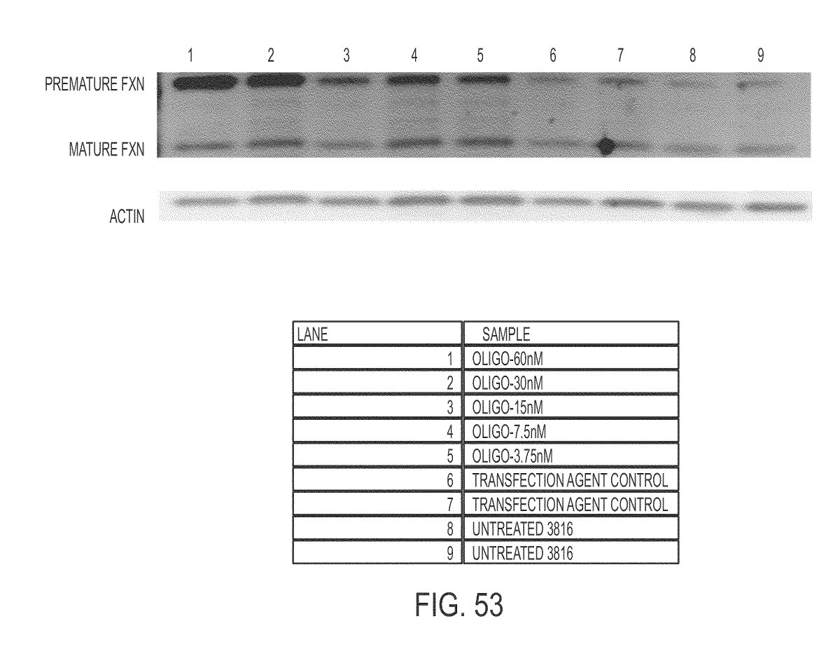

A further study with FXN-461 m02 oligo was performed. FXN-461 m02 dose response was measured with transfection to GM03816 cell line at the indicated concentrations. Abcam ab48281 antibody was used to measure premature and mature FXN protein levels. Actin was used as the loading control (Cell signaling, 8457). FXN protein levels were also upregulated strongly in the follow-up study (FIG. 53).

Next, further 3′-targeting FXN oligos (shown in Table 10) were designed to examine potential alternative 3′ locations based on public polyA-seq data. The FXN-375 oligo was used as the 5′ oligo and was combined with the further 3′-targeting FXN oligos. Transfection into GM03816 cells was done at a 30 nM concentration. FXN mRNA upregulation was observed in several of the oligo combinations and was highest with 3′ oligos FXN-527 and FXN-532 (FIG. 54).

A subset of the further 3′-targeting FXN oligos were screened with an alternate 5′ oligo (FXN-675) instead of the 375 oligo to examine reproducibility of 3′ oligo mediated upregulation of FXN mRNA. While differences are observed, similar 3′ oligos were identified as lead compounds with both 5′ oligos, e.g., FXN-654, FXN-663, FXN-666, FXN-668 and FXN-670 (FIG. 55).

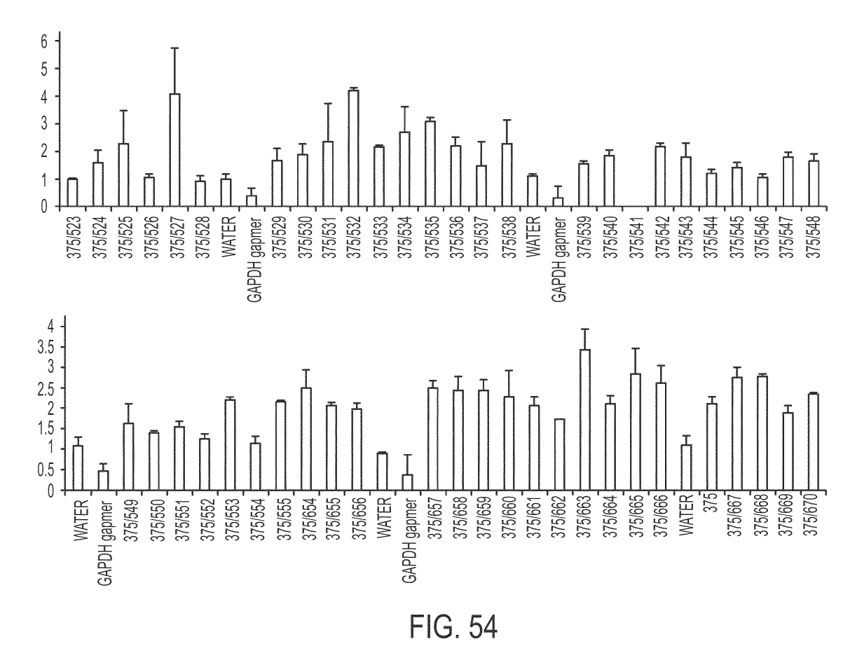

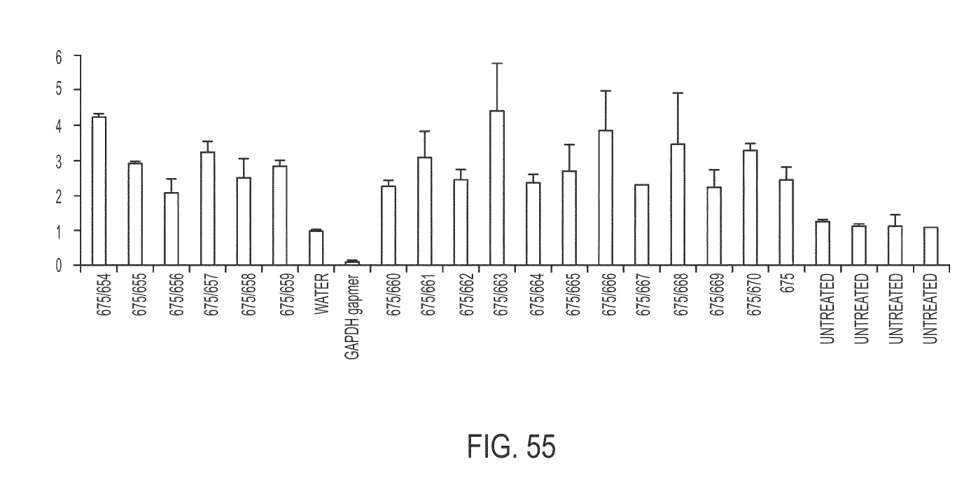

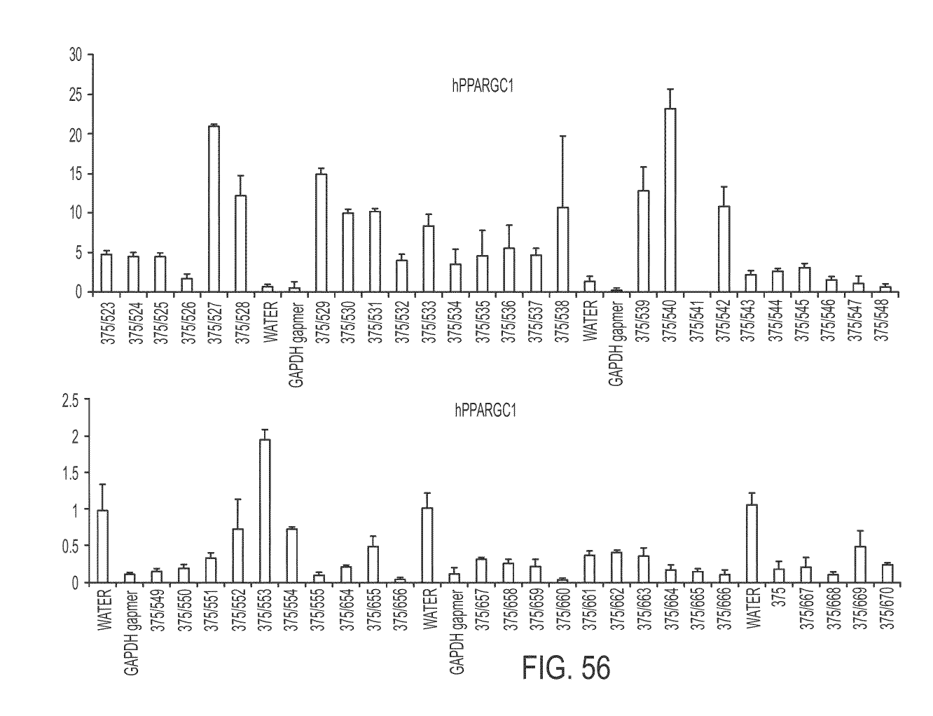

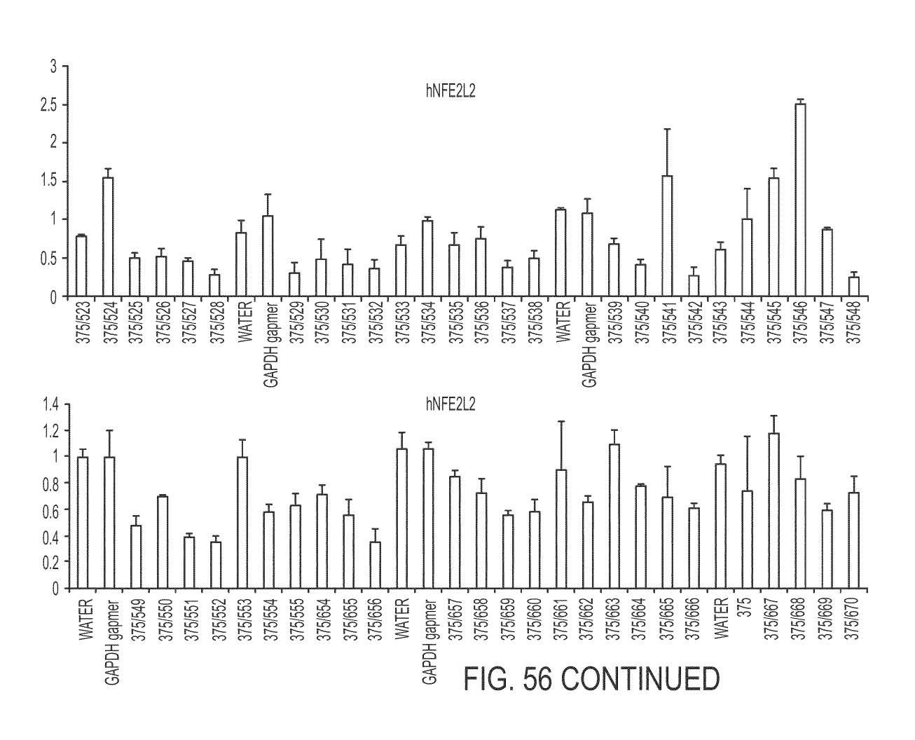

Expression changes of candidate FXN downstream genes, PPARGC1 and NFE2L2, were evaluated in the 3′ oligo study. The largest changes were observed with the PPARGC1 gene (FIG. 56).

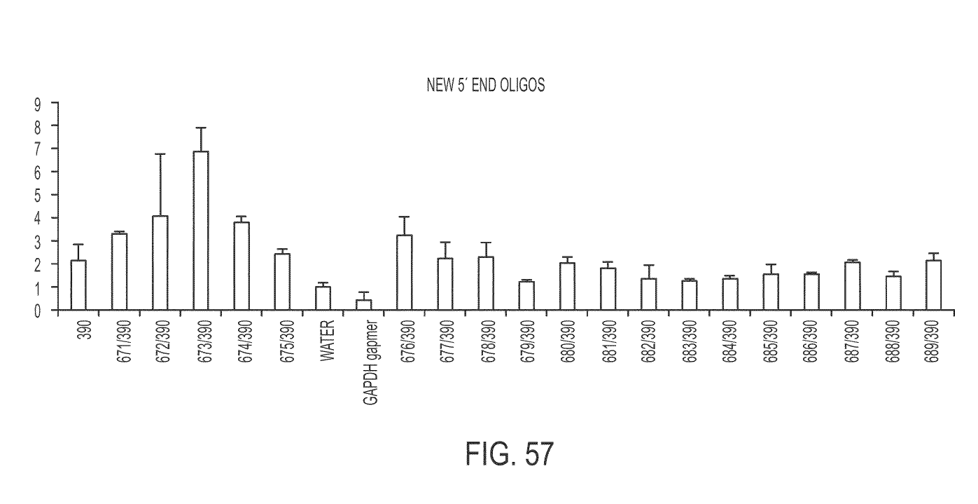

Next, further 5′-targeting FXN oligos were designed to examine potential alternative 5′ locations, and to examine oligos with shorter lengths. Transfection into GM03816 cells was done at a 30 nM concentration. The FXN-390 oligo was used as the 3′ oligo. FXN mRNA upregulation was highest with 5′ oligo FXN-673 (FIG. 57). Oligos 671-673 were 13 mer, 11 mer and 9 mer versions of FXN-375 (15 mer), respectively.

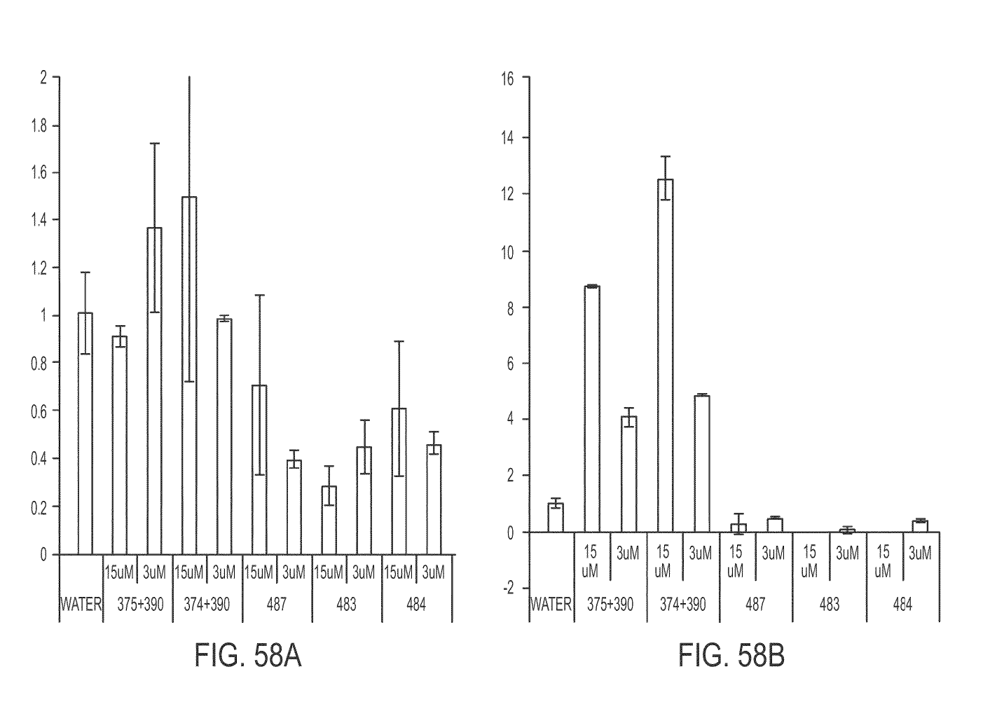

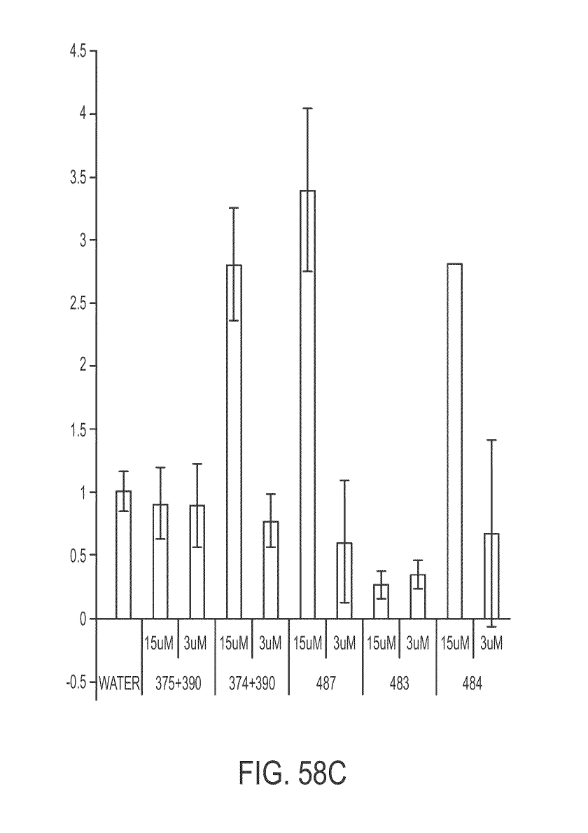

Subsequently, several 5′ (FXN-374, FXN-375), 3′ (FXN-390) and pseudo-circularization (483, 484, 487) FXN oligos were tested gymnotically in FRDA mouse model (Sarsero) fibroblasts for 4, 7 and 10 days in vitro. FXN mRNA levels were highest with the FXN-374+390 and FXN-375+390 combinations (FIG. 58A-C).

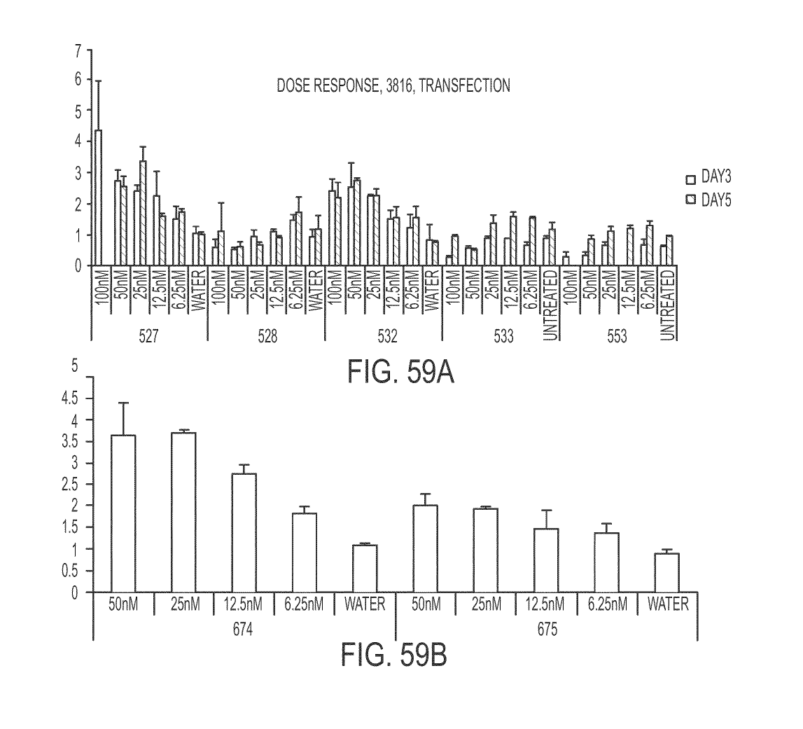

Next, various 3′ and 5′ FXN oligos (FXN-527, FXN-528, FXN-532, FXN-533, FXN-553, FXN-674, and FXN-675) were examined by transfection in GM03816 cells for dose-response patterns of FXN mRNA levels (FIGS. 59A and B). Oligos FXN-527, FXN-532, FXN-674, and FXN-675 showed a dose-dependent increase of FXN mRNA.

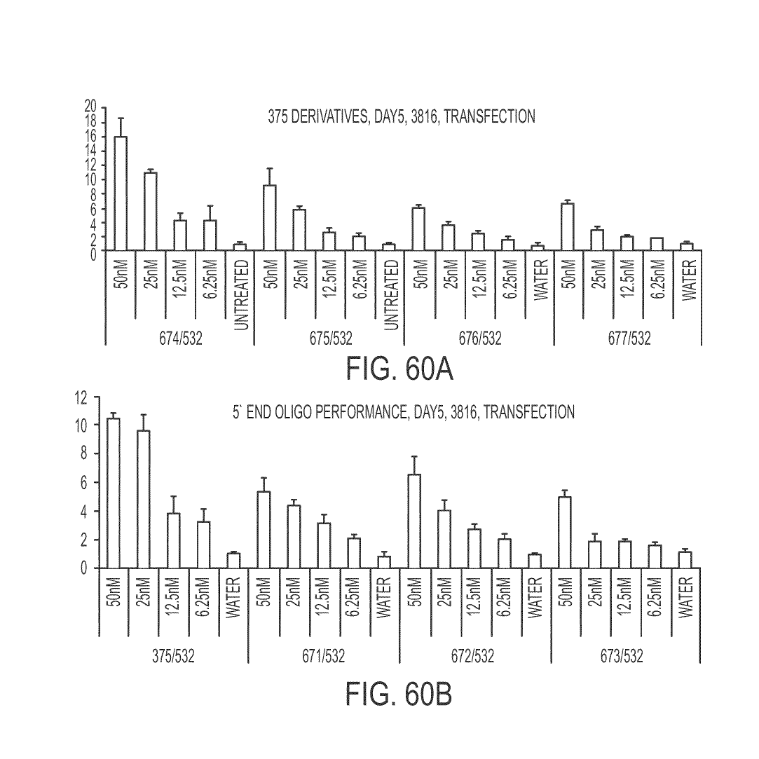

Subsequently, various 5′ FXN oligos were combined with a lead 3′ oligo, FXN-532. Dose response patterns of FXN mRNA were measured with transfection in GM03816 cells. All tested oligos showed a dose-dependent increase of FXN mRNA. Measurements were done at day5. FXN-674 is a 15 mer that overlaps with FXN-375 by 11 nucleotides. FXN-675, FXN-676 and FXN-677 are 13 mer, 11 mer and 9-mer versions of FXN-674, respectively. FXN-671, FXN-672 and FXN-673 are 13 mer, 11 mer and 9-mer versions of FXN-375, respectively (FIGS. 60A and B).

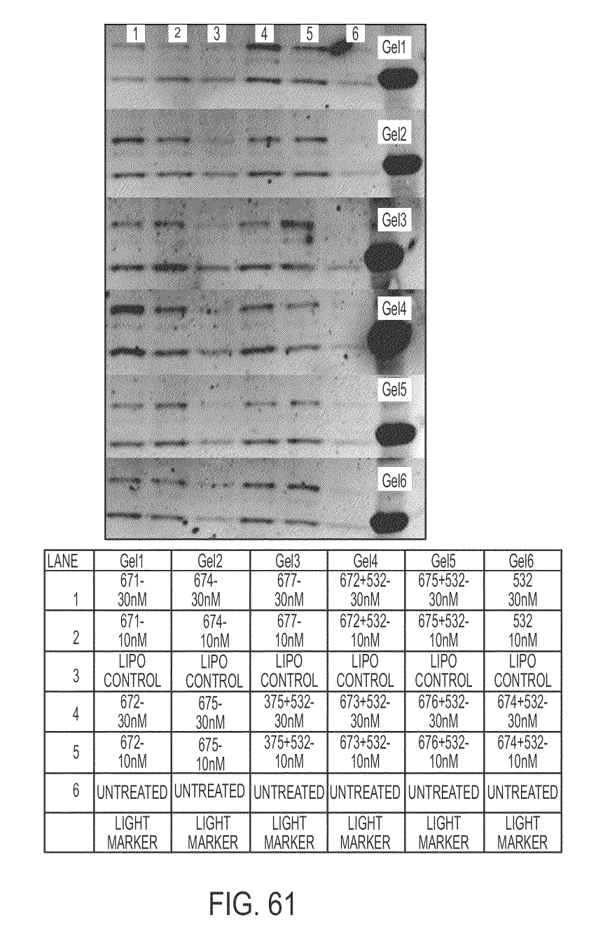

Next, 5′ oligos (FXN-375, FXN-671, FXN-672, FXN-673, FXN-674, FXN-675, FXN-676, and FXN-677) were tested alone or in combination with 3′ oligo FXN-532 for upregulation of FXN protein. The oligos were transfected either alone or in combinations to GM03816 cells at 30 nM and 10 nM concentrations. Measurements were taken at day 5. A Western blot was done with the Abcam (ab110328) antibody to detect premature and mature FXN protein. In general, FXN protein levels were upregulated in all cells treated with oligos, either alone or in combination (FIG. 61). The highest protein upregulation was observed with the FXN-672+532 combination (FIG. 61).

Several lead 5′ (FXN-374, FXN-375), 3′ (FXN-390), pseudo-circularization oligos (FXN-460: FXN-374+390; FXN-461: FXN-375+390) and multi-targeting oligos (FXN-460 MTO and FXN-461 MTO) are tested gymnotically in normal human cardiomyocytes for human FXN mRNA upregulation. Multitargeting Oligos (MTO) comprise 5′ and 3′ targeting oligos linked by a cleavable linker (e.g., oligo-dT linker (e.g., dTdTdTdTdT)). Oligos are incubated at multiple concentrations for 8 days, changing media and oligos at day4.

### Example 11

### Data for UTRN Oligos

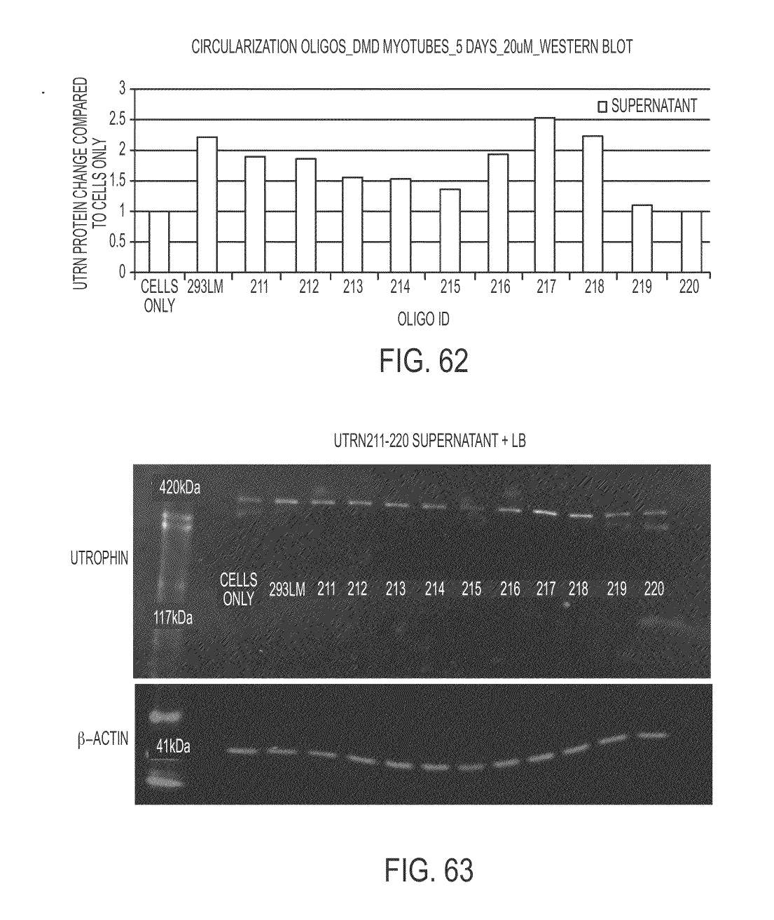

Pseudo-circularization oligos for Utrophin (UTRN-211-220) as shown in Table 7 were screened gymnotically in differentiated human patient Duchenne muscular dystrophy (DMD) myotubes. Westerns were done with the Mancho 5 antibody. UTRN protein western signal was normalized relative to beta-actin levels and untreated sample. Oligo UTRN-217 was shown to upregulate the level of UTRN protein compared to negative control oligo 293 LM and compared to cells only (FIGS. 62 and 63).

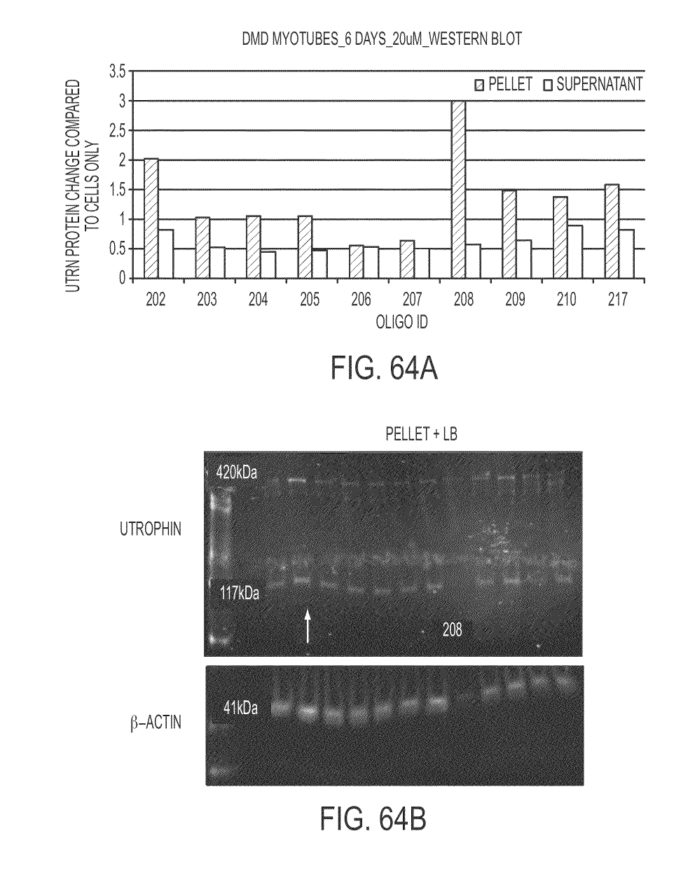

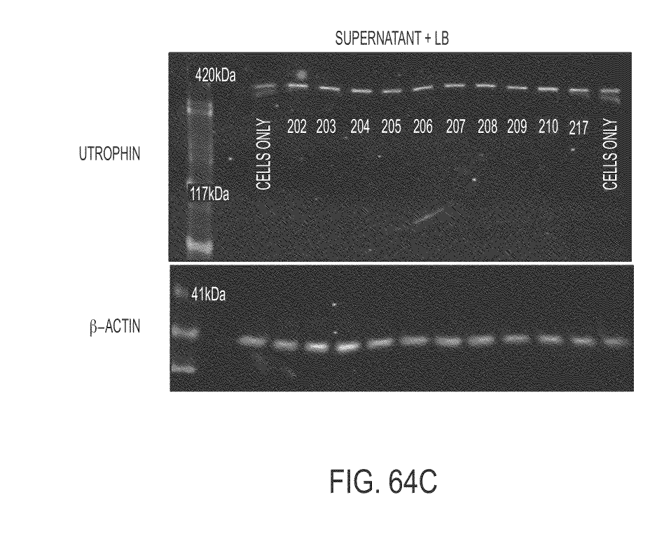

Next, UTRN 5′ and 3′ oligos were screened individually and gymnotically in differentiated human patient DMD myotubes. Samples were separated into pellet and supernatant through centrigfugation for Western analysis. Samples were lysed in SDS solution, kept on ice and then spun down to separate pellet and supernatant fractions. Westerns were done with the Mancho 5 antibody. UTRN protein western signal was normalized relative to beta-actin levels and untreated sample. Positive upregulation of UTRN protein was observed in the pellet of cells treated with UTRN-202, 208, 209, 210 and 217 oligos (FIG. 64A-C).

### Example 12

### Data for APOA1 Oligos

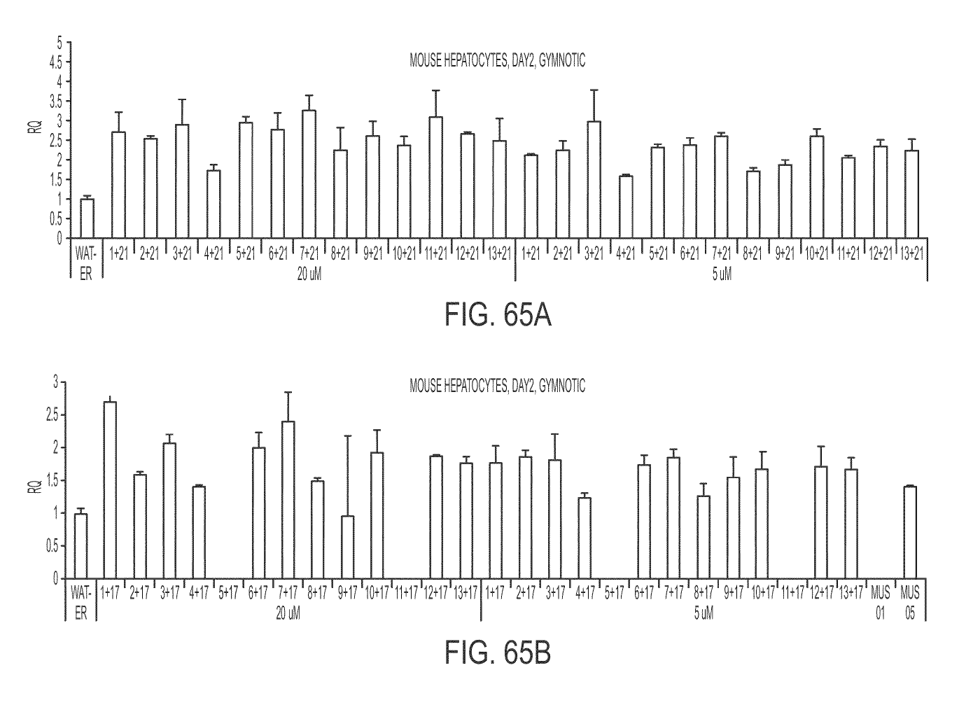

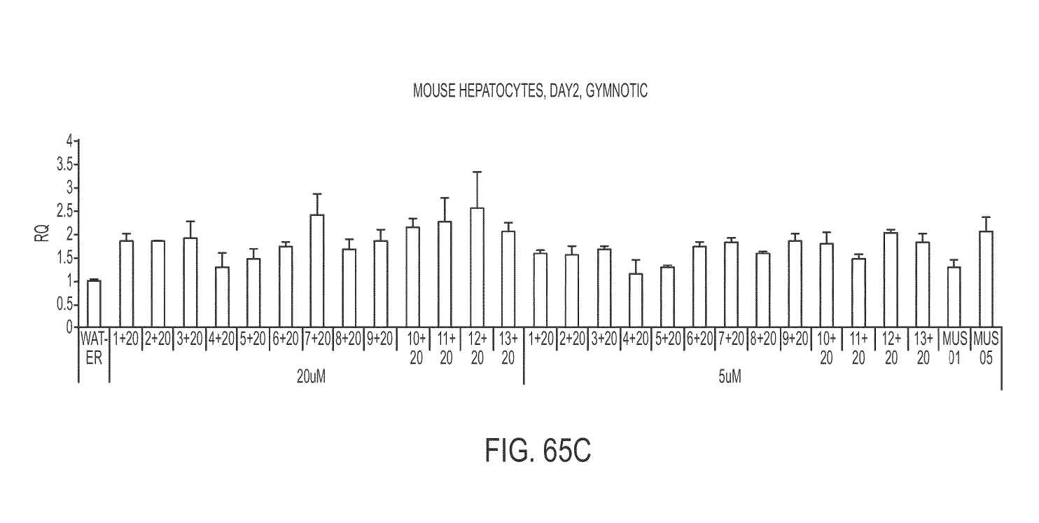

Mouse APOA1 5′ (APOA1_mus-1-13) and 3′ (APOA1_mus-21) oligo combinations were screened in duplicate in primary mouse hepatocytes gymnotically at 20 uM and 5 uM concentrations. APOA1 mRNA was measured and normalized relative to the water control well. Several of the tested oligos caused an upregulation of APOA1 compared to water (FIG. 65).

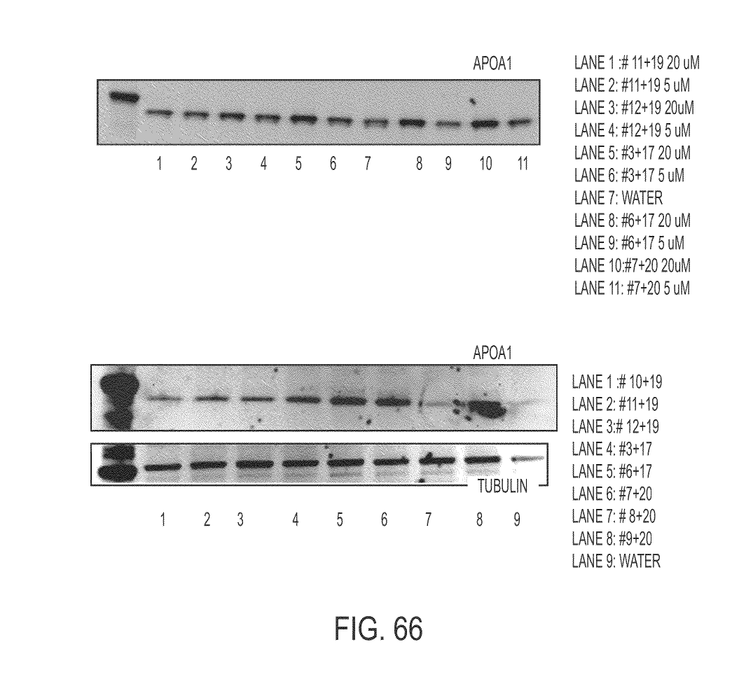

Next, mouse APOA1 5′ and 3′ oligo combinations were screened in primary mouse hepatocytes gymnotically to measure APOA1 protein levels. Measurements were taken at day 2. Abcam ab20453 was used as APOA1 antibody. Tubulin (ab125267) was used as loading control. Oligos APOA1_mus-3+17, APOA1_mus-6+17 and APOA1_mus-7+20 show dose-dependent APOA1 protein upregulation in both cell media and cell lysates (FIG. 66).

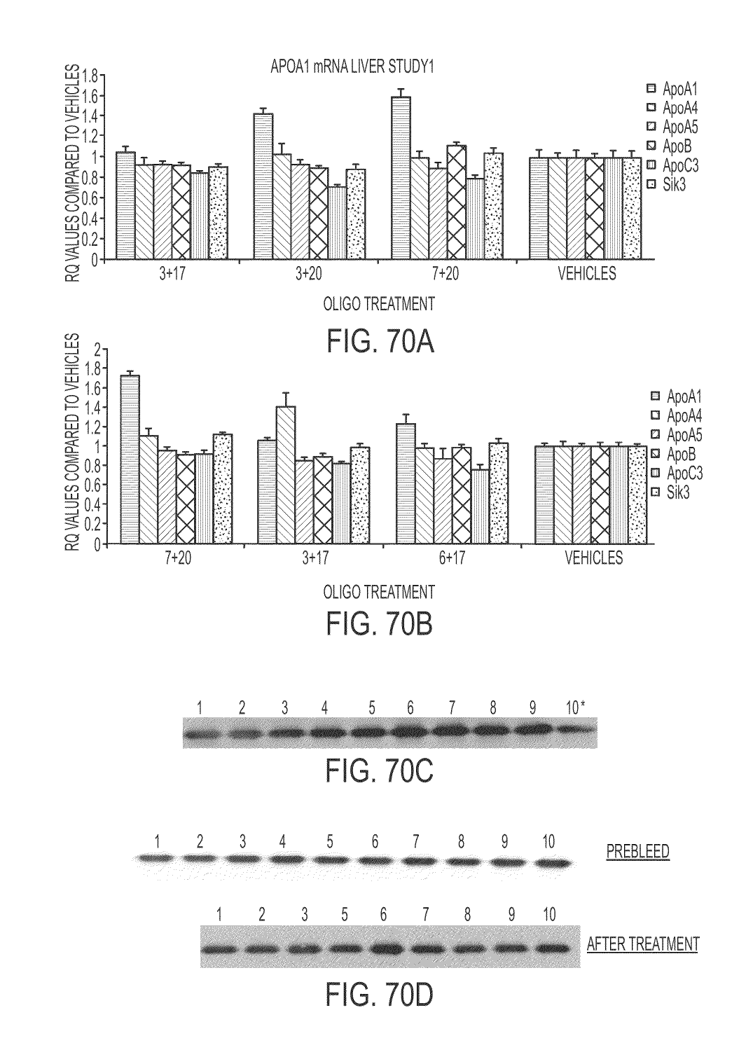

Subsequently, two mouse APOA1 5′ and 3′ oligo combinations (APOA1_mus-3+APOA1_mus-17 or APOA1_mus-7+APOA1_mus-20) were tested in vivo in mice. The oligo combinations were injected subcutaneously at days 1, 2 and 3 at 50 mg/kg for each oligo in the combinations tested. The vehicle (PBS) treatment was used as control. In a first study (FIG. 70A), collection was done at day 5, 2 days after the last dose. In a second study (FIG. 70B), collection was done at day 7, 4 days after the last dose. RNA measurements in liver in both studies (FIGS. 70A and B) suggest APOA1 mRNA upregulation of up to 80% with the 7+20 and 3+20 APOAA1 oligo combinations. The 5 genes in close proximity to APOA1 (APOC3, APOA4, APOA5, APOB, Sik3) were not significantly affected by oligo treatment.

Levels of APOA1 protein were also measured in the two in vivo studies. FIG. 70C shows APOA1 protein data from the first study for oligo combination 3+17. APOA1 protein upregulation was seen in blood plasma in all 4 treated animals. FIG. 70D shows APOA1 protein data from the second study for oligo combination 7+20. Pre-bleeding data from all 10 animals showed relatively equal levels of plasma APOA1 across animals before the start of treatments (top panel, FIG. 70D). Samples 5 and 10 showed upregulation of mouse APOA1 protein in plasma after treatment with oligo combination 7+20.

The lack of RNA changes (FIG. 70A) for oligo combination 3+17 in the presence of protein upregulation (FIG. 70C), as well as the upregulation of APOA1 in 2 out of 5 animals with oligo combination 7+20 treatment (FIG. 70D) may be due to the oligo treatment regimen and the collection points chosen.

### Example 13

### Additional Non-Coding RNA-Targeting Oligos

Table 11 provides further exemplary non-coding RNA 5′ and 3′ end targeting oligos.

TABLE 11
Oligonucleotides designed to target 5′ and
3′ ends of non-coding RNAs

| SEQ | Oligo | | Gene | Target | | Formatted |
|---|---|---|---|---|---|---|
| ID NO | Name | Base Sequence | Name | Region | Organism | Sequence |
| 720 | DINO-1 | TAGACACTTCCAGAA | DINO | 3′ | human | dTs; lnaAs; |
| | m02 | | | | | dGs; lnaAs; |
| | | | | | | dCs; lnaAs; |
| | | | | | | dCs; lnaTs; |
| | | | | | | dTs; lnaCs; |
| | | | | | | dCs; lnaAs; |
| | | | | | | dGs; lnaAs; |
| | | | | | | dA- |
| | | | | | | Sup |
| 721 | DINO-2 | TTCCAGAATTGTCCT | DINO | 3′ | human | dTs; lnaTs; |
| | m02 | | | | | dCs; lnaCs; |
| | | | | | | dAs; lnaGs; |
| | | | | | | dAs; lnaAs; |
| | | | | | | dTs; lnaTs; |
| | | | | | | dGs; lnaTs; |
| | | | | | | dCs; lnaCs; |
| | | | | | | dT- |
| | | | | | | Sup |
| 722 | DINO-3 | CAGAATTGTCCTTTA | DINO | 3′ | human | dCs; lnaAs; |
| | m02 | | | | | dGs; lnaAs; |
| | | | | | | dAs; lnaTs; |
| | | | | | | dTs; lnaGs; |
| | | | | | | dTs; lnaCs; |
| | | | | | | dCs; lnaTs; |
| | | | | | | dTs; lnaTs; |
| | | | | | | dA- |
| | | | | | | Sup |
| 723 | DINO-4 | CTGCTGGAACTCGGC | DINO | 5′ | human | dCs; lnaTs; |
| | m02 | | | | | dGs; lnaCs; |
| | | | | | | dTs; lnaGs; |
| | | | | | | dGs; lnaAs; |
| | | | | | | dAs; lnaCs; |
| | | | | | | dTs; lnaCs; |
| | | | | | | dGs; lnaGs; |
| | | | | | | dC- |
| | | | | | | Sup |
| 724 | DINO-5 | GGCCAGGCTCAGCTG | DINO | 5′ | human | dGs; lnaGs; |
| | m02 | | | | | dCs; lnaCs; |
| | | | | | | dAs; lnaGs; |
| | | | | | | dGs; lnaCs; |
| | | | | | | dTs; lnaCs; |
| | | | | | | dAs; lnaGs; |
| | | | | | | dCs; lnaTs; |
| | | | | | | dG- |
| | | | | | | Sup |
| 725 | DINO-6 | GCAGCCAGGAGCCTG | DINO | 5′ | human | dGs; lnaCs; |
| | m02 | | | | | dAs; lnaGs; |
| | | | | | | dCs; lnaCs; |
| | | | | | | dAs; lnaGs; |
| | | | | | | dGs; lnaAs; |
| | | | | | | dGs; lnaCs; |
| | | | | | | dCs; lnaTs; |
| | | | | | | dG- |
| | | | | | | Sup |
| 726 | DINO-7 | ACTCGGCCAGGCTCA | DINO | 5′ | human | dAs; lnaCs; |
| | m02 | | | | | dTs; lnaCs; |
| | | | | | | dGs; lnaGs; |
| | | | | | | dCs; lnaCs; |
| | | | | | | dAs; lnaGs; |
| | | | | | | dGs; lnaCs; |
| | | | | | | dTs; lnaCs; |
| | | | | | | dA- |
| | | | | | | Sup |
| 727 | DINO-8 | GCTGGCCTGCTGGAA | DINO | 5′ | human | dGs; lnaCs; |
| | m02 | | | | | dTs; lnaGs; |
| | | | | | | dGs; lnaCs; |
| | | | | | | dCs; lnaTs; |
| | | | | | | dGs; lnaCs; |
| | | | | | | dTs; lnaGs; |
| | | | | | | dGs; lnaAs; |
| | | | | | | dA- |
| | | | | | | Sup |
| 728 | HOTTIP-1 | TTTAAATTGTATCGG | HOTTIP | 3′ | human | dTs; lnaTs; |
| | m02 | | | | | dTs; lnaAs; |
| | | | | | | dAs; lnaAs; |
| | | | | | | dTs; lnaTs; |
| | | | | | | dGs; lnaTs; |
| | | | | | | dAs; lnaTs; |
| | | | | | | dCs; lnaGs; |
| | | | | | | dG- |
| | | | | | | Sup |
| 729 | HOTTIP-2 | ATTGTATCGGGCAAA | HOTTIP | 3′ | human | dAs; lnaTs; |
| | m02 | | | | | dTs; lnaGs; |
| | | | | | | dTs; lnaAs; |
| | | | | | | dTs; lnaCs; |
| | | | | | | dGs; lnaGs; |
| | | | | | | dGs; lnaCs; |
| | | | | | | dAs; lnaAs; |
| | | | | | | dA- |
| | | | | | | Sup |
| 730 | HOTTIP-3 | GATTAAAACAAAAGA | HOTTIP | 3′ | human | dGs; lnaAs; |
| | m02 | | | | | dTs; lnaTs; |
| | | | | | | dAs; lnaAs; |
| | | | | | | dAs; lnaAs; |
| | | | | | | dCs; lnaAs; |
| | | | | | | dAs; lnaAs; |
| | | | | | | dAs; lnaGs; |
| | | | | | | dA- |
| | | | | | | Sup |
| 731 | HOTTIP-4 | AAAACAAAAGAAACC | HOTTIP | 3′ | human | dAs; lnaAs; |
| | m02 | | | | | dAs; lnaAs; |
| | | | | | | dCs; lnaAs; |
| | | | | | | dAs; lnaAs; |
| | | | | | | dAs; lnaGs; |
| | | | | | | dAs; lnaAs; |
| | | | | | | dAs; lnaCs; |
| | | | | | | dC- |
| | | | | | | Sup |
| 732 | HOTTIP-5 | GGGATAAAGGAAGGG | HOTTIP | 5′ | human | dGs; lnaGs; |
| | m02 | | | | | dGs; lnaAs; |
| | | | | | | dTs; lnaAs; |
| | | | | | | dAs; lnaAs; |
| | | | | | | dGs; lnaGs; |
| | | | | | | dAs; lnaAs; |
| | | | | | | dGs; lnaGs; |
| | | | | | | dG- |
| | | | | | | Sup |
| 733 | HOTTIP-6 | CACTGGGATAAAGGA | HOTTIP | 5′ | human | dCs; lnaAs; |
| | m02 | | | | | dCs; lnaTs; |
| | | | | | | dGs; lnaGs; |
| | | | | | | dGs; lnaAs; |
| | | | | | | dTs; lnaAs; |
| | | | | | | dAs; lnaAs; |
| | | | | | | dGs; lnaGs; |
| | | | | | | dA- |
| | | | | | | Sup |
| 734 | HOTTIP-7 | GAGCCGCCCGCTTTG | HOTTIP | 5′ | human | dGs; lnaAs; |
| | m02 | | | | | dGs; lnaCs; |
| | | | | | | dCs; lnaGs; |
| | | | | | | dCs; lnaCs; |
| | | | | | | dCs; lnaGs; |
| | | | | | | dCs; lnaTs; |
| | | | | | | dTs; lnaTs; |
| | | | | | | dG- |
| | | | | | | Sup |
| 735 | HOTTIP-8 | TCTGGGCCCCACTG | HOTTIP | 5′ | human | dTs; lnaCs; |
| | m02 | | | | | dTs; lnaGs; |
| | | | | | | dGs; lnaGs; |
| | | | | | | dCs; lnaCs; |
| | | | | | | dCs; lnaCs; |
| | | | | | | dAs; lnaCs; |
| | | | | | | dTs; lnaG-Sup |
| 736 | NEST-1 | CAAAAGGTCTTAGCT | NEST | 3′ | human | dCs; lnaAs; |
| | m02 | | | | | dAs; lnaAs; |
| | | | | | | dAs; lnaGs; |
| | | | | | | dGs; lnaTs; |
| | | | | | | dCs; lnaTs; |
| | | | | | | dTs; lnaAs; |
| | | | | | | dGs; lnaCs; |
| | | | | | | dT- |
| | | | | | | Sup |
| 737 | NEST-2 | TAGCTATTATTACTG | NEST | 3′ | human | dTs; lnaAs; |
| | m02 | | | | | dGs; lnaCs; |
| | | | | | | dTs; lnaAs; |
| | | | | | | dTs; lnaTs; |
| | | | | | | dAs; lnaTs; |
| | | | | | | dTs; lnaAs; |
| | | | | | | dCs; lnaTs; |
| | | | | | | dG- |
| | | | | | | Sup |
| 738 | NEST-3 | ACTGTTGTTGTTTTA | NEST | 3′ | human | dAs; lnaCs; |
| | m02 | | | | | dTs; lnaGs; |
| | | | | | | dTs; lnaTs; |
| | | | | | | dGs; lnaTs; |
| | | | | | | dTs; lnaGs; |
| | | | | | | dTs; lnaTs; |
| | | | | | | dTs; lnaTs; |
| | | | | | | dA- |
| | | | | | | Sup |
| 739 | NEST-4 | ACCTTAGAGGTTGTA | NEST | 3′ | human | dAs; lnaCs; |
| | m02 | | | | | dCs; lnaTs; |
| | | | | | | dTs; lnaAs; |
| | | | | | | dGs; lnaAs; |
| | | | | | | dGs; lnaGs; |
| | | | | | | dTs; lnaTs; |
| | | | | | | dGs; lnaTs; |
| | | | | | | dA- |
| | | | | | | Sup |
| 740 | NEST-5 | TACCTGAAATTGCAG | NEST | 5′ | human | dTs; lnaAs; |
| | m02 | | | | | dCs; lnaCs; |
| | | | | | | dTs; lnaGs; |
| | | | | | | dAs; lnaAs; |
| | | | | | | dAs; lnaTs; |
| | | | | | | dTs; lnaGs; |
| | | | | | | dCs; lnaAs; |
| | | | | | | dG- |
| | | | | | | Sup |
| 741 | NEST-6 | GTCAGAAAAGCTACC | NEST | 5′ | human | dGs; lnaTs; |
| | m02 | | | | | dCs; lnaAs; |
| | | | | | | dGs; lnaAs; |
| | | | | | | dAs; lnaAs; |
| | | | | | | dAs; lnaGs; |
| | | | | | | dCs; lnaTs; |
| | | | | | | dAs; lnaCs; |
| | | | | | | dC- |
| | | | | | | Sup |
| 742 | NEST-7 | CACGCTTGGTGTGCA | NEST | 5′ | human | dCs; lnaAs; |
| | m02 | | | | | dCs; lnaGs; |
| | | | | | | dCs; lnaTs; |
| | | | | | | dTs; lnaGs; |
| | | | | | | dGs; lnaTs; |
| | | | | | | dGs; lnaTs; |
| | | | | | | dGs; lnaCs; |
| | | | | | | dA- |
| | | | | | | Sup |
| 743 | NEST-8 | CTGTGAATGTGTGAA | NEST | 5′ | human | dCs; lnaTs; |
| | m02 | | | | | dGs; lnaTs; |
| | | | | | | dGs; lnaAs; |
| | | | | | | dAs; lnaTs; |
| | | | | | | dGs; lnaTs; |
| | | | | | | dGs; lnaTs; |
| | | | | | | dGs; lnaAs; |
| | | | | | | dA- |
| | | | | | | Sup |
| 744 | NEST-9 | AACAGGAAGCACCTG | NEST | 5′ | human | dAs; lnaAs; |
| | m02 | | | | | dCs; lnaAs; |
| | | | | | | dGs; lnaGs; |
| | | | | | | dAs; lnaAs; |
| | | | | | | dGs; lnaCs; |
| | | | | | | dAs; lnaCs; |
| | | | | | | dCs; lnaTs; |
| | | | | | | dG- |
| | | | | | | Sup |

### Example 14

### Data from a Friedreich's Ataxia (FRDA) Mouse Model

Indicated 5′ (FXN-375, 380, 385), 3′ (FXN-398) and multi-targeting oligos (FXN-434: 375+398, FXN-436:385+398) were injected subcutaneously to the Sarsero FRDA mouse model. Vehicle (PBS) was injected as control. The sequences of FXN-434 and 436 are shown below in Table 12.

TABLE 12
Sequences for FXN-434 and FXN-436

| SEQ | Oligo | | Gene | Target | | Formatted |
|---|---|---|---|---|---|---|
| ID NO | Name | Base Sequence | Name | Region | Organism | Sequence |
| 745 | FXN-434 | CGCTCCGCCCTCCAGTTT | FXN | 5′ and 3′ | human | dCs; lnaGs; |
| | m02 | TTTTTTAGGAGGCAACA | | | | dCs; lnaTs; |
| | | CATT | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dG; dT; |
| | | | | | | dT; dT; |
| | | | | | | dT; dTs; |
| | | | | | | lnaTs; dTs; |
| | | | | | | lnaTs; dTs; |
| | | | | | | lnaAs; dGs; |
| | | | | | | lnaGs; dAs; |
| | | | | | | lnaGs; dGs; |
| | | | | | | lnaCs; dAs; |
| | | | | | | lnaAs; dCs; |
| | | | | | | lnaAs; dCs; |
| | | | | | | lnaAs; dTs; |
| | | | | | | lnaT- |
| | | | | | | Sup |
| 746 | FXN-436 | CGCTCCGCCCTCCAGCC | FXN | 5′ and 3′ | human | dCs; lnaGs; |
| | m02 | TTTTTTTTTAGGAGGCA | | | | dCs; lnaTs; |
| | | ACACATT | | | | dCs; lnaCs; |
| | | | | | | dGs; lnaCs; |
| | | | | | | dCs; lnaCs; |
| | | | | | | dTs; lnaCs; |
| | | | | | | dCs; lnaAs; |
| | | | | | | dGs; lnaCs; |
| | | | | | | dC; dT; |
| | | | | | | dT; dT; |
| | | | | | | dT; dTs; |
| | | | | | | lnaTs; dTs; |
| | | | | | | lnaTs; dTs; |
| | | | | | | lnaAs; dGs; |
| | | | | | | lnaGs; dAs; |
| | | | | | | lnaGs; dGs; |
| | | | | | | lnaCs; dAs; |
| | | | | | | lnaAs; dCs; |
| | | | | | | lnaAs; dCs; |
| | | | | | | lnaAs; dTs; |
| | | | | | | lnaT-Sup |

For short arm (SA) studies, oligos and control were injected at 25 mg/kg at day0 and day4. Tissues were collected at day7. For long arm (LA) studies, injections were done at the same dose at day0, day4, day7 and collections were done at day14. The human FXN and mouse FXN in the hearts and livers of this model were measured with QPCR and normalized to the PBS group. Each treatment group had 5 mice (n=5).

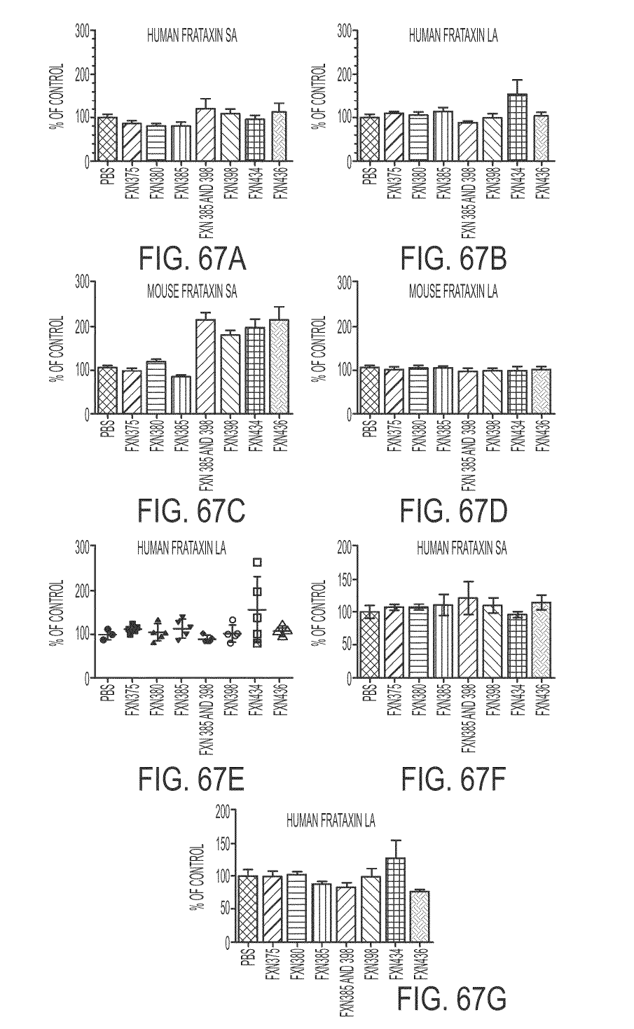

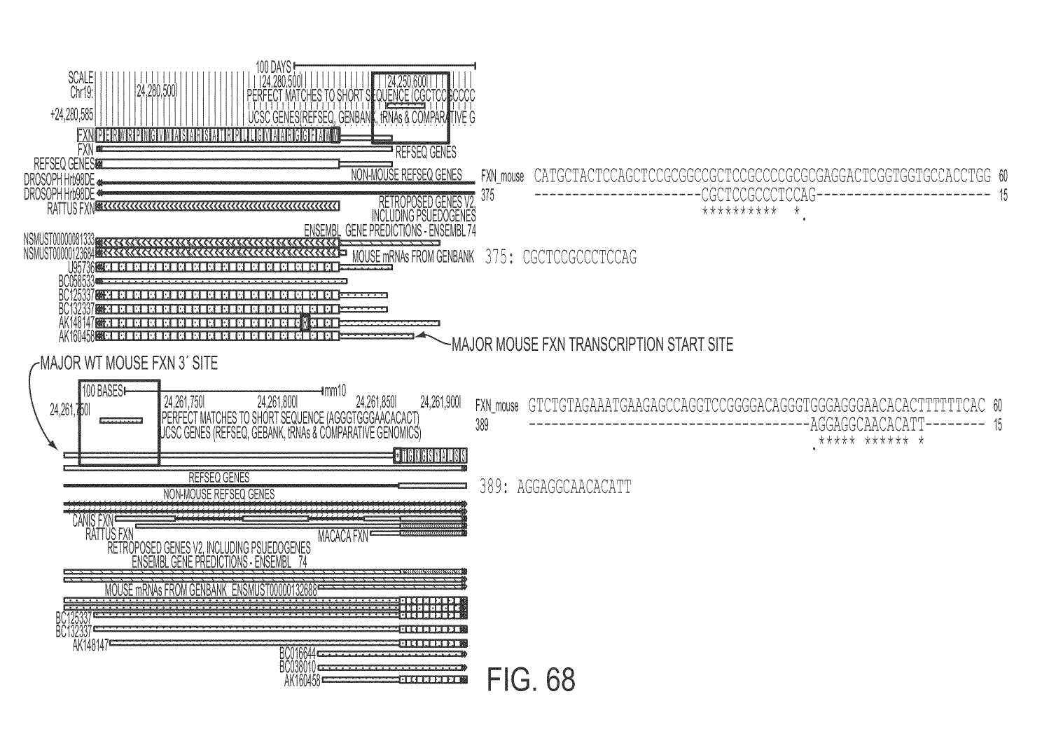

It was found that human FXN-targeting oligos upregulated mouse frataxin mRNA in heart in the short-arm study (FIG. 67). A slight but statistically insignificant upregulation trend was also present for human FXN in the long-arm study in liver and heart (FIG. 67). Two of the oligos, FXN-375 and 389, overlapped with the mouse FXN transcript, with some mismatches (FIG. 68). The major mouse FXN 3′ site was at chr19: 24261501. The major mouse FXN 5′ site is at chr19: 24280595. EST as well as RefSeq annotations suggested the potential binding of these oligos to mouse transcript. These data indicate that oligos containing mismatches to the FXN RNA transcript can still result in upregulation of FXN, showing that mismatches can be tolerated.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1-30. (canceled)

31. A method of increasing gene expression in a cell, the method comprising delivering to a cell an oligonucleotide comprising the general formula 5′-X₁-X₂-3′, wherein X₁ comprises 2 to 20 pyrimidine nucleotides that form base pairs with adenine; and X₂ comprises a region of complementarity that is complementary with at least 3 contiguous nucleotides of a poly-adenylated RNA transcript encoded by the gene, wherein the nucleotide at the 5′-end of the region of complementary of X₂ is complementary with the nucleotide of the RNA transcript that is immediately internal to the poly-adenylation junction of the RNA transcript.

32. The method of claim 31, wherein X₁ comprises 2 to 20 thymidines or uridines.

33. The method of claim 31, wherein the oligonucleotide comprises at least one modified internucleoside linkage.

34. The method of claim 31, wherein the oligonucleotide comprises at least one modified nucleotide.

35. The method of claim 31, wherein at least one nucleotide of the oligonucleotide comprises a 2′ O-methyl.

36. The method of claim 31, wherein the oligonucleotide comprises at least one ribonucleotide, at least one deoxyribonucleotide, at least one 2′-fluoro-deoxyribonucleotides or at least one bridged nucleotide.

37. The method of claim 36, wherein the bridged nucleotide is a LNA nucleotide, a cEt nucleotide or a ENA modified nucleotide.

38. The method of claim 31, wherein each nucleotide of the oligonucleotide is a LNA nucleotide.

39. The oligonucleotide of claim 31, wherein the nucleotides of the oligonucleotide comprise alternating deoxyribonucleotides and 2′-fluoro-deoxyribonucleotides, 2′-O-methyl nucleotides, or bridged nucleotides.

40. The method of claim 31, wherein the oligonucleotide is a mixmer.

41-142. (canceled)

143. The method of claim 31, wherein the RNA transcript is an mRNA, non-coding RNA, long non-coding RNA, miRNA, snoRNA, tRNAs, snRNAs, extracellular RNAs.

144-155. (canceled)
